(19) United States
(12) Patent Application Publication
(10) Pub. No.: US 2025/0262326 A1
(43) Pub. Date: Aug. 21, 2025

(54) EFFECTOR DOMAINS FOR CRISPR-CAS SYSTEMS

(71) Applicant: Duke University, Durham, NC (US)

(72) Inventors: Charles A. Gersbach, Chapel Hill, NC (US); Gabriel Butterfield, Durham, NC (US); Dahlia Rohm, Durham, NC (US); Nahid Iglesias, Durham, NC (US)

(21) Appl. No.: 18/856,563

(22) PCT Filed: Apr. 13, 2023
(86) PCT No.: PCT/US2023/018559

§ 371 Date: Oct. 11, 2024

Related U.S. Application Data
(60) Provisional application No. 63/342,027, filed on May 13, 2022.
(60) Provisional application No. 63/335,122, filed on Apr. 26, 2022.
(60) Provisional application No. 63/330,691, filed on Apr. 13, 2022.

(51) Int. Cl. A61K 48/00; C12N 9/22; C12N 15/11

(52) U.S. Cl. CPC A61K 48/0058; C12N 9/226; C12N 15/11; C12N 2310/20; C12N 2310/3513

(57) ABSTRACT

Disclosed herein are effector domains. The effector domains may be used with, for example, Cas proteins and CRISPR-Cas systems. The effectors may be used in combination with a Cas protein to form a fusion protein. The effectors may also be used in combination with an antibody that binds to a peptide epitope, wherein the peptide epitope is fused to a Cas protein. The compositions and methods comprising the effectors may be used to modulate gene expression.

### CROSS-REFERENCE TO RELATED APPLICATIONS

This application claims priority to U.S. Provisional Patent Application No. 63/330,691 filed Apr. 13, 2022, U.S. Provisional Patent Application No. 63/335,122 filed Apr. 26, 2022, and U.S. Provisional Patent Application No. 63/342,027 filed May 13, 2022, the entire contents of each of which are hereby incorporated by reference.

### STATEMENT REGARDING FEDERALLY SPONSORED RESEARCH

This invention was made with government support under grant 5U01AI146356 awarded by the National Institutes of Health. The government has certain rights in the invention.

### FIELD

This disclosure relates to compositions and methods including CRISPR-Cas systems with effector domains. The effector domains, which may be used, for example, in combination with a Cas protein, may be used to modulate gene expression.

### INTRODUCTION

Synthetic transcription factors have been engineered to control gene expression for many different medical and scientific applications in mammalian systems, including stimulating tissue regeneration, drug screening, compensating for genetic defects, activating silenced tumor suppressors, controlling stem cell differentiation, performing genetic screens, and creating synthetic gene circuits. These transcription factors can target promoters or enhancers of endogenous genes or be purposefully designed to recognize sequences orthogonal to mammalian genomes for transgene regulation.

Further, these synthetic transcription factors rely on naturally occurring or designed effector protein domains which modulate gene expression. However, the full spectrum of regulatory mechanisms employed in mammalian cells cannot be programmed with currently described effector domains. Broadening the set of available effectors will enable both more potent and more specific gene activation and repression.

### SUMMARY

In an aspect, the disclosure relates to a Cas effector. The Cas effector may include a first polypeptide comprising a Cas protein and at least one peptide epitope; and a second polypeptide comprising an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, and an antibody to the peptide epitope. In some embodiments, the effector is selected from MCRS1, OTUD7B, RelB, LDB1, NFKBIB, CITED2, PHF15, SS18L1, MLLT6, ASH2L, and GSK3A, or a combination thereof. In some embodiments, the effector is capable of increasing or decreasing expression of a gene. In some embodiments, the effector reduces expression of a target gene and is selected from MCRS1, OTUD7B, ZNF133, ZNF140, ZNF169, ZNF254, ZNF566, ZNF585A, ZNF689, ZNF765, and ZNF81, or a combination thereof. In some embodiments, the effector increases expression of a target gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof. In some embodiments, the first polypeptide comprises about 2 to about 50 peptide epitopes. In some embodiments, the first polypeptide comprises more than one copy of the peptide epitope and further comprises at least one linker in between adjacent copies of the peptide epitope. In some embodiments, the peptide epitope is GCN4 and comprises the amino acid sequence of SEQ ID NO: 85. In some embodiments, the first polypeptide comprises at least one peptide epitope at the N-terminus and/or at the C-terminus of the Cas protein. In some embodiments, the first polypeptide comprises an amino acid sequence having at least 80%, 85%, 90%, 95%, or 98% or greater identity to SEQ ID NO: 87 or 89, or any fragment thereof, or the first polypeptide comprises an amino acid sequence having one, two, three, four, five or more changes selected from amino acid substitutions, insertions, or deletions, relative to SEQ ID NO: 87 or 89, or any fragment thereof, or the first polypeptide comprises the amino acid sequence of SEQ ID NO: 87 or 89. In some embodiments, the antibody comprises the amino acid sequence of SEQ ID NO: 81. In some embodiments, the second polypeptide comprises an amino acid sequence having at least 80%, 85%, 90%, 95%, or 98% or greater identity to a sequence selected from SEQ ID NOs: 69, 71, 73, 75, 77, and 79, or any fragment thereof, or the second polypeptide comprises an amino acid sequence having one, two, three, four, five or more changes selected from amino acid substitutions, insertions, or deletions, relative to a sequence selected from SEQ ID NOs: 69, 71, 73, 75, 77, and 79, or any fragment thereof, or the second polypeptide comprises an amino acid sequence selected from SEQ ID NOs: 69, 71, 73, 75, 77, and 79.

In a further aspect, the disclosure relates to a Cas fusion protein. The Cas fusion protein may include two heterologous polypeptide domains, wherein the first polypeptide domain comprises a Cas protein, and wherein the second polypeptide domain comprises an effector selected from MCRS1, OTUD7B, RelB, LDB1, NFKBIB, and CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof. In some embodiments, the effector is selected from MCRS1, OTUD7B, RelB, LDB1, NFKBIB, CITED2, PHF15, SS18L1, MLLT6, ASH2L, and GSK3A, or a combination thereof. In some embodiments, the effector is capable of increasing or decreasing expression of a gene. In some embodiments, the effector reduces expression of a target gene and is selected from MCRS1, OTUD7B, ZNF133, ZNF140, ZNF169, ZNF254, ZNF566, ZNF585A, ZNF689, ZNF765, and ZNF81, or a combination thereof. In some embodiments, the effector increases expression of a target gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof. In some embodiments, the second polypeptide domain has transcription repression activity, transcription activation activity, de-ubiquitinase activity, p300 recruitment activity, enhancer looping mediation activity, or a combination thereof.

In some embodiments, the MCRS1 comprises an amino acid sequence having at least 80%, 85%, 90%, 95%, or 98% or greater identity to SEQ ID NO: 57 or any fragment thereof, and/or the MCRS1 comprises an amino acid sequence having one, two, three, four, five or more changes selected from amino acid substitutions, insertions, or deletions, relative to SEQ ID NO: 57, or any fragment thereof, and/or the MCRS1 comprises the amino acid sequence of SEQ ID NO: 57, and/or the MCRS1 is encoded by a polynucleotide comprising a sequence having at least 80%, 85%, 90%, 95%, or 98% or greater identity to SEQ ID NO: 58, or any fragment thereof, and/or the MCRS1 is encoded by a polynucleotide comprising a sequence having one, two, three, four, five or more changes selected from nucleotide substitutions, insertions, or deletions, relative to SEQ ID NO: 58, or any fragment thereof, and/or the MCRS1 is encoded by a polynucleotide comprising the sequence of SEQ ID NO: 58. In some embodiments, the OTUD7B comprises an amino acid sequence having at least 80%, 85%, 90%, 95%, or 98% or greater identity to a sequence selected from SEQ ID NO: 59, amino acids 167-440 of SEQ ID NO: 59, or amino acids 792-831 of SEQ ID NO: 59, or any fragment thereof, and/or the OTUD7B comprises an amino acid sequence having one, two, three, four, five or more changes selected from amino acid substitutions, insertions, or deletions, relative to a sequence selected from SEQ ID NO: 59, amino acids 167-440 of SEQ ID NO: 59, or amino acids 792-831 of SEQ ID NO: 59, or any fragment thereof, and/or the OTUD7B comprises the amino acid sequence selected from SEQ ID NO: 59, amino acids 167-440 of SEQ ID NO: 59, or amino acids 792-831 of SEQ ID NO: 59, and/or the OTUD7B is encoded by a polynucleotide comprising a sequence having at least 80%, 85%, 90%, 95%, or 98% or greater identity to SEQ ID NO: 60, or any fragment thereof, and/or the OTUD7B is encoded by a polynucleotide comprising a sequence having one, two, three, four, five or more changes selected from nucleotide substitutions, insertions, or deletions, relative to SEQ ID NO: 60, or any fragment thereof, and/or the OTUD7B is encoded by a polynucleotide comprising the sequence of SEQ ID NO: 60. In some embodiments, the RelB comprises an amino acid sequence having at least 80%, 85%, 90%, 95%, or 98% or greater identity to SEQ ID NO: 65, or any fragment thereof, and/or the RelB comprises an amino acid sequence having one, two, three, four, five or more changes selected from amino acid substitutions, insertions, or deletions, relative to SEQ ID NO: 65, or any fragment thereof, and/or the RelB comprises the amino acid sequence of SEQ ID NO: 65, and/or the RelB is encoded by a polynucleotide comprising a sequence having at least 80%, 85%, 90%, 95%, or 98% or greater identity to SEQ ID NO: 66 or any fragment thereof, and/or the RelB is encoded by a polynucleotide comprising a sequence having one, two, three, four, five or more changes selected from nucleotide substitutions, insertions, or deletions, relative to SEQ ID NO: 66, or any fragment thereof, and/or the RelB is encoded by a polynucleotide comprising the sequence of SEQ ID NO: 66. In some embodiments, the LDB1 comprises an amino acid sequence having at least 80%, 85%, 90%, 95%, or 98% or greater identity to SEQ ID NO: 61, or any fragment thereof, and/or the LDB1 comprises an amino acid sequence having one, two, three, four, five or more changes selected from amino acid substitutions, insertions, or deletions, relative to SEQ ID NO: 61, or any fragment thereof, and/or the LDB1 comprises the amino acid sequence of SEQ ID NO: 61, and/or the LDB1 is encoded by a polynucleotide comprising a sequence having at least 80%, 85%, 90%, 95%, or 98% or greater identity to SEQ ID NO: 62, or any fragment thereof, and/or the LDB1 is encoded by a polynucleotide comprising a sequence having one, two, three, four, five or more changes selected from nucleotide substitutions, insertions, or deletions, relative to SEQ ID NO: 62, or any fragment thereof, and/or the LDB1 is encoded by a polynucleotide comprising the sequence of SEQ ID NO: 62. In some embodiments, the NFKBIB comprises an amino acid sequence having at least 80%, 85%, 90%, 95%, or 98% or greater identity to SEQ ID NO: 63, or any fragment thereof, and/or the NFKBIB comprises an amino acid sequence having one, two, three, four, five or more changes selected from amino acid substitutions, insertions, or deletions, relative to SEQ ID NO: 63, or any fragment thereof, and/or the NFKBIB comprises the amino acid sequence of SEQ ID NO: 63, and/or the NFKBIB is encoded by a polynucleotide comprising a sequence having at least 80%, 85%, 90%, 95%, or 98% or greater identity to SEQ ID NO: 64, or any fragment thereof, and/or the NFKBIB is encoded by a polynucleotide comprising a sequence having one, two, three, four, five or more changes selected from nucleotide substitutions, insertions, or deletions, relative to SEQ ID NO: 64, or any fragment thereof, and/or the NFKBIB is encoded by a polynucleotide comprising the sequence of SEQ ID NO: 64. In some embodiments, the CITED2 comprises an amino acid sequence having at least 80%, 85%, 90%, 95%, or 98% or greater identity to SEQ ID NO: 67, or any fragment thereof, and/or the CITED2 comprises an amino acid sequence having one, two, three, four, five or more changes selected from amino acid substitutions, insertions, or deletions, relative to SEQ ID NO: 67, or any fragment thereof, and/or the CITED2 comprises the amino acid sequence of SEQ ID NO: 67, and/or the CITED2 is encoded by a polynucleotide comprising a sequence having at least 80%, 85%, 90%, 95%, or 98% or greater identity to SEQ ID NO: 68, or any fragment thereof, and/or the CITED2 is encoded by a polynucleotide comprising a sequence having one, two, three, four, five or more changes selected from nucleotide substitutions, insertions, or deletions, relative to SEQ ID NO: 68, or any fragment thereof, and/or the CITED2 is encoded by a polynucleotide comprising the sequence of SEQ ID NO: 68. In some embodiments, the PHF15 comprises an amino acid sequence having at least 80%, 85%, 90%, 95%, or 98% or greater identity to SEQ ID NO: 133, or any fragment thereof, and/or the PHF15 comprises an amino acid sequence having one, two, three, four, five or more changes selected from amino acid substitutions, insertions, or deletions, relative to SEQ ID NO: 133, or any fragment thereof, and/or the PHF15 comprises the amino acid sequence of SEQ ID NO: 133, and/or the PHF15 is encoded by a polynucleotide comprising a sequence having at least 80%, 85%, 90%, 95%, or 98% or greater identity to SEQ ID NO: 134, or any fragment thereof, and/or the PHF15 is encoded by a polynucleotide comprising a sequence having one, two, three, four, five or more changes selected from nucleotide substitutions, insertions, or deletions, relative to SEQ ID NO: 134, or any fragment thereof, and/or the PHF15 is encoded by a polynucleotide comprising the sequence of SEQ ID NO: 134. In some embodiments, the SS18L1 comprises an amino acid sequence having at least 80%, 85%, 90%, 95%, or 98% or greater identity to SEQ ID NO: 149, or any fragment thereof, and/or the SS18L1 comprises an amino acid sequence having one, two, three, four, five or more changes selected from amino acid substitutions, insertions, or deletions, relative to SEQ ID NO: 149, or any fragment thereof, and/or the SS18L1 comprises the amino acid sequence of SEQ ID NO: 149, and/or the SS18L1 is encoded by a polynucleotide comprising a sequence having at least 80%, 85%, 90%, 95%, or 98% or greater identity to SEQ ID NO: 150, or any fragment thereof, and/or the SS18L1 is encoded by a polynucleotide comprising a sequence having one, two, three, four, five or more changes selected from nucleotide substitutions, insertions, or deletions, relative to SEQ ID NO: 150, or any fragment thereof, and/or the SS18L1 is encoded by a polynucleotide comprising the sequence of SEQ ID NO: 150. In some embodiments, the MLLT6 comprises an amino acid sequence having at least 80%, 85%, 90%, 95%, or 98% or greater identity to SEQ ID NO: 127, or any fragment thereof, and/or the MLLT6 comprises an amino acid sequence having one, two, three, four, five or more changes selected from amino acid substitutions, insertions, or deletions, relative to SEQ ID NO: 127, or any fragment thereof, and/or the MLLT6 comprises the amino acid sequence of SEQ ID NO: 127, and/or the MLLT6 is encoded by a polynucleotide comprising a sequence having at least 80%, 85%, 90%, 95%, or 98% or greater identity to SEQ ID NO: 128, or any fragment thereof, and/or the MLLT6 is encoded by a polynucleotide comprising a sequence having one, two, three, four, five or more changes selected from nucleotide substitutions, insertions, or deletions, relative to SEQ ID NO: 128, or any fragment thereof, and/or the MLLT6 is encoded by a polynucleotide comprising the sequence of SEQ ID NO: 128. In some embodiments, the ASH2L comprises an amino acid sequence having at least 80%, 85%, 90%, 95%, or 98% or greater identity to SEQ ID NO: 103, or any fragment thereof, and/or the ASH2L comprises an amino acid sequence having one, two, three, four, five or more changes selected from amino acid substitutions, insertions, or deletions, relative to SEQ ID NO: 103, or any fragment thereof, and/or the ASH2L comprises the amino acid sequence of SEQ ID NO: 103, and/or the ASH2L is encoded by a polynucleotide comprising a sequence having at least 80%, 85%, 90%, 95%, or 98% or greater identity to SEQ ID NO: 104, or any fragment thereof, and/or the ASH2L is encoded by a polynucleotide comprising a sequence having one, two, three, four, five or more changes selected from nucleotide substitutions, insertions, or deletions, relative to SEQ ID NO: 104, or any fragment thereof, and/or the ASH2L is encoded by a polynucleotide comprising the sequence of SEQ ID NO: 104. In some embodiments, the GSK3A comprises an amino acid sequence having at least 80%, 85%, 90%, 95%, or 98% or greater identity to SEQ ID NO: 117, or any fragment thereof, and/or the GSK3A comprises an amino acid sequence having one, two, three, four, five or more changes selected from amino acid substitutions, insertions, or deletions, relative to SEQ ID NO: 117, or any fragment thereof, and/or the GSK3A comprises the amino acid sequence of SEQ ID NO: 117, and/or the GSK3A is encoded by a polynucleotide comprising a sequence having at least 80%, 85%, 90%, 95%, or 98% or greater identity to SEQ ID NO: 118, or any fragment thereof, and/or the GSK3A is encoded by a polynucleotide comprising a sequence having one, two, three, four, five or more changes selected from nucleotide substitutions, insertions, or deletions, relative to SEQ ID NO: 118, or any fragment thereof, and/or the GSK3A is encoded by a polynucleotide comprising the sequence of SEQ ID NO: 118. In some embodiments, the effector is selected from BCL7B, C20orf20, DMAP1, DYRK1B, EAF1, FOXR2, JAZF1, KAT7, KEAP1, MEAF6, MORF4L2, NFYC, PKIB, POLE4, PRKRIR, PYGO2, RANBP1, RPRD1B, SPIN1, TADA3, TAF6, TBPL1, VPS72, ZNF133, ZNF140, ZNF169, ZNF254, ZNF566, ZNF585A, ZNF689, ZNF765, and ZNF81, and wherein the effector comprises an amino acid sequence having at least 80%, 85%, 90%, 95%, or 98% or greater identity to a sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175, or any fragment thereof, and/or wherein the effector comprises an amino acid sequence having one, two, three, four, five or more changes selected from amino acid substitutions, insertions, or deletions, relative to a sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175, or any fragment thereof, and/or wherein the effector comprises an amino acid sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175, and/or wherein the effector is encoded by a polynucleotide comprising a sequence having at least 80%, 85%, 90%, 95%, or 98% or greater identity to a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176, or any fragment thereof, and/or wherein the effector is encoded by a polynucleotide comprising a sequence having one, two, three, four, five or more changes selected from nucleotide substitutions, insertions, or deletions, relative to a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176, or any fragment thereof, and/or wherein the effector is encoded by a polynucleotide comprising a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176. In some embodiments, the Cas protein comprises at least one amino acid mutation that knocks out nuclease activity of the Cas protein. In some embodiments, the at least one amino acid mutation is at least one of D10A and H840A. In some embodiments, the Cas protein comprises an amino acid sequence having at least 80%, 85%, 90%, 95%, or 98% or greater identity to one of SEQ ID NOs: 26-29, or any fragment thereof, or the Cas protein comprises an amino acid sequence having one, two, three, four, five or more changes selected from amino acid substitutions, insertions, or deletions, relative to one of SEQ ID NOs: 26-29, or any fragment thereof, or the Cas protein comprises the amino acid sequence of one of SEQ ID NOs: 26-29. In some embodiments, the Cas protein is encoded by a polynucleotide comprising a sequence having at least 80%, 85%, 90%, 95%, or 98% or greater identity to one of SEQ ID NOs: 30-31, or any fragment thereof, or the Cas protein is encoded by a polynucleotide comprising a sequence having one, two, three, four, five or more changes selected from nucleotide substitutions, insertions, or deletions, relative to one of SEQ ID NOs: 30-31, or any fragment thereof, or the Cas protein is encoded by a polynucleotide comprising the sequence of one of SEQ ID NOs: 30-31.

Another aspect of the disclosure provides a DNA targeting composition. The DNA targeting composition may include a Cas effector as detailed herein or a Cas fusion protein as detailed herein; and at least one guide RNA (gRNA) that targets the Cas protein to a target region of a target gene. In some embodiments, the gRNA targets the Cas protein to target region selected from a non-open chromatin region, an open chromatin region, a transcribed region of the target gene, a region upstream of a transcription start site of the target gene, a regulatory element of the target gene, an intron of the target gene, or an exon of the target gene. In some embodiments, the gRNA targets the Cas protein to a promoter of the target gene. In some embodiments, the target region is located between about 1 to about 1000 base pairs upstream of a transcription start site of the target gene. In some embodiments, the at least one gRNA comprises a sequence selected from SEQ ID NOs: 96-98 and 101-102, or the at least one gRNA is encoded by a polynucleotide comprising a sequence selected from SEQ ID NOs: 93-95 and 99-100, or the at least one gRNA targets and binds a polynucleotide comprising a sequence selected from SEQ ID NOs: 93-95 and 99-100 or a complement thereof, or a combination thereof. In some embodiments, the DNA targeting composition comprises two or more gRNAs, each gRNA binding to a different target region.

Another aspect of the disclosure provides an isolated polynucleotide sequence encoding a Cas effector as detailed herein or a Cas fusion protein as detailed herein, or a DNA targeting composition as detailed herein.

Another aspect of the disclosure provides a vector comprising an isolated polynucleotide sequence as detailed herein. In some embodiments, the vector is an adeno-associated virus (AAV) vector.

Another aspect of the disclosure provides a cell comprising a Cas effector as detailed herein or a Cas fusion protein as detailed herein, or a DNA targeting composition as detailed herein, or an isolated polynucleotide sequence as detailed herein, or a vector as detailed herein, or a combination thereof.

Another aspect of the disclosure provides a pharmaceutical composition. The pharmaceutical composition may include a Cas effector as detailed herein or a Cas fusion protein as detailed herein, or a DNA targeting composition as detailed herein, or an isolated polynucleotide sequence as detailed herein, or a vector as detailed herein, or a combination thereof.

Another aspect of the disclosure provides a method of modulating expression of a gene in a cell or in a subject. The method may include administering to the cell or the subject a DNA targeting composition as detailed herein, or an isolated polynucleotide sequence as detailed herein, or a vector as detailed herein, or a pharmaceutical composition as detailed herein, or a combination thereof. The method may include administering to the cell or the subject an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, or a polynucleotide encoding the effector. In some embodiments, the effector is targeted to the gene. In some embodiments, the effector is selected from MCRS1, OTUD7B, RelB, LDB1, NFKBIB, CITED2, PHF15, SS18L1, MLLT6, ASH2L, and GSK3A, or a combination thereof. In some embodiments, the effector is capable of increasing or decreasing expression of the gene. In some embodiments, the effector reduces expression of the gene and is selected from MCRS1, OTUD7B, ZNF133, ZNF140, ZNF169, ZNF254, ZNF566, ZNF585A, ZNF689, ZNF765, and ZNF81, or a combination thereof. In some embodiments, the effector increases expression of the gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof. In some embodiments, the expression of the gene is increased relative to a control. In some embodiments, the expression of the gene is decreased relative to a control. In some embodiments, the gene comprises the dystrophin gene, the CD25 gene, the B2M gene, or the TRAC gene. In some embodiments, the cell is a muscle cell or a T cell.

Another aspect of the disclosure provides a method of treating a disease in a subject. The method may include administering to the subject a DNA targeting composition as detailed herein, or an isolated polynucleotide sequence as detailed herein, or a vector as detailed herein, or a cell as detailed herein, or a pharmaceutical composition as detailed herein, or a combination thereof. The method may include administering to the subject an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, or a polynucleotide encoding the effector. In some embodiments, the effector is targeted to a gene. In some embodiments, the method treats a disease selected from Duchenne muscular dystrophy (DMD), Becker muscular dystrophy (BMD), and cancer.

The disclosure provides for other aspects and embodiments that will be apparent in light of the following detailed description and accompanying figures.

### BRIEF DESCRIPTION OF THE DRAWINGS

FIG. 1 is a graph showing the results from the individual testing of top repressor effectors from B2M screen. The graph displays the percent of cells in the low B2M bin, with higher numbers suggesting more potent repression. A non-targeting guide was also included as a control for non-specific repression. MCRS1 and OTUD7B both showed repression that was both greater than the steric effects of dCas9 alone and largely dependent on dCas9 targeting, rather than a non-specific effect.

Shown in FIG. 2A is the level of CD25 activation after delivery of each effector domain recruited by dCas9 in Jurkat cells. A non-targeting guide (gray bars) showed no effect on CD25, suggesting that each effector was specifically activating CD25 upon recruitment by dCas9. Shown in FIG. 2B is a zoomed-in view of data in FIG. 2A to show the specific activation by LDB1 and NFKBIB.

FIGS. 3A-3B are graphs showing the results for each effector in a screen for the ability to modulate expression of TetO with a GFP reporter. Log 2(fold change) and Log 10(Adjusted P Value) for each effector in the screen are plotted. Shown in FIG. 3A are results with a gRNA targeting TetO, and shown in FIG. 3B are results with a non-targeting gRNA. Effectors with Log 2(fold change)>1.1 and Adjusted P Value <0.01 were considered to be hits and are shown in filled black circles, while non-hits are shown in open gray circles. This threshold gave 41 hits in the targeting condition and only 1 hit in the non-targeting condition.

FIG. 4 shows GFP reporter expression in the TetO-GFP reporter screen in 293T cells for a subset of effectors, including PHF15, SS18L1, MLLT6, ASH2L, and GSK3A. 293T cells containing a GFP reporter were transduced with Lentivirus encoding a subset of effectors found to be hits in the high-throughput screen along with a targeting (black) or non-targeting (gray) sgRNA. The fold activation of GFP (shown above each pair of bars) was found to be greater than 1 for all effectors tested, while the dCas9 alone control showed the opposite trend, supporting the idea that even the small effects seen for some effectors are likely meaningful. All hit effectors tested did modulate GFP to some degree.

FIG. 5 is a graph showing activation of TetO with a GFP reporter in 293T cells by CITED2 and LDB1. 293T cells previously transduced with a TetO-GFP reporter were transfected with the indicated effector. Both LDB1 and CITED2 robustly activated GFP expression, demonstrating that activation by these effectors is not limited to CD25.

FIG. 6 is a graph showing activation of CD25 expression with either wild-type LDB1 or LDB1 with a deletion in its dimerization domain. Jurkat cells expressing dCas9-GCN4 and a CD25-targeting gRNA or non-targeting gRNA were transduced with the indicated effector-scFv fusion, and CD25 expression was analyzed by flow cytometry 10 days later. Only the intact LDB1 effector was able to activate CD25 expression.

### DETAILED DESCRIPTION

Disclosed herein is a set of novel effectors that may activate or repress gene expression when recruited to the gene, for example, via a Cas protein such as dCas9. As detailed herein, the human genome was screened for potential proteins that impact gene expression. The proteins may be referred to as effectors or effector domains. Several novel effectors were discovered, including MCRS1, OTUD7B, RelB, LDB1, NFKBIB, CITED2, ASH2L, GSK3A, MLLT6, PHF15, SS18L1, BCL7B, C20orf20, DMAP1, DYRK1B, EAF1, FOXR2, JAZF1, KAT7, KEAP1, MEAF6, MORF4L2, NFYC, PKIB, POLE4, PRKRIR, PYGO2, RANBP1, RPRD1B, SPIN1, TADA3, TAF6, TBPL1, VPS72, ZNF133, ZNF140, ZNF169, ZNF254, ZNF566, ZNF585A, ZNF689, ZNF765, and ZNF81. These effectors may be used in combination with a Cas protein, for example, to target a region of a gene or other DNA sequence. The effector and a Cas protein may form a fusion protein. In other embodiments, the effector is used in combination with an antibody, a peptide epitope is fused to a Cas protein, and binding of the antibody to the peptide epitope brings the effector proximal to the Cas protein. The effector and Cas protein may be used to modulate expression of a gene. The effector and Cas protein may also be used to treat various diseases.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Allogeneic” refers to any material derived from another subject of the same species. Allogeneic cells are genetically distinct and immunologically incompatible yet belong to the same species. Typically, “allogeneic” is used to define cells, such as stem cells, that are transplanted from a donor to a recipient of the same species.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Autologous” refers to any material derived from a subject and re-introduced to the same subject.

“Binding region” as used herein refers to the region within a target region that is recognized and bound by the CRISPR/Cas-based gene editing system.

The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Cancer” refers to all types of cancer or neoplasm or malignant tumors found in animals, including carcinoma, adenoma, melanoma, sarcoma, lymphoma, leukemia, blastoma, glioma, astrocytoma, mesothelioma, or a germ cell tumor. Cancer may include cancer of, for example, the colon, rectum, stomach, bladder, cervix, uterus, skin, epithelium, muscle, kidney, liver, lymph, bone, blood, ovary, prostate, lung, brain, head and neck, and/or breast. Cancer may include medullablastoma, non-small cell lung cancer, and/or mesothelioma. In embodiments detailed herein, the cancer includes leukemia. The term “leukemia” refers to broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid leukemia, myeloid granulocytic leukemia, myelomonocytic leukemia, Naegeli leukemia, plasma cell leukemia, plasmacytic leukemia, and promyelocytic leukemia. In some embodiments, the leukemia is chronic myeloid leukemia (CML). In some embodiments, the leukemia is acute myeloid leukemia (AML).

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as a percentage as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated.

“Target region” as used herein refers to the region of the target gene to which the CRISPR/Cas9-based gene editing or targeting system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto. A variant can be a polynucleotide sequence that is substantially identical over the full length of the full polynucleotide sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the polynucleotide sequence or a fragment thereof.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the amino acid sequence or a fragment thereof.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode, for example, a Cas9 protein or fusion protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. DNA TARGETING SYSTEMS

Provided herein are DNA Targeting Systems that may be used, for example, to modulate gene expression. A “DNA Targeting System” as used herein is a system capable of specifically targeting a particular region of DNA and modulating gene expression by binding to that region. Non-limiting examples of these systems are CRISPR-Cas-based systems, zinc finger (ZF)-based systems, and/or transcription activator-like effector (TALE)-based systems. The DNA Targeting System may be a nuclease system that acts through mutating or editing the target region (such as by insertion, deletion or substitution) or it may be a system that delivers a functional second polypeptide domain, such as an activator or repressor, to the target region.

Each of these systems comprises a DNA-binding portion or domain, such as a guide RNA, a ZF, or a TALE, that specifically recognizes and binds to a particular target region of a target DNA. The DNA-binding portion (for example, Cas protein, ZF, or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity, to form a fusion protein. In other embodiments, the DNA-binding portion is linked with a second protein domain using an antibody and peptide epitope, such as the Suntag recruitment system (Tanenbaum et al., Cell 2014, 159, 635-646, incorporated herein by reference in its entirety). Exemplary second polypeptide domains are detailed further below (see “Cas Fusion Protein”). For example, the DNA-binding portion can be linked to an activator and thus guide the activator to a specific target region of the target DNA. Similarly, the DNA-binding portion can be linked to a repressor and thus guide the repressor to a specific target region of the target DNA.

In some embodiments, the DNA-binding portion comprises a Cas protein, such as a Cas9 protein. Some CRISPR-Cas-based systems can operate to activate or repress expression using the Cas protein alone, not linked to an activator or repressor. For example, a nuclease-null Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor. Some CRISPR-Cas-based systems can operate to activate or repress expression using the Cas protein linked to a second protein domain, such as, for example, an activator or repressor. Some embodiments include a Cas protein linked to a second polypeptide domain such as an effector (see “Cas Fusion Protein”).

In other embodiments, a first polypeptide comprising a DNA-binding portion further comprises at least one peptide epitope, and a second polypeptide comprises an activator or repressor and an antibody to the peptide epitope. For example, some embodiments include a first polypeptide comprising a Cas protein and at least one peptide epitope, and a second polypeptide comprising the effector domain and an antibody to the peptide epitope (see “Cas Effector”).

### 3. CRISPR/CAS-BASED GENE EDITING SYSTEM

Provided herein are CRISPR/Cas9-based gene editing systems. The CRISPR/Cas-based gene editing system may be used to modulate expression of a gene and/or treat a disease. The CRISPR/Cas-based gene editing system may include a Cas protein or a fusion protein, and at least one gRNA, and may also be referred to as a “CRISPR-Cas system.” Other embodiments include a first polypeptide comprising a Cas protein and at least one peptide epitope, at least one gRNA, and a second polypeptide comprising the effector domain and an antibody to the peptide epitope.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas12a, Cas9, and Cascade proteins. Cas12a may also be referred to as “Cpf1.” Cas12a causes a staggered cut in double stranded DNA, while Cas9 produces a blunt cut. In some embodiments, the Cas protein comprises Cas12a. In some embodiments, the Cas protein comprises Cas9. Cas9 forms a complex with the 3′ end of the sgRNA (which may be referred interchangeably herein as “gRNA”), and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex. Cas12a systems include crRNA for successful targeting, whereas Cas9 systems include both crRNA and tracrRNA.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

In some embodiments, the Cas protein and/or the Cas fusion protein and/or Cas effector and/or gRNAs and/or Effector domains detailed herein may be used in compositions and methods for modulating expression of a gene. The Cas protein and/or the Cas fusion protein and/or Cas effector and/or Effector domains detailed herein may be targeted to the gene. The Cas protein and/or the Cas fusion protein and/or Cas effector and/or Effector domains detailed herein may be targeted to a regulatory element of the gene. Modulating may include, for example, increasing or enhancing expression of the gene, or reducing or inhibiting expression of the gene. The expression of the gene may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by about 5-95%, 10-90%, 15-85%, 20-80%, or 1.5-fold to 10-fold, relative to a control. The expression of the gene may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by about 5-95%, 10-90%, 15-85%, 20-80%, or 1.5-fold to 10-fold, relative to a control. The expression of the gene may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control.

a. Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 26. In certain embodiments, the Cas9 molecule is a Staphylococcus aureus Cas9 molecule (also referred herein as “SaCas9”). SaCas9 may comprise an amino acid sequence of SEQ ID NO: 27.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G; V=A or C or G) (SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 20).

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 28. A S. pyogenes Cas9 protein with D10A and H840A mutations may comprise an amino acid sequence of SEQ ID NO: 29. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 30. In certain embodiments, the mutant S. aureus Cas9 molecule comprises a N580A mutation. The nucleotide sequence encoding this mutant S. aureus Cas9 molecule is set forth in SEQ ID NO: 31.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 32. Exemplary codon optimized nucleic acid sequences encoding a Cas9 molecule of S. aureus, and optionally containing nuclear localization sequences (NLSs), are set forth in SEQ ID NOs: 33-39. Another exemplary codon optimized nucleic acid sequence encoding a Cas9 molecule of S. aureus comprises the nucleotides 1293-4451 of SEQ ID NO: 40.

b. Cas Fusion Protein

Alternatively or additionally, the CRISPR/Cas-based gene editing system can include a fusion protein. The fusion protein can comprise two heterologous polypeptide domains. The first polypeptide domain comprises a Cas protein or a mutated Cas protein. The first polypeptide domain is fused to at least one second polypeptide domain. The second polypeptide domain may comprise or also be referred to as an effector, or effector domain. The second polypeptide domain has a different activity that what is endogenous to Cas protein. For example, the second polypeptide domain may have an activity such as transcription activation activity, transcription repression activity, transcription release factor activity, histone modification activity, nuclease activity, nucleic acid association activity, histone methylase activity, DNA methylase activity, histone demethylase activity, DNA demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. In some embodiments, the fusion protein comprises more than one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)n, wherein n is an integer between 0 and 10 (SEQ ID NO: 21). In a GS linker, n can be adjusted to optimize the linker length and achieve appropriate separation of the functional domains. Other examples of linkers may include, for example, Gly-Gly-Gly-Gly-Gly (SEQ ID NO: 22), Gly-Gly-Ala-Gly-Gly (SEQ ID NO: 23), Gly/Ser rich linkers such as Gly-Gly-Gly-Gly-Ser-Ser-Ser (SEQ ID NO: 24) or GSGSG (SEQ ID NO: 91) or GSGSGGSGSGSGGSGSGGSGSG (SEQ ID NO: 92), or Gly/Ala rich linkers such as Gly-Gly-Gly-Gly-Ala-Ala-Ala (SEQ ID NO: 25).

c. Cas Effector

Alternatively or additionally, the CRISPR/Cas-based gene editing system can include a Cas effector. The Cas effector can include a first polypeptide comprising a Cas protein and at least one peptide epitope, and a second polypeptide comprising an effector and an antibody to the peptide epitope. Such systems are described in, for example, in Tanenbaum et al. (Cell 2014, 159, 635-646, incorporated herein by reference in its entirety) with reference to, for example, the Suntag recruitment system. For the Cas effector, the first polypeptide and the second polypeptide may be two separate polypeptides or chains.

The first polypeptide of the Cas effector may comprise about 2 to about 50 peptide epitopes, about 2 to about 40 peptide epitopes, about 2 to about 30 peptide epitopes, or about 3 to about 25 peptide epitopes. The first polypeptide may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 peptide epitopes. In some embodiments, the first polypeptide comprises about 5 peptide epitopes. In some embodiments, the first polypeptide comprises about 24 peptide epitopes. The first polypeptide may comprise at least one peptide epitope at the N-terminus and/or at the C-terminus of the Cas protein.

The peptide epitope may comprise any amino acid sequence that the antibody binds. The antibody may bind specifically to the peptide epitope. The peptide epitope may comprise an amino acid sequence that is not found in humans. In some embodiments, the peptide epitope comprises GCN4. GCN4 may comprise a peptide having an amino acid sequence of SEQ ID NO: 85 and may be encoded by a polynucleotide comprising SEQ ID NO: 86. The first polypeptide may comprise at least one linker N-terminal or C-terminal to the peptide epitope. The first polypeptide may comprise more than one copy of the peptide epitope and at least one linker in between adjacent copies of the peptide epitope. The linker may be, for example, selected from SEQ ID NOs: 21-24 and 91-92, as detailed above.

In some embodiments, the first polypeptide comprises dCas9-5X-GCN4 (SEQ ID NO: 87). dCas9-5X-GCN4 may be encoded by a polynucleotide comprising SEQ ID NO: 88. In some embodiments, the first polypeptide comprises dCas9-24X-GCN4 (SEQ ID NO: 89). dCas9-24X-GCN4 may be encoded by a polynucleotide comprising SEQ ID NO: 90 or a variant thereof. The first polypeptide may comprise an amino acid sequence having at least 80%, 85%, 90%, 95%, or 98% or greater identity to SEQ ID NO: 87 or 89, or any fragment thereof. The first polypeptide may comprise an amino acid sequence having one, two, three, four, five or more changes selected from amino acid substitutions, insertions, or deletions, relative to SEQ ID NO: 87 or 89, or any fragment thereof. The first polypeptide may comprise the amino acid sequence of SEQ ID NO: 87 or 89.

The second polypeptide of the Cas effector may comprise an effector (also referred to as an “effector domain”) and an antibody to the peptide epitope. The antibody may be any antibody that binds the peptide epitope. The antibody may specifically bind the peptide epitope. In some embodiments, the antibody comprises ScFv. ScFv may comprise the amino acid sequence of SEQ ID NO: 81 and may be encoded by a polynucleotide comprising SEQ ID NO: 82. The second polypeptide of the Cas effector may further comprise a reporter protein such as sfBFP. The sfBFP may comprise the amino acid sequence of SEQ ID NO: 83 and may be encoded by a polynucleotide comprising SEQ ID NO: 84. The reporter protein may be at the N-terminus and/or at the C-terminus of the effector. The reporter protein may be at the N-terminus and/or at the C-terminus of the antibody. The reporter protein may be in between the effector and the antibody in the polypeptide chain.

The effector has a different activity that what is endogenous to Cas protein. For example, the effector may have an activity such as transcription activation activity, transcription repression activity, transcription release factor activity, histone modification activity, nuclease activity, nucleic acid association activity, histone methylase activity, DNA methylase activity, histone demethylase activity, DNA demethylase activity, acetylation activity, and/or deacetylation activity. The activity of the effector may be direct or indirect. The effector may have this activity itself (direct), or it may recruit and/or interact with a polypeptide domain that has this activity (indirect). The effector may be at the C-terminal end of the antibody, or at the N-terminal end of the antibody, or a combination thereof. The second polypeptide of the Cas effector may include one or more than one effector. For example, the second polypeptide of the Cas effector may include an effector at the N-terminal end of the antibody as well as an effector at the C-terminal end of the antibody. In other embodiments, the second polypeptide of the Cas effector may include a single antibody and more than one (for example, two or three) effectors in tandem.

The linkage from the effector to the antibody, or from the Cas protein to the peptide epitope, can be through reversible or irreversible covalent linkage or through a non-covalent linkage, as long as the linker does not interfere with the function of the effector or antibody. For example, an antibody can be linked to an effector as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the second polypeptide of the Cas effector includes at least one linker. A linker may be included anywhere in the polypeptide sequence, for example, between the antibody and the effector. In some embodiments, the first polypeptide of the Cas effector includes at least one linker. A linker may be included anywhere in the polypeptide sequence, for example, between the Cas protein and the peptide epitope. A linker may be of any length and design to promote or restrict the mobility of components in the protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may comprise a sequence, for example, selected from SEQ ID NOs: 21-24 and 91-92, as detailed above.

In some embodiments, the second polypeptide comprises ScFv-sfBFP-MCRS1 (amino acid sequence comprising SEQ ID NO: 69, polynucleotide sequence comprising SEQ ID NO: 70), or ScFv-sfBFP-OTUD7B (amino acid sequence comprising SEQ ID NO: 71, polynucleotide sequence comprising SEQ ID NO: 72), or ScFv-sfBFP-LDB1 (amino acid sequence comprising SEQ ID NO: 73, polynucleotide sequence comprising SEQ ID NO: 74), or ScFv-sfBFP-NFKBIB (amino acid sequence comprising SEQ ID NO: 75, polynucleotide sequence comprising SEQ ID NO: 76), or ScFv-sfBFP-RelB (amino acid sequence comprising SEQ ID NO: 77, polynucleotide sequence comprising SEQ ID NO: 78), or ScFv-sfBFP-CITED2 (amino acid sequence comprising SEQ ID NO: 79, polynucleotide sequence comprising SEQ ID NO: 80). The first polypeptide may comprise an amino acid sequence having at least 80%, 85%, 90%, 95%, or 98% or greater identity to a sequence selected from SEQ ID NOs: 69, 71, 73, 75, 77, and 79, or any fragment thereof. The first polypeptide may comprise an amino acid sequence having one, two, three, four, five or more changes selected from amino acid substitutions, insertions, or deletions, relative to a sequence selected from SEQ ID NOs: 69, 71, 73, 75, 77, and 79, or any fragment thereof. The first polypeptide may comprise an amino acid sequence selected from SEQ ID NOs: 69, 71, 73, 75, 77, and 79.

d. Effector Domains

Further provided herein are novel effector domains. An effector (or “effector domain”) may modulate expression of gene it is targeted to. An effector may increase, enhance, decrease, or reduce the expression of a gene. The expression of the gene may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by about 5-95%, 10-90%, 15-85%, 20-80%, or 1.5-fold to 10-fold, relative to a control. The expression of the gene may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by about 5-95%, 10-90%, 15-85%, 20-80%, or 1.5-fold to 10-fold, relative to a control. The expression of the gene may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control.

As detailed above, a Cas fusion protein may comprise at least one effector as the second polypeptide. The second polypeptide of the Cas effector may comprise at least one effector. As also detailed above, at least one effector may be fused to at least one antibody for use in a Suntag recruitment system or a variation thereof. Effectors may include, for example, MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, or ZNF81, or a combination thereof. In some embodiments, the effector is selected from MCRS1, OTUD7B, RelB, LDB1, NFKBIB, CITED2, PHF15, SS18L1, MLLT6, ASH2L, and GSK3A. In some embodiments, the effector is selected from MCRS1, OTUD7B, RelB, LDB1, NFKBIB, and CITED2. In some embodiments, the second polypeptide domain or the effector has transcription repression activity, transcription activation activity, de-ubiquitinase activity, p300 recruitment activity, enhancer looping mediation activity, methylation activity, demethylation activity, acetylation activity, deacetylation activity, histone modification activity, histone acetylase activity, histone deacetylase activity, chromatin remodeling activity, chromatin looping modification activity, or a combination thereof.

In some embodiments, the effector reduces expression of a gene. Effectors that reduce expression of a gene may include MCRS1, OTUD7B, ZNF133, ZNF140, ZNF169, ZNF254, ZNF566, ZNF585A, ZNF689, ZNF765, and ZNF81. Effectors that reduce expression of a gene may be referred to as repressors.

In some embodiments, the effector increases or enhances expression of a gene. Effectors that increase or enhance expression of a gene may include RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72. Effectors that increase or enhance expression of a gene may be referred to as activators.

MCRS1 may comprise the amino acid sequence of SEQ ID NO: 57, encoded by a polynucleotide comprising the sequence of SEQ ID NO: 58. In some embodiments, the MCRS1 may comprise an amino acid sequence having at least 80%, 85%, 90%, 95%, or 98% or greater identity to SEQ ID NO: 57, or any fragment thereof. In some embodiments, the MCRS1 comprises an amino acid sequence having one, two, three, four, five or more changes selected from amino acid substitutions, insertions, or deletions, relative to SEQ ID NO: 57, or any fragment thereof. In some embodiments, the MCRS1 is encoded by a polynucleotide comprising a sequence having at least 80%, 85%, 90%, 95%, or 98% or greater identity to SEQ ID NO: 58, or any fragment thereof. In some embodiments, the MCRS1 is encoded by a polynucleotide comprising a sequence having one, two, three, four, five or more changes selected from nucleotide substitutions, insertions, or deletions, relative to SEQ ID NO: 58, or any fragment thereof.

OTUD7B may comprise the amino acid sequence of SEQ ID NO: 59, encoded by a polynucleotide comprising the sequence of SEQ ID NO: 60. In some embodiments, the OTUD7B comprises all of SEQ ID NO: 60 (“full OTUD7B”). OTUD7B may also comprise a fragment of SEQ ID NO: 60, such as a fragment comprising amino acids 167-440 or SEQ ID NP: 60, or a fragment comprising amino acids 792-831 of SEQ ID NO: 59. In some embodiments, the OTUD7B may comprise an amino acid sequence having at least 80%, 85%, 90%, 95%, or 98% or greater identity to SEQ ID NO: 59, or any fragment thereof. In some embodiments, the OTUD7B comprises an amino acid sequence having one, two, three, four, five or more changes selected from amino acid substitutions, insertions, or deletions, relative to SEQ ID NO: 59, or any fragment thereof. In some embodiments, the OTUD7B is encoded by a polynucleotide comprising a sequence having at least 80%, 85%, 90%, 95%, or 98% or greater identity to SEQ ID NO: 60 or any fragment thereof. In some embodiments, the OTUD7B is encoded by a polynucleotide comprising a sequence having one, two, three, four, five or more changes selected from nucleotide substitutions, insertions, or deletions, relative to SEQ ID NO: 60, or any fragment thereof.

LDB1 may comprise the amino acid sequence of SEQ ID NO: 61, encoded by a polynucleotide comprising the sequence of SEQ ID NO: 62. In some embodiments, the LDB1 may comprise an amino acid sequence having at least 80%, 85%, 90%, 95%, or 98% or greater identity to SEQ ID NO: 61, or any fragment thereof. In some embodiments, the LDB1 comprises an amino acid sequence having one, two, three, four, five or more changes selected from amino acid substitutions, insertions, or deletions, relative to SEQ ID NO: 61, or any fragment thereof. In some embodiments, the LDB1 is encoded by a polynucleotide comprising a sequence having at least 80%, 85%, 90%, 95%, or 98% or greater identity to SEQ ID NO: 62 or any fragment thereof. In some embodiments, the LDB1 is encoded by a polynucleotide comprising a sequence having one, two, three, four, five or more changes selected from nucleotide substitutions, insertions, or deletions, relative to SEQ ID NO: 62, or any fragment thereof.

NFKBIB may comprise the amino acid sequence of SEQ ID NO: 63, encoded by a polynucleotide comprising the sequence of SEQ ID NO: 64. In some embodiments, the NFKBIB may comprise an amino acid sequence having at least 80%, 85%, 90%, 95%, or 98% or greater identity to SEQ ID NO: 63, or any fragment thereof. In some embodiments, the NFKBIB comprises an amino acid sequence having one, two, three, four, five or more changes selected from amino acid substitutions, insertions, or deletions, relative to SEQ ID NO: 63, or any fragment thereof. In some embodiments, the NFKBIB is encoded by a polynucleotide comprising a sequence having at least 80%, 85%, 90%, 95%, or 98% or greater identity to SEQ ID NO: 64 or any fragment thereof. In some embodiments, the NFKBIB is encoded by a polynucleotide comprising a sequence having one, two, three, four, five or more changes selected from nucleotide substitutions, insertions, or deletions, relative to SEQ ID NO: 64, or any fragment thereof.

RelB may comprise the amino acid sequence of SEQ ID NO: 65, encoded by a polynucleotide comprising the sequence of SEQ ID NO: 66. In some embodiments, the RelB may comprise an amino acid sequence having at least 80%, 85%, 90%, 95%, or 98% or greater identity to SEQ ID NO: 65, or any fragment thereof. In some embodiments, the RelB comprises an amino acid sequence having one, two, three, four, five or more changes selected from amino acid substitutions, insertions, or deletions, relative to SEQ ID NO: 65, or any fragment thereof. In some embodiments, the RelB is encoded by a polynucleotide comprising a sequence having at least 80%, 85%, 90%, 95%, or 98% or greater identity to SEQ ID NO: 66 or any fragment thereof. In some embodiments, the RelB is encoded by a polynucleotide comprising a sequence having one, two, three, four, five or more changes selected from nucleotide substitutions, insertions, or deletions, relative to SEQ ID NO: 66, or any fragment thereof.

CITED2 may comprise the amino acid sequence of SEQ ID NO: 67, encoded by a polynucleotide comprising the sequence of SEQ ID NO: 68. In some embodiments, the CITED2 may comprise an amino acid sequence having at least 80%, 85%, 90%, 95%, or 98% or greater identity to SEQ ID NO: 67, or any fragment thereof. In some embodiments, the CITED2 comprises an amino acid sequence having one, two, three, four, five or more changes selected from amino acid substitutions, insertions, or deletions, relative to SEQ ID NO: 67, or any fragment thereof. In some embodiments, the CITED2 is encoded by a polynucleotide comprising a sequence having at least 80%, 85%, 90%, 95%, or 98% or greater identity to SEQ ID NO: 68 or any fragment thereof. In some embodiments, the CITED2 is encoded by a polynucleotide comprising a sequence having one, two, three, four, five or more changes selected from nucleotide substitutions, insertions, or deletions, relative to SEQ ID NO: 68, or any fragment thereof.

ASH2L may comprise the amino acid sequence of SEQ ID NO: 103, encoded by a polynucleotide comprising the sequence of SEQ ID NO: 104. In some embodiments, the ASH2L may comprise an amino acid sequence having at least 80%, 85%, 90%, 95%, or 98% or greater identity to SEQ ID NO: 103, or any fragment thereof. In some embodiments, the ASH2L comprises an amino acid sequence having one, two, three, four, five or more changes selected from amino acid substitutions, insertions, or deletions, relative to SEQ ID NO: 103, or any fragment thereof. In some embodiments, the ASH2L is encoded by a polynucleotide comprising a sequence having at least 80%, 85%, 90%, 95%, or 98% or greater identity to SEQ ID NO: 104, or any fragment thereof. In some embodiments, the ASH2L is encoded by a polynucleotide comprising a sequence having one, two, three, four, five or more changes selected from nucleotide substitutions, insertions, or deletions, relative to SEQ ID NO: 104, or any fragment thereof.

BCL7B may comprise the amino acid sequence of SEQ ID NO: 105, encoded by a polynucleotide comprising the sequence of SEQ ID NO: 106. In some embodiments, the BCL7B may comprise an amino acid sequence having at least 80%, 85%, 90%, 95%, or 98% or greater identity to SEQ ID NO: 105, or any fragment thereof. In some embodiments, the BCL7B comprises an amino acid sequence having one, two, three, four, five or more changes selected from amino acid substitutions, insertions, or deletions, relative to SEQ ID NO: 105, or any fragment thereof. In some embodiments, the BCL7B is encoded by a polynucleotide comprising a sequence having at least 80%, 85%, 90%, 95%, or 98% or greater identity to SEQ ID NO: 106, or any fragment thereof. In some embodiments, the BCL7B is encoded by a polynucleotide comprising a sequence having one, two, three, four, five or more changes selected from nucleotide substitutions, insertions, or deletions, relative to SEQ ID NO: 106, or any fragment thereof.

C20orf20 may comprise the amino acid sequence of SEQ ID NO: 107, encoded by a polynucleotide comprising the sequence of SEQ ID NO: 108. In some embodiments, the C20orf20 may comprise an amino acid sequence having at least 80%, 85%, 90%, 95%, or 98% or greater identity to SEQ ID NO: 107, or any fragment thereof. In some embodiments, the C20orf20 comprises an amino acid sequence having one, two, three, four, five or more changes selected from amino acid substitutions, insertions, or deletions, relative to SEQ ID NO: 107, or any fragment thereof. In some embodiments, the C20orf20 is encoded by a polynucleotide comprising a sequence having at least 80%, 85%, 90%, 95%, or 98% or greater identity to SEQ ID NO: 108, or any fragment thereof. In some embodiments, the C20orf20 is encoded by a polynucleotide comprising a sequence having one, two, three, four, five or more changes selected from nucleotide substitutions, insertions, or deletions, relative to SEQ ID NO: 108, or any fragment thereof.

DMAP1 may comprise the amino acid sequence of SEQ ID NO: 109, encoded by a polynucleotide comprising the sequence of SEQ ID NO: 110. In some embodiments, the DMAP1 may comprise an amino acid sequence having at least 80%, 85%, 90%, 95%, or 98% or greater identity to SEQ ID NO: 109, or any fragment thereof. In some embodiments, the DMAP1 comprises an amino acid sequence having one, two, three, four, five or more changes selected from amino acid substitutions, insertions, or deletions, relative to SEQ ID NO: 109, or any fragment thereof. In some embodiments, the DMAP1 is encoded by a polynucleotide comprising a sequence having at least 80%, 85%, 90%, 95%, or 98% or greater identity to SEQ ID NO: 110, or any fragment thereof. In some embodiments, the DMAP1 is encoded by a polynucleotide comprising a sequence having one, two, three, four, five or more changes selected from nucleotide substitutions, insertions, or deletions, relative to SEQ ID NO: 110, or any fragment thereof.

DYRK1B may comprise the amino acid sequence of SEQ ID NO: 111, encoded by a polynucleotide comprising the sequence of SEQ ID NO: 112. In some embodiments, the DYRK1B may comprise an amino acid sequence having at least 80%, 85%, 90%, 95%, or 98% or greater identity to SEQ ID NO: 111, or any fragment thereof. In some embodiments, the DYRK1B comprises an amino acid sequence having one, two, three, four, five or more changes selected from amino acid substitutions, insertions, or deletions, relative to SEQ ID NO: 111, or any fragment thereof. In some embodiments, the DYRK1B is encoded by a polynucleotide comprising a sequence having at least 80%, 85%, 90%, 95%, or 98% or greater identity to SEQ ID NO: 112, or any fragment thereof. In some embodiments, the DYRK1B is encoded by a polynucleotide comprising a sequence having one, two, three, four, five or more changes selected from nucleotide substitutions, insertions, or deletions, relative to SEQ ID NO: 112, or any fragment thereof.

EAF1 may comprise the amino acid sequence of SEQ ID NO: 113, encoded by a polynucleotide comprising the sequence of SEQ ID NO: 114. In some embodiments, the EAF1 may comprise an amino acid sequence having at least 80%, 85%, 90%, 95%, or 98% or greater identity to SEQ ID NO: 113, or any fragment thereof. In some embodiments, the EAF1 comprises an amino acid sequence having one, two, three, four, five or more changes selected from amino acid substitutions, insertions, or deletions, relative to SEQ ID NO: 113, or any fragment thereof. In some embodiments, the EAF1 is encoded by a polynucleotide comprising a sequence having at least 80%, 85%, 90%, 95%, or 98% or greater identity to SEQ ID NO: 114, or any fragment thereof. In some embodiments, the EAF1 is encoded by a polynucleotide comprising a sequence having one, two, three, four, five or more changes selected from nucleotide substitutions, insertions, or deletions, relative to SEQ ID NO: 114, or any fragment thereof.

FOXR2 may comprise the amino acid sequence of SEQ ID NO: 115, encoded by a polynucleotide comprising the sequence of SEQ ID NO: 116. In some embodiments, the FOXR2 may comprise an amino acid sequence having at least 80%, 85%, 90%, 95%, or 98% or greater identity to SEQ ID NO: 115, or any fragment thereof. In some embodiments, the FOXR2 comprises an amino acid sequence having one, two, three, four, five or more changes selected from amino acid substitutions, insertions, or deletions, relative to SEQ ID NO: 115, or any fragment thereof. In some embodiments, the FOXR2 is encoded by a polynucleotide comprising a sequence having at least 80%, 85%, 90%, 95%, or 98% or greater identity to SEQ ID NO: 116, or any fragment thereof. In some embodiments, the FOXR2 is encoded by a polynucleotide comprising a sequence having one, two, three, four, five or more changes selected from nucleotide substitutions, insertions, or deletions, relative to SEQ ID NO: 116, or any fragment thereof.

GSK3A may comprise the amino acid sequence of SEQ ID NO: 117, encoded by a polynucleotide comprising the sequence of SEQ ID NO: 118. In some embodiments, the GSK3A may comprise an amino acid sequence having at least 80%, 85%, 90%, 95%, or 98% or greater identity to SEQ ID NO: 117, or any fragment thereof. In some embodiments, the GSK3A comprises an amino acid sequence having one, two, three, four, five or more changes selected from amino acid substitutions, insertions, or deletions, relative to SEQ ID NO: 117, or any fragment thereof. In some embodiments, the GSK3A is encoded by a polynucleotide comprising a sequence having at least 80%, 85%, 90%, 95%, or 98% or greater identity to SEQ ID NO: 118, or any fragment thereof. In some embodiments, the GSK3A is encoded by a polynucleotide comprising a sequence having one, two, three, four, five or more changes selected from nucleotide substitutions, insertions, or deletions, relative to SEQ ID NO: 118, or any fragment thereof.

JAZF1 may comprise the amino acid sequence of SEQ ID NO: 119, encoded by a polynucleotide comprising the sequence of SEQ ID NO: 120. In some embodiments, the JAZF1 may comprise an amino acid sequence having at least 80%, 85%, 90%, 95%, or 98% or greater identity to SEQ ID NO: 119, or any fragment thereof. In some embodiments, the JAZF1 comprises an amino acid sequence having one, two, three, four, five or more changes selected from amino acid substitutions, insertions, or deletions, relative to SEQ ID NO: 119, or any fragment thereof. In some embodiments, the JAZF1 is encoded by a polynucleotide comprising a sequence having at least 80%, 85%, 90%, 95%, or 98% or greater identity to SEQ ID NO: 120, or any fragment thereof. In some embodiments, the JAZF1 is encoded by a polynucleotide comprising a sequence having one, two, three, four, five or more changes selected from nucleotide substitutions, insertions, or deletions, relative to SEQ ID NO: 120, or any fragment thereof.

KAT7 may comprise the amino acid sequence of SEQ ID NO: 121, encoded by a polynucleotide comprising the sequence of SEQ ID NO: 122. In some embodiments, the KAT7 may comprise an amino acid sequence having at least 80%, 85%, 90%, 95%, or 98% or greater identity to SEQ ID NO: 121, or any fragment thereof. In some embodiments, the KAT7 comprises an amino acid sequence having one, two, three, four, five or more changes selected from amino acid substitutions, insertions, or deletions, relative to SEQ ID NO: 121, or any fragment thereof. In some embodiments, the KAT7 is encoded by a polynucleotide comprising a sequence having at least 80%, 85%, 90%, 95%, or 98% or greater identity to SEQ ID NO: 122, or any fragment thereof. In some embodiments, the KAT7 is encoded by a polynucleotide comprising a sequence having one, two, three, four, five or more changes selected from nucleotide substitutions, insertions, or deletions, relative to SEQ ID NO: 122, or any fragment thereof.

KEAP1 may comprise the amino acid sequence of SEQ ID NO: 123, encoded by a polynucleotide comprising the sequence of SEQ ID NO: 124. In some embodiments, the KEAP1 may comprise an amino acid sequence having at least 80%, 85%, 90%, 95%, or 98% or greater identity to SEQ ID NO: 123, or any fragment thereof. In some embodiments, the KEAP1 comprises an amino acid sequence having one, two, three, four, five or more changes selected from amino acid substitutions, insertions, or deletions, relative to SEQ ID NO: 123, or any fragment thereof. In some embodiments, the KEAP1 is encoded by a polynucleotide comprising a sequence having at least 80%, 85%, 90%, 95%, or 98% or greater identity to SEQ ID NO: 124, or any fragment thereof. In some embodiments, the KEAP1 is encoded by a polynucleotide comprising a sequence having one, two, three, four, five or more changes selected from nucleotide substitutions, insertions, or deletions, relative to SEQ ID NO: 124, or any fragment thereof.

MEAF6 may comprise the amino acid sequence of SEQ ID NO: 125, encoded by a polynucleotide comprising the sequence of SEQ ID NO: 126. In some embodiments, the MEAF6 may comprise an amino acid sequence having at least 80%, 85%, 90%, 95%, or 98% or greater identity to SEQ ID NO: 125, or any fragment thereof. In some embodiments, the MEAF6 comprises an amino acid sequence having one, two, three, four, five or more changes selected from amino acid substitutions, insertions, or deletions, relative to SEQ ID NO: 125, or any fragment thereof. In some embodiments, the MEAF6 is encoded by a polynucleotide comprising a sequence having at least 80%, 85%, 90%, 95%, or 98% or greater identity to SEQ ID NO: 126, or any fragment thereof. In some embodiments, the MEAF6 is encoded by a polynucleotide comprising a sequence having one, two, three, four, five or more changes selected from nucleotide substitutions, insertions, or deletions, relative to SEQ ID NO: 126, or any fragment thereof.

MLLT6 may comprise the amino acid sequence of SEQ ID NO: 127, encoded by a polynucleotide comprising the sequence of SEQ ID NO: 128. In some embodiments, the MLLT6 may comprise an amino acid sequence having at least 80%, 85%, 90%, 95%, or 98% or greater identity to SEQ ID NO: 127, or any fragment thereof. In some embodiments, the MLLT6 comprises an amino acid sequence having one, two, three, four, five or more changes selected from amino acid substitutions, insertions, or deletions, relative to SEQ ID NO: 127, or any fragment thereof. In some embodiments, the MLLT6 is encoded by a polynucleotide comprising a sequence having at least 80%, 85%, 90%, 95%, or 98% or greater identity to SEQ ID NO: 128, or any fragment thereof. In some embodiments, the MLLT6 is encoded by a polynucleotide comprising a sequence having one, two, three, four, five or more changes selected from nucleotide substitutions, insertions, or deletions, relative to SEQ ID NO: 128, or any fragment thereof.

MORF4L2 may comprise the amino acid sequence of SEQ ID NO: 129, encoded by a polynucleotide comprising the sequence of SEQ ID NO: 130. In some embodiments, the MORF4L2 may comprise an amino acid sequence having at least 80%, 85%, 90%, 95%, or 98% or greater identity to SEQ ID NO: 129, or any fragment thereof. In some embodiments, the MORF4L2 comprises an amino acid sequence having one, two, three, four, five or more changes selected from amino acid substitutions, insertions, or deletions, relative to SEQ ID NO: 129, or any fragment thereof. In some embodiments, the MORF4L2 is encoded by a polynucleotide comprising a sequence having at least 80%, 85%, 90%, 95%, or 98% or greater identity to SEQ ID NO: 130, or any fragment thereof. In some embodiments, the MORF4L2 is encoded by a polynucleotide comprising a sequence having one, two, three, four, five or more changes selected from nucleotide substitutions, insertions, or deletions, relative to SEQ ID NO: 130, or any fragment thereof.

NFYC may comprise the amino acid sequence of SEQ ID NO: 131, encoded by a polynucleotide comprising the sequence of SEQ ID NO: 132. In some embodiments, the NFYC X may comprise an amino acid sequence having at least 80%, 85%, 90%, 95%, or 98% or greater identity to SEQ ID NO: 131, or any fragment thereof. In some embodiments, the NFYC comprises an amino acid sequence having one, two, three, four, five or more changes selected from amino acid substitutions, insertions, or deletions, relative to SEQ ID NO: 131, or any fragment thereof. In some embodiments, the NFYC is encoded by a polynucleotide comprising a sequence having at least 80%, 85%, 90%, 95%, or 98% or greater identity to SEQ ID NO: 132, or any fragment thereof. In some embodiments, the NFYC is encoded by a polynucleotide comprising a sequence having one, two, three, four, five or more changes selected from nucleotide substitutions, insertions, or deletions, relative to SEQ ID NO: 132, or any fragment thereof.

PHF15 may comprise the amino acid sequence of SEQ ID NO: 133, encoded by a polynucleotide comprising the sequence of SEQ ID NO: 134. In some embodiments, the PHF15 may comprise an amino acid sequence having at least 80%, 85%, 90%, 95%, or 98% or greater identity to SEQ ID NO: 133, or any fragment thereof. In some embodiments, the PHF15 comprises an amino acid sequence having one, two, three, four, five or more changes selected from amino acid substitutions, insertions, or deletions, relative to SEQ ID NO: 133, or any fragment thereof. In some embodiments, the PHF15 is encoded by a polynucleotide comprising a sequence having at least 80%, 85%, 90%, 95%, or 98% or greater identity to SEQ ID NO: 134, or any fragment thereof. In some embodiments, the PHF15 is encoded by a polynucleotide comprising a sequence having one, two, three, four, five or more changes selected from nucleotide substitutions, insertions, or deletions, relative to SEQ ID NO: 134, or any fragment thereof.

PKIB may comprise the amino acid sequence of SEQ ID NO: 135, encoded by a polynucleotide comprising the sequence of SEQ ID NO: 136. In some embodiments, the PKIB may comprise an amino acid sequence having at least 80%, 85%, 90%, 95%, or 98% or greater identity to SEQ ID NO: 135, or any fragment thereof. In some embodiments, the PKIB comprises an amino acid sequence having one, two, three, four, five or more changes selected from amino acid substitutions, insertions, or deletions, relative to SEQ ID NO: 135, or any fragment thereof. In some embodiments, the PKIB is encoded by a polynucleotide comprising a sequence having at least 80%, 85%, 90%, 95%, or 98% or greater identity to SEQ ID NO: 136, or any fragment thereof. In some embodiments, the PKIB is encoded by a polynucleotide comprising a sequence having one, two, three, four, five or more changes selected from nucleotide substitutions, insertions, or deletions, relative to SEQ ID NO: 136, or any fragment thereof.

POLE4 may comprise the amino acid sequence of SEQ ID NO: 137, encoded by a polynucleotide comprising the sequence of SEQ ID NO: 138. In some embodiments, the POLE4 may comprise an amino acid sequence having at least 80%, 85%, 90%, 95%, or 98% or greater identity to SEQ ID NO: 137, or any fragment thereof. In some embodiments, the POLE4 comprises an amino acid sequence having one, two, three, four, five or more changes selected from amino acid substitutions, insertions, or deletions, relative to SEQ ID NO: 137, or any fragment thereof. In some embodiments, the POLE4 is encoded by a polynucleotide comprising a sequence having at least 80%, 85%, 90%, 95%, or 98% or greater identity to SEQ ID NO: 138, or any fragment thereof. In some embodiments, the POLE4 is encoded by a polynucleotide comprising a sequence having one, two, three, four, five or more changes selected from nucleotide substitutions, insertions, or deletions, relative to SEQ ID NO: 138, or any fragment thereof.

PRKRIR may comprise the amino acid sequence of SEQ ID NO: 139, encoded by a polynucleotide comprising the sequence of SEQ ID NO: 140. In some embodiments, the PRKRIR may comprise an amino acid sequence having at least 80%, 85%, 90%, 95%, or 98% or greater identity to SEQ ID NO: 139, or any fragment thereof. In some embodiments, the PRKRIR comprises an amino acid sequence having one, two, three, four, five or more changes selected from amino acid substitutions, insertions, or deletions, relative to SEQ ID NO: 139, or any fragment thereof. In some embodiments, the PRKRIR is encoded by a polynucleotide comprising a sequence having at least 80%, 85%, 90%, 95%, or 98% or greater identity to SEQ ID NO: 140, or any fragment thereof. In some embodiments, the PRKRIR is encoded by a polynucleotide comprising a sequence having one, two, three, four, five or more changes selected from nucleotide substitutions, insertions, or deletions, relative to SEQ ID NO: 140, or any fragment thereof.

PYGO2 may comprise the amino acid sequence of SEQ ID NO: 141, encoded by a polynucleotide comprising the sequence of SEQ ID NO: 142. In some embodiments, the PYGO may comprise an amino acid sequence having at least 80%, 85%, 90%, 95%, or 98% or greater identity to SEQ ID NO: 141, or any fragment thereof. In some embodiments, the PYGO comprises an amino acid sequence having one, two, three, four, five or more changes selected from amino acid substitutions, insertions, or deletions, relative to SEQ ID NO: 141, or any fragment thereof. In some embodiments, the PYGO is encoded by a polynucleotide comprising a sequence having at least 80%, 85%, 90%, 95%, or 98% or greater identity to SEQ ID NO: 142, or any fragment thereof. In some embodiments, the PYGO is encoded by a polynucleotide comprising a sequence having one, two, three, four, five or more changes selected from nucleotide substitutions, insertions, or deletions, relative to SEQ ID NO: 142, or any fragment thereof.

RANBP1 may comprise the amino acid sequence of SEQ ID NO: 143, encoded by a polynucleotide comprising the sequence of SEQ ID NO: 144. In some embodiments, the RANBP may comprise an amino acid sequence having at least 80%, 85%, 90%, 95%, or 98% or greater identity to SEQ ID NO: 143, or any fragment thereof. In some embodiments, the RANBP comprises an amino acid sequence having one, two, three, four, five or more changes selected from amino acid substitutions, insertions, or deletions, relative to SEQ ID NO: 143, or any fragment thereof. In some embodiments, the RANBP is encoded by a polynucleotide comprising a sequence having at least 80%, 85%, 90%, 95%, or 98% or greater identity to SEQ ID NO: 144, or any fragment thereof. In some embodiments, the RANBP is encoded by a polynucleotide comprising a sequence having one, two, three, four, five or more changes selected from nucleotide substitutions, insertions, or deletions, relative to SEQ ID NO: 144, or any fragment thereof.

RPRD1B may comprise the amino acid sequence of SEQ ID NO: 145, encoded by a polynucleotide comprising the sequence of SEQ ID NO: 146. In some embodiments, the RPRD1B may comprise an amino acid sequence having at least 80%, 85%, 90%, 95%, or 98% or greater identity to SEQ ID NO: 145, or any fragment thereof. In some embodiments, the RPRD1B comprises an amino acid sequence having one, two, three, four, five or more changes selected from amino acid substitutions, insertions, or deletions, relative to SEQ ID NO: 145, or any fragment thereof. In some embodiments, the RPRD1B is encoded by a polynucleotide comprising a sequence having at least 80%, 85%, 90%, 95%, or 98% or greater identity to SEQ ID NO: 146, or any fragment thereof. In some embodiments, the RPRD1B is encoded by a polynucleotide comprising a sequence having one, two, three, four, five or more changes selected from nucleotide substitutions, insertions, or deletions, relative to SEQ ID NO: 146, or any fragment thereof.

SPIN1 may comprise the amino acid sequence of SEQ ID NO: 147, encoded by a polynucleotide comprising the sequence of SEQ ID NO: 148. In some embodiments, the SPIN1 may comprise an amino acid sequence having at least 80%, 85%, 90%, 95%, or 98% or greater identity to SEQ ID NO: 147, or any fragment thereof. In some embodiments, the SPIN1 comprises an amino acid sequence having one, two, three, four, five or more changes selected from amino acid substitutions, insertions, or deletions, relative to SEQ ID NO: 147, or any fragment thereof. In some embodiments, the SPIN1 is encoded by a polynucleotide comprising a sequence having at least 80%, 85%, 90%, 95%, or 98% or greater identity to SEQ ID NO: 148, or any fragment thereof. In some embodiments, the SPIN1 is encoded by a polynucleotide comprising a sequence having one, two, three, four, five or more changes selected from nucleotide substitutions, insertions, or deletions, relative to SEQ ID NO: 148, or any fragment thereof.

SS18L1 may comprise the amino acid sequence of SEQ ID NO: 149, encoded by a polynucleotide comprising the sequence of SEQ ID NO: 150. In some embodiments, the SS18L1 may comprise an amino acid sequence having at least 80%, 85%, 90%, 95%, or 98% or greater identity to SEQ ID NO: 149, or any fragment thereof. In some embodiments, the SS18L1 comprises an amino acid sequence having one, two, three, four, five or more changes selected from amino acid substitutions, insertions, or deletions, relative to SEQ ID NO: 149, or any fragment thereof. In some embodiments, the SS18L1 is encoded by a polynucleotide comprising a sequence having at least 80%, 85%, 90%, 95%, or 98% or greater identity to SEQ ID NO: 150, or any fragment thereof. In some embodiments, the SS18L1 is encoded by a polynucleotide comprising a sequence having one, two, three, four, five or more changes selected from nucleotide substitutions, insertions, or deletions, relative to SEQ ID NO: 150, or any fragment thereof.

TADA3 may comprise the amino acid sequence of SEQ ID NO: 151, encoded by a polynucleotide comprising the sequence of SEQ ID NO: 152. In some embodiments, the TADA3 may comprise an amino acid sequence having at least 80%, 85%, 90%, 95%, or 98% or greater identity to SEQ ID NO: 151, or any fragment thereof. In some embodiments, the TADA3 comprises an amino acid sequence having one, two, three, four, five or more changes selected from amino acid substitutions, insertions, or deletions, relative to SEQ ID NO: 151, or any fragment thereof. In some embodiments, the TADA3 is encoded by a polynucleotide comprising a sequence having at least 80%, 85%, 90%, 95%, or 98% or greater identity to SEQ ID NO: 152, or any fragment thereof. In some embodiments, the TADA3 is encoded by a polynucleotide comprising a sequence having one, two, three, four, five or more changes selected from nucleotide substitutions, insertions, or deletions, relative to SEQ ID NO: 152, or any fragment thereof.

TAF6 may comprise the amino acid sequence of SEQ ID NO: 153, encoded by a polynucleotide comprising the sequence of SEQ ID NO: 154. In some embodiments, the TAF6 may comprise an amino acid sequence having at least 80%, 85%, 90%, 95%, or 98% or greater identity to SEQ ID NO: 153, or any fragment thereof. In some embodiments, the TAF6 comprises an amino acid sequence having one, two, three, four, five or more changes selected from amino acid substitutions, insertions, or deletions, relative to SEQ ID NO: 153, or any fragment thereof. In some embodiments, the TAF6 is encoded by a polynucleotide comprising a sequence having at least 80%, 85%, 90%, 95%, or 98% or greater identity to SEQ ID NO: 154 or any fragment thereof. In some embodiments, the TAF6 is encoded by a polynucleotide comprising a sequence having one, two, three, four, five or more changes selected from nucleotide substitutions, insertions, or deletions, relative to SEQ ID NO: 154, or any fragment thereof.

TBPL1 may comprise the amino acid sequence of SEQ ID NO: 155, encoded by a polynucleotide comprising the sequence of SEQ ID NO: 156. In some embodiments, the TBPL1 may comprise an amino acid sequence having at least 80%, 85%, 90%, 95%, or 98% or greater identity to SEQ ID NO: 155, or any fragment thereof. In some embodiments, the TBPL1 comprises an amino acid sequence having one, two, three, four, five or more changes selected from amino acid substitutions, insertions, or deletions, relative to SEQ ID NO: 155, or any fragment thereof. In some embodiments, the TBPL1 is encoded by a polynucleotide comprising a sequence having at least 80%, 85%, 90%, 95%, or 98% or greater identity to SEQ ID NO: 156, or any fragment thereof. In some embodiments, the TBPL1 is encoded by a polynucleotide comprising a sequence having one, two, three, four, five or more changes selected from nucleotide substitutions, insertions, or deletions, relative to SEQ ID NO: 156, or any fragment thereof.

VPS72 may comprise the amino acid sequence of SEQ ID NO: 157, encoded by a polynucleotide comprising the sequence of SEQ ID NO: 158. In some embodiments, the VPS7 may comprise an amino acid sequence having at least 80%, 85%, 90%, 95%, or 98% or greater identity to SEQ ID NO: 157, or any fragment thereof. In some embodiments, the VPS7X comprises an amino acid sequence having one, two, three, four, five or more changes selected from amino acid substitutions, insertions, or deletions, relative to SEQ ID NO: 157, or any fragment thereof. In some embodiments, the VPS7 is encoded by a polynucleotide comprising a sequence having at least 80%, 85%, 90%, 95%, or 98% or greater identity to SEQ ID NO: 158, or any fragment thereof. In some embodiments, the VPS7 is encoded by a polynucleotide comprising a sequence having one, two, three, four, five or more changes selected from nucleotide substitutions, insertions, or deletions, relative to SEQ ID NO: 158, or any fragment thereof.

ZNF133 may comprise the amino acid sequence of SEQ ID NO: 159, encoded by a polynucleotide comprising the sequence of SEQ ID NO: 160. In some embodiments, the ZNF133 may comprise an amino acid sequence having at least 80%, 85%, 90%, 95%, or 98% or greater identity to SEQ ID NO: 159, or any fragment thereof. In some embodiments, the ZNF133 comprises an amino acid sequence having one, two, three, four, five or more changes selected from amino acid substitutions, insertions, or deletions, relative to SEQ ID NO: 159, or any fragment thereof. In some embodiments, the ZNF133 is encoded by a polynucleotide comprising a sequence having at least 80%, 85%, 90%, 95%, or 98% or greater identity to SEQ ID NO: 160, or any fragment thereof. In some embodiments, the ZNF133 is encoded by a polynucleotide comprising a sequence having one, two, three, four, five or more changes selected from nucleotide substitutions, insertions, or deletions, relative to SEQ ID NO: 160, or any fragment thereof.

ZNF140 may comprise the amino acid sequence of SEQ ID NO: 161, encoded by a polynucleotide comprising the sequence of SEQ ID NO: 162. In some embodiments, the ZNF140 may comprise an amino acid sequence having at least 80%, 85%, 90%, 95%, or 98% or greater identity to SEQ ID NO: 161, or any fragment thereof. In some embodiments, the ZNF140 comprises an amino acid sequence having one, two, three, four, five or more changes selected from amino acid substitutions, insertions, or deletions, relative to SEQ ID NO: 161, or any fragment thereof. In some embodiments, the ZNF140 is encoded by a polynucleotide comprising a sequence having at least 80%, 85%, 90%, 95%, or 98% or greater identity to SEQ ID NO: 162, or any fragment thereof. In some embodiments, the ZNF140 is encoded by a polynucleotide comprising a sequence having one, two, three, four, five or more changes selected from nucleotide substitutions, insertions, or deletions, relative to SEQ ID NO: 162, or any fragment thereof.

ZNF169 may comprise the amino acid sequence of SEQ ID NO: 163, encoded by a polynucleotide comprising the sequence of SEQ ID NO: 164. In some embodiments, the ZNF169 may comprise an amino acid sequence having at least 80%, 85%, 90%, 95%, or 98% or greater identity to SEQ ID NO: 163, or any fragment thereof. In some embodiments, the ZNF169 comprises an amino acid sequence having one, two, three, four, five or more changes selected from amino acid substitutions, insertions, or deletions, relative to SEQ ID NO: 163, or any fragment thereof. In some embodiments, the ZNF169 is encoded by a polynucleotide comprising a sequence having at least 80%, 85%, 90%, 95%, or 98% or greater identity to SEQ ID NO: 164, or any fragment thereof. In some embodiments, the ZNF169 is encoded by a polynucleotide comprising a sequence having one, two, three, four, five or more changes selected from nucleotide substitutions, insertions, or deletions, relative to SEQ ID NO: 164, or any fragment thereof.

ZNF254 may comprise the amino acid sequence of SEQ ID NO: 165, encoded by a polynucleotide comprising the sequence of SEQ ID NO: 166. In some embodiments, the ZNF254 may comprise an amino acid sequence having at least 80%, 85%, 90%, 95%, or 98% or greater identity to SEQ ID NO: 165, or any fragment thereof. In some embodiments, the ZNF254 comprises an amino acid sequence having one, two, three, four, five or more changes selected from amino acid substitutions, insertions, or deletions, relative to SEQ ID NO: 165, or any fragment thereof. In some embodiments, the ZNF254 is encoded by a polynucleotide comprising a sequence having at least 80%, 85%, 90%, 95%, or 98% or greater identity to SEQ ID NO: 166, or any fragment thereof. In some embodiments, the ZNF254 is encoded by a polynucleotide comprising a sequence having one, two, three, four, five or more changes selected from nucleotide substitutions, insertions, or deletions, relative to SEQ ID NO: 166, or any fragment thereof.

ZNF566 may comprise the amino acid sequence of SEQ ID NO: 167, encoded by a polynucleotide comprising the sequence of SEQ ID NO: 168. In some embodiments, the ZNF56 may comprise an amino acid sequence having at least 80%, 85%, 90%, 95%, or 98% or greater identity to SEQ ID NO: 167 or any fragment thereof. In some embodiments, the ZNF56 comprises an amino acid sequence having one, two, three, four, five or more changes selected from amino acid substitutions, insertions, or deletions, relative to SEQ ID NO: 167, or any fragment thereof. In some embodiments, the ZNF56X is encoded by a polynucleotide comprising a sequence having at least 80%, 85%, 90%, 95%, or 98% or greater identity to SEQ ID NO: 168, or any fragment thereof. In some embodiments, the ZNF56 is encoded by a polynucleotide comprising a sequence having one, two, three, four, five or more changes selected from nucleotide substitutions, insertions, or deletions, relative to SEQ ID NO: 168, or any fragment thereof.

ZNF585A may comprise the amino acid sequence of SEQ ID NO: 169, encoded by a polynucleotide comprising the sequence of SEQ ID NO: 170. In some embodiments, the ZNF585A may comprise an amino acid sequence having at least 80%, 85%, 90%, 95%, or 98% or greater identity to SEQ ID NO: 169, or any fragment thereof. In some embodiments, the ZNF585A comprises an amino acid sequence having one, two, three, four, five or more changes selected from amino acid substitutions, insertions, or deletions, relative to SEQ ID NO: 169, or any fragment thereof. In some embodiments, the ZNF585A is encoded by a polynucleotide comprising a sequence having at least 80%, 85%, 90%, 95%, or 98% or greater identity to SEQ ID NO: 170, or any fragment thereof. In some embodiments, the ZNF585A is encoded by a polynucleotide comprising a sequence having one, two, three, four, five or more changes selected from nucleotide substitutions, insertions, or deletions, relative to SEQ ID NO: 170, or any fragment thereof.

ZNF689 may comprise the amino acid sequence of SEQ ID NO: 171, encoded by a polynucleotide comprising the sequence of SEQ ID NO: 172. In some embodiments, the ZNF689 may comprise an amino acid sequence having at least 80%, 85%, 90%, 95%, or 98% or greater identity to SEQ ID NO: 171, or any fragment thereof. In some embodiments, the ZNF689 comprises an amino acid sequence having one, two, three, four, five or more changes selected from amino acid substitutions, insertions, or deletions, relative to SEQ ID NO: 171, or any fragment thereof. In some embodiments, the ZNF689 is encoded by a polynucleotide comprising a sequence having at least 80%, 85%, 90%, 95%, or 98% or greater identity to SEQ ID NO: 172, or any fragment thereof. In some embodiments, the ZNF689 is encoded by a polynucleotide comprising a sequence having one, two, three, four, five or more changes selected from nucleotide substitutions, insertions, or deletions, relative to SEQ ID NO: 172, or any fragment thereof.

ZNF765 may comprise the amino acid sequence of SEQ ID NO: 173, encoded by a polynucleotide comprising the sequence of SEQ ID NO: 174. In some embodiments, the ZNF765 may comprise an amino acid sequence having at least 80%, 85%, 90%, 95%, or 98% or greater identity to SEQ ID NO: 173, or any fragment thereof. In some embodiments, the ZNF765 comprises an amino acid sequence having one, two, three, four, five or more changes selected from amino acid substitutions, insertions, or deletions, relative to SEQ ID NO: 173, or any fragment thereof. In some embodiments, the ZNF765 is encoded by a polynucleotide comprising a sequence having at least 80%, 85%, 90%, 95%, or 98% or greater identity to SEQ ID NO: 174, or any fragment thereof. In some embodiments, the ZNF765 is encoded by a polynucleotide comprising a sequence having one, two, three, four, five or more changes selected from nucleotide substitutions, insertions, or deletions, relative to SEQ ID NO: 174, or any fragment thereof.

ZNF81 may comprise the amino acid sequence of SEQ ID NO: 175, encoded by a polynucleotide comprising the sequence of SEQ ID NO: 176. In some embodiments, the ZNF81 may comprise an amino acid sequence having at least 80%, 85%, 90%, 95%, or 98% or greater identity to SEQ ID NO: 175, or any fragment thereof. In some embodiments, the ZNF81 comprises an amino acid sequence having one, two, three, four, five or more changes selected from amino acid substitutions, insertions, or deletions, relative to SEQ ID NO: 175, or any fragment thereof. In some embodiments, the ZNF81 is encoded by a polynucleotide comprising a sequence having at least 80%, 85%, 90%, 95%, or 98% or greater identity to SEQ ID NO: 176, or any fragment thereof. In some embodiments, the ZNF81 is encoded by a polynucleotide comprising a sequence having one, two, three, four, five or more changes selected from nucleotide substitutions, insertions, or deletions, relative to SEQ ID NO: 176, or any fragment thereof.

Other examples of effectors, or second polypeptide domains of the Cas fusion protein, are detailed below.

### i) Transcription Activation Activity

The second polypeptide domain, or the effector, can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 41 or SEQ ID NO: 42. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 43, encoded by the polynucleotide of SEQ ID NO: 44. VPH may comprise a polypeptide having the amino acid sequence of SEQ ID NO: 53, encoded by a polynucleotide comprising the sequence of SEQ ID NO: 54. VPR may comprise a polypeptide having the amino acid sequence of SEQ ID NO: 55, encoded by a polynucleotide comprising the sequence of SEQ ID NO: 56.

### ii) Transcription Repression Activity

The second polypeptide domain, or the effector, can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4X repressor domain, MxiI repressor domain, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain, or the effector, has a KRAB domain activity, ERF repressor domain activity, MxiI repressor domain activity, SID4X repressor domain activity, Mad-SID repressor domain activity, DNMT3A or DNMT3L or fusion thereof activity, LSD1 histone demethylase activity, or TATA box binding protein activity. In some embodiments, the second polypeptide domain or the effector comprises KRAB. KRAB may comprise a polypeptide having the amino acid sequence of SEQ ID NO: 45, encoded by polynucleotide comprising the sequence of SEQ ID NO: 46. For example, the fusion protein may be S. pyogenes dCas9-KRAB (protein sequence comprising SEQ ID NO: 47; polynucleotide sequence comprising SEQ ID NO: 48). The fusion protein may be S. aureus dCas9-KRAB (protein sequence comprising SEQ ID NO: 49; polynucleotide sequence comprising SEQ ID NO: 50).

iii) Transcription Release Factor Activity

The second polypeptide domain, or the effector, can have transcription release factor activity. The second polypeptide domain, or the effector, can have eukaryotic release factor 1 (ERF1) activity or eukaryotic release factor 3 (ERF3) activity.

### iv) Histone Modification Activity

The second polypeptide domain, or the effector, can have histone modification activity. The second polypeptide domain, or the effector, can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 41 or SEQ ID NO: 42.

### v) Nuclease Activity

The second polypeptide domain, or the effector, can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

### vi) Nucleic Acid Association Activity

The second polypeptide domain, or the effector, can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

vii) Methylase Activity

The second polypeptide domain, or the effector, can have methylase activity, which involves transferring a methyl group to DNA, RNA, protein, small molecule, cytosine, or adenine. In some embodiments, the second polypeptide domain or the effector includes a DNA methyltransferase.

viii) Demethylase Activity

The second polypeptide domain, or the effector, can have demethylase activity. The second polypeptide domain or the effector can include an enzyme that removes methyl (CH3-) groups from nucleic acids, proteins (in particular histones), and other molecules. Alternatively, the second polypeptide or the effector can convert the methyl group to hydroxymethylcytosine in a mechanism for demethylating DNA. The second polypeptide or the effector can catalyze this reaction. For example, a second polypeptide that catalyzes this reaction can be Tet1, also known as Tet1CD (Ten-eleven translocation methylcytosine dioxygenase 1; amino acid sequence comprising SEQ ID NO: 51; polynucleotide sequence comprising SEQ ID NO: 52). In some embodiments, the second polypeptide domain or the effector has histone demethylase activity. In some embodiments, the second polypeptide domain or the effector has DNA demethylase activity.

e. Guide RNA (gRNA)

The CRISPR/Cas-based gene editing system may include at least one gRNA molecule. For example, the CRISPR/Cas-based gene editing system may include two gRNA molecules. The at least one gRNA molecule can bind and recognize a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 19 (RNA), which is encoded by a sequence comprising SEQ ID NO: 18 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target the Cas9 protein or fusion protein to a gene or a regulatory element thereof. The gRNA may target the Cas protein or fusion protein to a non-open chromatin region, an open chromatin region, a transcribed region of the target gene, a region upstream of a transcription start site of the target gene, a regulatory element of the target gene, an intron of the target gene, or an exon of the target gene, or a combination thereof. In some embodiments, the gRNA targets the Cas9 protein or fusion protein to a promoter of a gene. In some embodiments, the target region is located between about 1 to about 1000 base pairs upstream of a transcription start site of a target gene. In some embodiments, the DNA targeting composition comprises two or more gRNAs, each gRNA binding to a different target region.

The gRNA may target a region within or near a gene of interest. For example, the gRNA may target B2M or CD25 or TetO (see TABLE 3 and TABLE 4). The gRNA may target or bind to a regulatory region of a gene of interest. The gRNA may comprise a polynucleotide sequence comprising at least one of SEQ ID NOs: 96-98 and 101-102, or a complement thereof, or a variant thereof, or a truncation thereof. The gRNA may be encoded by a polynucleotide sequence comprising at least one of SEQ ID NOs: 93-95 and 99-100, or a complement thereof, or a variant thereof, or a truncation thereof. The gRNA may bind and target a polynucleotide sequence comprising at least one of SEQ ID NOs: 93-95 and 99-100, or a complement thereof, or a variant thereof, or a truncation thereof. A truncation may be 1, 2, 3, 4, 5, 6, 7, 8, or 9 nucleotides shorter than the sequence of any one of SEQ ID NOs: 93-102. In some embodiments, the gRNA targets or binds to a gene or regulatory element thereof that is related to a disease, such as, for example, Duchenne muscular dystrophy (DMD), Becker muscular dystrophy (BMD), and/or cancer.

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

f. Repair Pathways

The CRISPR/Cas9-based gene editing system may be used to introduce site-specific double strand breaks at targeted genomic loci. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

### i) Homology-Directed Repair (HDR)

Restoration of protein expression from a gene may involve homology-directed repair (HDR). A donor template may be administered to a cell. A donor sequence comprises a polynucleotide sequence to be inserted into a genome. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

### ii) Non-Homologous End Joining (NHEJ)

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 4. REPORTER PROTEIN

In some embodiments, the DNA targeting compositions or CRISPR/Cas9 systems include at least one reporter protein. For example, and as detailed above, the second polypeptide of the Cas effector may comprise a reporter protein such as sfBFP. A polynucleotide sequence encoding the reporter protein may be operably linked to the polynucleotide sequence encoding the Cas9 protein and/or Cas9 fusion protein and/or antibody and/or effector. The reporter protein may include any protein or peptide that is suitably detectable, such as, by fluorescence, chemiluminescence, enzyme activity such as beta galactosidase or alkaline phosphatase, and/or antibody binding detection. The reporter protein may comprise a fluorescent protein. The reporter protein may comprise a protein or peptide detectable with an antibody. For example, the reporter protein may comprise sfBFP, GFP, YFP, RFP, CFP, DsRed, luciferase, and/or Thy1.

### 5. GENETIC CONSTRUCTS

The CRISPR/Cas9-based gene editing system or any component thereof may be encoded by or comprised within one or more genetic constructs. The CRISPR/Cas9-based gene editing system may comprise one or more genetic constructs. The genetic construct, such as a plasmid or expression vector, may comprise a nucleic acid that encodes the CRISPR/Cas9-based gene editing system and/or at least one component thereof such as at lease one gRNA. In some embodiments, a genetic construct encodes at least one effector domain. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence. In some embodiments, a single genetic construct encodes at least one effector domain, at least one antibody, a Cas9 molecule or fusion protein, and at least one peptide epitope. In some embodiments, a first genetic construct encodes at least one effector domain and at least one antibody, and a second genetic construct encodes a Cas9 molecule or fusion protein and at least one peptide epitope.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The vector may also comprise a promoter that is operably linked to the CRISPR/Cas-based gene editing system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metallothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

### 6. PHARMACEUTICAL COMPOSITIONS

Further provided herein are pharmaceutical compositions comprising the above-described genetic constructs or gene editing systems. In some embodiments, the pharmaceutical composition may comprise about 1 ng to about 10 mg of DNA encoding the CRISPR/Cas-based gene editing system or at least one component thereof. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 7. ADMINISTRATION

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector lib devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and/or the Cas9 molecule or fusion protein and/or Cas effector and/or effector domain.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-γ producing killer dendritic cells (IKDC), memory T cells (TCMs), and effector T cells (Tes). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells.

### 8. KITS

Provided herein is a kit, which may be used to modulate gene expression. The kit comprises genetic constructs or a composition comprising the same, for modulating gene expression, as described above, and instructions for using said composition. In some embodiments, the kit includes at least one effector as detailed herein, or a polynucleotide encoding the at least one effector. The effector may be selected from, for example, MCRS1, OTUD7B, RelB, LDB1, NFKBIB, CITED2, ASH2L, GSK3A, MLLT6, PHF15, SS18L1, BCL7B, C20orf20, DMAP1, DYRK1B, EAF1, FOXR2, JAZF1, KAT7, KEAP1, MEAF6, MORF4L2, NFYC, PKIB, POLE4, PRKRIR, PYGO2, RANBP1, RPRD1B, SPIN1, TADA3, TAF6, TBPL1, VPS72, ZNF133, ZNF140, ZNF169, ZNF254, ZNF566, ZNF585A, ZNF689, ZNF765, and ZNF81. In some embodiments, the kit comprises at least one gRNA as detailed herein. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof for modulating gene expression may include a modified AAV vector that includes a gRNA molecule(s) and a Cas9 protein or fusion protein or Cas effector, as described above. The CRISPR/Cas-based gene editing system, as described above, may be included in the kit to specifically bind and target a particular region in a gene.

### 9. METHODS

a. Methods of Modulating Expression of a Gene

Provided herein are methods of modulating expression of a gene in a cell or in a subject. The methods may include administering to the cell or the subject a DNA targeting composition as detailed herein or at least one component thereof, or an isolated polynucleotide sequence as detailed herein, or a vector as detailed herein, or a pharmaceutical composition as detailed herein, or a combination thereof. In some embodiments, the method includes administering to a cell or subject an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, or a polynucleotide encoding the effector. In some embodiments, the effector is targeted to a gene or a regulatory element thereof.

In some embodiments, the expression of the gene is increased relative to a control. In some embodiments, wherein the expression of the gene is decreased relative to a control. In some embodiments, the gene comprises the dystrophin gene, or the CD25 gene, or the B2M gene, or the TRAC gene. In some embodiments, the cell is a muscle cell or a T cell.

In some embodiments, the gene is the dystrophin gene. Dystrophin is a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane. The dystrophin gene is 2.2 megabases at locus Xp21. The primary transcription measures about 2,400 kb with the mature mRNA being about 14 kb. 79 exons include approximately 2.2 million nucleotides and code for the protein which is over 3500 amino acids. Normal skeleton muscle tissue contains only small amounts of dystrophin, but its absence of abnormal expression leads to the development of severe and incurable symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

Duchenne muscular dystrophy (DMD) is the result of inherited or X-linked recessive spontaneous mutation(s) that cause nonsense or frame shift mutations in the dystrophin gene. DMD is a severe, highly debilitating and incurable muscle disease and is the most prevalent lethal heritable childhood disease and affects approximately one in 5,000 newborn males. DMD is characterized by muscle deterioration, progressive muscle weakness, often leading to mortality in subjects at age mid-twenties and premature death, due to the lack of a functional dystrophin gene. Most mutations are deletions in the dystrophin gene that disrupt the reading frame. Naturally occurring mutations and their consequences are relatively well understood for DMD. In-frame deletions that occur in the exon 45-55 regions contained within the rod domain can produce highly functional dystrophin proteins, and many carriers are asymptomatic or display mild symptoms. Exons 45-55 of dystrophin are a mutational hotspot. More than 60% of patients may be treated by targeting exons in this region of the dystrophin gene. Efforts have been made to restore the disrupted dystrophin reading frame in DMD patients by skipping non-essential exon(s) (e.g., exon 45 skipping) during mRNA splicing to produce internally deleted but functional dystrophin proteins. The deletion of internal dystrophin exon(s) (for example, deletion of exon 45) may retain the proper reading frame and can generate an internally truncated but partially functional dystrophin protein. Deletions between exons 45-55 of dystrophin can result in a phenotype that is much milder compared to DMD.

A dystrophin gene may be a mutant dystrophin gene. A dystrophin gene may be a wild-type dystrophin gene. A dystrophin gene may have a sequence that is functionally identical to a wild-type dystrophin gene, for example, the sequence may be codon-optimized but still encode for the same protein as the wild-type dystrophin. A mutant dystrophin gene may include one or more mutations relative to the wild-type dystrophin gene. Mutations may include, for example, nucleotide deletions, substitutions, additions, transversions, or combinations thereof. A mutation in the dystrophin gene may be a functional deletion of the dystrophin gene. In some embodiments, the mutation in the dystrophin gene comprises an insertion or deletion in the dystrophin gene that prevents protein expression from the dystrophin gene. Mutations may be in one or more exons and/or introns. Mutations may include deletions of all or parts of at least one intron and/or exon. An exon of a mutant dystrophin gene may be mutated or at least partially deleted from the dystrophin gene. An exon of a mutant dystrophin gene may be fully deleted. A mutant dystrophin gene may have a portion or fragment thereof that corresponds to the corresponding sequence in the wild-type dystrophin gene. In some embodiments, a disrupted dystrophin gene caused by a deleted or mutated exon can be restored in DMD patients by adding back the corresponding wild-type exon. In some embodiments, disrupted dystrophin caused by a deleted or mutated exon 52 can be restored in DMD patients by adding back in wild-type exon 52. In certain embodiments, addition of exon 52 to restore reading frame ameliorates the phenotype in DMD subjects, including DMD subjects with deletion mutations. In certain embodiments, one or more exons may be added and inserted into the disrupted dystrophin gene. The one or more exons may be added and inserted so as to restore the corresponding mutated or deleted exon(s) in dystrophin. The one or more exons may be added and inserted into the disrupted dystrophin gene in addition to adding back and inserting the exon 52. In certain embodiments, exon 52 of a dystrophin gene refers to the 52^{nd} exon of the dystrophin gene. Exon 52 is frequently adjacent to frame-disrupting deletions in DMD patients.

b. Methods of Treating a Disease

Provided herein are methods of treating a disease in a subject. The methods may include administering to the cell or the subject a DNA targeting composition as detailed herein or at least one component thereof, or an isolated polynucleotide sequence as detailed herein, or a vector as detailed herein, or a pharmaceutical composition as detailed herein, or a combination thereof. In some embodiments, the method includes administering to the subject an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, or a polynucleotide encoding the effector. In some embodiments, the effector is targeted to a gene or a regulatory element thereof.

In some embodiments, the disease is selected from Duchenne muscular dystrophy (DMD), Becker muscular dystrophy (BMD), and cancer.

### 10. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Effector Screen 1: Suntag System and B2M Expression

A library was generated including 3015 effector domains derived from a commercial ORFeome library. A version of the suntag system compatible with LR cloning to insert effectors was generated, and random barcodes were appended at high coverage. Effectors were then cloned in by LR cloning, intentionally bottlenecked at 100 k colonies to maintain a manageable number of barcodes. Barcodes were then mapped to effectors using nanopore sequencing.

The effect of each effector on gene expression was measured in pooled screens. Each effector from the library was recruited to dCas9 using a slightly modified version of the Suntag recruitment system (Tanenbaum et al., Cell 2014, 159, 635-646, incorporated herein by reference in its entirety). The modified version included a Cas9 protein fused to repeats of a GCN4 peptide epitope, a gRNA targeting the Cas9 to the target gene, and an antibody to the epitope fused to one effector from the library with the setup ScFV-sfBFP-[EFFECTOR]. For this experiment, the target gene was B2M. Lentivirus encoding the library was produced in 293T cells and titered based on sfBFP fluorescence of a dilution series in the cell type used in the screen. Cells were then transduced at a minimum of 200-fold coverage (600,000 cells for 3000 effectors). Cells were cultured for 10 days after transduction with the library. Cells were then subjected to fluorescence-activated cell sorting, and the top and bottom 10% by antibody staining for the target protein (B2M) were collected. Genomic DNA was purified, and the barcode cassette was amplified and sequenced on an Illumina MiSeq (San Diego, CA) to generate Log 2Fold Change and P-values. Calculations were performed by first summing all mapped barcodes for each effector in each condition. The gRNAs used are shown in TABLE 3.

TABLE 3
gRNA sequences.

| Target | DNA | RNA |
|---|---|---|
| B2M | GGGCCAGTCTGCAAAGCGAG | GGGCCAGUCUGCAAAGCGAG |
| | (SEQ ID NO: 93) | (SEQ ID NO: 96) |
| CD25 | TTATGGGCGTAGCTGAAGAA | UUAUGGGCGUAGCUGAAGAA |
| | (SEQ ID NO: 94) | (SEQ ID NO: 97) |
| Non-targeting | GTATGGAGGGCTGGATCTGC | GUAUGGAGGGCUGGAUCUGC |
| | (SEQ ID NO: 95) | (SEQ ID NO: 98) |

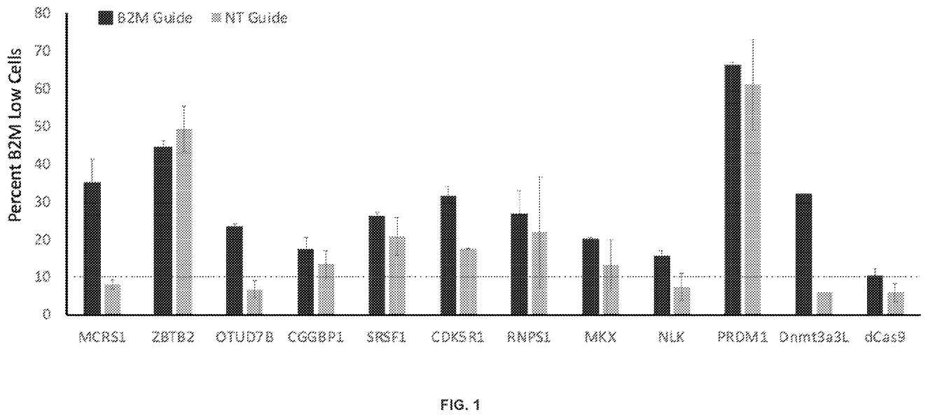

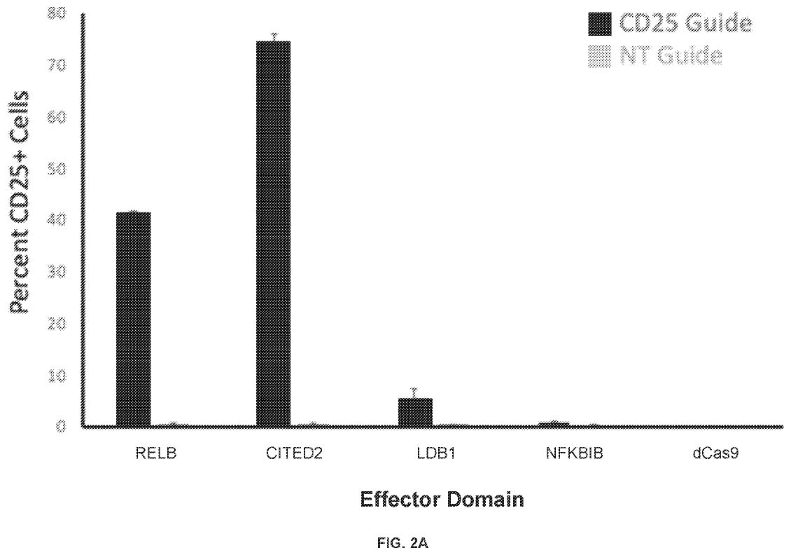

Novel effectors were discovered that activate or repress gene expression when recruited via dCas9 to a gene of interest. Hits shown in FIG. 1 were cloned individually, and lentivirus was produced. 293T cells encoding dCas9 and either a B2M-targeting gRNA or non-targeting gRNA were each transduced in duplicate. Cells were cultured for 10 days after transduction with the library. Cells were then stained for B2M and analyzed by flow cytometry.

The effectors resulting in significant increased or decreased expression of B2M with the targeting gRNA but not with the non-targeting gRNA included MCRS1, OTUD7B, RelB, LDB1, NFKBIB, and CITED2. Two novel hits were discovered in the first screen, MCRS1 and OTUD7B. Both appeared to repress gene expression when recruited to dCas9 at a target gene promoter, and both have not previously been used as dCas9 fusions. OTUD7B (also known as Cezanne) is a de-ubiquitinase which has previously been shown to be involved in DNA repair but not to repress gene expression (Mevissen et al, Nature 2016, 538, 402-405, incorporated herein by reference in its entirety). MCRS1 has been shown to bind the DAXX repressor, which may explain its repressive effect (Lin, D. Y. et al. J. Biol. Chem. 2002, 277, 25446-25456, incorporated herein by reference in its entirety).

FIG. 1 shows the percent of cells in the low B2M bin, with higher numbers suggesting more potent repression. The results shown in FIG. 1 were based on fold changes and p-values for all tested effectors targeted to B2M in 293T cells (TABLE 1). Cells were screened by B2M staining in flow cytometry, and fold changes were calculated between barcode counts recovered from cells collected in the top or bottom 10% B2M expression. A non-targeting guide was also included as a control for non-specific repression. MCRS1 and OTUD7B both showed repression that is both greater than the steric effects of dCas9 alone and largely dependent on dCas9 targeting, rather than a non-specific effect. Although other effectors also repressed B2M as predicted from the screen, these effects appeared to be non-specific.

### Example 2

### Effector Screen 2: Suntag System and CD25 Expression

A second screening experiment as detailed in Example 1 was completed, except examining CD25 expression instead of B2M, and these further experiments were completed to determine the fold changes and p-values for all tested effectors targeted to CD25 in Jurkat cells (TABLE 2). Cells were screened by CD25 staining in flow cytometry, and fold changes were calculated between barcode counts recovered from cells collected in the top or bottom 10%. Jurkat cell lines were generated by first transducing with lentiviral vectors encoding an sgRNA and dCas9 fused to a gcn4 peptide array that recruits the effector. A cell line with a CD25 targeting guide or a non-targeting guide was generated. These cell lines were then transduced with the indicated effectors fused to an scFv for recruitment to dCas9 (Tanenbaum et al., Cell 2014, 159, 635-646, incorporated herein by reference in its entirety).

Cells were cultured for 7 days after transduction with effector virus and stained for CD25 expression using a CD25 Monoclonal Antibody (BC96, PE-Cyanine7, eBioscience™, San Diego, CA). Only cells positive for the BFP fluorophore associated with the effector virus were included in the analysis of positive cells.

Shown in FIG. 2A is the level of CD25 activation after delivery of each effector domain recruited by dCas9 in Jurkat cells. A non-targeting guide (gray bars) showed no effect on CD25, suggesting that each effector is specifically activating CD25 upon recruitment by dCas9. Shown in FIG. 2B is a zoomed-in view of data in FIG. 2A to show the specific activation by LDB1 and NFKBIB.

### Example 3

### Effector Screen 3: High-Throughput TetO-GFP Screen

A cell line was constructed for use in a TetO-GFP reporter screen. 293T cells were first transduced with dCas9-GCN4, which recruited the ScFv fused to an effector, and subjected to blast selection (5 μg/mL). These cells were then transduced with lentivirus encoding a minimal CMV promoter driving GFP expression and flanked by 7 repeats of the Tet operator. Clonal cell lines were generated by plating of a limiting dilution in a 96-well plate. Twelve clonal cell lines were then tested for robust GFP induction upon delivery of ScFv-VPR (a known positive control), and the clone with the highest fold induction was chosen for the screen. This cell line was then transduced with lentivirus encoding both the TetO targeting and non-targeting (negative control) sgRNA along with iRFP. These transduced cells were then sorted for iRFP expression to generate pure populations expressing each sgRNA.

The TetO-GFP reporter cell lines (with either TetO targeting or non-targeting gRNA), were transduced at an MOI of 0.2 with lentivirus encoding the effector library. A total of 3.75 million cells were transduced with virus, giving 300-fold coverage (750,000 transductants) of the approximately 2500 effectors in the library. Cells were then cultured for three days, subjected to puromycin selection (0.5 μg/mL) for 3 days, and then allowed to expand for an additional 4 days before sorting the top 10% of GFP expressing cells. Genomic DNA was purified from the collected cells, the DNA encoding the effector barcodes was amplified by PCR, and the resulting amplicons were sequenced on an Illumina MiSeq (San Diego, CA). The barcode frequency in each sample was determined using custom python scripts, and the resulting barcode abundances were analyzed in the DESeq2 R package to calculate fold changes and p values between the input cells and the top 10% GFP expressing cells. This was performed for both the TetO-targeting gRNA and the non-targeting gRNA. The gRNAs used are shown in TABLE 4.

TABLE 4
gRNA sequences.

| Target | DNA | RNA |
|---|---|---|
| TetO | TACGTTCTCTATCACTGATA | UACGUUCUCUAUCACUGAUA |
| | (SEQ ID NO: 99) | (SEQ ID NO: 101) |
| Non-targeting | TATGGAGGGCTGGATCTGCG | UAUGGAGGGCUGGAUCUGCG |
| | (SEQ ID NO: 100) | (SEQ ID NO: 102) |

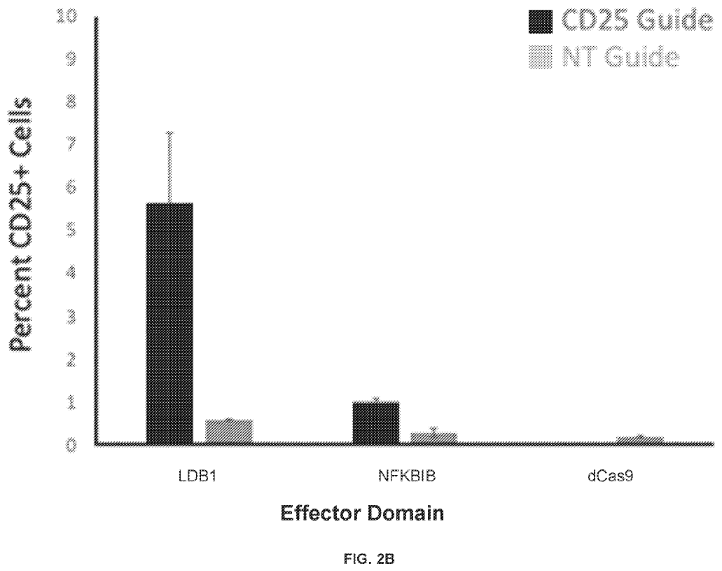

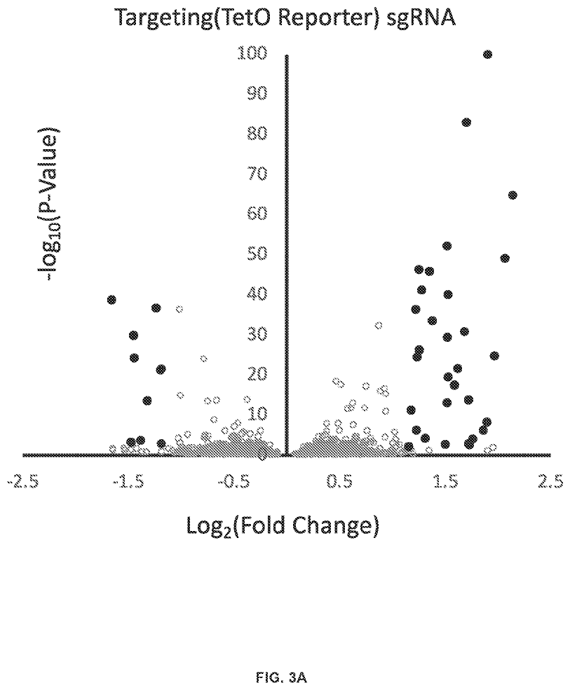

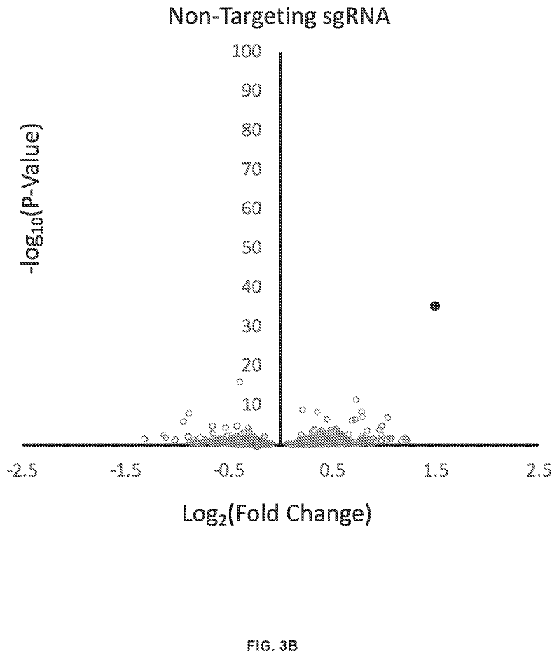

Shown in FIGS. 3A-3B are plots showing results for each effector in a screen for the ability to modulate GFP reporter expression. Log 2 (fold change) and Log 10 (Adjusted P Value) for each effector in the screen are plotted. Effectors with Log 2(fold change)>1.1 and Adjusted P Value <0.01 were considered to be hits and are shown in filled black circles, while non-hits are shown in open gray circles. This threshold gave 41 hits in the targeting condition and only 1 hit in the non-targeting condition, suggesting that it accurately filtered for legitimate hits. The 40 effector hits in the targeting condition that are not hits in the non-targeting (NT) condition included ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, which are disclosed herein as SEQ ID NOs: 103-176. These effectors showed significant increased or decreased expression of GFP with the targeting gRNA but not with the non-targeting gRNA.

### Example 4

### Effector Screen 4: Examining Subset of Effectors with TetO-GFP Reporter

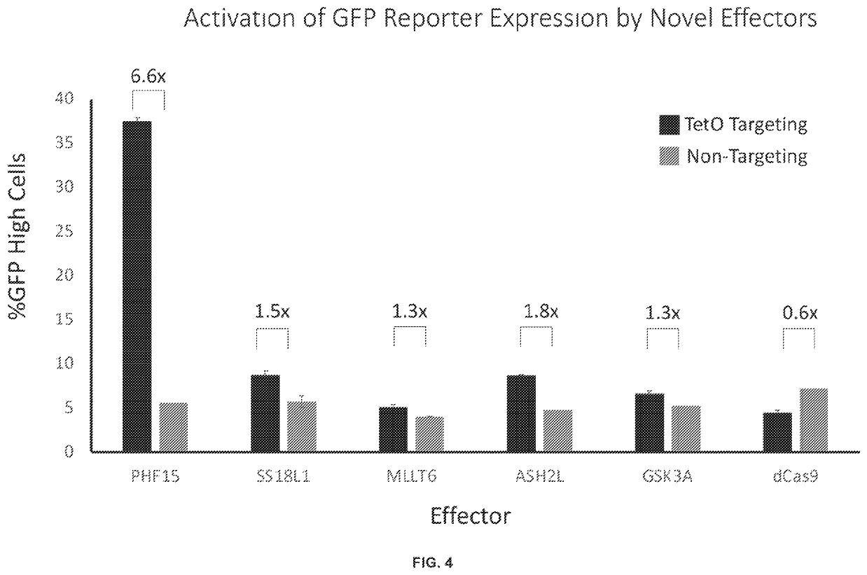

A subset of the effectors discovered as described in Example 3 was further examined using the same TetO-GFP reporter. As shown in FIG. 4, 293T cells containing a GFP reporter were transduced with Lentivirus encoding a subset of effectors (PHF15, SS18L1, MLLT6, ASH2L, and GSK3A) found to be hits in the high-throughput screen along with a targeting or non-targeting gRNA. The fold activation of GFP (shown above each pair of bars) was found to be greater than 1 for all effectors tested, while the dCas9 alone control showed the opposite trend, supporting the idea that even the small effects seen for some effectors are likely meaningful. All hit effectors tested did modulate GFP to some degree, suggesting that all effectors found to be hits in the high-throughput screen of Example 3 are likely to be modulators of gene expression.

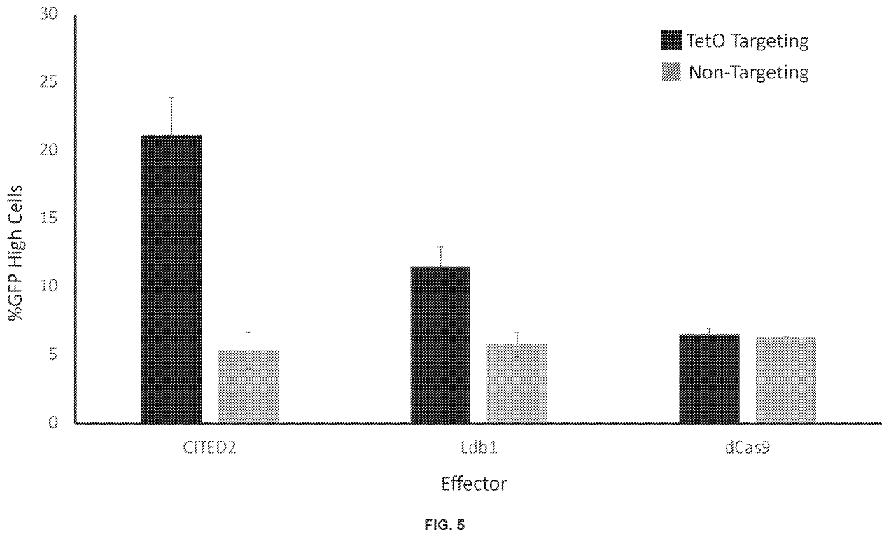

CITED2 and LDB1 were also examined for activation of GFP expression in 293T cells, with results shown in FIG. 5. 293T cells previously transduced with a TetO-GFP reporter were transfected with the indicated effector. Both LDB1 and CITED2 were able to robustly activate GFP expression, demonstrating that activation by these effectors was not limited to CD25, as shown in Example 2.

### Example 5

### Effect of LDB1 Dimerization Domain on Activation of Gene Expression

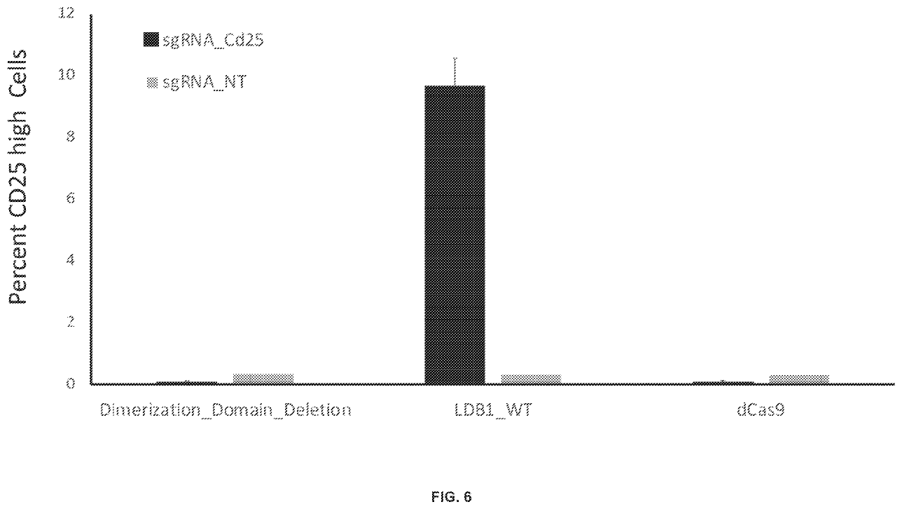

The LDB1 effector was examined using the CD25 expression system detailed in Example 2. Wild-type LDB1, as well as a mutant LDB1 with the dimerization domain deleted, were tested. Jurkat cells expressing dCas9-GCN4 and a CD25-targeting or non-targeting gRNA were transduced with the indicated effector-scFv fusion, and CD25 expression was analyzed by flow cytometry 10 days later. Results are shown in FIG. 6. Only the intact LDB1 effector was able to activate CD25. Activation of CD25 by LDB1 was dependent on the LDB1 dimerization domain. The dimerization domain deletion was a small deletion in the dimerization domain that was shown to be necessary for chromatin looping (Ivan Krivega, et al. Genes Dev. 2014, 28, 1278-90, incorporated herein by reference in its entirety), which suggested that LDB1 activated CD25 expression via a mechanism involving chromatin looping.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A Cas effector comprising: a first polypeptide comprising a Cas protein and at least one peptide epitope; and a second polypeptide comprising an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, and an antibody to the peptide epitope.

Clause 2. The Cas effector of clause 1, wherein the effector is selected from MCRS1, OTUD7B, RelB, LDB1, NFKBIB, CITED2, PHF15, SS18L1, MLLT6, ASH2L, and GSK3A, or a combination thereof.

Clause 3. The Cas effector of clause 1 or 2, wherein the effector is capable of increasing or decreasing expression of a gene.

Clause 4. The Cas effector of clause 3, wherein the effector reduces expression of a target gene and is selected from MCRS1, OTUD7B, ZNF133, ZNF140, ZNF169, ZNF254, ZNF566, ZNF585A, ZNF689, ZNF765, and ZNF81, or a combination thereof.

Clause 5. The Cas effector of clause 3, wherein the effector increases expression of a target gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof.

Clause 6. The Cas effector of any one of clauses 1-5, wherein the first polypeptide comprises about 2 to about 50 peptide epitopes.

Clause 7. The Cas effector of any one of clauses 1-6, wherein the first polypeptide comprises more than one copy of the peptide epitope and further comprises at least one linker in between adjacent copies of the peptide epitope.

Clause 8. The Cas effector of any one of clauses 1-7, wherein the peptide epitope is GCN4 and comprises the amino acid sequence of SEQ ID NO: 85.

Clause 9. The Cas effector of any one of clauses 1-8, wherein the first polypeptide comprises at least one peptide epitope at the N-terminus and/or at the C-terminus of the Cas protein.

Clause 10. The Cas effector of any one of clauses 1-9, wherein the first polypeptide comprises an amino acid sequence having at least 80%, 85%, 90%, 95%, or 98% or greater identity to SEQ ID NO: 87 or 89, or any fragment thereof, or wherein the first polypeptide comprises an amino acid sequence having one, two, three, four, five or more changes selected from amino acid substitutions, insertions, or deletions, relative to SEQ ID NO: 87 or 89, or any fragment thereof, or wherein the first polypeptide comprises the amino acid sequence of SEQ ID NO: 87 or 89.

Clause 11. The Cas effector of any one of clauses 1-10, wherein the antibody comprises the amino acid sequence of SEQ ID NO: 81.

Clause 12. The Cas effector of any one of clauses 1-11, wherein the second polypeptide comprises an amino acid sequence having at least 80%, 85%, 90%, 95%, or 98% or greater identity to a sequence selected from SEQ ID NOs: 69, 71, 73, 75, 77, and 79, or any fragment thereof, or wherein the second polypeptide comprises an amino acid sequence having one, two, three, four, five or more changes selected from amino acid substitutions, insertions, or deletions, relative to a sequence selected from SEQ ID NOs: 69, 71, 73, 75, 77, and 79, or any fragment thereof, or wherein the second polypeptide comprises an amino acid sequence selected from SEQ ID NOs: 69, 71, 73, 75, 77, and 79.

Clause 13. A Cas fusion protein comprising two heterologous polypeptide domains, wherein the first polypeptide domain comprises a Cas protein, and wherein the second polypeptide domain comprises an effector selected from MCRS1, OTUD7B, RelB, LDB1, NFKBIB, and CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof.

Clause 14. The Cas fusion protein of clause 13, wherein the effector is selected from MCRS1, OTUD7B, RelB, LDB1, NFKBIB, CITED2, PHF15, SS18L1, MLLT6, ASH2L, and GSK3A, or a combination thereof.

Clause 15. The Cas fusion protein of clause 13 or 14, wherein the effector is capable of increasing or decreasing expression of a gene.

Clause 16. The Cas fusion protein of clause 15, wherein the effector reduces expression of a target gene and is selected from MCRS1, OTUD7B, ZNF133, ZNF140, ZNF169, ZNF254, ZNF566, ZNF585A, ZNF689, ZNF765, and ZNF81, or a combination thereof.

Clause 17. The Cas fusion protein of clause 15, wherein the effector increases expression of a target gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof.

Clause 18. The Cas fusion protein of any one of clauses 13-17, wherein the second polypeptide domain has transcription repression activity, transcription activation activity, de-ubiquitinase activity, p300 recruitment activity, enhancer looping mediation activity, or a combination thereof.

Clause 19. The Cas effector of any one of clauses 1-12 or the Cas fusion protein of any one of clauses 13-18, wherein the MCRS1 comprises an amino acid sequence having at least 80%, 85%, 90%, 95%, or 98% or greater identity to SEQ ID NO: 57 or any fragment thereof, and/or wherein the MCRS1 comprises an amino acid sequence having one, two, three, four, five or more changes selected from amino acid substitutions, insertions, or deletions, relative to SEQ ID NO: 57, or any fragment thereof, and/or wherein the MCRS1 comprises the amino acid sequence of SEQ ID NO: 57, and/or wherein the MCRS1 is encoded by a polynucleotide comprising a sequence having at least 80%, 85%, 90%, 95%, or 98% or greater identity to SEQ ID NO: 58, or any fragment thereof, and/or wherein the MCRS1 is encoded by a polynucleotide comprising a sequence having one, two, three, four, five or more changes selected from nucleotide substitutions, insertions, or deletions, relative to SEQ ID NO: 58, or any fragment thereof, and/or wherein the MCRS1 is encoded by a polynucleotide comprising the sequence of SEQ ID NO: 58.

Clause 20. The Cas effector of any one of clauses 1-12 or the Cas fusion protein of any one of clauses 13-18, wherein the OTUD7B comprises an amino acid sequence having at least 80%, 85%, 90%, 95%, or 98% or greater identity to a sequence selected from SEQ ID NO: 59, amino acids 167-440 of SEQ ID NO: 59, or amino acids 792-831 of SEQ ID NO: 59, or any fragment thereof, and/or wherein the OTUD7B comprises an amino acid sequence having one, two, three, four, five or more changes selected from amino acid substitutions, insertions, or deletions, relative to a sequence selected from SEQ ID NO: 59, amino acids 167-440 of SEQ ID NO: 59, or amino acids 792-831 of SEQ ID NO: 59, or any fragment thereof, and/or wherein the OTUD7B comprises the amino acid sequence selected from SEQ ID NO: 59, amino acids 167-440 of SEQ ID NO: 59, or amino acids 792-831 of SEQ ID NO: 59, and/or wherein the OTUD7B is encoded by a polynucleotide comprising a sequence having at least 80%, 85%, 90%, 95%, or 98% or greater identity to SEQ ID NO: 60, or any fragment thereof, and/or wherein the OTUD7B is encoded by a polynucleotide comprising a sequence having one, two, three, four, five or more changes selected from nucleotide substitutions, insertions, or deletions, relative to SEQ ID NO: 60, or any fragment thereof, and/or wherein the OTUD7B is encoded by a polynucleotide comprising the sequence of SEQ ID NO: 60.

Clause 21. The Cas effector of any one of clauses 1-12 or the Cas fusion protein of any one of clauses 13-18, wherein the RelB comprises an amino acid sequence having at least 80%, 85%, 90%, 95%, or 98% or greater identity to SEQ ID NO: 65, or any fragment thereof, and/or wherein the RelB comprises an amino acid sequence having one, two, three, four, five or more changes selected from amino acid substitutions, insertions, or deletions, relative to SEQ ID NO: 65, or any fragment thereof, and/or wherein the RelB comprises the amino acid sequence of SEQ ID NO: 65, and/or wherein the RelB is encoded by a polynucleotide comprising a sequence having at least 80%, 85%, 90%, 95%, or 98% or greater identity to SEQ ID NO: 66 or any fragment thereof, and/or wherein the RelB is encoded by a polynucleotide comprising a sequence having one, two, three, four, five or more changes selected from nucleotide substitutions, insertions, or deletions, relative to SEQ ID NO: 66, or any fragment thereof, and/or wherein the RelB is encoded by a polynucleotide comprising the sequence of SEQ ID NO: 66.

Clause 22. The Cas effector of any one of clauses 1-12 or the Cas fusion protein of any one of clauses 13-18, wherein the LDB1 comprises an amino acid sequence having at least 80%, 85%, 90%, 95%, or 98% or greater identity to SEQ ID NO: 61, or any fragment thereof, and/or wherein the LDB1 comprises an amino acid sequence having one, two, three, four, five or more changes selected from amino acid substitutions, insertions, or deletions, relative to SEQ ID NO: 61, or any fragment thereof, and/or wherein the LDB1 comprises the amino acid sequence of SEQ ID NO: 61, and/or wherein the LDB1 is encoded by a polynucleotide comprising a sequence having at least 80%, 85%, 90%, 95%, or 98% or greater identity to SEQ ID NO: 62, or any fragment thereof, and/or wherein the LDB1 is encoded by a polynucleotide comprising a sequence having one, two, three, four, five or more changes selected from nucleotide substitutions, insertions, or deletions, relative to SEQ ID NO: 62, or any fragment thereof, and/or wherein the LDB1 is encoded by a polynucleotide comprising the sequence of SEQ ID NO: 62.

Clause 23. The Cas effector of any one of clauses 1-12 or the Cas fusion protein of any one of clauses 13-18, wherein the NFKBIB comprises an amino acid sequence having at least 80%, 85%, 90%, 95%, or 98% or greater identity to SEQ ID NO: 63, or any fragment thereof, and/or wherein the NFKBIB comprises an amino acid sequence having one, two, three, four, five or more changes selected from amino acid substitutions, insertions, or deletions, relative to SEQ ID NO: 63, or any fragment thereof, and/or wherein the NFKBIB comprises the amino acid sequence of SEQ ID NO: 63, and/or wherein the NFKBIB is encoded by a polynucleotide comprising a sequence having at least 80%, 85%, 90%, 95%, or 98% or greater identity to SEQ ID NO: 64, or any fragment thereof, and/or wherein the NFKBIB is encoded by a polynucleotide comprising a sequence having one, two, three, four, five or more changes selected from nucleotide substitutions, insertions, or deletions, relative to SEQ ID NO: 64, or any fragment thereof, and/or wherein the NFKBIB is encoded by a polynucleotide comprising the sequence of SEQ ID NO: 64.

Clause 24. The Cas effector of any one of clauses 1-12 or the Cas fusion protein of any one of clauses 13-18, wherein the CITED2 comprises an amino acid sequence having at least 80%, 85%, 90%, 95%, or 98% or greater identity to SEQ ID NO: 67, or any fragment thereof, and/or wherein the CITED2 comprises an amino acid sequence having one, two, three, four, five or more changes selected from amino acid substitutions, insertions, or deletions, relative to SEQ ID NO: 67, or any fragment thereof, and/or wherein the CITED2 comprises the amino acid sequence of SEQ ID NO: 67, and/or wherein the CITED2 is encoded by a polynucleotide comprising a sequence having at least 80%, 85%, 90%, 95%, or 98% or greater identity to SEQ ID NO: 68, or any fragment thereof, and/or wherein the CITED2 is encoded by a polynucleotide comprising a sequence having one, two, three, four, five or more changes selected from nucleotide substitutions, insertions, or deletions, relative to SEQ ID NO: 68, or any fragment thereof, and/or wherein the CITED2 is encoded by a polynucleotide comprising the sequence of SEQ ID NO: 68.

Clause 25. The Cas effector of any one of clauses 1-12 or the Cas fusion protein of any one of clauses 13-18, wherein the PHF15 comprises an amino acid sequence having at least 80%, 85%, 90%, 95%, or 98% or greater identity to SEQ ID NO: 133, or any fragment thereof, and/or wherein the PHF15 comprises an amino acid sequence having one, two, three, four, five or more changes selected from amino acid substitutions, insertions, or deletions, relative to SEQ ID NO: 133, or any fragment thereof, and/or wherein the PHF15 comprises the amino acid sequence of SEQ ID NO: 133, and/or wherein the PHF15 is encoded by a polynucleotide comprising a sequence having at least 80%, 85%, 90%, 95%, or 98% or greater identity to SEQ ID NO: 134, or any fragment thereof, and/or wherein the PHF15 is encoded by a polynucleotide comprising a sequence having one, two, three, four, five or more changes selected from nucleotide substitutions, insertions, or deletions, relative to SEQ ID NO: 134, or any fragment thereof, and/or wherein the PHF15 is encoded by a polynucleotide comprising the sequence of SEQ ID NO: 134.

Clause 26. The Cas effector of any one of clauses 1-12 or the Cas fusion protein of any one of clauses 13-18, wherein the SS18L1 comprises an amino acid sequence having at least 80%, 85%, 90%, 95%, or 98% or greater identity to SEQ ID NO: 149, or any fragment thereof, and/or wherein the SS18L1 comprises an amino acid sequence having one, two, three, four, five or more changes selected from amino acid substitutions, insertions, or deletions, relative to SEQ ID NO: 149, or any fragment thereof, and/or wherein the SS18L1 comprises the amino acid sequence of SEQ ID NO: 149, and/or wherein the SS18L1 is encoded by a polynucleotide comprising a sequence having at least 80%, 85%, 90%, 95%, or 98% or greater identity to SEQ ID NO: 150, or any fragment thereof, and/or wherein the SS18L1 is encoded by a polynucleotide comprising a sequence having one, two, three, four, five or more changes selected from nucleotide substitutions, insertions, or deletions, relative to SEQ ID NO: 150, or any fragment thereof, and/or wherein the SS18L1 is encoded by a polynucleotide comprising the sequence of SEQ ID NO: 150.

Clause 27. The Cas effector of any one of clauses 1-12 or the Cas fusion protein of any one of clauses 13-18, wherein the MLLT6 comprises an amino acid sequence having at least 80%, 85%, 90%, 95%, or 98% or greater identity to SEQ ID NO: 127, or any fragment thereof, and/or wherein the MLLT6 comprises an amino acid sequence having one, two, three, four, five or more changes selected from amino acid substitutions, insertions, or deletions, relative to SEQ ID NO: 127, or any fragment thereof, and/or wherein the MLLT6 comprises the amino acid sequence of SEQ ID NO: 127, and/or wherein the MLLT6 is encoded by a polynucleotide comprising a sequence having at least 80%, 85%, 90%, 95%, or 98% or greater identity to SEQ ID NO: 128, or any fragment thereof, and/or wherein the MLLT6 is encoded by a polynucleotide comprising a sequence having one, two, three, four, five or more changes selected from nucleotide substitutions, insertions, or deletions, relative to SEQ ID NO: 128, or any fragment thereof, and/or wherein the MLLT6 is encoded by a polynucleotide comprising the sequence of SEQ ID NO: 128.

Clause 28. The Cas effector of any one of clauses 1-12 or the Cas fusion protein of any one of clauses 13-18, wherein the ASH2L comprises an amino acid sequence having at least 80%, 85%, 90%, 95%, or 98% or greater identity to SEQ ID NO: 103, or any fragment thereof, and/or wherein the ASH2L comprises an amino acid sequence having one, two, three, four, five or more changes selected from amino acid substitutions, insertions, or deletions, relative to SEQ ID NO: 103, or any fragment thereof, and/or wherein the ASH2L comprises the amino acid sequence of SEQ ID NO: 103, and/or wherein the ASH2L is encoded by a polynucleotide comprising a sequence having at least 80%, 85%, 90%, 95%, or 98% or greater identity to SEQ ID NO: 104, or any fragment thereof, and/or wherein the ASH2L is encoded by a polynucleotide comprising a sequence having one, two, three, four, five or more changes selected from nucleotide substitutions, insertions, or deletions, relative to SEQ ID NO: 104, or any fragment thereof, and/or wherein the ASH2L is encoded by a polynucleotide comprising the sequence of SEQ ID NO: 104.

Clause 29. The Cas effector of any one of clauses 1-12 or the Cas fusion protein of any one of clauses 13-18, wherein the GSK3A comprises an amino acid sequence having at least 80%, 85%, 90%, 95%, or 98% or greater identity to SEQ ID NO: 117, or any fragment thereof, and/or wherein the GSK3A comprises an amino acid sequence having one, two, three, four, five or more changes selected from amino acid substitutions, insertions, or deletions, relative to SEQ ID NO: 117, or any fragment thereof, and/or wherein the GSK3A comprises the amino acid sequence of SEQ ID NO: 117, and/or wherein the GSK3A is encoded by a polynucleotide comprising a sequence having at least 80%, 85%, 90%, 95%, or 98% or greater identity to SEQ ID NO: 118, or any fragment thereof, and/or wherein the GSK3A is encoded by a polynucleotide comprising a sequence having one, two, three, four, five or more changes selected from nucleotide substitutions, insertions, or deletions, relative to SEQ ID NO: 118, or any fragment thereof, and/or wherein the GSK3A is encoded by a polynucleotide comprising the sequence of SEQ ID NO: 118.

Clause 30. The Cas effector of any one of clauses 1-12 or the Cas fusion protein of any one of clauses 13-18, wherein the effector is selected from BCL7B, C20orf20, DMAP1, DYRK1B, EAF1, FOXR2, JAZF1, KAT7, KEAP1, MEAF6, MORF4L2, NFYC, PKIB, POLE4, PRKRIR, PYGO2, RANBP1, RPRD1B, SPIN1, TADA3, TAF6, TBPL1, VPS72, ZNF133, ZNF140, ZNF169, ZNF254, ZNF566, ZNF585A, ZNF689, ZNF765, and ZNF81, and wherein the effector comprises an amino acid sequence having at least 80%, 85%, 90%, 95%, or 98% or greater identity to a sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175, or any fragment thereof, and/or wherein the effector comprises an amino acid sequence having one, two, three, four, five or more changes selected from amino acid substitutions, insertions, or deletions, relative to a sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175, or any fragment thereof, and/or wherein the effector comprises an amino acid sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175, and/or wherein the effector is encoded by a polynucleotide comprising a sequence having at least 80%, 85%, 90%, 95%, or 98% or greater identity to a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176, or any fragment thereof, and/or wherein the effector is encoded by a polynucleotide comprising a sequence having one, two, three, four, five or more changes selected from nucleotide substitutions, insertions, or deletions, relative to a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176, or any fragment thereof, and/or wherein the effector is encoded by a polynucleotide comprising a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176.

Clause 31. The Cas effector of any one of clauses 1-12 and 19-31 or the Cas fusion protein of clause any one of clauses 13-31, wherein the Cas protein comprises at least one amino acid mutation that knocks out nuclease activity of the Cas protein.

Clause 32. The Cas effector or the Cas fusion protein of clause 31, wherein the at least one amino acid mutation is at least one of D10A and H840A.

Clause 33. The Cas effector of any one of clauses 1-12 and 19-32 or the Cas fusion protein of any one of clauses 13-32, wherein the Cas protein comprises an amino acid sequence having at least 80%, 85%, 90%, 95%, or 98% or greater identity to one of SEQ ID NOs: 26-29, or any fragment thereof, or wherein the Cas protein comprises an amino acid sequence having one, two, three, four, five or more changes selected from amino acid substitutions, insertions, or deletions, relative to one of SEQ ID NOs: 26-29, or any fragment thereof, or wherein the Cas protein comprises the amino acid sequence of one of SEQ ID NOs: 26-29.

Clause 34. The Cas effector of any one of clauses 1-12 and 19-33 or the Cas fusion protein of any one of clauses 13-33, wherein the Cas protein is encoded by a polynucleotide comprising a sequence having at least 80%, 85%, 90%, 95%, or 98% or greater identity to one of SEQ ID NOs: 30-31, or any fragment thereof, or wherein the Cas protein is encoded by a polynucleotide comprising a sequence having one, two, three, four, five or more changes selected from nucleotide substitutions, insertions, or deletions, relative to one of SEQ ID NOs: 30-31, or any fragment thereof, or wherein the Cas protein is encoded by a polynucleotide comprising the sequence of one of SEQ ID NOs: 30-31.

Clause 35. A DNA targeting composition comprising: the Cas effector of any one of clauses 1-12 and 19-34 or the Cas fusion protein of any one of clauses 13-34; and at least one guide RNA (gRNA) that targets the Cas protein to a target region of a target gene.

Clause 36. The DNA targeting composition of clause 35, wherein the gRNA targets the Cas protein to target region selected from a non-open chromatin region, an open chromatin region, a transcribed region of the target gene, a region upstream of a transcription start site of the target gene, a regulatory element of the target gene, an intron of the target gene, or an exon of the target gene.

Clause 37. The DNA targeting composition of clause 35 or 36, wherein the gRNA targets the Cas protein to a promoter of the target gene.

Clause 38. The DNA targeting composition of any one of clauses 35-37, wherein the target region is located between about 1 to about 1000 base pairs upstream of a transcription start site of the target gene.

Clause 39. The DNA targeting composition of any one of clauses 35-38, wherein the at least one gRNA comprises a sequence selected from SEQ ID NOs: 96-98 and 101-102, or wherein the at least one gRNA is encoded by a polynucleotide comprising a sequence selected from SEQ ID NOs: 93-95 and 99-100, or wherein the at least one gRNA targets and binds a polynucleotide comprising a sequence selected from SEQ ID NOs: 93-95 and 99-100 or a complement thereof, or a combination thereof.

Clause 40. The DNA targeting composition of any one of clauses 35-39, wherein the DNA targeting composition comprises two or more gRNAs, each gRNA binding to a different target region.

Clause 41. An isolated polynucleotide sequence encoding the Cas effector of any one of clauses 1-12 and 19-34 or the Cas fusion protein of any one of clauses 13-34, or the DNA targeting composition of any one of clauses 35-40.

Clause 42. A vector comprising: the isolated polynucleotide sequence of clause 41.

Clause 43. The vector of clause 42, wherein the vector is an adeno-associated virus (AAV) vector.

Clause 44. A cell comprising: the Cas effector of any one of clauses 1-12 and 19-34 or the Cas fusion protein of any one of clauses 13-34, or the DNA targeting composition of any one of clauses 35-40, or the isolated polynucleotide sequence of clause 41, or the vector of clause 42 or 43, or a combination thereof.

Clause 45. A pharmaceutical composition comprising: the Cas effector of any one of clauses 1-12 and 19-34 or the Cas fusion protein of any one of clauses 13-34, or the DNA targeting composition of any one of clauses 35-40, or the isolated polynucleotide sequence of clause 41, or the vector of clause 42 or 43, or a combination thereof.

Clause 46. A method of modulating expression of a gene in a cell or in a subject, the method comprising administering to the cell or the subject the DNA targeting composition of any one of clauses 35-40, or the isolated polynucleotide sequence of clause 41, or the vector of clause 42 or 43, or the pharmaceutical composition of clause 45, or a combination thereof.

Clause 47. A method of modulating expression of a gene in a cell or in a subject, the method comprising administering to the cell or the subject an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, or a polynucleotide encoding the effector.

Clause 48. The method of clause 47, wherein the effector is targeted to the gene.

Clause 49. The method of clause 47 or 48, wherein the effector is selected from MCRS1, OTUD7B, RelB, LDB1, NFKBIB, CITED2, PHF15, SS18L1, MLLT6, ASH2L, and GSK3A, or a combination thereof.

Clause 50. The method of any one of clauses 47-49, wherein the effector is capable of increasing or decreasing expression of the gene.

Clause 51. The method of clause 50, wherein the effector reduces expression of the gene and is selected from MCRS1, OTUD7B, ZNF133, ZNF140, ZNF169, ZNF254, ZNF566, ZNF585A, ZNF689, ZNF765, and ZNF81, or a combination thereof.

Clause 52. The method of clause 50, wherein the effector increases expression of the gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof.

Clause 53. The method of any one of clauses 46-50 and 52, wherein the expression of the gene is increased relative to a control.

Clause 54. The method of any one of clauses 46-51, wherein the expression of the gene is decreased relative to a control.

Clause 55. The method of any one of clauses 46-54, wherein the gene comprises the dystrophin gene, the CD25 gene, the B2M gene, or the TRAC gene.

Clause 56. The method of any one of clauses 46-55, wherein the cell is a muscle cell or a T cell.

Clause 57. A method of treating a disease in a subject, the method comprising administering to the subject the DNA targeting composition of any one of clauses 35-40, or the isolated polynucleotide sequence of clause 41, or the vector of clause 42 or 43, or the cell of clause 44, or the pharmaceutical composition of clause 45, or a combination thereof.

Clause 58. A method of treating a disease in a subject, the method comprising administering to the subject an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, or a polynucleotide encoding the effector.

Clause 59. The method of clause 58, wherein the effector is targeted to a gene.

Clause 60. The method of any one of clauses 46-59, wherein the method treats a disease selected from Duchenne muscular dystrophy (DMD), Becker muscular dystrophy (BMD), and cancer.

SEQUENCES

SEQ ID NO: 1
NRG (R = A or G; N can be any nucleotide residue, e.g., any of A, G, C, or T)
SEQ ID NO: 2
NGG (N can be any nucleotide residue, e.g., any of A, G, C, or T)
SEQ ID NO: 3
NAG (N can be any nucleotide residue, e.g., any of A, G, C, or T)
SEQ ID NO: 4
NGGNG (N can be any nucleotide residue, e.g., any of A, G, C, or T)
SEQ ID NO: 5
NNAGAAW (W = A or T; N can be any nucleotide residue, e.g., any of A, G, C, or T)
SEQ ID NO: 6
NAAR (R = A or G; N can be any nucleotide residue, e.g., any of A, G, C, or T)
SEQ ID NO: 7
NNGRR (R = A or G; N can be any nucleotide residue, e.g., any of A, G, C, or T)
SEQ ID NO: 8
NNGRRN (R = A or G; N can be any nucleotide residue, e.g., any of A, G, C, or T)
SEQ ID NO: 9
NNGRRT (R = A or G; N can be any nucleotide residue, e.g., any of A, G, C, or T)
SEQ ID NO: 10
NNGRRV (R = A or G; N can be any nucleotide residue, e.g., any of A, G, C, or T; V = A or
C or G)
SEQ ID NO: 11
NNNNGATT (N can be any nucleotide residue, e.g., any of A, G, C, or T)
SEQ ID NO: 12
NNNNGNNN (N can be any nucleotide residue, e.g., any of A, G, C, or T)
SEQ ID NO: 13
NGA (N can be any nucleotide residue, e.g., any of A, G, C, or T)
SEQ ID NO: 14
NNNRRT (R = A or G; N can be any nucleotide residue, e.g., any of A, G, C, or T)
SEQ ID NO: 15
ATTCCT
SEQ ID NO: 16
NGAN (N can be any nucleotide residue, e.g., any of A, G, C, or T)
SEQ ID NO: 17
NGNG (N can be any nucleotide residue, e.g., any of A, G, C, or T)
SEQ ID NO: 18
DNA sequence of the gRNA constant region
gtttaagagctatgctggaaacagcatagcaagtttaaataaggctagtccgttatcaacttgaaaaa
gtggcaccgagtcggtgc
SEQ ID NO: 19
RNA sequence of the gRNA constant region
guuuaagagcuaugcuggaaacagcauagcaaguuuaaauaaggcuaguccguuaucaacuugaaaaa
guggcaccgagucggugc
SEQ ID NO: 20
SV40 NLS (Pro-Lys-Lys-Lys-Arg-Lys-Val)
SEQ ID NO: 21
GS linker (Gly-Gly-Gly-Gly-Ser)ₙ, wherein n is an integer between 0 and 10
SEQ ID NO: 22
Gly-Gly-Gly-Gly-Gly
SEQ ID NO: 23
Gly-Gly-Ala-Gly-Gly
SEQ ID NO: 24
Gly-Gly-Gly-Gly-Ser-Ser-Ser
SEQ ID NO: 25
Gly-Gly-Gly-Gly-Ala-Ala-Ala
SEQ ID NO: 26
Streptococcus pyogenes Cas9
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNEDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKORTEDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITORKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKOLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 27
Staphylococcus aureus Cas9
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVOKAYHOLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
SEQ ID NO: 28
Streptococcus pyogenes Cas9 (with D10A)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESELVEEDKKHERHPIFGNIVDEVAYHEKYPTIY
HLRKKLVDSTDKADLRLIYLALAHMIKFRGHFLIEGDLNPDNSDVDKLFIQLVQTYNOLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKORTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 29
Streptococcus pyogenes Cas9 (with D10A, H840A)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTEDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKOLKEDYFKKIECFDSVEISGVEDRFNASLGTYHDLLKIIKDKDFLDNEEN
EDILEDIVLTLTLFEDREMIEERLKTYAHLFDDKVMKOLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKOLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 30
Polynucleotide sequence of D10A mutant of S. aureus Cas9

| | | | | | |
|---|---|---|---|---|---|
| atgaaaagga | actacattct | ggggctggcc | atcgggatta | caagcgtggg | gtatgggatt |
| attgactatg | aaacaaggga | cgtgatcgac | gcaggcgtca | gactgttcaa | ggaggccaac |
| gtggaaaaca | atgagggacg | gagaagcaag | aggggagcca | ggcgcctgaa | acgacggaga |
| aggcacagaa | tccagagggt | gaagaaactg | ctgttcgatt | acaacctgct | gaccgaccat |
| tctgagctga | gtggaattaa | tccttatgaa | gccagggtga | aaggcctgag | tcagaagctg |
| tcagaggaag | agttttccgc | agctctgctg | cacctggcta | agcgccgagg | agtgcataac |
| gtcaatgagg | tggaagagga | caccggcaac | gagctgtcta | caaaggaaca | gatctcacgc |
| aatagcaaag | ctctggaaga | gaagtatgtc | gcagagctgc | agctggaacg | gctgaagaaa |
| gatggcgagg | tgagagggtc | aattaatagg | ttcaagacaa | gcgactacgt | caaagaagcc |
| aagcagctgc | tgaaagtgca | gaaggcttac | caccagctgg | atcagagctt | catcgatact |
| tatatcgacc | tgctggagac | tcggagaacc | tactatgagg | gaccaggaga | agggagcccc |
| ttcggatgga | aagacatcaa | ggaatggtac | gagatgctga | tgggacattg | cacctatttt |
| ccagaagagc | tgagaagcgt | caagtacgct | tataacgcag | atctgtacaa | cgccctgaat |
| gacctgaaca | acctggtcat | caccagggat | gaaaacgaga | aactggaata | ctatgagaag |
| ttccagatca | tcgaaaacgt | gtttaagcag | aagaaaaagc | ctacactgaa | acagattgct |
| aaggagatcc | tggtcaacga | agaggacatc | aagggctacc | gggtgacaag | cactggaaaa |
| ccagagttca | ccaatctgaa | agtgtatcac | gatattaagg | acatcacagc | acggaaagaa |
| atcattgaga | acgccgaact | gctggatcag | attgctaaga | tcctgactat | ctaccagagc |
| tccgaggaca | tccaggaaga | gctgactaac | ctgaacagcg | agctgaccca | ggaagagatc |
| gaacagatta | gtaatctgaa | ggggtacacc | ggaacacaca | acctgtccct | gaaagctatc |
| aatctgattc | tggatgagct | gtggcataca | aacgacaatc | agattgcaat | ctttaaccgg |
| ctgaagctgg | tcccaaaaaa | ggtggacctg | agtcagcaga | aagagatccc | aaccacactg |
| gtggacgatt | tcattctgtc | acccgtggtc | aagcggagct | tcatccagag | catcaaagtg |
| atcaacgcca | tcatcaagaa | gtacggcctg | cccaatgata | tcattatcga | gctggctagg |
| gagaagaaca | gcaaggacgc | acagaagatg | atcaatgaga | tgcagaaacg | aaaccggcag |
| accaatgaac | gcattgaaga | gattatccga | actaccggga | aagagaacgc | aaagtacctg |
| attgaaaaaa | tcaagctgca | cgatatgcag | gagggaaagt | gtctgtattc | tctggaggcc |
| atccccctgg | aggacctgct | gaacaatcca | ttcaactacg | aggtcgatca | tattatcccc |
| agaagcgtgt | ccttcgacaa | ttcctttaac | aacaaggtgc | tggtcaagca | ggaagagaac |
| tctaaaaagg | gcaataggac | tcctttccag | tacctgtcta | gttcagattc | caagatctct |
| tacgaaacct | ttaaaaagca | cattctgaat | ctggccaaag | gaaagggccg | catcagcaag |
| accaaaaagg | agtacctgct | ggaagagcgg | gacatcaaca | gattctccgt | ccagaaggat |
| tttattaacc | ggaatctggt | ggacacaaga | tacgctactc | gcggcctgat | gaatctgctg |
| cgatcctatt | tccgggtgaa | caatctggat | gtgaaagtca | agtccatcaa | cggcgggttc |
| acatcttttc | tgaggcgcaa | atggaagttt | aaaaaggagc | gcaacaaagg | gtacaagcac |
| catgccgaag | atgctctgat | tatcgcaaat | gccgacttca | tctttaagga | gtggaaaaag |
| ctggacaaag | ccaagaaagt | gatggagaac | cagatgttcg | aagagaagca | ggccgaatct |
| atgcccgaaa | tcgagacaga | acaggagtac | aaggagattt | tcatcactcc | tcaccagatc |
| aagcatatca | aggatttcaa | ggactacaag | tactctcacc | gggtggataa | aaagcccaac |
| agagagctga | tcaatgacac | cctgtatagt | acaagaaaag | acgataaggg | gaataccctg |
| attgtgaaca | atctgaacgg | actgtacgac | aaagataatg | acaagctgaa | aaagctgatc |
| aacaaaagtc | ccgagaagct | gctgatgtac | caccatgatc | ctcagacata | tcagaaactg |
| aagctgatta | tggagcagta | cggcgacgag | aagaacccac | tgtataagta | ctatgaagag |
| actgggaact | acctgaccaa | gtatagcaaa | aaggataatg | gccccgtgat | caagaagatc |
| aagtactatg | ggaacaagct | gaatgcccat | ctggacatca | cagacgatta | ccctaacagt |
| cgcaacaagg | tggtcaagct | gtcactgaag | ccatacagat | tcgatgtcta | tctggacaac |
| ggcgtgtata | aatttgtgac | tgtcaagaat | ctggatgtca | tcaaaaagga | gaactactat |
| gaagtgaata | gcaagtgcta | cgaagaggct | aaaaagctga | aaaagattag | caaccaggca |
| gagttcatcg | cctcctttta | caacaacgac | ctgattaaga | tcaatggcga | actgtatagg |
| gtcatcgggg | tgaacaatga | tctgctgaac | cgcattgaag | tgaatatgat | tgacatcact |
| taccgagagt | atctggaaaa | catgaatgat | aagcgccccc | ctcgaattat | caaaacaatt |
| gcctctaaga | ctcagagtat | caaaaagtac | tcaaccgaca | ttctgggaaa | cctgtatgag |
| gtgaagagca | aaaagcaccc | tcagattatc | aaaaagggc | | |

SEQ ID NO: 31
Polynucleotide sequence of N580A mutant of S. aureus Cas9

| | | | | | |
|---|---|---|---|---|---|
| atgaaaagga | actacattct | ggggctggac | atcgggatta | caagcgtggg | gtatgggatt |
| attgactatg | aaacaaggga | cgtgatcgac | gcaggcgtca | gactgttcaa | ggaggccaac |
| gtggaaaaca | atgagggacg | gagaagcaag | aggggagcca | ggcgcctgaa | acgacggaga |
| aggcacagaa | tccagagggt | gaagaaactg | ctgttcgatt | acaacctgct | gaccgaccat |
| tctgagctga | gtggaattaa | tccttatgaa | gccagggtga | aaggcctgag | tcagaagctg |
| tcagaggaag | agttttccgc | agctctgctg | cacctggcta | agcgccgagg | agtgcataac |
| gtcaatgagg | tggaagagga | caccggcaac | gagctgtcta | caaaggaaca | gatctcacgc |
| aatagcaaag | ctctggaaga | gaagtatgtc | gcagagctgc | agctggaacg | gctgaagaaa |
| gatggcgagg | tgagagggtc | aattaatagg | ttcaagacaa | gcgactacgt | caaagaagcc |
| aagcagctgc | tgaaagtgca | gaaggcttac | caccagctgg | atcagagctt | catcgatact |
| tatatcgacc | tgctggagac | tcggagaacc | tactatgagg | gaccaggaga | agggagcccc |
| ttcggatgga | aagacatcaa | ggaatggtac | gagatgctga | tgggacattg | cacctatttt |
| ccagaagagc | tgagaagcgt | caagtacgct | tataacgcag | atctgtacaa | cgccctgaat |
| gacctgaaca | acctggtcat | caccagggat | gaaaacgaga | aactggaata | ctatgagaag |
| ttccagatca | tcgaaaacgt | gtttaagcag | aagaaaaagc | ctacactgaa | acagattgct |
| aaggagatcc | tggtcaacga | agaggacatc | aagggctacc | gggtgacaag | cactggaaaa |
| ccagagttca | ccaatctgaa | agtgtatcac | gatattaagg | acatcacagc | acggaaagaa |
| atcattgaga | acgccgaact | gctggatcag | attgctaaga | tcctgactat | ctaccagagc |
| tccgaggaca | tccaggaaga | gctgactaac | ctgaacagcg | agctgaccca | ggaagagatc |
| gaacagatta | gtaatctgaa | ggggtacacc | ggaacacaca | acctgtccct | gaaagctatc |
| aatctgattc | tggatgagct | gtggcataca | aacgacaatc | agattgcaat | ctttaaccgg |
| ctgaagctgg | tcccaaaaaa | ggtggacctg | agtcagcaga | aagagatccc | aaccacactg |
| gtggacgatt | tcattctgtc | acccgtggtc | aagcggagct | tcatccagag | catcaaagtg |
| atcaacgcca | tcatcaagaa | gtacggcctg | cccaatgata | tcattatcga | gctggctagg |
| gagaagaaca | gcaaggacgc | acagaagatg | atcaatgaga | tgcagaaacg | aaaccggcag |
| accaatgaac | gcattgaaga | gattatccga | actaccggga | aagagaacgc | aaagtacctg |
| attgaaaaaa | tcaagctgca | cgatatgcag | gagggaaagt | gtctgtattc | tctggaggcc |
| atccccctgg | aggacctgct | gaacaatcca | ttcaactacg | aggtcgatca | tattatcccc |
| agaagcgtgt | ccttcgacaa | ttcctttaac | aacaaggtgc | tggtcaagca | ggaagaggcc |
| tctaaaaagg | gcaataggac | tcctttccag | tacctgtcta | gttcagattc | caagatctct |
| tacgaaacct | ttaaaaagca | cattctgaat | ctggccaaag | gaaagggccg | catcagcaag |
| accaaaaagg | agtacctgct | ggaagagcgg | gacatcaaca | gattctccgt | ccagaaggat |
| tttattaacc | ggaatctggt | ggacacaaga | tacgctactc | gcggcctgat | gaatctgctg |
| cgatcctatt | tccgggtgaa | caatctggat | gtgaaagtca | agtccatcaa | cggcgggttc |
| acatcttttc | tgaggcgcaa | atggaagttt | aaaaaggagc | gcaacaaagg | gtacaagcac |
| catgccgaag | atgctctgat | tatcgcaaat | gccgacttca | tctttaagga | gtggaaaaag |
| ctggacaaag | ccaagaaagt | gatggagaac | cagatgttcg | aagagaagca | ggccgaatct |
| atgcccgaaa | tcgagacaga | acaggagtac | aaggagattt | tcatcactcc | tcaccagatc |
| aagcatatca | aggatttcaa | ggactacaag | tactctcacc | gggtggataa | aaagcccaac |
| agagagctga | tcaatgacac | cctgtatagt | acaagaaaag | acgataaggg | gaataccctg |
| attgtgaaca | atctgaacgg | actgtacgac | aaagataatg | acaagctgaa | aaagctgatc |
| aacaaaagtc | ccgagaagct | gctgatgtac | caccatgatc | ctcagacata | tcagaaactg |
| aagctgatta | tggagcagta | cggcgacgag | aagaacccac | tgtataagta | ctatgaagag |
| actgggaact | acctgaccaa | gtatagcaaa | aaggataatg | gccccgtgat | caagaagatc |
| aagtactatg | ggaacaagct | gaatgcccat | ctggacatca | cagacgatta | ccctaacagt |
| cgcaacaagg | tggtcaagct | gtcactgaag | ccatacagat | tcgatgtcta | tctggacaac |
| ggcgtgtata | aatttgtgac | tgtcaagaat | ctggatgtca | tcaaaaagga | gaactactat |
| gaagtgaata | gcaagtgcta | cgaagaggct | aaaaagctga | aaaagattag | caaccaggca |
| gagttcatcg | cctcctttta | caacaacgac | ctgattaaga | tcaatggcga | actgtatagg |
| gtcatcgggg | tgaacaatga | tctgctgaac | cgcattgaag | tgaatatgat | tgacatcact |
| taccgagagt | atctggaaaa | catgaatgat | aagcgccccc | ctcgaattat | caaaacaatt |
| gcctctaaga | ctcagagtat | caaaaagtac | tcaaccgaca | ttctgggaaa | cctgtatgag |
| gtgaagagca | aaaagcaccc | tcagattatc | aaaaagggc | | |

SEQ ID NO: 32
codon optimized polynucleotide encoding S. pyogenes Cas9

| | | | | | |
|---|---|---|---|---|---|
| atggataaaa | agtacagcat | cgggctggac | atcggtacaa | actcagtggg | gtgggccgtg |
| attacggacg | agtacaaggt | accctccaaa | aaatttaaag | tgctgggtaa | cacggacaga |
| cactctataa | agaaaaatct | tattggagcc | ttgctgttcg | actcaggcga | gacagccgaa |
| gccacaaggt | tgaagcggac | cgccaggagg | cggtatacca | ggagaaagaa | ccgcatatgc |
| tacctgcaag | aaatcttcag | taacgagatg | gcaaaggttg | acgatagctt | tttccatcgc |
| ctggaagaat | cctttcttgt | tgaggaagac | aagaagcacg | aacggcaccc | catctttggc |
| aatattgtcg | acgaagtggc | atatcacgaa | aagtacccga | ctatctacca | cctcaggaag |
| aagctggtgg | actctaccga | taaggcggac | ctcagactta | tttatttggc | actcgcccac |
| atgattaaat | ttagaggaca | tttcttgatc | gagggcgacc | tgaacccgga | caacagtgac |
| gtcgataagc | tgttcatcca | acttgtgcag | acctacaatc | aactgttcga | agaaaaccct |
| ataaatgctt | caggagtcga | cgctaaagca | atcctgtccg | cgcgcctctc | aaaatctaga |
| agacttgaga | atctgattgc | tcagttgccc | ggggaaaaga | aaaatggatt | gtttggcaac |
| ctgatcgccc | tcagtctcgg | actgacccca | aatttcaaaa | gtaacttcga | cctggccgaa |
| gacgctaagc | tccagctgtc | caaggacaca | tacgatgacg | acctcgacaa | tctgctggcc |
| cagattgggg | atcagtacgc | cgatctcttt | ttggcagcaa | agaacctgtc | cgacgccatc |
| ctgttgagcg | atatcttgag | agtgaacacc | gaaattacta | aagcacccct | tagcgcatct |
| atgatcaagc | ggtacgacga | gcatcatcag | gatctgaccc | tgctgaaggc | tcttgtgagg |
| caacagctcc | ccgaaaaata | caaggaaatc | ttctttgacc | agagcaaaaa | cggctacgct |
| ggctatatag | atggtggggc | cagtcaggag | gaattctata | aattcatcaa | gcccattctc |
| gagaaaatgg | acggcacaga | ggagttgctg | gtcaaactta | acagggagga | cctgctgcgg |
| aagcagcgga | cctttgacaa | cgggtctatc | ccccaccaga | ttcatctggg | cgaactgcac |
| gcaatcctga | ggaggcagga | ggatttttat | ccttttctta | aagataaccg | cgagaaaata |
| gaaaagattc | ttacattcag | gatcccgtac | tacgtgggac | ctctcgcccg | gggcaattca |
| cggtttgcct | ggatgacaag | gaagtcagag | gagactatta | caccttggaa | cttcgaagaa |
| gtggtggaca | agggtgcatc | tgcccagtct | ttcatcgagc | ggatgacaaa | ttttgacaag |
| aacctcccta | atgagaaggt | gctgcccaaa | cattctctgc | tctacgagta | ctttaccgtc |
| tacaatgaac | tgactaaagt | caagtacgtc | accgagggaa | tgaggaagcc | ggcattcctt |
| agtggagaac | agaagaaggc | gattgtagac | ctgttgttca | agaccaacag | gaaggtgact |
| gtgaagcaac | ttaaagaaga | ctactttaag | aagatcgaat | gttttgacag | tgtggaaatt |
| tcaggggttg | aagaccgctt | caatgcgtca | ttggggactt | accatgatct | tctcaagatc |
| ataaaggaca | aagacttcct | ggacaacgaa | gaaaatgagg | atattctcga | agacatcgtc |
| ctcaccctga | ccctgttcga | agacagggaa | atgatagaag | agcgcttgaa | aacctatgcc |
| cacctcttcg | acgataaagt | tatgaagcag | ctgaagcgca | ggagatacac | aggatgggga |
| agattgtcaa | ggaagctgat | caatggaatt | agggataaac | agagtggcaa | gaccatactg |
| gatttcctca | aatctgatgg | cttcgccaat | aggaacttca | tgcaactgat | tcacgatgac |
| tctcttacct | tcaaggagga | cattcaaaag | gctcaggtga | gcgggcaggg | agactccctt |
| catgaacaca | tcgcgaattt | ggcaggttcc | cccgctatta | aaaagggcat | ccttcaaact |
| gtcaaggtgg | tggatgaatt | ggtcaaggta | atgggcagac | ataagccaga | aaatattgtg |
| atcgagatgg | cccgcgaaaa | ccagaccaca | cagaagggcc | agaaaaatag | tagagagcgg |
| atgaagagga | tcgaggaggg | catcaaagag | ctgggatctc | agattctcaa | agaacacccc |
| gtagaaaaca | cacagctgca | gaacgaaaaa | ttgtacttgt | actatctgca | gaacggcaga |
| gacatgtacg | tcgaccaaga | acttgatatt | aatagactgt | ccgactatga | cgtagaccat |
| atcgtgcccc | agtccttcct | gaaggacgac | tccattgata | acaaagtctt | gacaagaagc |
| gacaagaaca | ggggtaaaag | tgataatgtg | cctagcgagg | aggtggtgaa | aaaaatgaag |
| aactactggc | gacagctgct | taatgcaaag | ctcattacac | aacggaagtt | cgataatctg |
| acgaaagcag | agagaggtgg | cttgtctgag | ttggacaagg | cagggtttat | taagcggcag |
| ctggtggaaa | ctaggcagat | cacaaagcac | gtggcgcaga | ttttggacag | ccggatgaac |
| acaaaatacg | acgaaaatga | taaactgata | cgagaggtca | aagttatcac | gctgaaaagc |
| aagctggtgt | ccgattttcg | gaaagacttc | cagttctaca | aagttcgcga | gattaataac |
| taccatcatg | ctcacgatgc | gtacctgaac | gctgttgtcg | ggaccgcctt | gataaagaag |
| tacccaaagc | tggaatccga | gttcgtatac | ggggattaca | aagtgtacga | tgtgaggaaa |
| atgatagcca | agtccgagca | ggagattgga | aaggccacag | ctaagtactt | cttttattct |
| aacatcatga | atttttttaa | gacggaaatt | accctggcca | acggagagat | cagaaagcgg |
| ccccttatag | agacaaatgg | tgaaacaggt | gaaatcgtct | gggataaggg | cagggatttc |
| gctactgtga | ggaaggtgct | gagtatgcca | caggtaaata | tcgtgaaaaa | aaccgaagta |
| cagaccggag | gattttccaa | ggaaagcatt | ttgcctaaaa | gaaactcaga | caagctcatc |
| gcccgcaaga | aagattggga | ccctaagaaa | tacgggggat | ttgactcacc | caccgtagcc |
| tattctgtgc | tggtggtagc | taaggtggaa | aaaggaaagt | ctaagaagct | gaagtccgtg |
| aaggaactct | tgggaatcac | tatcatggaa | agatcatcct | ttgaaaagaa | ccctatcgat |
| ttcctggagg | ctaagggtta | caaggaggtc | aagaaagacc | tcatcattaa | actgccaaaa |
| tactctctct | tcgagctgga | aaatggcagg | aagagaatgt | tggccagcgc | cggagagctg |
| caaaagggaa | acgagcttgc | tctgccctcc | aaatatgtta | attttctcta | tctcgcttcc |
| cactatgaaa | agctgaaagg | gtctcccgaa | gataacgagc | agaagcagct | gttcgtcgaa |
| cagcacaagc | actatctgga | tgaaataatc | gaacaaataa | gcgagttcag | caaaagggtt |
| atcctggcgg | atgctaattt | ggacaaagta | ctgtctgctt | ataacaagca | ccgggataag |
| cctattaggg | aacaagccga | gaatataatt | cacctcttta | cactcacgaa | tctcggagcc |
| cccgccgcct | tcaaatactt | tgatacgact | atcgaccgga | aacggtatac | cagtaccaaa |
| gaggtcctcg | atgccaccct | catccaccag | tcaattactg | gcctgtacga | aacacggatc |
| gacctctctc | aactgggcgg | cgactag | | | |

SEQ ID NO: 33
codon optimized nucleic acid sequences encoding S. aureus Cas9

| | | | | | |
|---|---|---|---|---|---|
| atgaaaagga | actacattct | ggggctggac | atcgggatta | caagcgtggg | gtatgggatt |
| attgactatg | aaacaaggga | cgtgatcgac | gcaggcgtca | gactgttcaa | ggaggccaac |
| gtggaaaaca | atgagggacg | gagaagcaag | aggggagcca | ggcgcctgaa | acgacggaga |
| aggcacagaa | tccagagggt | gaagaaactg | ctgttcgatt | acaacctgct | gaccgaccat |
| tctgagctga | gtggaattaa | tccttatgaa | gccagggtga | aaggcctgag | tcagaagctg |
| tcagaggaag | agttttccgc | agctctgctg | cacctggcta | agcgccgagg | agtgcataac |
| gtcaatgagg | tggaagagga | caccggcaac | gagctgtcta | caaaggaaca | gatctcacgc |
| aatagcaaag | ctctggaaga | gaagtatgtc | gcagagctgc | agctggaacg | gctgaagaaa |
| gatggcgagg | tgagagggtc | aattaatagg | ttcaagacaa | gcgactacgt | caaagaagcc |
| aagcagctgc | tgaaagtgca | gaaggcttac | caccagctgg | atcagagctt | catcgatact |
| tatatcgacc | tgctggagac | tcggagaacc | tactatgagg | gaccaggaga | agggagcccc |
| ttcggatgga | aagacatcaa | ggaatggtac | gagatgctga | tgggacattg | cacctatttt |
| ccagaagagc | tgagaagcgt | caagtacgct | tataacgcag | atctgtacaa | cgccctgaat |
| gacctgaaca | acctggtcat | caccagggat | gaaaacgaga | aactggaata | ctatgagaag |
| ttccagatca | tcgaaaacgt | gtttaagcag | aagaaaaagc | ctacactgaa | acagattgct |
| aaggagatcc | tggtcaacga | agaggacatc | aagggctacc | gggtgacaag | cactggaaaa |
| ccagagttca | ccaatctgaa | agtgtatcac | gatattaagg | acatcacagc | acggaaagaa |
| atcattgaga | acgccgaact | gctggatcag | attgctaaga | tcctgactat | ctaccagagc |
| tccgaggaca | tccaggaaga | gctgactaac | ctgaacagcg | agctgaccca | ggaagagatc |
| gaacagatta | gtaatctgaa | ggggtacacc | ggaacacaca | acctgtccct | gaaagctatc |
| aatctgattc | tggatgagct | gtggcataca | aacgacaatc | agattgcaat | ctttaaccgg |
| ctgaagctgg | tcccaaaaaa | ggtggacctg | agtcagcaga | aagagatccc | aaccacactg |
| gtggacgatt | tcattctgtc | acccgtggtc | aagcggagct | tcatccagag | catcaaagtg |
| atcaacgcca | tcatcaagaa | gtacggcctg | cccaatgata | tcattatcga | gctggctagg |
| gagaagaaca | gcaaggacgc | acagaagatg | atcaatgaga | tgcagaaacg | aaaccggcag |
| accaatgaac | gcattgaaga | gattatccga | actaccggga | aagagaacgc | aaagtacctg |
| attgaaaaaa | tcaagctgca | cgatatgcag | gagggaaagt | gtctgtattc | tctggaggcc |
| tccccctgg | aggacctgct | gaacaatcca | ttcaactacg | aggtcgatca | tattatcccc |
| agaagcgtgt | ccttcgacaa | ttcctttaac | aacaaggtgc | tggtcaagca | ggaagagaac |
| tctaaaaagg | gcaataggac | tcctttccag | tacctgtcta | gttcagattc | caagatctct |
| tacgaaacct | ttaaaaagca | cattctgaat | ctggccaaag | gaaagggccg | catcagcaag |
| accaaaaagg | agtacctgct | ggaagagcgg | gacatcaaca | gattctccgt | ccagaaggat |
| tttattaacc | ggaatctggt | ggacacaaga | tacgctactc | gcggcctgat | gaatctgctg |
| cgatcctatt | tccgggtgaa | caatctggat | gtgaaagtca | agtccatcaa | cggcgggttc |
| acatcttttc | tgaggcgcaa | atggaagttt | aaaaaggagc | gcaacaaagg | gtacaagcac |
| catgccgaag | atgctctgat | tatcgcaaat | gccgacttca | tctttaagga | gtggaaaaag |
| ctggacaaag | ccaagaaagt | gatggagaac | cagatgttcg | aagagaagca | ggccgaatct |
| atgcccgaaa | tcgagacaga | acaggagtac | aaggagattt | tcatcactcc | tcaccagatc |
| aagcatatca | aggatttcaa | ggactacaag | tactctcacc | gggtggataa | aaagcccaac |
| agagagctga | tcaatgacac | cctgtatagt | acaagaaaag | acgataaggg | gaataccctg |
| attgtgaaca | atctgaacgg | actgtacgac | aaagataatg | acaagctgaa | aaagctgatc |
| aacaaaagtc | ccgagaagct | gctgatgtac | caccatgatc | ctcagacata | tcagaaactg |
| aagctgatta | tggagcagta | cggcgacgag | aagaacccac | tgtataagta | ctatgaagag |
| actgggaact | acctgaccaa | gtatagcaaa | aaggataatg | gccccgtgat | caagaagatc |
| aagtactatg | ggaacaagct | gaatgcccat | ctggacatca | cagacgatta | ccctaacagt |
| cgcaacaagg | tggtcaagct | gtcactgaag | ccatacagat | tcgatgtcta | tctggacaac |
| ggcgtgtata | aatttgtgac | tgtcaagaat | ctggatgtca | tcaaaaagga | gaactactat |
| gaagtgaata | gcaagtgcta | cgaagaggct | aaaaagctga | aaaagattag | caaccaggca |
| gagttcatcg | cctcctttta | caacaacgac | ctgattaaga | tcaatggcga | actgtatagg |
| gtcatcgggg | tgaacaatga | tctgctgaac | cgcattgaag | tgaatatgat | tgacatcact |
| taccgagagt | atctggaaaa | catgaatgat | aagcgccccc | ctcgaattat | caaaacaatt |
| gcctctaaga | ctcagagtat | caaaaagtac | tcaaccgaca | ttctgggaaa | cctgtatgag |
| gtgaagagca | aaaagcaccc | tcagattatc | aaaaagggc | | |

SEQ ID NO: 34
codon optimized nucleic acid sequences encoding S. aureus Cas9

| | | | | | |
|---|---|---|---|---|---|
| atgaagcgga | actacatcct | gggcctggac | atcggcatca | ccagcgtggg | ctacggcatc |
| atcgactacg | agacacggga | cgtgatcgat | gccggcgtgc | ggctgttcaa | agaggccaac |
| gtggaaaaca | acgagggcag | gcggagcaag | agaggcgcca | gaaggctgaa | gcggcggagg |
| cggcatagaa | tccagagagt | gaagaagctg | ctgttcgact | acaacctgct | gaccgaccac |
| agcgagctga | gcggcatcaa | cccctacgag | gccagagtga | agggcctgag | ccagaagctg |
| agcgaggaag | agttctctgc | cgccctgctg | cacctggcca | agagaagagg | cgtgcacaac |
| gtgaacgagg | tggaagagga | caccggcaac | gagctgtcca | ccaaagagca | gatcagccgg |
| aacagcaagg | ccctggaaga | gaaatacgtg | gccgaactgc | agctggaacg | gctgaagaaa |
| gacggcgaag | tgcggggcag | catcaacaga | ttcaagacca | gcgactacgt | gaaagaagcc |
| aaacagctgc | tgaaggtgca | gaaggcctac | caccagctgg | accagagctt | catcgacacc |
| tacatcgacc | tgctggaaac | ccggcggacc | tactatgagg | gacctggcga | gggcagcccc |
| ttcggctgga | aggacatcaa | agaatggtac | gagatgctga | tgggccactg | cacctacttc |
| cccgaggaac | tgcggagcgt | gaagtacgcc | tacaacgccg | acctgtacaa | cgccctgaac |
| gacctgaaca | atctcgtgat | caccagggac | gagaacgaga | agctggaata | ttacgagaag |
| ttccagatca | tcgagaacgt | gttcaagcag | aagaagaagc | ccaccctgaa | gcagatcgcc |
| aaagaaatcc | tcgtgaacga | agaggatatt | aagggctaca | gagtgaccag | caccggcaag |
| cccgagttca | ccaacctgaa | ggtgtaccac | gacatcaagg | acattaccgc | ccggaaagag |
| attattgaga | acgccgagct | gctggatcag | attgccaaga | tcctgaccat | ctaccagagc |
| agcgaggaca | tccaggaaga | actgaccaat | ctgaactccg | agctgaccca | ggaagagatc |
| gagcagatct | ctaatctgaa | gggctatacc | ggcacccaca | acctgagcct | gaaggccatc |
| aacctgatcc | tggacgagct | gtggcacacc | aacgacaacc | agatcgctat | cttcaaccgg |
| ctgaagctgg | tgcccaagaa | ggtggacctg | tcccagcaga | aagagatccc | caccaccctg |
| gtggacgact | tcatcctgag | ccccgtcgtg | aagagaagct | tcatccagag | catcaaagtg |
| atcaacgcca | tcatcaagaa | gtacggcctg | cccaacgaca | tcattatcga | gctggcccgc |
| gagaagaact | ccaaggacgc | ccagaaaatg | atcaacgaga | tgcagaagcg | gaaccggcag |
| accaacgagc | ggatcgagga | aatcatccgg | accaccggca | aagagaacgc | caagtacctg |
| atcgagaaga | tcaagctgca | cgacatgcag | gaaggcaagt | gcctgtacag | cctggaagcc |
| atccctctgg | aagatctgct | gaacaacccc | ttcaactatg | aggtggacca | catcatcccc |
| agaagcgtgt | ccttcgacaa | cagcttcaac | aacaaggtgc | tcgtgaagca | ggaagaaaac |
| agcaagaagg | gcaaccggac | cccattccag | tacctgagca | gcagcgacag | caagatcagc |
| tacgaaacct | tcaagaagca | catcctgaat | ctggccaagg | gcaagggcag | aatcagcaag |
| accaagaaag | agtatctgct | ggaagaacgg | gacatcaaca | ggttctccgt | gcagaaagac |
| ttcatcaacc | ggaacctggt | ggataccaga | tacgccacca | gaggcctgat | gaacctgctg |
| cggagctact | tcagagtgaa | caacctggac | gtgaaagtga | agtccatcaa | tggcggcttc |
| accagctttc | tgcggcggaa | gtggaagttt | aagaaagagc | ggaacaaggg | gtacaagcac |
| cacgccgagg | acgccctgat | cattgccaac | gccgatttca | tottcaaaga | gtggaagaaa |
| ctggacaagg | ccaaaaaagt | gatggaaaac | cagatgttcg | aggaaaagca | ggccgagagc |
| atgcccgaga | tcgaaaccga | gcaggagtac | aaagagatct | tcatcacccc | ccaccagatc |
| aagcacatta | aggacttcaa | ggactacaag | tacagccacc | gggtggacaa | gaagcctaat |
| agagagctga | ttaacgacac | cctgtactcc | acccggaagg | acgacaaggg | caacaccctg |
| atcgtgaaca | atctgaacgg | cctgtacgac | aaggacaatg | acaagctgaa | aaagctgatc |
| aacaagagcc | ccgaaaagct | gctgatgtac | caccacgacc | cccagaccta | ccagaaactg |
| aagctgatta | tggaacagta | cggcgacgag | aagaatcccc | tgtacaagta | ctacgaggaa |
| accgggaact | acctgaccaa | gtactccaaa | aaggacaacg | gccccgtgat | caagaagatt |
| aagtattacg | gcaacaaact | gaacgcccat | ctggacatca | ccgacgacta | ccccaacagc |
| agaaacaagg | tcgtgaagct | gtccctgaag | ccctacagat | tcgacgtgta | cctggacaat |
| ggcgtgtaca | agttcgtgac | cgtgaagaat | ctggatgtga | tcaaaaaaga | aaactactac |
| gaagtgaata | gcaagtgcta | tgaggaagct | aagaagctga | agaagatcag | caaccaggcc |
| gagtttatcg | cctccttcta | caacaacgat | ctgatcaaga | tcaacggcga | gctgtataga |
| gtgatcggcg | tgaacaacga | cctgctgaac | cggatcgaag | tgaacatgat | cgacatcacc |
| taccgcgagt | acctggaaaa | catgaacgac | aagaggcccc | ccaggatcat | taagacaatc |
| gcctccaaga | cccagagcat | taagaagtac | agcacagaca | ttctgggcaa | cctgtatgaa |
| gtgaaatcta | agaagcaccc | tcagatcatc | aaaaagggc | | |

SEQ ID NO: 35
codon optimized nucleic acid sequence encoding S. aureus Cas9

| | | | | | |
|---|---|---|---|---|---|
| atgaagcgca | actacatcct | cggactggac | atcggcatta | cctccgtggg | atacggcatc |
| atcgattacg | aaactaggga | tgtgatcgac | gctggagtca | ggctgttcaa | agaggcgaac |
| gtggagaaca | acgaggggcg | gcgctcaaag | aggggggccc | gccggctgaa | gcgccgccgc |
| agacatagaa | tccagcgcgt | gaagaagctg | ctgttcgact | acaaccttct | gaccgaccac |
| tccgaacttt | ccggcatcaa | cccatatgag | gctagagtga | agggattgtc | ccaaaagctg |
| tccgaggaag | agttctccgc | cgcgttgctc | cacctcgcca | agcgcagggg | agtgcacaat |
| gtgaacgaag | tggaagaaga | taccggaaac | gagctgtcca | ccaaggagca | gatcagccgg |
| aactccaagg | ccctggaaga | gaaatacgtg | gcggaactgc | aactggagcg | gctgaagaaa |
| gacggagaag | tgcgcggctc | gatcaaccgc | ttcaagacct | cggactacgt | gaaggaggcc |
| aagcagctcc | tgaaagtgca | aaaggcctat | caccaacttg | accagtcctt | tatcgatacc |
| tacatcgatc | tgctcgagac | tcggcggact | tactacgagg | gtccagggga | gggctcccca |
| tttggttgga | aggatattaa | ggagtggtac | gaaatgctga | tgggacactg | cacatacttc |
| cctgaggagc | tgcggagcgt | gaaatacgca | tacaacgcag | acctgtacaa | cgcgctgaac |
| gacctgaaca | atctcgtgat | cacccgggac | gagaacgaaa | agctcgagta | ttacgaaaag |
| ttccagatta | ttgagaacgt | gttcaaacag | aagaagaagc | cgacactgaa | gcagattgcc |
| aaggaaatcc | tcgtgaacga | agaggacatc | aagggctatc | gagtgacctc | aacgggaaag |
| ccggagttca | ccaatctgaa | ggtctaccac | gacatcaaag | acattaccgc | ccggaaggag |
| atcattgaga | acgcggagct | gttggaccag | attgcgaaga | ttctgaccat | ctaccaatcc |
| tccgaggata | ttcaggaaga | actcaccaac | ctcaacagcg | aactgaccca | ggaggagata |
| gagcaaatct | ccaacctgaa | gggctacacc | ggaactcata | acctgagcct | gaaggccatc |
| aacttgatcc | tggacgagct | gtggcacacc | aacgataacc | agatcgctat | tttcaatcgg |
| ctgaagctgg | tccccaagaa | agtggacctc | tcacaacaaa | aggagatccc | tactaccctt |
| gtggacgatt | tcattctgtc | ccccgtggtc | aagagaagct | tcatacagtc | aatcaaagtg |
| atcaatgcca | ttatcaagaa | atacggtctg | cccaacgaca | ttatcattga | gctcgcccgc |
| gagaagaact | cgaaggacgc | ccagaagatg | attaacgaaa | tgcagaagag | gaaccgacag |
| actaacgaac | ggatcgaaga | aatcatccgg | accaccggga | aggaaaacgc | gaagtacctg |
| atcgaaaaga | tcaagctcca | tgacatgcag | gaaggaaagt | gtctgtactc | gctggaggcc |
| attccgctgg | aggacttgct | gaacaaccct | tttaactacg | aagtggatca | tatcattccg |
| aggagcgtgt | cattcgacaa | ttccttcaac | aacaaggtcc | tcgtgaagca | ggaggaaaac |
| tcgaagaagg | gaaaccgcac | gccgttccag | tacctgagca | gcagcgactc | caagatttcc |
| tacgaaacct | tcaagaagca | catcctcaac | ctggcaaagg | ggaagggtcg | catctccaag |
| accaagaagg | aatatctgct | ggaagaaaga | gacatcaaca | gattctccgt | gcaaaaggac |
| ttcatcaacc | gcaacctcgt | ggatactaga | tacgctactc | ggggtctgat | gaacctcctg |
| agaagctact | ttagagtgaa | caatctggac | gtgaaggtca | agtcgattaa | cggaggtttc |
| acctccttcc | tgcggcgcaa | gtggaagttc | aagaaggaac | ggaacaaggg | ctacaagcac |
| cacgccgagg | acgccctgat | cattgccaac | gccgacttca | tottcaaaga | atggaagaaa |
| cttgacaagg | ctaagaaggt | catggaaaac | cagatgttcg | aagaaaagca | ggccgagtct |
| atgcctgaaa | tcgagactga | acaggagtac | aaggaaatct | ttattacgcc | acaccagatc |
| aaacacatca | aggatttcaa | ggattacaag | tactcacatc | gcgtggacaa | aaagccgaac |
| agggaactga | tcaacgacac | cctctactcc | acccggaagg | atgacaaagg | gaataccctc |
| atcgtcaaca | accttaacgg | cctgtacgac | aaggacaacg | ataagctgaa | gaagctcatt |
| aacaagtcgc | ccgaaaagtt | gctgatgtac | caccacgacc | ctcagactta | ccagaagctc |
| aagctgatca | tggagcagta | tggggacgag | aaaaacccgt | tgtacaagta | ctacgaagaa |
| actgggaatt | atctgactaa | gtactccaag | aaagataacg | gccccgtgat | taagaagatt |
| aagtactacg | gcaacaagct | gaacgcccat | ctggacatca | ccgatgacta | ccctaattcc |
| cgcaacaagg | tcgtcaagct | gagcctcaag | ccctaccggt | ttgatgtgta | ccttgacaat |
| ggagtgtaca | agttcgtgac | tgtgaagaac | cttgacgtga | tcaagaagga | gaactactac |
| gaagtcaact | ccaagtgcta | cgaggaagca | aagaagttga | agaagatctc | gaaccaggcc |
| gagttcattg | cctccttcta | taacaacgac | ctgattaaga | tcaacggcga | actgtaccgc |
| gtcattggcg | tgaacaacga | tctcctgaac | cgcatcgaag | tgaacatgat | cgacatcact |
| taccgggaat | acctggagaa | tatgaacgac | aagcgcccgc | cccggatcat | taagactatc |
| gcctcaaaga | cccagtcgat | caagaagtac | agcaccgaca | tcctgggcaa | cctgtacgag |
| gtcaaatcga | agaagcaccc | ccagatcatc | aagaaggga | | |

SEQ ID NO: 36
codon optimized nucleic acid sequence encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 37
codon optimized nucleic acid sequence encoding S. aureus Cas9

| | | | | | |
|---|---|---|---|---|---|
| accggtgcca | ccatgtaccc | atacgatgtt | ccagattacg | cttcgccgaa | gaaaaagcgc |
| aaggtcgaag | cgtccatgaa | aaggaactac | attctggggc | tggacatcgg | gattacaagc |
| gtggggtatg | ggattattga | ctatgaaaca | agggacgtga | tcgacgcagg | cgtcagactg |
| ttcaaggagg | ccaacgtgga | aaacaatgag | ggacggagaa | gcaagagggg | agccaggcgc |
| ctgaaacgac | ggagaaggca | cagaatccag | agggtgaaga | aactgctgtt | cgattacaac |
| ctgctgaccg | accattctga | gctgagtgga | attaatcctt | atgaagccag | ggtgaaaggc |
| ctgagtcaga | agctgtcaga | ggaagagttt | tccgcagctc | tgctgcacct | ggctaagcgc |
| cgaggagtgc | ataacgtcaa | tgaggtggaa | gaggacaccg | gcaacgagct | gtctacaaag |
| gaacagatct | cacgcaatag | caaagctctg | gaagagaagt | atgtcgcaga | gctgcagctg |
| gaacggctga | agaaagatgg | cgaggtgaga | gggtcaatta | ataggttcaa | gacaagcgac |
| tacgtcaaag | aagccaagca | gctgctgaaa | gtgcagaagg | cttaccacca | gctggatcag |
| agcttcatcg | atacttatat | cgacctgctg | gagactcgga | gaacctacta | tgagggacca |
| ggagaaggga | gccccttcgg | atggaaagac | atcaaggaat | ggtacgagat | gctgatggga |
| cattgcacct | attttccaga | agagctgaga | agcgtcaagt | acgcttataa | cgcagatct |
| tacaacgccc | tgaatgacct | gaacaacctg | gtcatcacca | gggatgaaaa | cgagaaactg |
| gaatactatg | agaagttcca | gatcatcgaa | aacgtgttta | agcagaagaa | aaagcctaca |
| ctgaaacaga | ttgctaagga | gatcctggtc | aacgaagagg | acatcaaggg | ctaccgggtg |
| acaagcactg | gaaaaccaga | gttcaccaat | ctgaaagtgt | atcacgatat | taaggacatc |
| acagcacgga | aagaaatcat | tgagaacgcc | gaactgctgg | atcagattgc | taagatcctg |
| actatctacc | agagctccga | ggacatccag | gaagagctga | ctaacctgaa | cagcgagctg |
| acccaggaag | agatcgaaca | gattagtaat | ctgaaggggt | acaccggaac | acacaacctg |
| tccctgaaag | ctatcaatct | gattctggat | gagctgtggc | atacaaacga | caatcagatt |
| gcaatcttta | accggctgaa | gctggtccca | aaaaaggtgg | acctgagtca | gcagaaagag |
| atcccaacca | cactggtgga | cgatttcatt | ctgtcacccg | tggtcaagcg | gagcttcatc |
| cagagcatca | aagtgatcaa | cgccatcatc | aagaagtacg | gcctgcccaa | tgatatcatt |
| atcgagctgg | ctagggagaa | gaacagcaag | gacgcacaga | agatgatcaa | tgagatgcag |
| aaacgaaacc | ggcagaccaa | tgaacgcatt | gaagagatta | tccgaactac | cgggaaagag |
| aacgcaaagt | acctgattga | aaaaatcaag | ctgcacgata | tgcaggaggg | aaagtgtctg |
| tattctctgg | aggccatccc | cctggaggac | ctgctgaaca | atccattcaa | ctacgaggtc |
| gatcatatta | tccccagaag | cgtgtccttc | gacaattcct | ttaacaacaa | ggtgctggtc |
| aagcaggaag | agaactctaa | aaagggcaat | aggactcctt | tccagtacct | gtctagttca |
| gattccaaga | tctcttacga | aacctttaaa | aagcacattc | tgaatctggc | caaaggaaag |
| ggccgcatca | gcaagaccaa | aaaggagtac | ctgctggaag | agcgggacat | caacagattc |
| tccgtccaga | aggattttat | taaccggaat | ctggtggaca | caagatacgc | tactcgcggc |
| ctgatgaatc | tgctgcgatc | ctatttccgg | gtgaacaatc | tggatgtgaa | agtcaagtcc |
| atcaacggcg | ggttcacatc | ttttctgagg | cgcaaatgga | agtttaaaaa | ggagcgcaac |
| aaagggtaca | agcaccatgc | cgaagatgct | ctgattatcg | caaatgccga | cttcatcttt |
| aaggagtgga | aaaagctgga | caaagccaag | aaagtgatgg | agaaccagat | gttcgaagag |
| aagcaggccg | aatctatgcc | cgaaatcgag | acagaacagg | agtacaagga | gattttcatc |
| actcctcacc | agatcaagca | tatcaaggat | ttcaaggact | acaagtactc | tcaccgggtg |
| gataaaaagc | ccaacagaga | gctgatcaat | gacaccctgt | atagtacaag | aaaagacgat |
| aaggggaata | ccctgattgt | gaacaatctg | aacggactgt | acgacaaaga | taatgacaag |
| ctgaaaaagc | tgatcaacaa | aagtcccgag | aagctgctga | tgtaccacca | tgatcctcag |
| acatatcaga | aactgaagct | gattatggag | cagtacggcg | acgagaagaa | cccactgtat |
| aagtactatg | aagagactgg | gaactacctg | accaagtata | gcaaaaagga | taatggcccc |
| gtgatcaaga | agatcaagta | ctatgggaac | aagctgaatg | cccatctgga | catcacagac |
| gattacccta | acagtcgcaa | caaggtggtc | aagctgtcac | tgaagccata | cagattcgat |
| gtctatctgg | acaacggcgt | gtataaattt | gtgactgtca | agaatctgga | tgtcatcaaa |
| aaggagaact | actatgaagt | gaatagcaag | tgctacgaag | aggctaaaaa | gctgaaaaag |
| attagcaacc | aggcagagtt | catcgcctcc | ttttacaaca | acgacctgat | taagatcaat |
| ggcgaactgt | atagggtcat | cggggtgaac | aatgatctgc | tgaaccgcat | tgaagtgaat |
| atgattgaca | tcacttaccg | agagtatctg | gaaaacatga | atgataagcg | cccccctcga |
| attatcaaaa | caattgcctc | taagactcag | agtatcaaaa | agtactcaac | cgacattctg |
| ggaaacctgt | atgaggtgaa | gagcaaaaag | caccctcaga | ttatcaaaaa | gggctaagaa |
| ttc | | | | | |

SEQ ID NO: 38
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagagcc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 39
codon optimized nucleic acid sequences encoding S. aureus Cas9
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 40
Vector (pDO242) encoding codon optimized nucleic acid sequence encoding
S. aureus Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
SEQ ID NO: 41
Human p300 (with L553M mutation) protein
MAENVVEPGPPSAKRPKLSSPALSASASDGTDFGSLEDLEHDLPDELINSTELGLINGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMENNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIWPAGFVCDGCLKKSARTRKENKFSAKR
LPSTRLGTFLENRVNDFLRRQNHPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
SEQ ID NO: 42
Human p300 Core Effector protein (aa 1048-1664 of SEQ ID NO: 41)
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDFLRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
SEQ ID NO: 43
VP64-dCas9-VP64 protein
RADALDDFDLDMLGSDALDDFDLDMLGSDALDDFDLDMLGSDALDDEDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNEDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDELDNEENEDILE
DIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYEDTTIDRKRYTSTKEVLDATLIHQSITGLYETRIDLSQL
GGDSRADPKKKRKVASRADALDDFDLDMLGSDALDDFDLDMLGSDALDDFDLDMLGSDALDDEDLDML
I
SEQ ID NO: 44
VP64-dCas9-VP64 DNA
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcaccettacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
SEQ ID NO: 45
Polypeptide sequence of KRAB protein
RTLVTFKDVFVDFTREEWKLLDTAQQILYRNVMLENYKNLVSLGYQLTKPDVILRLEKGEEP
WLV
SEQ ID NO: 46
Polynucleotide sequence for KRAB
cggacactggtgaccttcaaggatgtgtttgtggacttcaccagggaggagtggaagctgct
ggacactgctcagcagatcctgtacagaaatgtgatgctggagaactataagaacctggttt
ccttgggttatcagcttactaagccagatgtgatcctccggttggagaagggagaagagccc
tggctggtg
SEQ ID NO: 47
Polypeptide sequence of Streptococcus pyogenes dCas9-KRAB protein
MDYKDHDGDYKDHDIDYKDDDDKMAPKKKRKVGRGMDKKYSIGLAIGTNSVGWAVITDEYKVPSKKFK
VLGNTDRHSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMIKERGHEL
IEGDLNPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAHLFDD
KVMKQLKRRRYTGWGRLSRKLINGIRDKQSGKTILDELKSDGFANRNFMQLIHDDSLTFKEDIQKAQV
SGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSRADPKKKRKVASDAKSLTAWSRTL
VTFKDVFVDFTREEWKLLDTAQQILYRNVMLENYKNLVSLGYQLTKPDVILRLEKGEEPWLVEREIHQ
ETHPDSETAFEIKSSVPKKKRKV
SEQ ID NO: 48
Polynucleotide sequence encoding Streptococcus pyogenes dCas9-KRAB
atggactacaaagaccatgacggtgattataaagatcatgacatcgattacaaggatgacgatgacaa
gatggcccccaagaagaagaggaaggtgggccgcggaatggacaagaagtactccattgggctcgcca
tcggcacaaacagcgtcggctgggccgtcattacggacgagtacaaggtgccgagcaaaaaattcaaa
gttctgggcaataccgatcgccacagcataaagaagaacctcattggcgccctcctgttcgactccgg
ggaaaccgccgaagccacgcggctcaaaagaacagcacggcgcagatatacccgcagaaagaatcgga
tctgctacctgcaggagatctttagtaatgagatggctaaggtggatgactctttcttccataggctg
gaggagtcctttttggtggaggaggataaaaagcacgagcgccacccaatctttggcaatatcgtgga
cgaggtggcgtaccatgaaaagtacccaaccatatatcatctgaggaagaagcttgtagacagtactg
ataaggctgacttgcggttgatctatctcgcgctggcgcatatgatcaaatttcggggacacttcctc
atcgagggggacctgaacccagacaacagcgatgtcgacaaactctttatccaactggttcagactta
caatcagcttttcgaagagaacccgatcaacgcatccggagttgacgccaaagcaatcctgagcgcta
ggctgtccaaatcccggcggctcgaaaacctcatcgcacagctccctggggagaagaagaacggcctg
tttggtaatcttatcgccctgtcactcgggctgacccccaactttaaatctaacttcgacctggccga
agatgccaagcttcaactgagcaaagacacctacgatgatgatctcgacaatctgctggcccagatcg
gcgaccagtacgcagacctttttttggcggcaaagaacctgtcagacgccattctgctgagtgatatt
ctgcgagtgaacacggagatcaccaaagctccgctgagcgctagtatgatcaagcgctatgatgagca
ccaccaagacttgactttgctgaaggcccttgtcagacagcaactgcctgagaagtacaaggaaattt
tcttcgatcagtctaaaaatggctacgccggatacattgacggcggagcaagccaggaggaattttac
aaatttattaagcccatcttggaaaaaatggacggcaccgaggagctgctggtaaagcttaacagaga
agatctgttgcgcaaacagcgcactttcgacaatggaagcatcccccaccagattcacctgggcgaac
tgcacgctatcctcaggcggcaagaggatttctacccctttttgaaagataacagggaaaagattgag
aaaatcctcacatttcggataccctactatgtaggccccctcgcccggggaaattccagattcgcgtg
gatgactcgcaaatcagaagagaccatcactccctggaacttcgaggaagtcgtggataagggggcct
ctgcccagtccttcatcgaaaggatgactaactttgataaaaatctgcctaacgaaaaggtgcttcct
aaacactctctgctgtacgagtacttcacagtttataacgagctcaccaaggtcaaatacgtcacaga
agggatgagaaagccagcattcctgtctggagagcagaagaaagctatcgtggacctcctcttcaaga
cgaaccggaaagttaccgtgaaacagctcaaagaagactatttcaaaaagattgaatgtttcgactct
gttgaaatcagcggagtggaggatcgcttcaacgcatccctgggaacgtatcacgatctcctgaaaat
cattaaagacaaggacttcctggacaatgaggagaacgaggacattcttgaggacattgtcctcaccc
ttacgttgtttgaagatagggagatgattgaagaacgcttgaaaacttacgctcatctcttcgacgac
aaagtcatgaaacagctcaagaggcgccgatatacaggatgggggcggctgtcaagaaaactgatcaa
tgggatccgagacaagcagagtggaaagacaatcctggattttcttaagtccgatggatttgccaacc
ggaacttcatgcagttgatccatgatgactctctcacctttaaggaggacatccagaaagcacaagtt
tctggccagggggacagtcttcacgagcacatcgctaatcttgcaggtagcccagctatcaaaaaggg
aatactgcagaccgttaaggtcgtggatgaactcgtcaaagtaatgggaaggcataagcccgagaata
tcgttatcgagatggcccgagagaaccaaactacccagaagggacagaagaacagtagggaaaggatg
aagaggattgaagagggtataaaagaactggggtcccaaatccttaaggaacacccagttgaaaacac
ccagcttcagaatgagaagctctacctgtactacctgcagaacggcagggacatgtacgtggatcagg
aactggacatcaatcggctctccgactacgacgtggatgccatcgtgccccagtcttttctcaaagat
gattctattgataataaagtgttgacaagatccgataaaaatagagggaagagtgataacgtcccctc
agaagaagttgtcaagaaaatgaaaaattattggcggcagctgctgaacgccaaactgatcacacaac
ggaagttcgataatctgactaaggctgaacgaggtggcctgtctgagttggataaagccggcttcatc
aaaaggcagcttgttgagacacgccagatcaccaagcacgtggcccaaattctcgattcacgcatgaa
caccaagtacgatgaaaatgacaaactgattcgagaggtgaaagttattactctgaagtctaagctgg
tctcagatttcagaaaggactttcagttttataaggtgagagagatcaacaattaccaccatgcgcat
gatgcctacctgaatgcagtggtaggcactgcacttatcaaaaaatatcccaagcttgaatctgaatt
tgtttacggagactataaagtgtacgatgttaggaaaatgatcgcaaagtctgagcaggaaataggca
aggccaccgctaagtacttcttttacagcaatattatgaattttttcaagaccgagattacactggcc
aatggagagattcggaagcgaccacttatcgaaacaaacggagaaacaggagaaatcgtgtgggacaa
gggtagggatttcgcgacagtccggaaggtcctgtccatgccgcaggtgaacatcgttaaaaagaccg
aagtacagaccggaggcttctccaaggaaagtatcctcccgaaaaggaacagcgacaagctgatcgca
cgcaaaaaagattgggaccccaagaaatacggcggattcgattctcctacagtcgcttacagtgtact
ggttgtggccaaagtggagaaagggaagtctaaaaaactcaaaagcgtcaaggaactgctgggcatca
caatcatggagcgatcaagcttcgaaaaaaaccccatcgactttctcgaggcgaaaggatataaagag
gtcaaaaaagacctcatcattaagcttcccaagtactctctctttgagcttgaaaacggccggaaacg
aatgctcgctagtgcgggcgagctgcagaaaggtaacgagctggcactgccctctaaatacgttaatt
tcttgtatctggccagccactatgaaaagctcaaagggtctcccgaagataatgagcagaagcagctg
ttcgtggaacaacacaaacactaccttgatgagatcatcgagcaaataagcgaattctccaaaagagt
gatcctcgccgacgctaacctcgataaggtgctttctgcttacaataagcacagggataagcccatca
gggagcaggcagaaaacattatccacttgtttactctgaccaacttgggcgcgcctgcagccttcaag
tacttcgacaccaccatagacagaaagcggtacacctctacaaaggaggtcctggacgccacactgat
tcatcagtcaattacggggctctatgaaacaagaatcgacctctctcagctcggtggagacagcaggg
ctgaccccaagaagaagaggaaggtggctagcgatgctaagtcactgactgcctggtcccggacactg
gtgaccttcaaggatgtgtttgtggacttcaccagggaggagtggaagctgctggacactgctcagca
gatcctgtacagaaatgtgatgctggagaactataagaacctggtttccttgggttatcagcttacta
agccagatgtgatcctccggttggagaagggagaagagccctggctggtggagagagaaattcaccaa
gagacccatcctgattcagagactgcatttgaaatcaaatcatcagttccgaaaaagaaacgcaaagt
ttga
SEQ ID NO: 49
Polypeptide sequence of Staphylococcus aureus dCas9-KRAB protein
MAPKKKRKVGIHGVPAAKRNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYAYNADLY
NALNDLNNLVITRDENEKLEYYEKFQIIENVFKQKKKPTLKQIAKEILVNEEDIKGYRVTSTGKPEFT
NLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIKV
INAIIKKYGLPNDIIIELAREKNSKDAQKMINEMQKRNRQTNERIEEIIRTTGKENAKYLIEKIKLHD
MQEGKCLYSLEAIPLEDLLNNPFNYEVDHIIPRSVSFDNSFNNKVLVKQEEASKKGNRTPFQYLSSSD
SKISYETFKKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLDKAKKVMENQM
FEEKQAESMPEIETEQEYKEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKNLDVIKK
ENYYEVNSKCYEEAKKLKKISNQAEFIASFYNNDLIKINGELYRVIGVNNDLLNRIEVNMIDITYREY
LENMNDKRPPRIIKTIASKTQSIKKYSTDILGNLYEVKSKKHPQIIKKGKRPAATKKAGQAKKKKGSD
AKSLTAWSRTLVTFKDVFVDFTREEWKLLDTAQQILYRNVMLENYKNLVSLGYQLTKPDVILRLEKGE
EPWLVEREIHQETHPDSETAFEIKSSVPKKKRKV
SEQ ID NO: 50
Polynucleotide sequence of Staphylococcus aureus dCas9-KRAB protein
atggccccaaagaagaagcggaaggtcggtatccacggagtcccagcagccaagcggaactacatcct
gggcctggccatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaagccagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaagggatccgat
gctaagtcactgactgcctggtcccggacactggtgaccttcaaggatgtgtttgtggacttcaccag
ggaggagtggaagctgctggacactgctcagcagatcctgtacagaaatgtgatgctggagaactata
agaacctggtttccttgggttatcagcttactaagccagatgtgatcctccggttggagaagggagaa
gagccctggctggtggagagagaaattcaccaagagacccatcctgattcagagactgcatttgaaat
caaatcatcagttccgaaaaagaaacgcaaagtt
SEQ ID NO: 51
Polypeptide sequence of Tet1CD
LPTCSCLDRVIQKDKGPYYTHLGAGPSVAAVREIMENRYGQKGNAIRIEIVVYTGKEGKSSHGCPIAK
WVLRRSSDEEKVLCLVRQRTGHHCPTAVMVVLIMVWDGIPLPMADRLYTELTENLKSYNGHPTDRRCT
LNENRTCTCQGIDPETCGASFSFGCSWSMYFNGCKFGRSPSPRRFRIDPSSPLHEKNLEDNLQSLATR
LAPIYKQYAPVAYQNQVEYENVARECRLGSKEGRPFSGVTACLDFCAHPHRDIHNMNNGSTVVCTLTR
EDNRSLGVIPQDEQLHVLPLYKLSDTDEFGSKEGMEAKIKSGAIEVLAPRRKKRTCFTQPVPRSGKKR
AAMMTEVLAHKIRAVEKKPIPRIKRKNNSTTTNNSKPSSLPTLGSNTETVQPEVKSETEPHFILKSSD
NTKTYSLMPSAPHPVKEASPGFSWSPKTASATPAPLKNDATASCGFSERSSTPHCTMPSGRLSGANAA
AADGPGISQLGEVAPLPTLSAPVMEPLINSEPSTGVTEPLTPHQPNHQPSFLTSPQDLASSPMEEDEQ
HSEADEPPSDEPLSDDPLSPAEEKLPHIDEYWSDSEHIFLDANIGGVAIAPAHGSVLIECARRELHAT
TPVEHPNRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWV
SEQ ID NO: 52
Polynucleotide sequence of Tet1CD
CTGCCCACCTGCAGCTGTCTTGATCGAGTTATACAAAAAGACAAAGGCCCATATTATACACACCTTGG
GGCAGGACCAAGTGTTGCTGCTGTCAGGGAAATCATGGAGAATAGGTATGGTCAAAAAGGAAACGCAA
TAAGGATAGAAATAGTAGTGTACACCGGTAAAGAAGGGAAAAGCTCTCATGGGTGTCCAATTGCTAAG
TGGGTTTTAAGAAGAAGCAGTGATGAAGAAAAAGTTCTTTGTTTGGTCCGGCAGCGTACAGGCCACCA
CTGTCCAACTGCTGTGATGGTGGTGCTCATCATGGTGTGGGATGGCATCCCTCTTCCAATGGCCGACC
GGCTATACACAGAGCTCACAGAGAATCTAAAGTCATACAATGGGCACCCTACCGACAGAAGATGCACC
CTCAATGAAAATCGTACCTGTACATGTCAAGGAATTGATCCAGAGACTTGTGGAGCTTCATTCTCTTT
TGGCTGTTCATGGAGTATGTACTTTAATGGCTGTAAGTTTGGTAGAAGCCCAAGCCCCAGAAGATTTA
GAATTGATCCAAGCTCTCCCTTACATGAAAAAAACCTTGAAGATAACTTACAGAGTTTGGCTACACGA
TTAGCTCCAATTTATAAGCAGTATGCTCCAGTAGCTTACCAAAATCAGGTGGAATATGAAAATGTTGC
CCGAGAATGTCGGCTTGGCAGCAAGGAAGGTCGACCCTTCTCTGGGGTCACTGCTTGCCTGGACTTCT
GTGCTCATCCCCACAGGGACATTCACAACATGAATAATGGAAGCACTGTGGTTTGTACCTTAACTCGA
GAAGATAACCGCTCTTTGGGTGTTATTCCTCAAGATGAGCAGCTCCATGTGCTACCTCTTTATAAGCT
TTCAGACACAGATGAGTTTGGCTCCAAGGAAGGAATGGAAGCCAAGATCAAATCTGGGGCCATCGAGG
TCCTGGCACCCCGCCGCAAAAAAAGAACGTGTTTCACTCAGCCTGTTCCCCGTTCTGGAAAGAAGAGG
GCTGCGATGATGACAGAGGTTCTTGCACATAAGATAAGGGCAGTGGAAAAGAAACCTATTCCCCGAAT
CAAGCGGAAGAATAACTCAACAACAACAAACAACAGTAAGCCTTCGTCACTGCCAACCTTAGGGAGTA
ACACTGAGACCGTGCAACCTGAAGTAAAAAGTGAAACCGAACCCCATTTTATCTTAAAAAGTTCAGAC
AACACTAAAACTTATTCGCTGATGCCATCCGCTCCTCACCCAGTGAAAGAGGCATCTCCAGGCTTCTC
CTGGTCCCCGAAGACTGCTTCAGCCACACCAGCTCCACTGAAGAATGACGCAACAGCCTCATGCGGGT
TTTCAGAAAGAAGCAGCACTCCCCACTGTACGATGCCTTCGGGAAGACTCAGTGGTGCCAATGCTGCA
GCTGCTGATGGCCCTGGCATTTCACAGCTTGGCGAAGTGGCTCCTCTCCCCACCCTGTCTGCTCCTGT
GATGGAGCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGATGAGCAG
CATTCTGAAGCAGATGAGCCTCCATCAGACGAACCCCTATCTGATGACCCCCTGTCACCTGCTGAGGA
GAAATTGCCCCACATTGATGAGTATTGGTCAGACAGTGAGCACATCTTTTTGGATGCAAATATTGGTG
GGGTGGCCATCGCACCTGCTCACGGCTCGGTTTTGATTGAGTGTGCCCGGCGAGAGCTGCACGCTACC
ACTCCTGTTGAGCACCCCAACCGTAATCATCCAACCCGCCTCTCCCTTGTCTTTTACCAGCACAAAAA
CCTAAATAAGCCCCAACATGGTTTTGAACTAAACAAGATTAAGTTTGAGGCTAAAGAAGCTAAGAATA
AGAAAATGAAGGCCTCAGAGCAAAAAGACCAGGCAGCTAATGAAGGTCCAGAACAGTCCTCTGAAGTA
AATGAATTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC
SEQ ID NO: 53
Protein sequence for VPH
DALDDFDLDMLGSDALDDFDLDMLGSDALDDFDLDMLGSDALDDFDLDMLGSLPSASVEFEGSGGPSG
QISNQALALAPSSAPVLAQTMVPSSAMVPLAQPPAPAPVLTPGPPQSLSAPVPKSTQAGEGTLSEALL
HLQFDADEDLGALLGNSTDPGVFTDLASVDNSEFQQLLNQGVSMSHSTAEPMLMEYPEAITRLVTGSQ
RPPDPAPTPLGTSGLPNGLSGDEDFSSIADMDFSALLSQISSSGQGGGGSGFSVDTSALLDLFSPSVT
VPDMSLPDLDSSLASIQELLSPQEPPRPPEAENSSPDSGKQLVHYTAQPLFLLDPGSVDTGSNDLPVL
FELGEGSYFSEGDGFAEDPTISLLTGSEPPKAKDPTVS
SEQ ID NO: 54
DNA sequence for VPH
Gatgctttagacgattttgacttagatatgcttggttcagacgcgttagacgacttcgacctagacat
gttaggctcagatgcattggacgacttcgatttagatatgttgggctccgatgccctagatgactttg
atctagatatgctagggtcactacccagcgccagcgtcgagttcgaaggcagcggcgggccttcaggg
cagatcagcaaccaggccctggctctggcccctagctccgctccagtgctggcccagactatggtgcc
ctctagtgctatggtgcctctggcccagccacctgctccagcccctgtgctgaccccaggaccacccc
agtcactgagcgccccagtgcccaagtctacacaggccggcgaggggactctgagtgaagctctgctg
cacctgcagttcgacgctgatgaggacctgggagctctgctggggaacagcaccgatcccggagtgtt
cacagatctggcctccgtggacaactctgagtttcagcagctgctgaatcagggcgtgtccatgtctc
atagtacagccgaaccaatgctgatggagtaccccgaagccattacccggctggtgaccggcagccag
cggccccccgaccccgctccaactcccctgggaaccagcggcctgcctaatgggctgtccggagatga
agacttctcaagcatcgctgatatggactttagtgccctgctgtcacagatttcctctagtgggcagg
gaggaggtggaagcggcttcagcgtggacaccagtgccctgctggacctgttcagcccctcggtgacc
gtgcccgacatgagcctgcctgaccttgacagcagcctggccagtatccaagagctcctgtctcccca
ggagccccccaggcctcccgaggcagagaacagcagcccggattcagggaagcagctggtgcactaca
cagcgcagccgctgttcctgctggaccccggctccgtggacaccgggagcaacgacctgccggtgctg
tttgagctgggagagggctcctacttctccgaaggggacggcttcgccgaggaccccaccatctccct
gctgacaggctcggagcctcccaaagccaaggaccccactgtctcc
SEQ ID NO: 55
Protein sequence for VPR
DALDDFDLDMLGSDALDDFDLDMLGSDALDDFDLDMLGSDALDDFDLDMLGSPKKKRKVGSQYLPDTD
DRHRIEEKRKRTYETFKSIMKKSPFSGPTDPRPPPRRIAVPSRSSASVPKPAPQPYPFTSSLSTINYD
EFPTMVFPSGQISQASALAPAPPQVLPQAPAPAPAPAMVSALAQAPAPVPVLAPGPPQAVAPPAPKPT
QAGEGTLSEALLQLQFDDEDLGALLGNSTDPAVFTDLASVDNSEFQQLLNQGIPVAPHTTEPMLMEYP
EAITRLVTGAQRPPDPAPAPLGAPGLPNGLLSGDEDFSSIADMDFSALLSQISSGSGSGSRDSREGMF
LPKPEAGSAISDVFEGREVCQPKRIRPFHPPGSPWANRPLPASLAPTPTGPVHEPVGSLTPAPVPQPL
DPAPAVTPEASHLLEDPDEETSQAVKALREMADTVIPQKEEAAICGQMDLSHPPPRGHLDELTTTLES
MTEDLNLDSPLTPELNEILDTFLNDECLLHAMHISTGLSIFDTSLF
SEQ ID NO: 56
DNA sequence for VPR
gatgctttagacgattttgacttagatatgcttggttcagacgcgttagacgacttcgacctagacat
gttaggctcagatgcattggacgacttcgatttagatatgttgggctccgatgccctagatgactttg
atctagatatgctaggtagtcccaaaaagaagaggaaagtgggatcccagtatctgcccgacacagat
gatagacaccgaatcgaagagaaacgcaagcgaacgtatgaaaccttcaaatcgatcatgaagaaatc
gcccttctcgggtccgaccgatcccaggcccccaccgagaaggattgcggtcccgtcccgctcgtcgg
ccagcgtgccgaagcctgcgccgcagccctaccccttcacgtcgagcctgagcacaatcaattatgac
gagttcccgacgatggtgttcccctcgggacaaatctcacaagcctcggcgctcgcaccagcgcctcc
ccaagtccttccgcaagcgcctgccccagcgcctgcaccggcaatggtgtccgccctcgcacaggccc
ctgcgcccgtccccgtgctcgcgcctggaccgccccaggcggtcgctccaccggctccgaagccgacg
caggccggagagggaacactctccgaagcacttcttcaactccagtttgatgacgaggatcttggagc
actccttggaaactcgacagaccctgcggtgtttaccgacctcgcgtcagtagataactccgaatttc
agcagcttttgaaccagggtatcccggtcgcgccacatacaacggagcccatgttgatggaatacccc
gaagcaatcacgagacttgtgacgggagcgcagcggcctcccgatcccgcacccgcacctttgggggc
acctggcctccctaacggacttttgagcggcgacgaggatttctcctccatcgccgatatggatttct
cagccttgctgtcacagatttccagcggctctggcagcggcagccgggattccagggaagggatgttt
ttgccgaagcctgaggccggctccgctattagtgacgtgtttgagggccgcgaggtgtgccagccaaa
acgaatccggccatttcatcctccaggaagtccatgggccaaccgcccactccccgccagcctcgcac
caacaccaaccggtccagtacatgagccagtcgggtcactgaccccggcaccagtccctcagccactg
gatccagcgcccgcagtgactcccgaggccagtcacctgttggaggatcccgatgaagagacgagcca
ggctgtcaaagcccttcgggagatggccgatactgtgattccccagaaggaagaggctgcaatctgtg
gccaaatggacctttcccatccgcccccaaggggccatctggatgagctgacaaccacacttgagtcc
atgaccgaggatctgaacctggactcacccctgaccccggaattgaacgagattctggataccttcct
gaacgacgagtgcctcttgcatgccatgcatatcagcacaggactgtccatcttcgacacatctctgt
tt
SEQ ID NO: 57
Protein sequence for MCRS1
YKKAGSTMDKDSQGLLDSSLMASGTASRSEDEESLAGQKRASSQALGTIPKRRSSSRFIKRK
KFDDELVESSLAKSSTRAKGASGVEPGRCSGSEPSSSEKKKVSKAPSTPVPPSPAPAPGLTK
RVKKSKQPLQVTKDLGRWKPADDLLLINAVLQTNDLTSVHLGVKESCRFTLREVQERWYALL
YDPVISKLACQAMRQLHPEAIAAIQSKALFSKAEEQLLSKVGSTSQPTLETFQDLLHRHPDA
FYLARTAKALQAHWQLMKQYYLLEDQTVQPLPKGDQVLNFSDAEDLIDDSKLKDMRDEVLEH
ELMVADRRQKREIRQLEQELHKWQVLVDSITGMSSPDFDNQTLAVLRGRMVRYLMRSREITL
GRATKDNQIDVDLSLEGPAWKISRKQGVIKLKNNGDFFIANEGRRPIYIDGRPVLCGSKWRL
SNNSVVEIASLRFVFLINQDLIALIRAEAAKITPQLDPAFL
SEQ ID NO: 58
DNA sequence for MCRS1
gtacaaaaaagcaggctccaccatggacaaagattctcaggggctgctagattcatccctga
tggcatcaggcactgccagccgctcagaggatgaggagtcactggcagggcagaagcgagcc
tcctcccaggccttgggcaccatccctaaacggagaagctcctccaggttcatcaagaggaa
gaagttcgatgatgagctggtggagagcagcctggcaaaatcttctacccgggcaaaggggg
ccagtggggtggaaccagggcgctgttcggggagtgaaccctcctccagtgagaagaagaag
gtatccaaagcccccagcactcctgtgccacccagcccagccccagcccctggactcaccaa
gcgtgtgaagaagagtaaacagccacttcaggtgaccaaggatctgggccgctggaagcctg
cagatgacctcctgctcataaatgctgtgttgcagaccaacgacctgacctccgtccacctg
ggcgtgaaattcagctgccgcttcacccttcgggaggtccaggagcgttggtacgccctgct
ctacgatcctgtcatctccaagttggcctgtcaggccatgaggcagctgcacccagaggcta
ttgcagccatccagagcaaggccctgtttagcaaggctgaggagcagctgctgagcaaagtg
ggatcgaccagccagcccaccttggagaccttccaggacctgctgcacagacaccctgatgc
cttctacctggcccgtaccgcgaaggccctgcaggcccactggcagctcatgaagcagtatt
acctgctggaggaccagacagtgcagccgctgcccaaaggggaccaagtgctgaacttctct
gatgcagaggacctgattgatgacagtaagctcaaggacatgcgagatgaggtcctggaaca
tgagctgatggtggctgaccggcgccagaagcgagagattcggcagctggaacaggaactgc
ataagtggcaggtgctagtggacagcatcacaggcatgagctctccggacttcgacaaccag
acactggcagtgctgcggggccgcatggtgcggtacctgatgcgctcgcgtgagatcaccct
gggcagagcaaccaaggataaccagattgatgtggacctgtctctggagggtccggcctgga
agatatcccggaaacaaggtgtcatcaagctgaagaacaacggtgatttcttcattgccaat
gagggtcgacggcccatctacatcgatggacggccggtgctctgtggctccaaatggcgcct
cagcaacaactctgtggtggagatcgccagcctgcgattcgtcttccttatcaaccaggacc
tcattgccctcatcagggctgaggctgccaagatcacaccacagttggacccagctttcttg
tac
SEQ ID NO: 59
Protein sequence for OTUD7B
MTLDMDAVLSDFVRSTGAEPGLARDLLEGKNWDVNAALSDFEQLRQVHAGNLPPSESEGSGG
SRTPEKGFSDREPTRPPRPILQRQDDIVQEKRLSRGISHASSSIVSLARSHVSSNGGGGGSN
EHPLEMPICAFQLPDLTVYNEDERSFIERDLIEQSMLVALEQAGRLNWWVSVDPTSQRLLPL
ATTGDGNCLLHAASLGMWGFHDRDLMLRKALYALMEKGVEKEALKRRWRWQQTQQNKESGLV
YTEDEWQKEWNELIKLASSEPRMHLGINGANCGGVESSEEPVYESLEEFHVFVLAHVLRRPI
VVVADTMLRDSGGEAFAPIPFGGIYLPLEVPASQCHRSPLVLAYDQAHFSALVSMEQKENTK
EQAVIPLTDSEYKLLPLHFAVDPGKGWEWGKDDSDNVRLASVILSLEVKLHLLHSYMNVKWI
PLSSDAQAPLAQPESPTASAGDEPRSTPESGDSDKESVGSSSTSNEGGRRKEKSKRDREKDK
KRADSVANKLGSFGKTLGSKLKKNMGGLMHSKGSKPGGVGTGLGGSSGTETLEKKKKNSLKS
WKGGKEEAAGDGPVSEKPPAESVGNGGSKYSQEVMQSLSILRTAMQGEGKFIFVGTLKMGHR
HQYQEEMIQRYLSDAEERFLAEQKQKEAERKIMNGGIGGGPPPAKKPEPDAREEQPTGPPAE
SRAMAFSTGYPGDFTIPRPSGGGVHCQEPRRQLAGGPCVGGLPPYATFPRQCPPGRPYPHQD
SIPSLEPGSHSKDGLHRGALLPPPYRVADSYSNGYREPPEPDGWAGGLRGLPPTQTKCKQPN
CSFYGHPETNNFCSCCYREELRRREREPDGELLVHRFLDPAFLY
SEQ ID NO: 60
DNA sequence for OTUD7B
gtacaaaaaagcaggctccaccatgaccctggacatggatgctgttctgtcagattttgtcc
gttccacaggagcagagccagggctagcgcgagatctcctagaaggaaagaattgggatgtg
aatgccgccctcagtgattttgaacagctacgtcaagtccatgctggaaacctacccccatc
ctttagtgaggggagtggtggctccaggacccctgaaaaagggttttctgacagagagccta
ctcgccctccccgacccatcctccagcggcaggatgacatcgttcaagaaaaacgcctgtct
aggggcatctcccacgccagctccagcattgtttccctggcccggtcccatgtctcctccaa
tggtgggggtggggggagcaatgagcaccccctggaaatgcccatctgtgccttccagcttc
cagatctcactgtatacaatgaagacttccgcagcttcatagagagagacctcattgagcag
tccatgctggttgccttggaacaggcagggcgtttgaactggtgggtgagtgtggatcccac
ctctcagaggctgcttcctttggcaactactggagatgggaactgcctcctgcatgcagcct
cccttggaatgtggggtttccatgatcgggacttgatgctgcggaaagctttgtatgcactg
atggagaagggagttgagaaggaagcgttgaaaaggcgctggaggtggcagcagacacagca
gaataaagagtcagggctggtatacacagaagatgaatggcagaaggagtggaatgaactga
tcaagcttgcctcaagtgaaccccgaatgcatctaggtaccaatggagccaactgtggtggg
gtggagagttctgaggagcctgtatatgagagccttgaagagtttcacgtctttgtccttgc
tcatgtgcttaggaggcccatagtcgtcgtggcagacaccatgctgagggactccggagggg
aagcatttgcccctattccctttggaggaatctatctgcctttggaggtcccagccagccag
tgtcaccgctcccctctggtgctcgcctatgatcaggcccacttttctgcactcgtgtccat
ggagcagaaggagaataccaaggaacaagctgtgatcccacttacagattcagagtataagc
tgctgcccttgcactttgctgtggaccctggaaagggctgggagtggggcaaagatgatagt
gacaatgtccgattggccagtgtaattctgtccctagaggtcaaattgcatctgctgcatag
ctacatgaatgtgaagtggatcccactgtcctctgatgcacaggctcctctggcccagcctg
agtcccccaccgcctcagctggagatgagccccggtccactcctgagtctggagactcagac
aaggagtcagttggcagcagttccaccagcaacgagggcggccggcggaaggagaagtcaaa
gcgagatcgggagaaggacaagaagagagcagattctgtggctaacaaactgggcagctttg
gcaaaaccttgggcagcaagctcaagaagaacatggggggcctgatgcacagcaagggttca
aagcctggaggggtggggacagggttgggaggaagcagcggcactgagacactggagaagaa
gaagaaaaactcactgaagagctggaagggtggcaaggaggaggcagctggggatgggcctg
tgtctgagaagcccccagctgagtctgttggtaacggagggagcaagtatagccaggaggtg
atgcaaagcctgagcattctgaggactgccatgcaaggggaggggaagtttatttttgttgg
aaccctgaagatgggtcaccgtcaccagtatcaggaggaaatgatccagcgctacctttctg
atgctgaggagagattcctggcagaacagaagcagaaggaggcagagaggaagatcatgaat
ggaggaatagggggtggccctcctccagccaaaaagccagagccagatgctagggaagagca
gccgaccggtcccccagcagagtccagggcaatggcattttccactggctaccctggggact
ttactatccctcggccgtctgggggcggagtccactgccaggaaccccggaggcagttggca
gggggtccatgtgtcgggggcctaccaccatatgccaccttccccagacagtgccctcctgg
gcgaccctacccccaccaggacagcatcccttctctggagccaggcagccactctaaggatg
gacttcacaggggtgccttgttaccacccccctaccgagtggctgattcctatagcaatggc
tacagagagccccctgagccagatggatgggctggaggtctccggggccttcccccaactca
gaccaaatgcaaacaaccgaactgcagcttctatggacaccctgagacaaacaacttctgtt
cctgttgttacagggaagaactgaggaggagggagcgggaaccggatggggagctcctggtg
cacaggttcttggacccagctttcttgtac
SEQ ID NO: 61
Protein sequence for LDB1
MLDRDVGPTPMYPPTYLEPGIGRHTPYGNQTDYRIFELNKRLQNWTEECDNLWWDAFTTEFF
EDDAMLTITFCLEDGPKRYTIGRTLIPRYFRSIFEGGATELYYVLKHPKEAFHSNFVSLDCD
QGSMVTQHGKPMFTQVCVEGRLYLEFMFDDMMRIKTWHFSIRQHRELIPRSILAMHAQDPQM
LDQLSKNITRCGLSNSTLNYLRLCVILEPMQELMSRHKTYSLSPRDCLKTCLFQKWQRMVAP
PAEPTRQQPSKRRKRKMSGGSTMSSGGGNTNNSNSKKKSPASTFALSSQVPDVMVVGEPTLM
GGEFGDEDERLITRLENTQFDAANGIDDEDSENNSPALGANSPWNSKPPSSQESKSENPTSQ
ASQLDPAFLY
SEQ ID NO: 62
DNA sequence for LDB1
atgctggatagggatgtgggcccaactcccatgtatccgcctacatacctggagccagggat
tgggaggcacacaccatatggcaaccaaactgactacagaatatttgagcttaacaaacggc
ttcagaactggacagaggagtgtgacaatctctggtgggatgcattcacgactgagttcttt
gaggatgatgccatgttgaccatcactttctgcctggaggatggaccaaagagatataccat
tggccggaccctgatcccacgctacttccgcagcatctttgaggggggtgctacggagctgt
actatgttcttaagcaccccaaggaggcattccacagcaactttgtgtccctcgactgtgac
cagggcagcatggtgacccagcatggcaagcccatgttcacccaggtgtgtgtggagggccg
gttgtacctggagttcatgtttgacgacatgatgcggataaagacgtggcacttcagcatcc
ggcagcaccgagagctcatcccccgcagcatccttgccatgcatgcccaagacccccagatg
ttggatcagctctccaaaaacatcactcggtgtgggctgtccaattccactctcaactacct
ccgactctgtgtgatactcgagcccatgcaagagctcatgtcacgccacaagacctacagcc
tcagcccccgcgactgcctcaagacctgccttttccagaagtggcagcgcatggtagcaccc
cctgcggagcccacacgtcagcagcccagcaaacggcggaaacggaagatgtcagggggcag
caccatgagctctggtggtggcaacaccaacaacagcaacagcaagaagaagagcccagcta
gcaccttcgccctctccagccaggtacctgatgtgatggtggtgggggagcccaccctgatg
ggcggggagttcggggacgaggacgagaggctcatcacccggctggagaacacccagtttga
cgcagccaacggcattgacgacgaggacagctttaacaactcccctgcactgggcgccaaca
gcccctggaacagcaagcctccgtccagccaagaaagcaaatcggagaaccccacgtcacag
gcctcccagttggacccagctttcttgtac
SEQ ID NO: 63
Protein sequence for NFKBIB
MAGVACLGKAADADEWCDSGLGSLGPDAAAPGGPGLGAELGPGLSWAPLVFGYVTEDGDTAL
HLAVIHQHEPFLDFLLGFSAGTEYMDLQNDLGQTALHLAAILGETSTVEKLYAAGAGLCVAE
RRGHTALHLACRVGAHACARALLQPRPRRPREAPDTYLAQGPDRTPDTNHTPVALYPDSDLE
KEEEESEEDWKLQLEAENYEGHTPLHVAVIHKDVEMVRLLRDAGADLDKPEPTCGRSPLHLA
VEAQAADVLELLLRAGANPAARMYGGRTPLGSAMLRPNPILARLLRAHGAPEPEGEDEKSGP
CSSSSDSDSGDEGDEYDDIVVHSSRSQTRLPPTPASKPLPDDPRPV
SEQ ID NO: 64
DNA sequence for NFKBIB
ATGGCTGGGGTCGCGTGCTTGGGAAAAGCTGCCGACGCAGATGAATGGTGCGACAGCGGCCT
GGGCTCCCTGGGTCCGGACGCAGCGGCCCCCGGAGGACCTGGGTTGGGCGCGGAGTTGGGCC
CGGGGCTGTCGTGGGCTCCCCTCGTCTTCGGCTACGTCACTGAGGATGGGGACACGGCACTG
CACTTGGCTGTGATTCATCAGCATGAACCCTTCCTGGATTTTCTTCTAGGCTTCTCGGCCGG
CACTGAGTACATGGACCTGCAGAATGACCTAGGCCAGACAGCCCTGCACCTGGCAGCCATCC
TGGGGGAGACATCCACGGTGGAGAAGCTGTACGCAGCAGGCGCCGGGCTGTGTGTGGCGGAG
CGTAGGGGCCACACGGCGCTGCACCTGGCCTGCCGTGTGGGGGCACACGCCTGTGCCCGTGC
CCTGCTTCAGCCCCGCCCCCGGCGCCCCAGGGAAGCCCCCGACACCTACCTCGCTCAGGGCC
CTGACCGTACTCCCGACACCAACCATACCCCTGTCGCCTTGTACCCCGATTCCGACTTGGAG
AAGGAAGAAGAGGAGAGTGAGGAGGACTGGAAGCTGCAGCTGGAGGCTGAAAACTACGAGGG
CCACACCCCACTCCACGTGGCCGTTATCCACAAAGATGTGGAGATGGTCCGGCTGCTCCGAG
ATGCTGGAGCTGACCTTGACAAACCGGAGCCCACGTGCGGCCGGAGCCCCCTTCATTTGGCA
GTGGAGGCCCAGGCAGCCGATGTGCTGGAGCTTCTCCTGAGGGCAGGCGCGAACCCTGCTGC
CCGCATGTACGGTGGCCGCACCCCACTCGGCAGTGCCATGCTCCGGCCCAACCCCATCCTCG
CCCGCCTCCTCCGTGCACACGGAGCCCCTGAGCCCGAGGGCGAGGACGAGAAATCCGGCCCC
TGCAGCAGCAGTAGCGACAGCGACAGCGGAGACGAGGGCGATGAATACGACGACATTGTGGT
TCACAGCAGCCGCAGCCAAACCCGGCTGCCTCCCACCCCAGCCTCAAAACCTCTTCCTGACG
ACCCCCGCCCCGTGTGA
SEQ ID NO: 65
Protein sequence for RelB
MLRSGPASGPSVPTGRAMPSRRVARPPAAPELGALGSPDLSSLSLAVSRSTDELEIIDEYIK
ENGFGLDGGQPGPGEGLPRLVSRGAASLSTVTLGPVAPPATPPPWGCPLGRLVSPAPGPGPQ
PHLVITEQPKQRGMRFRYECEGRSAGSILGESSTEASKTLPAIELRDCGGLREVEVTACLVW
KDWPHRVHPHSLVGKDCTDGICRVRLRPHVSPRHSENNLGIQCVRKKEIEAAIERKIQLGID
PYNAGSLKNHQEVDMNVVRICFQASYRDQQGQMRRMDPVLSEPVYDKKSTNTSELRICRINK
ESGPCTGGEELYLLCDKVQKEDISVVFSRASWEGRADESQADVHRQIAIVEKTPPYEDLEIV
EPVTVNVFLQRLTDGVCSEPLPFTYLPRDHDSYGVDKKRKRGMPDVLGELNSSDPHGIESKR
RKKKPAILDHFLPNHGSGPFLPPSALLPDPDFFSGTVSLPGLEPPGGPDLLDDGFAYDPTAP
TLFTMLDLLPPAPPHASAVVCSGGAGAVVGETPGPEPLTLDSYQAPGPGDGGTASLVGSNMF
PNHYREAAFGGGLLSPGPEAT
SEQ ID NO: 66
DNA sequence for RelB
ATGCTTCGGTCTGGGCCAGCCTCTGGGCCGTCCGTCCCCACTGGCCGGGCCATGCCGAGTCG
CCGCGTCGCCAGACCGCCGGCTGCGCCGGAGCTGGGGGCCTTAGGGTCCCCCGACCTCTCCT
CACTCTCGCTCGCCGTTTCCAGGAGCACAGATGAATTGGAGATCATCGACGAGTACATCAAG
GAGAACGGCTTCGGCCTGGACGGGGGACAGCCGGGCCCGGGCGAGGGGCTGCCACGCCTGGT
GTCTCGCGGGGCTGCGTCCCTGAGCACGGTCACCCTGGGCCCTGTGGCGCCCCCAGCCACGC
CGCCGCCTTGGGGCTGCCCCCTGGGCCGACTAGTGTCCCCAGCGCCGGGCCCGGGCCCGCAG
CCGCACCTGGTCATCACGGAGCAGCCCAAGCAGCGCGGCATGCGCTTCCGCTACGAGTGCGA
GGGCCGCTCGGCCGGCAGCATCCTTGGGGAGAGCAGCACCGAGGCCAGCAAGACGCTGCCCG
CCATCGAGCTCCGGGATTGTGGAGGGCTGCGGGAGGTGGAGGTGACTGCCTGCCTGGTGTGG
AAGGACTGGCCTCACCGAGTCCACCCCCACAGCCTCGTGGGGAAAGACTGCACCGACGGCAT
CTGCAGGGTGCGGCTCCGGCCTCACGTCAGCCCCCGGCACAGTTTTAACAACCTGGGCATCC
AGTGTGTGAGGAAGAAGGAGATTGAGGCTGCCATTGAGCGGAAGATTCAACTGGGCATTGAC
CCCTACAACGCTGGGTCCCTGAAGAACCATCAGGAAGTAGACATGAATGTGGTGAGGATCTG
CTTCCAGGCCTCATATCGGGACCAGCAGGGACAGATGCGCCGGATGGATCCTGTGCTTTCCG
AGCCCGTCTATGACAAGAAATCCACAAACACATCAGAGCTGCGGATTTGCCGAATTAACAAG
GAAAGCGGGCCGTGCACCGGTGGCGAGGAGCTCTACTTGCTCTGCGACAAGGTGCAGAAAGA
GGACATATCAGTGGTGTTCAGCAGGGCCTCCTGGGAAGGTCGGGCTGACTTCTCCCAGGCCG
ACGTGCACCGCCAGATTGCCATTGTGTTCAAGACGCCGCCCTACGAGGACCTGGAGATTGTC
GAGCCCGTGACAGTCAACGTCTTCCTGCAGCGGCTCACCGATGGGGTCTGCAGCGAGCCATT
GCCTTTCACGTACCTGCCTCGCGACCATGACAGCTACGGCGTGGACAAGAAGCGGAAACGGG
GGATGCCCGACGTCCTTGGGGAGCTGAACAGCTCTGACCCCCATGGCATCGAGAGCAAACGG
CGGAAGAAAAAGCCGGCCATCCTGGACCACTTCCTGCCCAACCACGGCTCAGGCCCGTTCCT
CCCGCCGTCAGCCCTGCTGCCAGACCCTGACTTCTTCTCTGGCACCGTGTCCCTGCCCGGCC
TGGAGCCCCCTGGCGGGCCTGACCTCCTGGACGATGGCTTTGCCTACGACCCTACGGCCCCC
ACACTCTTCACCATGCTGGACCTGCTGCCCCCGGCACCGCCACACGCTAGCGCTGTTGTGTG
CAGCGGAGGTGCCGGGGCCGTGGTTGGGGAGACCCCCGGCCCTGAACCACTGACACTGGACT
CGTACCAGGCCCCGGGCCCCGGGGATGGAGGCACCGCCAGCCTTGTGGGCAGCAACATGTTC
CCCAATCATTACCGCGAGGCGGCCTTTGGGGGCGGCCTCCTATCCCCGGGGCCTGAAGCCAC
GTAG
SEQ ID NO: 67
Protein sequence for CITED2
MADHMMAMNHGRFPDGTNGLHHHPAHRMGMGQFPSPHHHQQQQPQHAFNALMGEHIHYGAGN
MNATSGIRHAMGPGTVNGGHPPSALAPAARENNSQFMGPPVASQGGSLPASMQLQKLNNQYF
NHHPYPHNHYMPDLHPAAGHQMNGTNQHERDCNPKHSGGSSTPGGSGGSSTPGGSGSSSGGG
AGSSNSGGGSGSGNMPASVAHVPAAMLPPNVIDTDFIDEEVLMSLVIEMGLDRIKELPELWL
GQNEFDFMTDFVCKQQPSRVSCLDPAFLY
SEQ ID NO: 68
DNA sequence for CITED2
atggcagaccatatgatggcaatgaaccacgggcgcttccccgacggcaccaatgggctgca
ccatcaccctgcccaccgcatgggcatggggcagttcccgagcccccatcaccaccagcagc
agcagccccagcacgccttcaacgccctaatgggcgagcacatacactacggcgcgggcaac
atgaatgccacgagcggcatcaggcatgcgatggggccggggactgtgaacggagggcaccc
cccgagcgcgctggcccccgcggccaggtttaacaactcccagttcatgggtcccccggtgg
ccagccagggaggctccctgccggccagcatgcagctgcagaagctcaacaaccagtatttc
aaccatcacccctacccccacaaccactacatgccggatttgcaccctgctgcaggccacca
gatgaacgggacaaaccagcacttccgagattgcaaccccaagcacagcggcggcagcagca
cccccggcggctcgggcggcagcagcacccccggcggctctggcagcagctcgggcggcggc
gcgggcagcagcaacagcggcggcggcagcggcagcggcaacatgcccgcctccgtggccca
cgtccccgctgcaatgctgccgcccaatgtcatagacactgatttcatcgacgaggaagttc
ttatgtccttggtgatagaaatgggtttggaccgcatcaaggagctgcccgaactctggctg
gggcaaaacgagtttgattttatgacggacttcgtgtgcaaacagcagcccagcagagtgag
ctgtttggacccagctttcttgtac
SEQ ID NO: 69
Protein sequence for ScFv-sfBFP-MCRS1

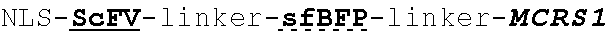

MGPKKKRKVGGMGPDIVMTQSPSSLSASVGDRVTITCRSSTGAVTTSNYASWV
QEKPGKLFKGLIGGTNNRAPGVPSRFSGSLIGDKATLTISSLQPEDFATYFCA
LWYSNHWVFGQGTKVELKRGGGGSGGGGSGGGGSSGGGSEVKLLESGGGLVQP
GGSLKLSCAVSGFSLTDYGVNWVRQAPGRGLEWIGVIWGDGITDYNSALKDRE
IISKDNGKNTVYLQMSKVRSDDTALYYCVTGLEDYWGQGTLVTVSSYPYDVPD

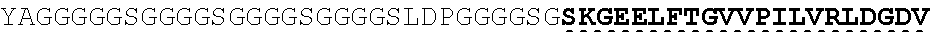

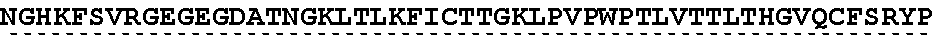

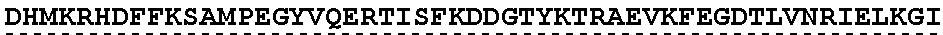

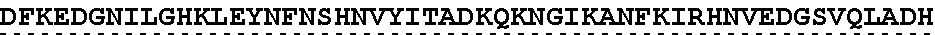

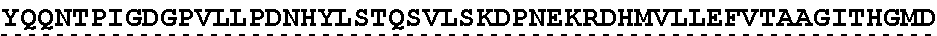

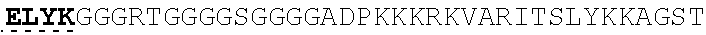

MDKDSQGLLDSSLMASGTASRSEDEESLAGQKRASSQALGTIPKRRSSSRFIK
RKKFDDELVESSLAKSSTRAKGASGVEPGRCSGSEPSSSEKKKVSKAPSTPVP
PSPAPAPGLTKRVKKSKQPLQVTKDLGRWKPADDLLLINAVLQTNDLTSVHLG
VKFSCRFTLREVQERWYALLYDPVISKLACQAMRQLHPEAIAAIQSKALFSKA
EEQLLSKVGSTSQPTLETFQDLLHRHPDAFYLARTAKALQAHWQLMKQYYLLE
DQTVQPLPKGDQVLNFSDAEDLIDDSKLKDMRDEVLEHELMVADRRQKREIRQ
LEQELHKWQVLVDSITGMSSPDEDNQTLAVLRGRMVRYLMRSREITLGRATKD
NQIDVDLSLEGPAWKISRKQGVIKLKNNGDFFIANEGRRPIYIDGRPVLCGSK
WRLSNNSVVEIASLRFVFLINQDLIALIRAEAAKITPQLDPAFL
SEQ ID NO: 70
DNA sequence for ScFv-sfBFP-MCRS1
atgggtCCCAAGAAAAAGAGAAAGGTCggtggcatgggccccgacatcgtgatgacccagag
ccccagcagcctgagcgccagcgtgggcgaccgcgtgaccatcacctgccgcagcagcaccg
gcgccgtgaccaccagcaactacgccagctgggtgcaggagaagcccggcaagctgttcaag
ggcctgatcggcggcaccaacaaccgcgcccccggcgtgcccagccgcttcagcggcagcct
gatcggcgacaaggccaccctgaccatcagcagcctgcagcccgaggacttcgccacctact
tctgcgccctgtggtacagcaaccactgggtgttcggccagggcaccaaggtggagctgaag
cgcggcggcggTggAagcggAggcggTggGTCTggTggAggcggcagcTCIggcggAggcag
cgaggtgaagctgctggagagcggcggcggcctggtgcagcccggcggcagcctgaagctga
gctgcgccgtgagcggcttcagcctgaccgactacggcgtgaactgggtgcgccaggccccc
ggccgcggcctggagtggatcggcgtgatctggggcgacggcatcaccgactacaacagcgc
cctgaaggaccgcttcatcatcagcaaggacaacggcaagaacaccgtgtacctgcagatga
gcaaggtgcgcagcgacgacaccgccctgtactactgcgtgaccggcctgttcgactactgg
ggccagggcaccctggtgaccgtgagcagctacccatacgatgttccagattacgctggtgg
aggcggaggttctgggggaggaggtagtggcggtggtggttcaggaggcggcggaagcttgg
atccaggtggaggtggaagcggtagcaaaggagaagaacttttcactggagttgtcccaatt
cttgttgaattagatggtgatgttaatgggcacaaattttctgtccgtggagagggtgaagg
tgatgctacaaacggaaaactcaccettaaatttatttgcactactggaaaactacctgttc
cgtggccaacacttgtcactactctgacccatggtgttcaatgcttttcccgttatccggat
cacatgaaacggcatgactttttcaagagtgccatgcccgaaggttatgtacaggaacgcac
tatatctttcaaagatgacgggacctacaagacgcgtgctgaagtcaagtttgaaggtgata
cccttgttaatcgtatcgagttaaagggtattgattttaaagaagatggaaacattcttgga
cacaaactcgagtacaactttaactcacacaatgtatacatcacggcagacaaacaaaagaa
tggaatcaaagctaacttcaaaattcgccacaacgttgaagatggttccgttcaactagcag
accattatcaacaaaatactccaattggcgatggccctgtccttttaccagacaaccattac
ctgtcgacacaatctgtcctttcgaaagatcccaacgaaaagcgtgaccacatggtccttct
tgagtttgtaactgctgctgggattacacatggcatggatgagctctacaaaggtggaggtc
ggaccggtggcggtggcagcggtggaggcggtGCTGACCCCAAGAAGAAGAGGAAGGTGGCT
AGGATCACAAGTTTgtacaaaaaagcaggctccaccgtacaaaaaagcaggctccaccatgg
acaaagattctcaggggctgctagattcatccctgatggcatcaggcactgccagccgctca
gaggatgaggagtcactggcagggcagaagcgagcctcctcccaggccttgggcaccatccc
taaacggagaagctcctccaggttcatcaagaggaagaagttcgatgatgagctggtggaga
gcagcctggcaaaatcttctacccgggcaaagggggccagtggggggaaccagggcgctgt
tcggggagtgaaccctcctccagtgagaagaagaaggtatccaaagcccccagcactcctgt
gccacccagcccagccccagcccctggactcaccaagcgtgtgaagaagagtaaacagccac
ttcaggtgaccaaggatctgggccgctggaagcctgcagatgacctcctgctcataaatgct
gtgttgcagaccaacgacctgacctccgtccacctgggcgtgaaattcagctgccgcttcac
ccttcgggaggtccaggagcgttggtacgccctgctctacgatcctgtcatctccaagttgg
cctgtcaggccatgaggcagctgcacccagaggctattgcagccatccagagcaaggccctg
tttagcaaggctgaggagcagctgctgagcaaagtgggatcgaccagccagcccaccttgga
gaccttccaggacctgctgcacagacaccctgatgccttctacctggcccgtaccgcgaagg
ccctgcaggcccactggcagctcatgaagcagtattacctgctggaggaccagacagtgcag
ccgctgcccaaaggggaccaagtgctgaacttctctgatgcagaggacctgattgatgacag
taagctcaaggacatgcgagatgaggtcctggaacatgagctgatggtggctgaccggcgcc
agaagcgagagattcggcagctggaacaggaactgcataagtggcaggtgctagtggacagc
atcacaggcatgagctctccggacttcgacaaccagacactggcagtgctgcggggccgcat
ggtgcggtacctgatgcgctcgcgtgagatcaccctgggcagagcaaccaaggataaccaga
ttgatgtggacctgtctctggagggtccggcctggaagatatcccggaaacaaggtgtcatc
aagctgaagaacaacggtgatttcttcattgccaatgagggtcgacggcccatctacatcga
tggacggccggtgctctgtggctccaaatggcgcctcagcaacaactctgtggtggagatcg
ccagcctgcgattcgtcttccttatcaaccaggacctcattgccctcatcagggctgaggct
gccaagatcacaccacagttggacccagctttcttgtac
SEQ ID NO: 71
Protein sequence for ScFv-sfBFP-OTUD7B

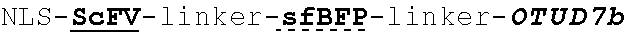

MGPKKKRKVGGMGPDIVMTQSPSSLSASVGDRVTITCRSSTGAVTTSNYASWV
QEKPGKLFKGLIGGTNNRAPGVPSRFSGSLIGDKATLTISSLQPEDFATYFCA
LWYSNHWVFGQGTKVELKRGGGGSGGGGSGGGGSSGGGSEVKLLESGGGLVQP
GGSLKLSCAVSGFSLTDYGVNWVRQAPGRGLEWIGVIWGDGITDYNSALKDRF
IISKDNGKNTVYLQMSKVRSDDTALYYCVTGLEDYWGQGTLVTVSSYPYDVPD

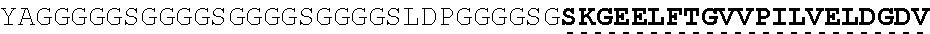

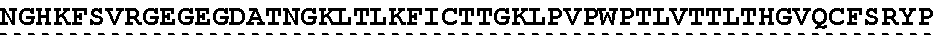

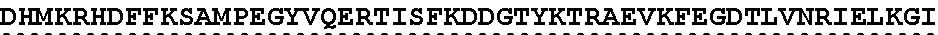

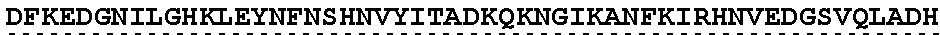

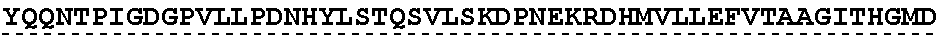

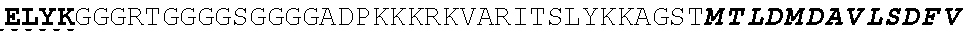

RSTGAEPGLARDLLEGKNWDVNAALSDFEQLRQVHAGNLPPSFSEGSGGSRTP
EKGFSDREPTRPPRPILQRQDDIVQEKRLSRGISHASSSIVSLARSHVSSNGG
GGGSNEHPLEMPICAFQLPDLTVYNEDERSFIERDLIEQSMLVALEQAGRLNW
WVSVDPTSQRLLPLATTGDGNCLLHAASLGMWGFHDRDLMLRKALYALMEKGV
EKEALKRRWRWQQTQQNKESGLVYTEDEWQKEWNELIKLASSEPRMHLGTNGA
NCGGVESSEEPVYESLEEFHVFVLAHVLRRPIVVVADTMLRDSGGEAFAPIPF
GGIYLPLEVPASQCHRSPLVLAYDQAHFSALVSMEQKENTKEQAVIPLTDSEY
KLLPLHFAVDPGKGWEWGKDDSDNVRLASVILSLEVKLHLLHSYMNVKWIPLS
SDAQAPLAQPESPTASAGDEPRSTPESGDSDKESVGSSSTSNEGGRRKEKSKR
DREKDKKRADSVANKLGSFGKTLGSKLKKNMGGLMHSKGSKPGGVGTGLGGSS
GTETLEKKKKNSLKSWKGGKEEAAGDGPVSEKPPAESVGNGGSKYSQEVMQSL
SILRTAMQGEGKFIFVGTLKMGHRHQYQEEMIQRYLSDAEERFLAEQKQKEAE
RKIMNGGIGGGPPPAKKPEPDAREEQPTGPPAESRAMAFSTGYPGDFTIPRPS
GGGVHCQEPRRQLAGGPCVGGLPPYATFPRQCPPGRPYPHQDSIPSLEPGSHS
KDGLHRGALLPPPYRVADSYSNGYREPPEPDGWAGGLRGLPPTQTKCKQPNCS
FYGHPETNNFCSCCYREELRRREREPDGELLVHRFLDPAFLYKVV
SEQ ID NO: 72
DNA sequence for ScFv-sfBFP-OTUD7B
atgggtCCCAAGAAAAAGAGAAAGGTCggtggcatgggccccgacatcgtgatgacccagag
ccccagcagcctgagcgccagcgtgggcgaccgcgtgaccatcacctgccgcagcagcaccg
gcgccgtgaccaccagcaactacgccagctgggtgcaggagaagcccggcaagctgttcaag
ggcctgatcggcggcaccaacaaccgcgcccccggcgtgcccagccgcttcagcggcagcct
gatcggcgacaaggccaccctgaccatcagcagcctgcagcccgaggacttcgccacctact
tctgcgccctgtggtacagcaaccactgggtgttcggccagggcaccaaggtggagctgaag
cgcggcggcggTggAagcggAggcggTggGTCTggTggAggcggcagcTCTggcggAggcag
cgaggtgaagctgctggagagcggcggcggcctggtgcagcccggcggcagcctgaagctga
gctgcgccgtgagcggcttcagcctgaccgactacggcgtgaactgggtgcgccaggccccc
ggccgcggcctggagtggatcggcgtgatctggggcgacggcatcaccgactacaacagcgc
cctgaaggaccgcttcatcatcagcaaggacaacggcaagaacaccgtgtacctgcagatga
gcaaggtgcgcagcgacgacaccgccctgtactactgcgtgaccggcctgttcgactactgg
ggccagggcaccctggtgaccgtgagcagctacccatacgatgttccagattacgctggtgg
aggcggaggttctgggggaggaggtagtggcggtggtggttcaggaggcggcggaagcttgg
atccaggtggaggtggaagcggtagcaaaggagaagaacttttcactggagttgtcccaatt
cttgttgaattagatggtgatgttaatgggcacaaattttctgtccgtggagagggtgaagg
tgatgctacaaacggaaaactcaccettaaatttatttgcactactggaaaactacctgttc
cgtggccaacacttgtcactactctgacccatggtgttcaatgcttttcccgttatccggat
cacatgaaacggcatgactttttcaagagtgccatgcccgaaggttatgtacaggaacgcac
tatatctttcaaagatgacgggacctacaagacgcgtgctgaagtcaagtttgaaggtgata
cccttgttaatcgtatcgagttaaagggtattgattttaaagaagatggaaacattcttgga
cacaaactcgagtacaactttaactcacacaatgtatacatcacggcagacaaacaaaagaa
tggaatcaaagctaacttcaaaattcgccacaacgttgaagatggttccgttcaactagcag
accattatcaacaaaatactccaattggcgatggccctgtccttttaccagacaaccattac
ctgtcgacacaatctgtcctttcgaaagatcccaacgaaaagcgtgaccacatggtccttct
tgagtttgtaactgctgctgggattacacatggcatggatgagctctacaaaggtggaggtc
ggaccggtggcggtggcagcggtggaggcggtGCTGACCCCAAGAAGAAGAGGAAGGTGGCT
AGGATCACAAGTTTgtacaaaaaagcaggctccaccgtacaaaaaagcaggctccaccatga
ccctggacatggatgctgttctgtcagattttgtccgttccacaggagcagagccagggcta
gcgcgagatctcctagaaggaaagaattgggatgtgaatgccgccctcagtgattttgaaca
gctacgtcaagtccatgctggaaacctacccccatcctttagtgaggggagtggtggctcca
ggacccctgaaaaagggttttctgacagagagcctactcgccctccccgacccatcctccag
cggcaggatgacatcgttcaagaaaaacgcctgtctaggggcatctcccacgccagctccag
cattgtttccctggcccggtcccatgtctcctccaatggtgggggtggggggagcaatgagc
accccctggaaatgcccatctgtgccttccagcttccagatctcactgtatacaatgaagac
ttccgcagcttcatagagagagacctcattgagcagtccatgctggttgccttggaacaggc
agggcgtttgaactggtgggtgagtgtggatcccacctctcagaggctgcttcctttggcaa
ctactggagatgggaactgcctcctgcatgcagcctcccttggaatgtggggtttccatgat
cgggacttgatgctgcggaaagctttgtatgcactgatggagaagggagttgagaaggaagc
gttgaaaaggcgctggaggtggcagcagacacagcagaataaagagtcagggctggtataca
cagaagatgaatggcagaaggagtggaatgaactgatcaagcttgcctcaagtgaaccccga
atgcatctaggtaccaatggagccaactgtggtggggtggagagttctgaggagcctgtata
tgagagccttgaagagtttcacgtctttgtccttgctcatgtgcttaggaggcccatagtcg
tcgtggcagacaccatgctgagggactccggaggggaagcatttgcccctattccctttgga
ggaatctatctgcctttggaggtcccagccagccagtgtcaccgctcccctctggtgctcgc
ctatgatcaggcccacttttctgcactcgtgtccatggagcagaaggagaataccaaggaac
aagctgtgatcccacttacagattcagagtataagctgctgcccttgcactttgctgtggac
cctggaaagggctgggagtggggcaaagatgatagtgacaatgtccgattggccagtgtaat
tctgtccctagaggtcaaattgcatctgctgcatagctacatgaatgtgaagtggatcccac
tgtcctctgatgcacaggctcctctggcccagcctgagtcccccaccgcctcagctggagat
gagccccggtccactcctgagtctggagactcagacaaggagtcagttggcagcagttccac
cagcaacgagggcggccggcggaaggagaagtcaaagcgagatcgggagaaggacaagaaga
gagcagattctgtggctaacaaactgggcagctttggcaaaaccttgggcagcaagctcaag
aagaacatggggggcctgatgcacagcaagggttcaaagcctggaggggtggggacagggtt
gggaggaagcagcggcactgagacactggagaagaagaagaaaaactcactgaagagctgga
agggtggcaaggaggaggcagctggggatgggcctgtgtctgagaagcccccagctgagtct
gttggtaacggagggagcaagtatagccaggaggtgatgcaaagcctgagcattctgaggac
tgccatgcaaggggaggggaagtttatttttgttggaaccctgaagatgggtcaccgtcacc
agtatcaggaggaaatgatccagcgctacctttctgatgctgaggagagattcctggcagaa
cagaagcagaaggaggcagagaggaagatcatgaatggaggaatagggggtggccctcctcc
agccaaaaagccagagccagatgctagggaagagcagccgaccggtcccccagcagagtcca
gggcaatggcattttccactggctaccctggggactttactatccctcggccgtctgggggc
ggagtccactgccaggaaccccggaggcagttggcagggggtccatgtgtcgggggcctacc
accatatgccaccttccccagacagtgccctcctgggcgaccctacccccaccaggacagca
tcccttctctggagccaggcagccactctaaggatggacttcacaggggtgccttgttacca
cccccctaccgagtggctgattcctatagcaatggctacagagagccccctgagccagatgg
atgggctggaggtctccggggccttcccccaactcagaccaaatgcaaacaaccgaactgca
gcttctatggacaccctgagacaaacaacttctgttcctgttgttacagggaagaactgagg
aggagggagcgggaaccggatggggagctcctggtgcacaggttcttggacccagctttctt
gtac
SEQ ID NO: 73
Protein sequence for ScFv-sfBFP-LDB1

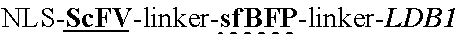

MGPKKKRKVGGMGPDIVMTQSPSSLSASVGDRVTITCRSSTGAVTTSNYASWV
QEKPGKLFKGLIGGINNRAPGVPSRFSGSLIGDKATLTISSLOPEDFATYFCA
LWYSNHWVFGQGTKVELKRGGGGSGGGGSGGGGSSGGGSEVKLLESGGGLVQP
GGSLKLSCAVSGFSLTDYGVNWVRQAPGRGLEWIGVIWGDGITDYNSALKDRF
IISKDNGKNTVYLQMSKVRSDDTALYYCVTGLEDYWGQGTLVTVSSYPYDVPD

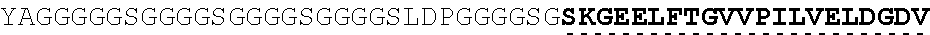

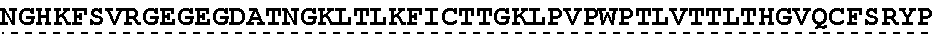

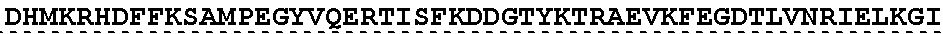

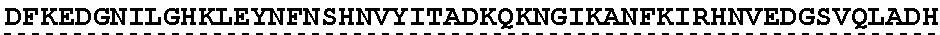

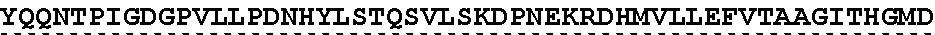

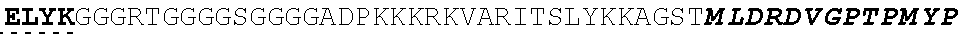

PTYLEPGIGRHTPYGNQTDYRIFELNKRLQNWTEECDNLWWDAFTTEFFEDDA
MLTITFCLEDGPKRYTIGRTLIPRYFRSIFEGGATELYYVLKHPKEAFHSNFV
SLDCDQGSMVTQHGKPMFTQVCVEGRLYLEFMFDDMMRIKTWHESIRQHRELI
PRSILAMHAQDPQMLDQLSKNITRCGLSNSTLNYLRLCVILEPMQELMSRHKT
YSLSPRDCLKTCLFQKWQRMVAPPAEPTRQQPSKRRKRKMSGGSTMSSGGGNT
NNSNSKKKSPASTFALSSQVPDVMVVGEPTLMGGEFGDEDERLITRLENTQFD
AANGIDDEDSFNNSPALGANSPWNSKPPSSQESKSENPTSQASQLDPAFLY
SEQ ID NO: 74
DNA sequence for ScFv-sfBFP-LDB1
atgggtCCCAAGAAAAAGAGAAAGGTCggtggcatgggccccgacatcgtgatgacccagag
ccccagcagcctgagcgccagcgtgggcgaccgcgtgaccatcacctgccgcagcagcaccg
gcgccgtgaccaccagcaactacgccagctgggtgcaggagaagcccggcaagctgttcaag
ggcctgatcggcggcaccaacaaccgcgcccccggcgtgcccagccgcttcagcggcagcct
gatcggcgacaaggccaccctgaccatcagcagcctgcagcccgaggacttcgccacctact
tctgcgccctgtggtacagcaaccactgggtgttcggccagggcaccaaggtggagctgaag
cgcggcggcggTggAagcggAggcggTggGTCTggTggAggcggcagcTCTggcggAggcag
cgaggtgaagctgctggagagcggcggcggcctggtgcagcccggcggcagcctgaagctga
gctgcgccgtgagcggcttcagcctgaccgactacggcgtgaactgggtgcgccaggccccc
ggccgcggcctggagtggatcggcgtgatctggggcgacggcatcaccgactacaacagcgc
cctgaaggaccgcttcatcatcagcaaggacaacggcaagaacaccgtgtacctgcagatga
gcaaggtgcgcagcgacgacaccgccctgtactactgcgtgaccggcctgttcgactactgg
ggccagggcaccctggtgaccgtgagcagctacccatacgatgttccagattacgctggtgg
aggcggaggttctgggggaggaggtagtggcggtggtggttcaggaggcggcggaagcttgg
atccaggtggaggtggaagcggtagcaaaggagaagaacttttcactggagttgtcccaatt
cttgttgaattagatggtgatgttaatgggcacaaattttctgtccgtggagagggtgaagg
tgatgctacaaacggaaaactcaccettaaatttatttgcactactggaaaactacctgttc
cgtggccaacacttgtcactactctgacccatggtgttcaatgcttttcccgttatccggat
cacatgaaacggcatgactttttcaagagtgccatgcccgaaggttatgtacaggaacgcac
tatatctttcaaagatgacgggacctacaagacgcgtgctgaagtcaagtttgaaggtgata
cccttgttaatcgtatcgagttaaagggtattgattttaaagaagatggaaacattcttgga
cacaaactcgagtacaactttaactcacacaatgtatacatcacggcagacaaacaaaagaa
tggaatcaaagctaacttcaaaattcgccacaacgttgaagatggttccgttcaactagcag
accattatcaacaaaatactccaattggcgatggccctgtccttttaccagacaaccattac
ctgtcgacacaatctgtcctttcgaaagatcccaacgaaaagcgtgaccacatggtccttct
tgagtttgtaactgctgctgggattacacatggcatggatgagctctacaaaggtggaggtc
ggaccggtggcggtggcagcggtggaggcggtGCTGACCCCAAGAAGAAGAGGAAGGTGGCT
AGGATCACAAGTTTgtacaaaaaagcaggctccaccatgctggatagggatgtgggcccaac
tcccatgtatccgcctacatacctggagccagggattgggaggcacacaccatatggcaacc
aaactgactacagaatatttgagcttaacaaacggcttcagaactggacagaggagtgtgac
aatctctggtgggatgcattcacgactgagttctttgaggatgatgccatgttgaccatcac
tttctgcctggaggatggaccaaagagatataccattggccggaccctgatcccacgctact
tccgcagcatctttgaggggggtgctacggagctgtactatgttcttaagcaccccaaggag
gcattccacagcaactttgtgtccctcgactgtgaccagggcagcatggtgacccagcatgg
caagcccatgttcacccaggtgtgtgtggagggccggttgtacctggagttcatgtttgacg
acatgatgcggataaagacgtggcacttcagcatccggcagcaccgagagctcatcccccgc
agcatccttgccatgcatgcccaagacccccagatgttggatcagctctccaaaaacatcac
tcggtgtgggctgtccaattccactctcaactacctccgactctgtgtgatactcgagccca
tgcaagagctcatgtcacgccacaagacctacagcctcagcccccgcgactgcctcaagacc
tgccttttccagaagtggcagcgcatggtagcaccccctgcggagcccacacgtcagcagcc
cagcaaacggcggaaacggaagatgtcagggggcagcaccatgagctctggtggtggcaaca
ccaacaacagcaacagcaagaagaagagcccagctagcaccttcgccctctccagccaggta
cctgatgtgatggtggtgggggagcccaccctgatgggcggggagttcggggacgaggacga
gaggctcatcacccggctggagaacacccagtttgacgcagccaacggcattgacgacgagg
acagctttaacaactcccctgcactgggcgccaacagcccctggaacagcaagcctccgtcc
agccaagaaagcaaatcggagaaccccacgtcacaggcctcccagttggacccagctttctt
gtac
SEQ ID NO: 75
Protein sequence for ScFv-sfBFP-NFKBIB

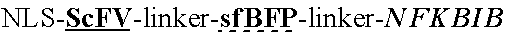

MGPKKKRKVGGMGPDIVMTQSPSSLSASVGDRVTITCRSSTGAVTTSNYASWV
QEKPGKLFKGLIGGINNRAPGVPSRFSGSLIGDKATLTISSLOPEDFATYFCA
LWYSNHWVFGQGTKVELKRGGGGSGGGGSGGGGSSGGGSEVKLLESGGGLVQP
GGSLKLSCAVSGFSLTDYGVNWVRQAPGRGLEWIGVIWGDGITDYNSALKDRF
IISKDNGKNTVYLQMSKVRSDDTALYYCVTGLEDYWGQGTLVTVSSYPYDVPD

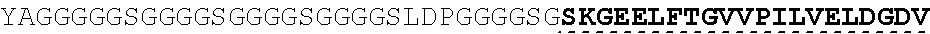

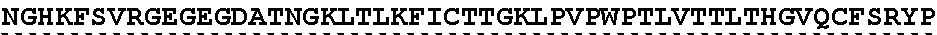

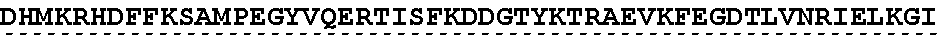

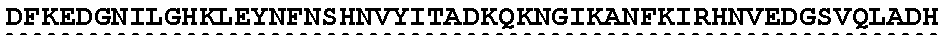

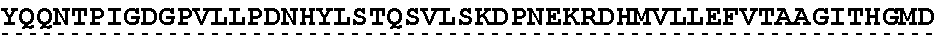

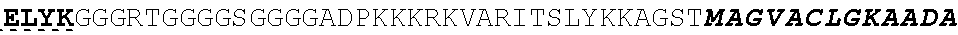

DEWCDSGLGSLGPDAAAPGGPGLGAELGPGLSWAPLVFGYVTEDGDTALHLAV
IHQHEPFLDFLLGESAGTEYMDLQNDLGQTALHLAAILGETSTVEKLYAAGAG
LCVAERRGHTALHLACRVGAHACARALLQPRPRRPREAPDTYLAQGPDRTPDT
NHTPVALYPDSDLEKEEEESEEDWKLQLEAENYEGHTPLHVAVIHKDVEMVRL
LRDAGADLDKPEPTCGRSPLHLAVEAQAADVLELLLRAGANPAARMYGGRTPL
GSAMLRPNPILARLLRAHGAPEPEGEDEKSGPCSSSSDSDSGDEGDEYDDIVV
HSSRSQTRLPPTPASKPLPDDPRPV
SEQ ID NO: 76
DNA sequence for ScFv-sfBFP-NFKBIB
atgggtCCCAAGAAAAAGAGAAAGGTCggtggcatgggccccgacatcgtgatgacccagag
ccccagcagcctgagcgccagcgtgggcgaccgcgtgaccatcacctgccgcagcagcaccg
gcgccgtgaccaccagcaactacgccagctgggtgcaggagaagcccggcaagctgttcaag
ggcctgatcggcggcaccaacaaccgcgcccccggcgtgcccagccgcttcagcggcagcct
gatcggcgacaaggccaccctgaccatcagcagcctgcagcccgaggacttcgccacctact
tctgcgccctgtggtacagcaaccactgggtgttcggccagggcaccaaggtggagctgaag
cgcggcggcggTggAagcggAggcggTggGTCTggTggAggcggcagcTCTggcggAggcag
cgaggtgaagctgctggagagcggcggcggcctggtgcagcccggcggcagcctgaagctga
gctgcgccgtgagcggcttcagcctgaccgactacggcgtgaactgggtgcgccaggccccc
ggccgcggcctggagtggatcggcgtgatctggggcgacggcatcaccgactacaacagcgc
cctgaaggaccgcttcatcatcagcaaggacaacggcaagaacaccgtgtacctgcagatga
gcaaggtgcgcagcgacgacaccgccctgtactactgcgtgaccggcctgttcgactactgg
ggccagggcaccctggtgaccgtgagcagctacccatacgatgttccagattacgctggtgg
aggcggaggttctgggggaggaggtagtggcggtggtggttcaggaggcggcggaagcttgg
atccaggtggaggtggaagcggtagcaaaggagaagaacttttcactggagttgtcccaatt
cttgttgaattagatggtgatgttaatgggcacaaattttctgtccgtggagagggtgaagg
tgatgctacaaacggaaaactcaccettaaatttatttgcactactggaaaactacctgttc
cgtggccaacacttgtcactactctgacccatggtgttcaatgcttttcccgttatccggat
cacatgaaacggcatgactttttcaagagtgccatgcccgaaggttatgtacaggaacgcac
tatatctttcaaagatgacgggacctacaagacgcgtgctgaagtcaagtttgaaggtgata
cccttgttaatcgtatcgagttaaagggtattgattttaaagaagatggaaacattcttgga
cacaaactcgagtacaactttaactcacacaatgtatacatcacggcagacaaacaaaagaa
tggaatcaaagctaacttcaaaattcgccacaacgttgaagatggttccgttcaactagcag
accattatcaacaaaatactccaattggcgatggccctgtccttttaccagacaaccattac
ctgtcgacacaatctgtcctttcgaaagatcccaacgaaaagcgtgaccacatggtccttct
tgagtttgtaactgctgctgggattacacatggcatggatgagctctacaaaggtggaggtc
ggaccggtggcggtggcagcggtggaggcggtGCTGACCCCAAGAAGAAGAGGAAGGTGGCT
AGGATCACAAGTTTgtacaaaaaagcaggctccaccATGGCTGGGGTCGCGTGCTTGGGAAA
AGCTGCCGACGCAGATGAATGGTGCGACAGCGGCCTGGGCTCCCTGGGTCCGGACGCAGCGG
CCCCCGGAGGACCTGGGTTGGGCGCGGAGTTGGGCCCGGGGCTGTCGTGGGCTCCCCTCGTC
TTCGGCTACGTCACTGAGGATGGGGACACGGCACTGCACTTGGCTGTGATTCATCAGCATGA
ACCCTTCCTGGATTTTCTTCTAGGCTTCTCGGCCGGCACTGAGTACATGGACCTGCAGAATG
ACCTAGGCCAGACAGCCCTGCACCTGGCAGCCATCCTGGGGGAGACATCCACGGTGGAGAAG
CTGTACGCAGCAGGCGCCGGGCTGTGTGTGGCGGAGCGTAGGGGCCACACGGCGCTGCACCT
GGCCTGCCGTGTGGGGGCACACGCCTGTGCCCGTGCCCTGCTTCAGCCCCGCCCCCGGCGCC
CCAGGGAAGCCCCCGACACCTACCTCGCTCAGGGCCCTGACCGTACTCCCGACACCAACCAT
ACCCCTGTCGCCTTGTACCCCGATTCCGACTTGGAGAAGGAAGAAGAGGAGAGTGAGGAGGA
CTGGAAGCTGCAGCTGGAGGCTGAAAACTACGAGGGCCACACCCCACTCCACGTGGCCGTTA
TCCACAAAGATGTGGAGATGGTCCGGCTGCTCCGAGATGCTGGAGCTGACCTTGACAAACCG
GAGCCCACGTGCGGCCGGAGCCCCCTTCATTTGGCAGTGGAGGCCCAGGCAGCCGATGTGCT
GGAGCTTCTCCTGAGGGCAGGCGCGAACCCTGCTGCCCGCATGTACGGTGGCCGCACCCCAC
TCGGCAGTGCCATGCTCCGGCCCAACCCCATCCTCGCCCGCCTCCTCCGTGCACACGGAGCC
CCTGAGCCCGAGGGCGAGGACGAGAAATCCGGCCCCTGCAGCAGCAGTAGCGACAGCGACAG
CGGAGACGAGGGCGATGAATACGACGACATTGTGGTTCACAGCAGCCGCAGCCAAACCCGGC
TGCCTCCCACCCCAGCCTCAAAACCTCTTCCTGACGACCCCCGCCCCGTGTGA
SEQ ID NO: 77
Protein sequence for ScFv-sfBFP-RelB

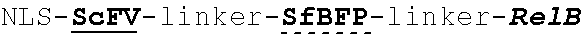

MGPKKKRKVGGMGPDIVMTQSPSSLSASVGDRVTITCRSSTGAVTTSNYASWV
QEKPGKLFKGLIGGTNNRAPGVPSRFSGSLIGDKATLTISSLQPEDFATYFCA
LWYSNHWVFGQGTKVELKRGGGGSGGGGSGGGGSSGGGSEVKLLESGGGLVQP
GGSLKLSCAVSGFSLTDYGVNWVRQAPGRGLEWIGVIWGDGITDYNSALKDRF
IISKDNGKNTVYLQMSKVRSDDTALYYCVTGLEDYWGQGTLVTVSSYPYDVPD

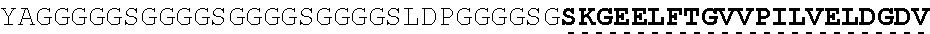

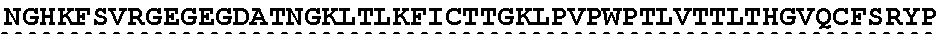

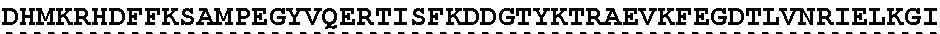

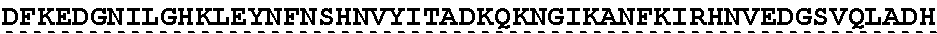

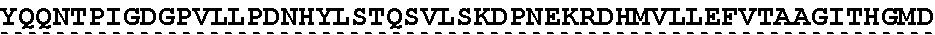

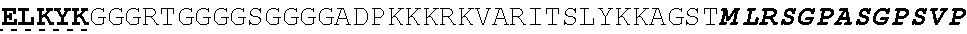

TGRAMPSRRVARPPAAPELGALGSPDLSSLSLAVSRSTDELEIIDEYIKENGF
GLDGGQPGPGEGLPRLVSRGAASLSTVTLGPVAPPATPPPWGCPLGRLVSPAP
GPGPQPHLVITEQPKQRGMRFRYECEGRSAGSILGESSTEASKTLPAIELRDC
GGLREVEVTACLVWKDWPHRVHPHSLVGKDCTDGICRVRLRPHVSPRHSENNL
GIQCVRKKEIEAAIERKIQLGIDPYNAGSLKNHQEVDMNVVRICFQASYRDQQ
GQMRRMDPVLSEPVYDKKSTNTSELRICRINKESGPCTGGEELYLLCDKVQKE
DISVVFSRASWEGRADFSQADVHRQIAIVFKTPPYEDLEIVEPVTVNVFLQRL
TDGVCSEPLPFTYLPRDHDSYGVDKKRKRGMPDVLGELNSSDPHGIESKRRKK
KPAILDHELPNHGSGPFLPPSALLPDPDFFSGTVSLPGLEPPGGPDLLDDGFA
YDPTAPTLFTMLDLLPPAPPHASAVVCSGGAGAVVGETPGPEPLTLDSYQAPG
PGDGGTASLVGSNMFPNHYREAAFGGGLLSPGPEAT
SEQ ID NO: 78
DNA sequence for ScFv-sfBFP-RelB
atgggtCCCAAGAAAAAGAGAAAGGTCggtggcatgggccccgacatcgtgatgacccagag
ccccagcagcctgagcgccagcgtgggcgaccgcgtgaccatcacctgccgcagcagcaccg
gcgccgtgaccaccagcaactacgccagctgggtgcaggagaagcccggcaagctgttcaag
ggcctgatcggcggcaccaacaaccgcgcccccggcgtgcccagccgcttcagcggcagcct
gatcggcgacaaggccaccctgaccatcagcagcctgcagcccgaggacttcgccacctact
tctgcgccctgtggtacagcaaccactgggtgttcggccagggcaccaaggtggagctgaag
cgcggcggcggTggAagcggAggcggTggGTCTggTggAggcggcagcTCTggcggAggcag
cgaggtgaagctgctggagagcggcggcggcctggtgcagcccggcggcagcctgaagctga
gctgcgccgtgagcggcttcagcctgaccgactacggcgtgaactgggtgcgccaggccccc
ggccgcggcctggagtggatcggcgtgatctggggcgacggcatcaccgactacaacagcgc
cctgaaggaccgcttcatcatcagcaaggacaacggcaagaacaccgtgtacctgcagatga
gcaaggtgcgcagcgacgacaccgccctgtactactgcgtgaccggcctgttcgactactgg
ggccagggcaccctggtgaccgtgagcagctacccatacgatgttccagattacgctggtgg
aggcggaggttctgggggaggaggtagtggcggtggtggttcaggaggcggcggaagcttgg
atccaggtggaggtggaagcggtagcaaaggagaagaacttttcactggagttgtcccaatt
cttgttgaattagatggtgatgttaatgggcacaaattttctgtccgtggagagggtgaagg
tgatgctacaaacggaaaactcaccettaaatttatttgcactactggaaaactacctgttc
cgtggccaacacttgtcactactctgacccatggtgttcaatgcttttcccgttatccggat
cacatgaaacggcatgactttttcaagagtgccatgcccgaaggttatgtacaggaacgcac
tatatctttcaaagatgacgggacctacaagacgcgtgctgaagtcaagtttgaaggtgata
cccttgttaatcgtatcgagttaaagggtattgattttaaagaagatggaaacattcttgga
cacaaactcgagtacaactttaactcacacaatgtatacatcacggcagacaaacaaaagaa
tggaatcaaagctaacttcaaaattcgccacaacgttgaagatggttccgttcaactagcag
accattatcaacaaaatactccaattggcgatggccctgtccttttaccagacaaccattac
ctgtcgacacaatctgtcctttcgaaagatcccaacgaaaagcgtgaccacatggtccttct
tgagtttgtaactgctgctgggattacacatggcatggatgagctctacaaaggtggaggtc
ggaccggtggcggtggcagcggtggaggcggtGCTGACCCCAAGAAGAAGAGGAAGGTGGCT
AGGATCACAAGTTTgtacaaaaaagcaggctccaccATGCTTCGGTCTGGGCCAGCCTCTGG
GCCGTCCGTCCCCACTGGCCGGGCCATGCCGAGTCGCCGCGTCGCCAGACCGCCGGCTGCGC
CGGAGCTGGGGGCCTTAGGGTCCCCCGACCTCTCCTCACTCTCGCTCGCCGTTTCCAGGAGC
ACAGATGAATTGGAGATCATCGACGAGTACATCAAGGAGAACGGCTTCGGCCTGGACGGGGG
ACAGCCGGGCCCGGGCGAGGGGCTGCCACGCCTGGTGTCTCGCGGGGCTGCGTCCCTGAGCA
CGGTCACCCTGGGCCCTGTGGCGCCCCCAGCCACGCCGCCGCCTTGGGGCTGCCCCCTGGGC
CGACTAGTGTCCCCAGCGCCGGGCCCGGGCCCGCAGCCGCACCTGGTCATCACGGAGCAGCC
CAAGCAGCGCGGCATGCGCTTCCGCTACGAGTGCGAGGGCCGCTCGGCCGGCAGCATCCTTG
GGGAGAGCAGCACCGAGGCCAGCAAGACGCTGCCCGCCATCGAGCTCCGGGATTGTGGAGGG
CTGCGGGAGGTGGAGGTGACTGCCTGCCTGGTGTGGAAGGACTGGCCTCACCGAGTCCACCC
CCACAGCCTCGTGGGGAAAGACTGCACCGACGGCATCTGCAGGGTGCGGCTCCGGCCTCACG
TCAGCCCCCGGCACAGTTTTAACAACCTGGGCATCCAGTGTGTGAGGAAGAAGGAGATTGAG
GCTGCCATTGAGCGGAAGATTCAACTGGGCATTGACCCCTACAACGCTGGGTCCCTGAAGAA
CCATCAGGAAGTAGACATGAATGTGGTGAGGATCTGCTTCCAGGCCTCATATCGGGACCAGC
AGGGACAGATGCGCCGGATGGATCCTGTGCTTTCCGAGCCCGTCTATGACAAGAAATCCACA
AACACATCAGAGCTGCGGATTTGCCGAATTAACAAGGAAAGCGGGCCGTGCACCGGTGGCGA
GGAGCTCTACTTGCTCTGCGACAAGGTGCAGAAAGAGGACATATCAGTGGTGTTCAGCAGGG
CCTCCTGGGAAGGTCGGGCTGACTTCTCCCAGGCCGACGTGCACCGCCAGATTGCCATTGTG
TTCAAGACGCCGCCCTACGAGGACCTGGAGATTGTCGAGCCCGTGACAGTCAACGTCTTCCT
GCAGCGGCTCACCGATGGGGTCTGCAGCGAGCCATTGCCTTTCACGTACCTGCCTCGCGACC
ATGACAGCTACGGCGTGGACAAGAAGCGGAAACGGGGGATGCCCGACGTCCTTGGGGAGCTG
AACAGCTCTGACCCCCATGGCATCGAGAGCAAACGGCGGAAGAAAAAGCCGGCCATCCTGGA
CCACTTCCTGCCCAACCACGGCTCAGGCCCGTTCCTCCCGCCGTCAGCCCTGCTGCCAGACC
CTGACTTCTTCTCTGGCACCGTGTCCCTGCCCGGCCTGGAGCCCCCTGGCGGGCCTGACCTC
CTGGACGATGGCTTTGCCTACGACCCTACGGCCCCCACACTCTTCACCATGCTGGACCTGCT
GCCCCCGGCACCGCCACACGCTAGCGCTGTTGTGTGCAGCGGAGGTGCCGGGGCCGTGGTTG
GGGAGACCCCCGGCCCTGAACCACTGACACTGGACTCGTACCAGGCCCCGGGCCCCGGGGAT
GGAGGCACCGCCAGCCTTGTGGGCAGCAACATGTTCCCCAATCATTACCGCGAGGCGGCCTT
TGGGGGCGGCCTCCTATCCCCGGGGCCTGAAGCCACGTAG
SEQ ID NO: 79
Protein sequence for ScFv-sfBFP-CITED2

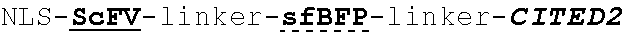

MGPKKKRKVGGMGPDIVMTQSPSSLSASVGDRVTITCRSSTGAVTTSNYASWV
QEKPGKLFKGLIGGINNRAPGVPSRFSGSLIGDKATLTISSLOPEDFATYFCA
LWYSNHWVFGQGTKVELKRGGGGSGGGGSGGGGSSGGGSEVKLLESGGGLVQP
GGSLKLSCAVSGFSLTDYGVNWVRQAPGRGLEWIGVIWGDGITDYNSALKDRE
IISKDNGKNTVYLQMSKVRSDDTALYYCVTGLEDYWGQGTLVTVSSYPYDVPD

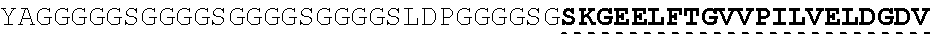

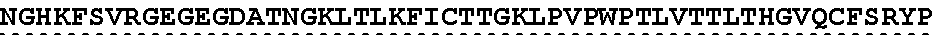

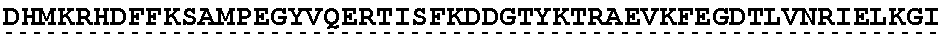

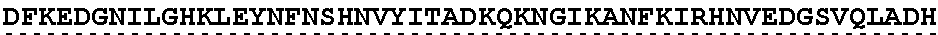

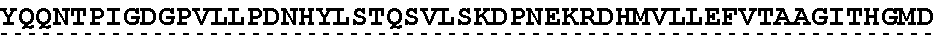

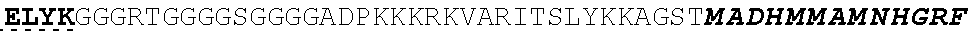

PDGTNGLHHHPAHRMGMGQFPSPHHHQQQQPQHAFNALMGEHIHYGAGNMNAT
SGIRHAMGPGTVNGGHPPSALAPAARFNNSQFMGPPVASQGGSLPASMQLQKL
NNQYFNHHPYPHNHYMPDLHPAAGHQMNGTNQHERDCNPKHSGGSSTPGGSGG
SSTPGGSGSSSGGGAGSSNSGGGSGSGNMPASVAHVPAAMLPPNVIDTDFIDE
EVLMSLVIEMGLDRIKELPELWLGQNEEDFMTDFVCKQQPSRVSCLDPAFLY
SEQ ID NO: 80
DNA sequence for ScFv-sfBFP-CITED2
atgggtCCCAAGAAAAAGAGAAAGGTCggtggcatgggccccgacatcgtgatgacccagag
ccccagcagcctgagcgccagcgtgggcgaccgcgtgaccatcacctgccgcagcagcaccg
gcgccgtgaccaccagcaactacgccagctgggtgcaggagaagcccggcaagctgttcaag
ggcctgatcggcggcaccaacaaccgcgcccccggcgtgcccagccgcttcagcggcagcct
gatcggcgacaaggccaccctgaccatcagcagcctgcagcccgaggacttcgccacctact
tctgcgccctgtggtacagcaaccactgggtgttcggccagggcaccaaggtggagctgaag
cgcggcggcggTggAagcggAggcggTggGTCTggTggAggcggcagcTCTggcggAggcag
cgaggtgaagctgctggagagcggcggcggcctggtgcagcccggcggcagcctgaagctga
gctgcgccgtgagcggcttcagcctgaccgactacggcgtgaactgggtgcgccaggccccc
ggccgcggcctggagtggatcggcgtgatctggggcgacggcatcaccgactacaacagcgc
cctgaaggaccgcttcatcatcagcaaggacaacggcaagaacaccgtgtacctgcagatga
gcaaggtgcgcagcgacgacaccgccctgtactactgcgtgaccggcctgttcgactactgg
ggccagggcaccctggtgaccgtgagcagctacccatacgatgttccagattacgctggtgg
aggcggaggttctgggggaggaggtagtggcggtggtggttcaggaggcggcggaagcttgg
atccaggtggaggtggaagcggtagcaaaggagaagaacttttcactggagttgtcccaatt
cttgttgaattagatggtgatgttaatgggcacaaattttctgtccgtggagagggtgaagg
tgatgctacaaacggaaaactcaccettaaatttatttgcactactggaaaactacctgttc
cgtggccaacacttgtcactactctgacccatggtgttcaatgcttttcccgttatccggat
cacatgaaacggcatgactttttcaagagtgccatgcccgaaggttatgtacaggaacgcac
tatatctttcaaagatgacgggacctacaagacgcgtgctgaagtcaagtttgaaggtgata
cccttgttaatcgtatcgagttaaagggtattgattttaaagaagatggaaacattcttgga
cacaaactcgagtacaactttaactcacacaatgtatacatcacggcagacaaacaaaagaa
tggaatcaaagctaacttcaaaattcgccacaacgttgaagatggttccgttcaactagcag
accattatcaacaaaatactccaattggcgatggccctgtccttttaccagacaaccattac
ctgtcgacacaatctgtcctttcgaaagatcccaacgaaaagcgtgaccacatggtccttct
tgagtttgtaactgctgctgggattacacatggcatggatgagctctacaaaggtggaggtc
ggaccggtggcggtggcagcggtggaggcggtGCTGACCCCAAGAAGAAGAGGAAGGTGGCT
AGGATCACAAGTTTgtacaaaaaagcaggctccaccatggcagaccatatgatggcaatgaa
ccacgggcgcttccccgacggcaccaatgggctgcaccatcaccctgcccaccgcatgggca
tggggcagttcccgagcccccatcaccaccagcagcagcagccccagcacgccttcaacgcc
ctaatgggcgagcacatacactacggcgcgggcaacatgaatgccacgagcggcatcaggca
tgcgatggggccggggactgtgaacggagggcaccccccgagcgcgctggcccccgcggcca
ggtttaacaactcccagttcatgggtcccccggtggccagccagggaggctccctgccggcc
agcatgcagctgcagaagctcaacaaccagtatttcaaccatcacccctacccccacaacca
ctacatgccggatttgcaccctgctgcaggccaccagatgaacgggacaaaccagcacttcc
gagattgcaaccccaagcacagcggcggcagcagcacccccggcggctcgggcggcagcagc
acccccggcggctctggcagcagctcgggcggcggcgcgggcagcagcaacagcggcggcgg
cagcggcagcggcaacatgcccgcctccgtggcccacgtccccgctgcaatgctgccgccca
atgtcatagacactgatttcatcgacgaggaagttcttatgtccttggtgatagaaatgggt
ttggaccgcatcaaggagctgcccgaactctggctggggcaaaacgagtttgattttatgac
ggacttcgtgtgcaaacagcagcccagcagagtgagctgtttggacccagctttcttgtac
SEQ ID NO: 81
Protein sequence for scFv
MGPDIVMTQSPSSLSASVGDRVTITCRSSTGAVTTSNYASWVQEKPGKLFKGLIGGTNNRAP
GVPSRFSGSLIGDKATLTISSLQPEDFATYFCALWYSNHWVFGQGTKVELKRGGGGSGGGGS
GGGGSSGGGSEVKLLESGGGLVQPGGSLKLSCAVSGFSLTDYGVNWVRQAPGRGLEWIGVIW
GDGITDYNSALKDRFIISKDNGKNTVYLQMSKVRSDDTALYYCVTGLFDYWGQGTLVTVSS
SEQ ID NO: 82
DNA sequence for scFv
atgggccccgacatcgtgatgacccagagccccagcagcctgagcgccagcgtgggcgaccg
cgtgaccatcacctgccgcagcagcaccggcgccgtgaccaccagcaactacgccagctggg
tgcaggagaagcccggcaagctgttcaagggcctgatcggcggcaccaacaaccgcgccccc
ggcgtgcccagccgcttcagcggcagcctgatcggcgacaaggccaccctgaccatcagcag
cctgcagcccgaggacttcgccacctacttctgcgccctgtggtacagcaaccactgggtgt
tcggccagggcaccaaggtggagctgaagcgcggcggcggTggAagcggAggcggTggGTCT
ggTggAggcggcagcTCTggcggAggcagcgaggtgaagctgctggagagcggcggcggcct
ggtgcagcccggcggcagcctgaagctgagctgcgccgtgagcggcttcagcctgaccgact
acggcgtgaactgggtgcgccaggcccccggccgcggcctggagtggatcggcgtgatctgg
ggcgacggcatcaccgactacaacagcgccctgaaggaccgcttcatcatcagcaaggacaa
cggcaagaacaccgtgtacctgcagatgagcaaggtgcgcagcgacgacaccgccctgtact
actgcgtgaccggcctgttcgactactggggccagggcaccctggtgaccgtgagcagctac
ccatacgatgttccagattacgctggtggaggcggaggttctgggggaggaggtagtggcgg
tggtggttcaggaggcggcggaagcttggatccaggtggaggtggaagcggt
SEQ ID NO: 83
Protein sequence for sfBFP
SKGEELFTGVVPILVELDGDVNGHKFSVRGEGEGDATNGKLTLKFICTTGKLPVPWPTLVTT
LTHGVQCFSRYPDHMKRHDFFKSAMPEGYVQERTISFKDDGTYKTRAEVKFEGDTLVNRIEL
KGIDFKEDGNILGHKLEYNFNSHNVYITADKQKNGIKANFKIRHNVEDGSVQLADHYQQNTP
IGDGPVLLPDNHYLSTQSVLSKDPNEKRDHMVLLEFVTAAGITHGMDELYK
SEQ ID NO: 84
DNA sequence for sfBFP
agcaaaggagaagaacttttcactggagttgtcccaattcttgttgaattagatggtgatgt
taatgggcacaaattttctgtccgtggagagggtgaaggtgatgctacaaacggaaaactca
cccttaaatttatttgcactactggaaaactacctgttccgtggccaacacttgtcactact
ctgacccatggtgttcaatgcttttcccgttatccggatcacatgaaacggcatgacttttt
caagagtgccatgcccgaaggttatgtacaggaacgcactatatctttcaaagatgacggga
cctacaagacgcgtgctgaagtcaagtttgaaggtgatacccttgttaatcgtatcgagtta
aagggtattgattttaaagaagatggaaacattcttggacacaaactcgagtacaactttaa
ctcacacaatgtatacatcacggcagacaaacaaaagaatggaatcaaagctaacttcaaaa
ttcgccacaacgttgaagatggttccgttcaactagcagaccattatcaacaaaatactcca
attggcgatggccctgtccttttaccagacaaccattacctgtcgacacaatctgtcctttc
gaaagatcccaacgaaaagcgtgaccacatggtccttcttgagtttgtaactgctgctggga
ttacacatggcatggatgagctctacaaa
SEQ ID NO: 85
Protein sequence for GCN4 Peptide (which is bound by ScFV)
EELLSKNYHLENEVARLKK
SEQ ID NO: 86
DNA sequence for GCN4 (one example of a sequence for GCN4)
gaggagcttctgagcaaaaactatcacctcgaaaacgaggttgcgcgactgaagaaa
SEQ ID NO: 87
Protein sequence for dCas9-5X-GCN4 (GCN4 is underlined)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTEDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRENASLGTYHDLLKIIKDKDELDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSD
NVPSEEVVKKMKNYWRQLLNAKLITQRKEDNLTKAERGGLSELDKAGFIKRQLVETRQITKH
VAQILDSRMNTKYDENDKLIREVKVITLKSKLVSDERKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGESKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYEDTTIDRKRYTSTKEVLDATLIHQSITGLYETRIDLSQ
LGGDSRADPKKKRKVASDGIGSGSNGSSGSNGPTDAAEEELLSKNYHLENEVARLKKGSGSG
GSGSGSGGSGSGGSGSGEELLSKNYHLENEVARLKKGSGSGGSGSGSGGSGSGGSGSGEELL
SKNYHLENEVARLKKGSGSGGSGSGSGGSGSGGSGSGEELLSKNYHLENEVARLKKGSGSGG
SGSGSGGSGSGGSGSGEELLSKNYHLENEVARLKKGSGSGGSGSGSGGSGSGGSGSG
SEQ ID NO: 88
DNA sequence for dCas9-5X-GCN4
ATGGACAAGAAGTACTCCATTGGGCTCGCCATCGGCACAAACAGCGTCGGCTGGGCCGTCAT
TACGGACGAGTACAAGGTGCCGAGCAAAAAATTCAAAGTTCTGGGCAATACCGATCGCCACA
GCATAAAGAAGAACCTCATTGGCGCCCTCCTGTTCGACTCCGGGGAAACCGCCGAAGCCACG
CGGCTCAAAAGAACAGCACGGCGCAGATATACCCGCAGAAAGAATCGGATCTGCTACCtgca
GGAGATCTTTAGTAATGAGATGGCTAAGGTGGATGACTCTTTCTTCCATAGGCTGGAGGAGT
CCTTTTTGGTGGAGGAGGATAAAAAGCACGAGCGCCACCCAATCTTTGGCAATATCGTGGAC
GAGGTGGCGTACCATGAAAAGTACCCAACCATATATCATCTGAGGAAGAAGCTTGTAGACAG
TACTGATAAGGCTGACTTGCGGTTGATCTATCTCGCGCTGGCGCATATGATCAAATTTCGGG
GACACTTCCTCATCGAGGGGGACCTGAACCCAGACAACAGCGATGTCGACAAACTCTTTATC
CAACTGGTTCAGACTTACAATCAGCTTTTCGAAGAGAACCCGATCAACGCATCCGGAGTTGA
CGCCAAAGCAATCCTGAGCGCTAGGCTGTCCAAATCCCGGCGGCTCGAAAACCTCATCGCAC
AGCTCCCTGGGGAGAAGAAGAACGGCCTGTTTGGTAATCTTATCGCCCTGTCACTCGGGCTG
ACCCCCAACTTTAAATCTAACTTCGACCTGGCCGAAGATGCCAAGCTTCAACTGAGCAAAGA
CACCTACGATGATGATCTCGACAATCTGCTGGCCCAGATCGGCGACCAGTACGCAGACCTTT
TTTTGGCGGCAAAGAACCTGTCAGACGCCATTCTGCTGAGTGATATTCTGCGAGTGAACACG
GAGATCACCAAAGCTCCGCTGAGCGCTAGTATGATCAAGCGCTATGATGAGCACCACCAAGA
CTTGACTTTGCTGAAGGCCCTTGTCAGACAGCAACTGCCTGAGAAGTACAAGGAAATTTTCT
TCGATCAGTCTAAAAATGGCTACGCCGGATACATTGACGGCGGAGCAAGCCAGGAGGAATTT
TACAAATTTATTAAGCCCATCTTGGAAAAAATGGACGGCACCGAGGAGCTGCTGGTAAAGCT
TAACAGAGAAGATCTGTTGCGCAAACAGCGCACTTTCGACAATGGAAGCATCCCCCACCAGA
TTCACCTGGGCGAACTGCACGCTATCCTCAGGCGGCAAGAGGATTTCTACCCCTTTTTGAAA
GATAACAGGGAAAAGATTGAGAAAATCCTCACATTTCGGATACCCTACTATGTAGGCCCCCT
CGCCCGGGGAAATTCCAGATTCGCGTGGATGACTCGCAAATCAGAAGAGACCATCACTCCCT
GGAACTTCGAGGAAGTCGTGGATAAGGGGGCCTCTGCCCAGTCCTTCATCGAAAGGATGACT
AACTTTGATAAAAATCTGCCTAACGAAAAGGTGCTTCCTAAACACTCTCTGCTGTACGAGTA
CTTCACAGTTTATAACGAGCTCACCAAGGTCAAATACGTCACAGAAGGGATGAGAAAGCCAG
CATTCCTGTCTGGAGAGCAGAAGAAAGCTATCGTGGACCTCCTCTTCAAGACGAACCGGAAA
GTTACCGTGAAACAGCTCAAAGAAGACTATTTCAAAAAGATTGAATGTTTCGACTCTGTTGA
AATCAGCGGAGTGGAGGATCGCTTCAACGCATCCCTGGGAACGTATCACGATCTCCTGAAAA
TCATTAAAGACAAGGACTTCCTGGACAATGAGGAGAACGAGGACATTCTTGAGGACATTGTC
CTCACCCTTACGTTGTTTGAAGATAGGGAGATGATTGAAGAACGCTTGAAAACTTACGCTCA
TCTCTTCGACGACAAAGTCATGAAACAGCTCAAGAGGCGCCGATATACAGGATGGGGGCGGC
TGTCAAGAAAACTGATCAATGGgatcCGAGACAAGCAGAGTGGAAAGACAATCCTGGATTTT
CTTAAGTCCGATGGATTTGCCAACCGGAACTTCATGCAGTTGATCCATGATGACTCTCTCAC
CTTTAAGGAGGACATCCAGAAAGCACAAGTTTCTGGCCAGGGGGACAGTCTTCACGAGCACA
TCGCTAATCTTGCAGGTAGCCCAGCTATCAAAAAGGGAATACTGCAGACCGTTAAGGTCGTG
GATGAACTCGTCAAAGTAATGGGAAGGCATAAGCCCGAGAATATCGTTATCGAGATGGCCCG
AGAGAACCAAACTACCCAGAAGGGACAGAAGAACAGTAGGGAAAGGATGAAGAGGATTGAAG
AGGGTATAAAAGAACTGGGGTCCCAAATCCTTAAGGAACACCCAGTTGAAAACACCCAGCTT
CAGAATGAGAAGCTCTACCTGTACTACCTGCAGAACGGCAGGGACATGTACGTGGATCAGGA
ACTGGACATCAATCGGCTCTCCGACTACGACGTGGATGCCATCGTGCCCCAGTCTTTTCTCA
AAGATGATTCTATTGATAATAAAGTGTTGACAAGATCCGATAAAAATAGAGGGAAGAGTGAT
AACGTCCCCTCAGAAGAAGTTGTCAAGAAAATGAAAAATTATTGGCGGCAGCTGCTGAACGC
CAAACTGATCACACAACGGAAGTTCGATAATCTGACTAAGGCTGAACGAGGTGGCCTGTCTG
AGTTGGATAAAGCCGGCTTCATCAAAAGGCAGCTTGTTGAGACACGCCAGATCACCAAgcac
GTGGCCCAAATTCTCGATTCACGCATGAACACCAAGTACGATGAAAATGACAAACTGATTCG
AGAGGTGAAAGTTATTACTCTGAAGTCTAAGCTGGTCTCAGATTTCAGAAAGGACTTTCAGT
TTTATAAGGTGAGAGAGATCAACAATTACCACCATGCGCATGATGCCTACCTGAATGCAGTG
GTAGGCACTGCACTTATCAAAAAATATCCCAAGCTTGAATCTGAATTTGTTTACGGAGACTA
TAAAGTGTACGATGTTAGGAAAATGATCGCAAAGTCTGAGCAGGAAATAGGCAAGGCCACCG
CTAAGTACTTCTTTTACAGCAATATTATGAATTTTTTCAAGACCGAGATTACACTGGCCAAT
GGAGAGATTCGGAAGCGACCACTTATCGAAACAAACGGAGAAACAGGAGAAATCGTGTGGGA
CAAGGGTAGGGATTTCGCGACAGTCCGGAAGGTCCTGTCCATGCCGCAGGTGAACATCGTTA
AAAAGACCGAAGTACAGACCGGAGGCTTCTCCAAGGAAAGTATCCTCCCGAAAAGGAACAGC
GACAAGCTGATCGCACGCAAAAAAGATTGGGACCCCAAGAAATACGGCGGATTCGATTCTCC
TACAGTCGCTTACAGTGTACTGGTTGTGGCCAAAGTGGAGAAAGGGAAGTCTAAAAAACTCA
AAAGCGTCAAGGAACTGCTGGGCATCACAATCATGGAGCGATCAAGCTTCGAAAAAAACCCC
ATCGACTTTCTCGAGGCGAAAGGATATAAAGAGGTCAAAAAAGACCTCATCATTAAGCTTCC
CAAGTACTCTCTCTTTGAGCTTGAAAACGGCCGGAAACGAATGCTCGCTAGTGCGGGCGAGC
TGCAGAAAGGTAACGAGCTGGCACTGCCCTCTAAATACGTTAATTTCTTGTATCTGGCCAGC
CACTATGAAAAGCTCAAAGGGTCTCCCGAAGATAATGAGCAGAAGCAGCTGTTCGTGGAACA
ACACAAACACTACCTTGATGAGATCATCGAGCAAATAAGCGAATTCTCCAAAAGAGTGATCC
TCGCCGACGCTAACCTCGATAAGGTGCTTTCTGCTTACAATAAGCACAGGGATAAGCCCATC
AGGGAGCAGGCAGAAAACATTATCCACTTGTTTACTCTGACCAACTTGGGCGCGCCTGCAGC
CTTCAAGTACTTCGACACCACCATAGACAGAAAGCGGTACACCTCTACAAAGGAGGTCCTGG
ACGCCACACTGATTCATCAGTCAATTACGGGGCTCTATGAAACAAGAATCGACCTCTCTCAG
CTCGGTGGAGACAGCAGGGCTGACCCCAAGAAGAAGAGGAAGGTGGctagCgacggcattgg
tagtgggagcaacggcagcagcggatccaacggtccgactgacgccgcggaagaggagcttc
tgagcaaaaactatcacctcgaaaacgaggttgcgcgactgaagaaaggaagcgggtccggt
ggaagtggctccggatctggaggttctggcagcggaggtagcggcagtggcgaagagctcct
tagtaagaactatcatctggaaaatgaggtagcgcgcttaaagaaagggtcgggaagtggcg
gcagcggaagtgggagtggagggagcggttctggcggttccggcagtggagaggagttgctg
tctaagaactaccacttagaaaacgaagtcgcacggctaaaaaaaggttccggctccggcgg
ctccggttctggaagcgggggctcgggatcaggtggatctggatcaggagaggaattgcttt
ccaaaaactaccaccttgagaatgaggtggccaggttaaagaaggggagcggctcggggggt
agtggatcggggtcgggcgggtcaggaagcggtggtagcggatctggggaggagctgctctc
gaagaattaccatttggagaacgaagtggcgagactaaagaag
SEQ ID NO: 89
Protein sequence for dCas9-24X-GCN4 (GCN4 is underlined)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTEDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRENASLGTYHDLLKIIKDKDELDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSD
NVPSEEVVKKMKNYWRQLLNAKLITQRKEDNLTKAERGGLSELDKAGFIKRQLVETRQITKH
VAQILDSRMNTKYDENDKLIREVKVITLKSKLVSDERKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYEDTTIDRKRYTSTKEVLDATLIHQSITGLYETRIDLSQ
LGGDAYPYDVPDYASLGSGSPKKKRKVEDPKKKRKVDGIGSGSNGSSGSNGPGGSGGGGSGG
EELLSKNYHLENEVARLKKGSGSGEELLSKNYHLENEVARLKKGSGSGEELLSKNYHLENEV
ARLKKGSGSGEELLSKNYHLENEVARLKKGSGSGEELLSKNYHLENEVARLKKGSGSGEELL
SKNYHLENEVARLKKGSGSGEELLSKNYHLENEVARLKKGSGSGEELLSKNYHLENEVARLK
KGSGSGEELLSKNYHLENEVARLKKGSGSGEELLSKNYHLENEVARLKKGSGSGEELLSKNY
HLENEVARLKKGSGSGEELLSKNYHLENEVARLKKGSGSGEELLSKNYHLENEVARLKKGSG
SGEELLSKNYHLENEVARLKKGSGSGEELLSKNYHLENEVARLKKGSGSGEELLSKNYHLEN
EVARLKKGSGSGEELLSKNYHLENEVARLKKGSGSGEELLSKNYHLENEVARLKKGSGSGEE
LLSKNYHLENEVARLKKGSGSGEELLSKNYHLENEVARLKKGSGSGEELLSKNYHLENEVAR
LKKGSGSGEELLSKNYHLENEVARLKKGSGSGEELLSKDYHLENEVARLKKGSGSGEELLSK
NYHLENEVARLKK
SEQ ID NO: 90
DNA sequence for dCas9-24X-GCN4
atggacaagaagtacagcatcggcctggccatcggcaccaactctgtgggctgggccgtgat
caccgacgagtacaaggtgcccagcaagaaattcaaggtgctgggcaacaccgaccggcaca
gcatcaagaagaacctgatcggcgccctgctgttcgacagcggagaaacagccgaggccacc
cggctgaagagaaccgccagaagaagatacaccagacggaagaaccggatctgctatctgca
agagatcttcagcaacgagatggccaaggtggacgacagcttcttccacagactggaagagt
ccttcctggtggaagaggataagaagcacgagcggcaccccatcttcggcaacatcgtggac
gaggtggcctaccacgagaagtaccccaccatctaccacctgagaaagaaactggtggacag
caccgacaaggccgacctgcggctgatctatctggccctggcccacatgatcaagttccggg
gccacttcctgatcgagggcgacctgaaccccgacaacagcgacgtggacaagctgttcatc
cagctggtgcagacctacaaccagctgttcgaggaaaaccccatcaacgccagcggcgtgga
cgccaaggccatcctgtctgccagactgagcaagagcagacggctggaaaatctgatcgccc
agctgcccggcgagaagaagaatggcctgttcggcaacctgattgccctgagcctgggcctg
acccccaacttcaagagcaacttcgacctggccgaggatgccaaactgcagctgagcaagga
cacctacgacgacgacctggacaacctgctggcccagatcggcgaccagtacgccgacctgt
ttctggccgccaagaacctgtccgacgccatcctgctgagcgacatcctgagagtgaacacc
gagatcaccaaggcccccctgagcgcctctatgatcaagagatacgacgagcaccaccagga
cctgaccctgctgaaagctctcgtgcggcagcagctgcctgagaagtacaaagagattttct
tcgaccagagcaagaacggctacgccggctacatcgatggcggagccagccaggaagagttc
tacaagttcatcaagcccatcctggaaaagatggacggcaccgaggaactgctcgtgaagct
gaacagagaggacctgctgcggaagcagcggaccttcgacaacggcagcatcccccaccaga
tccacctgggagagctgcacgccattctgcggcggcaggaagatttttacccattcctgaag
gacaaccgggaaaagatcgagaagatcctgaccttccgcatcccctactacgtgggccctct
ggccaggggaaacagcagattcgcctggatgaccagaaagagcgaggaaaccatcaccccct
ggaacttcgaggaagtggtggacaagggcgccagcgcccagagcttcatcgagcggatgacc
aacttcgataagaacctgcccaacgagaaggtgctgcccaagcacagcctgctgtacgagta
cttcaccgtgtacaacgagctgaccaaagtgaaatacgtgaccgagggaatgagaaagcccg
ccttcctgagcggcgagcagaaaaaagccatcgtggacctgctgttcaagaccaaccggaaa
gtgaccgtgaagcagctgaaagaggactacttcaagaaaatcgagtgcttcgactccgtgga
aatctccggcgtggaagatcggttcaacgcctccctgggcacataccacgatctgctgaaaa
ttatcaaggacaaggacttcctggacaatgaggaaaacgaggacattctggaagatatcgtg
ctgaccctgacactgtttgaggacagagagatgatcgaggaacggctgaaaacctatgccca
cctgttcgacgacaaagtgatgaagcagctgaagcggcggagatacaccggctggggcaggc
tgagccggaagctgatcaacggcatccgggacaagcagtccggcaagacaatcctggatttc
ctgaagtccgacggcttcgccaacagaaacttcatgcagctgatccacgacgacagcctgac
ctttaaagaggacatccagaaagcccaggtgtccggccagggcgatagcctgcacgagcaca
ttgccaatctggccggcagccccgccattaagaagggcatcctgcagacagtgaaggtggtg
gacgagctcgtgaaagtgatgggccggcacaagcccgagaacatcgtgatcgaaatggccag
agagaaccagaccacccagaagggacagaagaacagccgcgagagaatgaagcggatcgaag
agggcatcaaagagctgggcagccagatcctgaaagaacaccccgtggaaaacacccagctg
cagaacgagaagctgtacctgtactacctgcagaatgggcgggatatgtacgtggaccagga
actggacatcaaccggctgtccgactacgatgtggacgctatcgtgcctcagagctttctga
aggacgactccatcgataacaaagtgctgactcggagcgacaagaaccggggcaagagcgac
aacgtgccctccgaagaggtcgtgaagaagatgaagaactactggcgccagctgctgaatgc
caagctgattacccagaggaagttcgacaatctgaccaaggccgagagaggcggcctgagcg
aactggataaggccggcttcatcaagagacagctggtggaaacccggcagatcacaaagcac
gtggcacagatcctggactcccggatgaacactaagtacgacgagaacgacaaactgatccg
ggaagtgaaagtgatcaccctgaagtccaagctggtgtccgatttccggaaggatttccagt
tttacaaagtgcgcgagatcaacaactaccaccacgcccacgacgcctacctgaacgccgtc
gtgggaaccgccctgatcaaaaagtaccctaagctggaaagcgagttcgtgtacggcgacta
caaggtgtacgacgtgcggaagatgatcgccaagagcgagcaggaaatcggcaaggctaccg
ccaagtacttcttctacagcaacatcatgaactttttcaagaccgagattaccctggccaac
ggcgagatccggaagcggcctctgatcgagacaaacggcgaaacaggcgagatcgtgtggga
taagggccgggactttgccaccgtgcggaaagtgctgtctatgccccaagtgaatatcgtga
aaaagaccgaggtgcagacaggcggcttcagcaaagagtctatcctgcccaagaggaacagc
gacaagctgatcgccagaaagaaggactgggaccctaagaagtacggcggcttcgacagccc
caccgtggcctattctgtgctggtggtggccaaagtggaaaagggcaagtccaagaaactga
agagtgtgaaagagctgctggggatcaccatcatggaaagaagcagcttcgagaagaatccc
atcgactttctggaagccaagggctacaaagaagtgaaaaaggacctgatcatcaagctgcc
taagtactccctgttcgagctggaaaacggccggaagagaatgctggcctctgccggcgaac
tgcagaagggaaacgaactggccctgccctccaaatatgtgaacttcctgtacctggccagc
cactatgagaagctgaagggctcccccgaggataatgagcagaaacagctgtttgtggaaca
gcacaaacactacctggacgagatcatcgagcagatcagcgagttctccaagagagtgatcc
tggccgacgctaatctggacaaggtgctgagcgcctacaacaagcacagagacaagcctatc
agagagcaggccgagaatatcatccacctgtttaccctgaccaatctgggagcccctgccgc
cttcaagtactttgacaccaccatcgaccggaagaggtacaccagcaccaaagaggtgctgg
acgccaccctgatccaccagagcatcaccggcctgtacgagacacggatcgacctgtctcag
ctgggaggcgacgcctatccctatgacgtgcccgattatgccagcctgggcagcggctcccc
caagaaaaaacgcaaggtggaagatcctaagaaaaagcggaaagtggacggcattggtagtg
ggagcaacggcagcagcggatccaacggtccgggtggatctggaggtggaggttctggagga
gaagaacttttgagcaagaattatcatcttgagaacgaagtggctcgtcttaagaaaggttc
tggcagtggagaagaactgctttcaaagaattaccacctggaaaatgaggtagctagactga
aaaaggggagcggaagtggggaggagttgctgagcaaaaattatcatttggagaacgaagta
gcacgactaaagaaagggtccggatcgggtgaggagttactctcgaaaaattatcatctcga
aaacgaagtggctcggctaaaaaagggcagtggttctggagaagagctattatctaaaaact
accacctcgaaaatgaggtggcacgcttaaaaaagggaagtggcagtggtgaagagctacta
tccaagaattatcatcttgagaacgaggtagcgcgtttgaagaagggttccggctcaggaga
ggaactgctctcgaagaactatcatcttgaaaatgaggtcgctcgattaaaaaagggatcgg
gcagtggtgaggaactactttcaaagaattaccacctcgaaaacgaagtagctcgattaaag
aaaggttcagggtcgggtgaagaattactgagtaaaaattatcatctggaaaatgaggtagc
gagactaaaaaaggggagtggttctggcgaagagttgctatcgaaaaattatcatcttgaga
acgaagttgctaggctcaaaaagggctcaggctcaggcgaggagttgctctcgaaaaactac
cacttggaaaatgaggtcgcgaggttgaaaaaggggagcgggtcgggcgaggagttattgag
caaaaactatcatttagagaacgaagtcgcgcgcttaaagaaaggctcgggctcgggcgaag
aactcttatcgaagaactaccacctcgaaaatgaggtcgccaggttgaaaaagggcagtggc
agcggggaggaactcttgagcaagaactaccacttggagaatgaggtcgcgagattgaagaa
agggtcggggagcggcgaggaattgctcagcaagaattatcatttggagaacgaagtcgcca
ggctcaagaaaggctcggggtcgggggaggaattgttgagtaaaaactaccacttggaaaat
gaagtcgccaggctcaaaaaagggagtgggagcggcgaagagttattgagcaaaaattacca
cttggagaacgaagtggcaaggctcaagaaagggagcggcagcggggaggagctcttatcga
agaactaccacttagagaatgaagtcgcccgcttgaagaaaggctcggggagcggggaagag
ctcttgagcaagaactaccacttggaaaatgaggtggcgcgcttgaagaaagggagcgggag
cggggaagagttactatctaagaattatcatctcgagaacgaggtggctcgactaaagaagg
gctccggcagtggggaggaactcctgtcgaagaactatcatcttgaaaatgaggttgcaaga
cttaaaaaggggtccggatcaggtgaggaactactcagtaagaattaccacctggaaaacga
agttgcacgtttgaagaaaggatcaggatcaggcgaagaactgctctcaaaagattatcatt
tggaaaatgaggttgcacgtttaaaaaagggaagtggcagtggtgaggaacttctgtcgaaa
aattatcatctcgagaatgaagtagcccgacttaaaaag
SEQ ID NO: 91
Linker, peptide
GSGSG
SEQ ID NO: 92
Linker, peptide
GSGSGGSGSGSGGSGSGGSGSG
SEQ ID NO: 103
ASH2L protein sequence
MDTQAGSVDEENGRQLGEVELQCGICTKWFTADTFGIDTSSCLPFMTNYSFHCNVCHHSG
NTYFLRKQANLKEMCLSALANLTWQSRTQDEHPKTMFSKDKDIIPFIDKYWECMTTRQRP
GKMTWPNNIVKTMSKERDVFLVKEHPDPGSKDPEEDYPKFGLLDQDLSNIGPAYDNQKQS
SAVSTSGNLNGGIAAGSSGKGRGAKRKQQDGGTTGTTKKARSDPLFSAQRLPPHGYPLEH
PFNKDGYRYILAEPDPHAPDPEKLELDCWAGKPIPGDLYRACLYERVLLALHDRAPQLKI
SDDRLTVVGEKGYSMVRASHGVRKGAWYFEITVDEMPPDTAARLGWSQPLGNLQAPLGYD
KFSYSWRSKKGTKFHQSIGKHYSSGYGQGDVLGFYINLPEDTETAKSLPDTYKDKALIKE
KSYLYFEEKDFVDKAEKSLKQTPHSEIIFYKNGVNQGVAYKDIFEGVYFPAISLYKSCTV
SINFGPCFKYPPKDLTYRPMSDMGWGAVVEHTLADVLYHVETEVDGRRSPPWEP
SEQ ID NO: 104
ASH2L DNA sequence
ATGGATACTCAGGCGGGCTCCGTGGATGAAGAGAATGGCCGACAGTTGGGTGAGGTAGAGCT
GCAATGTGGGATTTGTACAAAATGGTTCACGGCTGACACATTTGGCATAGATACCTCATCCT
GTCTACCTTTCATGACCAACTACAGTTTTCATTGCAACGTCTGCCATCACAGTGGGAATACC
TATTTCCTCCGGAAGCAAGCAAACTTGAAGGAAATGTGCCTTAGTGCTTTGGCCAACCTGAC
ATGGCAGTCCCGAACACAGGATGAACATCCGAAGACAATGTTCTCCAAAGATAAGGATATTA
TACCATTTATTGATAAATACTGGGAGTGCATGACAACCAGACAGAGACCTGGGAAAATGACT
TGGCCAAATAACATTGTTAAAACAATGAGTAAAGAAAGAGATGTATTCTTGGTAAAGGAACA
CCCAGATCCAGGCAGTAAAGATCCAGAAGAAGATTACCCCAAATTTGGACTTTTGGATCAGG
ACCTTAGTAACATTGGTCCTGCTTATGACAACCAAAAACAGAGCAGTGCTGTGTCTACTAGT
GGGAATTTAAATGGGGGAATTGCAGCAGGAAGCAGCGGAAAAGGACGAGGAGCCAAGCGCAA
ACAGCAGGATGGAGGGACCACAGGGACCACCAAGAAGGCCCGGAGTGACCCTTTGTTTTCTG
CTCAGCGCCTTCCCCCTCATGGCTACCCATTGGAACACCCGTTTAACAAAGATGGCTATCGG
TATATTCTAGCTGAGCCTGATCCGCACGCCCCTGACCCCGAGAAGCTGGAACTTGACTGCTG
GGCAGGAAAACCTATTCCTGGAGACCTCTACAGAGCCTGCTTGTATGAACGGGTTTTGTTAG
CCCTACATGATCGAGCTCCCCAGTTAAAGATCTCAGATGACCGGCTGACTGTGGTTGGAGAG
AAGGGCTACTCTATGGTGAGGGCCTCTCATGGAGTACGGAAGGGTGCCTGGTATTTTGAAAT
CACTGTGGATGAGATGCCACCAGATACCGCTGCCAGACTGGGTTGGTCCCAGCCCCTAGGAA
ACCTTCAAGCTCCTTTAGGTTATGATAAATTTAGCTATTCTTGGCGGAGCAAAAAGGGAACC
AAGTTCCACCAGTCCATTGGCAAACACTACTCTTCTGGCTATGGACAGGGAGACGTCCTGGG
ATTTTATATTAATCTTCCTGAAGACACAGAGACAGCCAAGTCATTGCCAGACACATACAAAG
ATAAGGCTTTGATAAAATTCAAGAGTTATTTGTATTTTGAGGAAAAAGACTTTGTGGATAAA
GCAGAGAAGAGCCTGAAGCAGACTCCCCATAGTGAGATAATATTTTATAAAAATGGTGTCAA
TCAAGGTGTGGCTTACAAAGATATTTTTGAGGGGGTTTACTTCCCAGCCATCTCACTGTACA
AGAGCTGCACGGTTTCCATTAACTTTGGACCATGCTTCAAGTATCCTCCGAAGGATCTCACT
TACCGCCCTATGAGTGACATGGGCTGGGGCGCCGTGGTAGAGCACACCCTGGCTGACGTCTT
GTATCACGTGGAGACAGAAGTGGATGGGAGGCGCAGTCCCCCATGGGAACCCTGA
SEQ ID NO: 105
BCL7B protein sequence
MSGRSVRAETRSRAKDDIKKVMAAIEKVRKWEKKWVTVGDTSLRIFKWVPVTDSKEKEKS
KSNSSAAREPNGFPSDASANSSLLLEFQDENSNQSSVSDVYQLKVDSSTNSSPSPQQSES
LSPAHTSDERTDDSQPPTLGQEILEEPSLPSSEVADEPPTLTKEEPVPLETQVVEEEEDS
GAPPLKRFCVDQPTVPQTASESL
SEQ ID NO: 106
BCL7B DNA sequence
atgtcgggccggtcggtccgggcggagacccgcagccgggccaaggacgacatcaagaaggt
gatggcggccatcgagaaagtgcggaaatgggagaagaagtgggtgactgtgggtgacacgt
ccctgaggatatttaagtgggttcctgtgacagacagcaaggagaaagaaaagtcaaaatcg
aacagttcagcagcccgagaacctaatggctttccttctgatgcctcagccaattcctctct
ccttcttgaattccaggacgaaaacagcaaccagagttccgtgtctgacgtctatcagctta
aggtggacagcagcaccaactcaagccccagcccccagcagagtgagtccctgagcccagca
cacacctccgacttccgcacggatgactcccagcccccaacgctgggccaggagatcctgga
ggagccctccctgccctcctcggaagttgctgatgaacctcctaccctcaccaaggaagaac
cagttccactagagacacaggtcgttgaggaagaggaagactcaggtgccccgcccctgaag
cgcttctgtgtggaccaacccacagtgccgcagacggcgtcagaaagcttg
SEQ ID NO: 107
C20orf20 protein sequence
MGEAEVGGGGAAGDKGPGEAATSPAEETVVWSPEVEVCLFHAMLGHKPVGVNRHFHMICI
RDKFSQNIGRQVPSKVIWDHLSTMYDMQALHESEILPEPNPERNEVLPEEIIQEVREGKV
MIEEEMKEEMKEDVDPHNGADDVESSSGSLGKASEKSSKDKEKNSSDLGCKEGADKRKRS
RVTDKVLTANSNPSSPSAAKRRRT
SEQ ID NO: 108
C20orf20 DNA sequence
ATGGGAGAGGCCGAGGTGGGCGGCGGGGGCGCCGCAGGCGACAAGGGCCCGGGGGAGGCGGC
CACCAGCCCGGCGGAGGAGACAGTGGTGTGGAGCCCCGAGGTGGAGGTGTGCCTCTTCCACG
CCATGCTGGGCCACAAGCCCGTCGGTGTGAACCGACACTTCCACATGATTTGTATTCGGGAC
AAGTTCAGCCAGAACATCGGGCGGCAGGTCCCATCCAAGGTCATCTGGGACCATCTGAGCAC
CATGTACGACATGCAGGCGCTGCATGAGTCTGAGATTCTTCCATTCCCGAATCCAGAGAGGA
ACTTCGTCCTTCCAGAAGAGATCATTCAGGAGGTCCGAGAAGGAAAAGTGATGATAGAAGAG
GAGATGAAAGAGGAGATGAAGGAAGACGTGGACCCCCACAATGGGGCTGACGATGTTTTTTC
ATCTTCAGGGAGTTTGGGGAAAGCATCAGAAAAATCCAGCAAAGACAAAGAGAAGAACTCCT
CAGACTTGGGGTGCAAAGAAGGCGCAGACAAGCGGAAGCGCAGCCGGGTCACCGACAAAGTC
CTGACCGCAAACAGCAACCCTTCCAGTCCCAGTGCTGCCAAGCGGCGCCGCACGTAG
SEQ ID NO: 109
DMAP1 protein sequence
MATGADVRDILELGGPEGDAASGTISKKDIINPDKKKSKKSSETLTFKRPEGMHREVYAL
LYSDKKDAPPLLPSDTGQGYRTVKAKLGSKKVRPWKWMPFTNPARKDGAMFFHWRRAAEE
GKDYPFARFNKTVQVPVYSEQEYQLYLHDDAWTKAETDHLEDLSRREDLRFVVIHDRYDH
QQFKKRSVEDLKERYYHICAKLANVRAVPGTDLKIPVFDAGHERRRKEQLERLYNRTPEQ
VAEEEYLLQELRKIEARKKEREKRSQDLQKLITAADTTAEQRRTERKAPKKKLPQKKEAE
KPAVPETAGIKFPDFKSAGVTLRSQRMKLPSSVGQKKIKALEQMLLELGVELSPTPTEEL
VHMFNELRSDLVLLYELKQACANCEYELQMLRHRHEALARAGVLGGPATPASGPGPASAE
PAVTEPGLGPDPKDTIIDVVGAPLTPNSRKRRESASSSSSVKKAKKPL
SEQ ID NO: 110
DMAP1 DNA sequence
atggctacgggcgcggatgtacgggacattctagaactcgggggtccagaaggggatgcagc
ctctgggaccatcagcaagaaggacattatcaacccggacaagaaaaaatccaagaagtcct
ctgagacactgactttcaagaggcccgagggcatgcaccgggaagtctatgccttgctctac
tctgacaagaaggatgcacccccactgctacccagtgacactggccagggataccgtacagt
gaaggccaagttgggctccaagaaggtgcggccttggaagtggatgccattcaccaacccgg
cccgcaaggacggagcaatgttcttccactggcgacgtgcagcggaggagggcaaggactac
ccctttgccaggttcaataagactgtgcaggtgcctgtgtactcggagcaggagtaccagct
ttatctccacgatgatgcttggactaaggcagaaactgaccacctctttgacctcagccgcc
gctttgacctgcgttttgttgttatccatgaccggtatgaccaccagcagttcaagaagcgt
tctgtggaagacctgaaggagcggtactaccacatctgtgctaagcttgccaacgtgcgggc
tgtgccaggcacagaccttaagataccagtatttgatgctgggcacgaacgacggcggaagg
aacagcttgagcgtctctacaaccggaccccagagcaggtggcagaggaggagtacctgcta
caggagctgcgcaagattgaggcccggaagaaggagcgggagaaacgcagccaggacctgca
gaagctgatcacagcggcagacaccactgcagagcagcggcgcacggaacgcaaggccccca
aaaagaagctaccccagaaaaaggaggctgagaagccggctgttcctgagactgcaggcatc
aagtttccagacttcaagtctgcaggtgtcacgctgcggagccaacggatgaagctgccaag
ctctgtgggacagaagaagatcaaggccctggaacagatgctgctggagcttggtgtggagc
tgagcccgacacctacggaggagctggtgcacatgttcaatgagctgcgaagcgacctggtg
ctgctctacgagctcaagcaggcctgtgccaactgcgagtatgagctgcagatgctgcggca
ccgtcatgaggcactggcccgggctggtgtgctagggggccctgccacaccagcatcaggcc
caggcccggcctctgctgagccggcagtgactgaacccggacttggtcctgaccccaaggac
accatcattgatgtggtgggcgcacccctcacgcccaattcgagaaagcgacgggagtcggc
ctccagctcatcttccgtgaagaaagccaagaagccgttg
SEQ ID NO: 111
DYRK1B protein sequence
MAVPPGHGPFSGFPGPQEHTQVLPDVRLLPRRLPLAFRDATSAPLRKLSVDLIKTYKHIN
EVYYAKKKRRAQQAPPQDSSNKKEKKVLNHGYDDDNHDYIVRSGERWLERYEIDSLIGKG
SFGQVVKAYDHQTQELVAIKIIKNKKAFLNQAQIELRLLELMNQHDTEMKYYIVHLKRHF
MFRNHLCLVFELLSYNLYDLLRNTHFRGVSLNLTRKLAQQLCTALLFLATPELSIIHCDL
KPENILLCNPKRSAIKIVDFGSSCQLGQRIYQYIQSRFYRSPEVLLGTPYDLAIDMWSLG
CILVEMHTGEPLESGSNEVDQMNRIVEVLGIPPAAMLDQAPKARKYFERLPGGGWTLRRT
KELRKDYQGPGTRRLQEVLGVQTGGPGGRRAGEPGHSPADYLRFQDLVLRMLEYEPAARI
SPLGALQHGFFRRTADEATNTGPAGSSASTSPAPLDTCPSSSTASSISSSGGSSGSSSDN
RTYRYSNRYCGGPGPPITDCEMNSPQVPPSQPLRPWAGGDVPHKTHQAPASASSLPGTGA
QLPPQPRYLGRPPSPTSPPPPELMDVSLVGGPADCSPPHPAPAPQHPAASALRTRMTGGR
PPLPPPDDPATLGPHLGLRGVPQSTAASSL
SEQ ID NO: 112
DYRK1B DNA sequence
atggccgtcccaccgggccatggtcccttctctggcttcccagggccccaggagcacacgca
ggtattgcctgatgtgcggctactgcctcggaggctgcccctggccttccgggatgcaacct
cagccccgctgcgtaagctctctgtggacctcatcaagacctacaagcacatcaatgaggta
tactatgcgaagaagaagcggcgggcccagcaggcgccaccccaggattcgagcaacaagaa
ggagaagaaggtcctgaaccatggttatgatgacgacaaccatgactacatcgtgcgcagtg
gcgagcgctggctggagcgctacgaaattgactcgctcattggcaaaggctcctttggccag
gtggtgaaagcctatgatcatcagacccaggagcttgtggccatcaagatcatcaagaacaa
aaaggctttcctgaaccaggcccagattgagctgcggctgctggagctgatgaaccagcatg
acacggagatgaagtactatatagtacacctgaagcggcacttcatgttccggaaccacctg
tgcctggtatttgagctgctgtcctacaacctgtacgacctcctgcgcaacacccacttccg
cggcgtctcgctgaacctgacccggaagctggcgcagcagctctgcacggcactgctctttc
tggccacgcctgagctcagcatcattcactgcgacctcaagcccgaaaacatcttgctgtgc
aaccccaagcgcagcgccatcaagattgtggacttcggcagctcctgccagcttggccagag
gatctaccagtatatccagagccgcttctaccgctcacctgaggtgctcctgggcacaccct
acgacctggccattgacatgtggtccctgggctgcatccttgtggagatgcacaccggagag
cccctcttcagtggctccaatgaggtcgaccagatgaaccgcattgtggaggtgctgggcat
cccaccggccgccatgctggaccaggcgcccaaggctcgcaagtactttgaacggctgcctg
ggggtggctggaccctacgaaggacgaaagaactcaggaaggattaccagggccccgggaca
cggcggctgcaggaggtgctgggcgtgcagacgggcgggcccgggggccggcgggcggggga
gccgggccacagccccgccgactacctccgcttccaggacctggtgctgcgcatgctggagt
atgagcccgccgcccgcatcagccccctgggggctctgcagcacggcttcttccgccgcacg
gccgacgaggccaccaacacgggcccggcaggcagcagtgcctccacctcgcccgcgcccct
cgacacctgcccctcttccagcaccgccagctccatctccagttctggaggctccagtggct
cctccagtgacaaccggacctaccgctacagcaaccgatattgtgggggccctgggccccct
atcacagactgtgagatgaacagcccccaggtcccaccctcccagccgctgcggccctgggc
agggggtgatgtgccccacaagacacatcaagcccctgcctctgcctcgtcactgcctggga
ccggggcccagttacccccccagccccgataccttggtcgtcccccatcaccaacctcacca
ccacccccggagctgatggatgtgagcctggtgggcggccctgctgactgctccccacctca
cccagcgcctgccccccagcacccggctgcctcagccctccggactcggatgactggaggtc
gtccacccctcccgcctcctgatgaccctgccactctggggcctcacctgggcctccgtggt
gtaccccagagcacagcagccagctcgttg
SEQ ID NO: 113
EAF1 protein sequence
MNGTANPLLDREEHCLRLGESFEKRPRASFHTIRYDFKPASIDTSCEGELQVGKGDEVTI
TLPHIPGSTPPMTVFKGNKRPYQKDCVLIINHDTGEFVLEKLSSSIQVKKTRAEGSSKIQ
ARMEQQPTRPPQTSQPPPPPPPMPFRAPTKPPVGPKTSPLKDNPSPEPQLDDIKRELRAE
VDIIEQMSSSSGSSSSDSESSSGSDDDSSSSGGEDNGPASPPQPSHQQPYNSRPAVANGT
SRPQGSNQLMNALRNDLQLSESGSDSDD
SEQ ID NO: 114
EAF1 DNA sequence
ATGAATGGGACCGCAAACCCGCTGCTGGACCGCGAGGAACATTGCCTGAGGCTCGGGGAGAG
CTTCGAGAAGCGGCCGCGGGCCTCCTTCCACACTATTCGTTATGATTTTAAACCAGCATCTA
TAGACACTTCCTGTGAAGGAGAGCTTCAAGTTGGCAAAGGAGATGAAGTCACAATTACACTT
CCACATATCCCTGGATCCACACCACCCATGACTGTGTTCAAGGGGAACAAACGGCCTTACCA
GAAAGACTGTGTGCTTATTATTAATCATGACACTGGTGAATTTGTGCTGGAAAAACTCAGTA
GCAGCATTCAGGTGAAGAAAACAAGAGCTGAGGGCAGCAGTAAAATCCAGGCCCGAATGGAA
CAGCAGCCCACTCGTCCTCCACAGACGTCACAGCCACCACCACCTCCACCACCTATGCCATT
CAGAGCTCCAACGAAGCCTCCAGTTGGACCCAAAACTTCTCCCTTGAAAGATAACCCCTCAC
CTGAACCTCAGTTGGATGACATCAAAAGAGAGCTGAGGGCTGAAGTTGACATTATTGAACAA
ATGAGCAGCAGCAGTGGGAGCAGCTCTTCAGACTCTGAGAGCTCTTCGGGAAGTGATGACGA
TAGCTCCAGCAGTGGAGGCGAGGACAATGGCCCAGCCTCTCCTCCGCAGCCTTCACACCAGC
AGCCCTACAACAGTAGGCCTGCCGTTGCCAATGGAACCAGCCGGCCACAAGGAAGCAACCAG
CTCATGAACGCCCTCAGAAATGACTTGCAGTTGAGTGAGTCTGGCAGTGACAGTGATGACTA
G
SEQ ID NO: 115
FOXR2 protein sequence
MDLKLKDCEFWYSLHGQVPGLLDWDMRNELFLPCTTDQCSLAEQILAKYRVGVMKPPEMP
QKRRPSPDGDGPPCEPNLWMWVDPNILCPLGSQEAPKPSGKEDLTNISPFPQPPQKDEGS
NCSEDKVVESLPSSSSEQSPLQKQGIHSPSDFELTEEEAEEPDDNSLQSPEMKCYQSQKL
WQINNQEKSWQRPPLNCSHLIALALRNNPHCGLSVQEIYNFTRQHFPFFWTAPDGWKSTI
HYNLCFLDSFEKVPDSLKDEDNARPRSCLWKLTKEGHRRFWEETRVLAFAQRERIQECMS
QPELLTSLFDL
SEQ ID NO: 116
FOXR2 DNA sequence
ATGGACTTAAAACTAAAAGACTGTGAATTTTGGTATAGTCTCCATGGCCAGGTCCCAGGGCT
GCTGGACTGGGACATGAGGAATGAGTTATTTCTGCCTTGTACCACAGACCAGTGCTCTTTAG
CTGAGCAAATCCTTGCCAAATACAGAGTCGGAGTAATGAAGCCCCCAGAAATGCCTCAGAAG
AGGAGACCCAGTCCTGATGGAGATGGTCCTCCCTGTGAACCCAATCTGTGGATGTGGGTGGA
CCCCAATATCCTGTGCCCCCTTGGCAGCCAGGAGGCCCCAAAGCCCAGTGGAAAAGAGGATC
TGACAAACATTTCTCCTTTCCCTCAGCCCCCACAAAAAGACGAAGGGTCTAACTGCTCAGAG
GACAAAGTGGTAGAGTCTCTGCCATCTTCCTCCAGTGAGCAGTCTCCTTTACAGAAGCAGGG
TATCCATTCCCCCAGTGACTTTGAGCTCACAGAAGAGGAGGCTGAGGAACCAGACGACAACT
CCCTCCAGTCCCCTGAAATGAAATGTTACCAGAGCCAGAAACTATGGCAAATCAACAACCAA
GAGAAGTCCTGGCAAAGGCCCCCTCTCAATTGTAGCCACCTTATTGCCCTAGCATTAAGAAA
CAACCCCCACTGTGGCCTCAGTGTGCAGGAGATCTACAATTTCACCCGACAGCATTTCCCCT
TTTTCTGGACAGCTCCGGATGGCTGGAAGAGCACCATTCATTACAACCTCTGCTTCCTGGAC
AGCTTTGAGAAGGTGCCAGACAGCCTTAAGGATGAAGATAATGCAAGACCTCGCTCTTGCCT
TTGGAAGCTCACTAAGGAGGGGCACCGCCGCTTTTGGGAGGAGACTCGTGTCTTAGCCTTTG
CTCAAAGGGAGAGAATCCAAGAGTGCATGAGTCAGCCAGAGTTGTTGACCTCTCTCTTTGAT
CTTTGA
SEQ ID NO: 117
GSK3A protein sequence
MSGGGPSGGGPGGSGRARTSSFAEPGGGGGGGGGGPGGSASGPGGTGGGKASVGAMGGGV
GASSSGGGPGGSGGGGSGGPGAGTSFPPPGVKLGRDSGKVTTVVATLGQGPERSQEVAYT
DIKVIGNGSFGVVYQARLAETRELVAIKKVLQDKRFKNRELQIMRKLDHCNIVRLRYFFY
SSGEKKDELYLNLVLEYVPETVYRVARHFTKAKLTIPILYVKVYMYQLFRSLAYIHSQGV
CHRDIKPQNLLVDPDTAVLKLCDFGSAKQLVRGEPNVSYICSRYYRAPELIFGATDYTSS
IDVWSAGCVLAELLLGQPIFPGDSGVDQLVEIIKVLGTPTREQIREMNPNYTEFKFPQIK
AHPWTKVFKSRTPPEAIALCSSLLEYTPSSRLSPLEACAHSFFDELRCLGTQLPNNRPLP
PLENFSAGELSIQPSLNAILIPPHLRSPAGTTTLTPSSQALTETPTSSDWQSTDATPTLT
NSSL
SEQ ID NO: 118
GSK3A DNA sequence
atgagcggcggcgggccttcgggaggcggccctgggggctcgggcagggcgcggactagctc
gttcgcggagcccggcggcggaggcggaggaggcggcggcggccccggaggctcggcctccg
gcccaggcggcaccggcggcggaaaggcatctgtcggggccatgggtgggggcgtcggggcc
tcgagctccgggggtggacccggcggcagcggcggaggaggcagcggaggccccggcgcagg
cactagcttcccgccgcccggggtgaagctgggccgtgacagcgggaaggtgaccacagtcg
tagccactctaggccaaggcccagagcgctcccaagaagtggcttacacggacatcaaagtg
attggcaatggctcatttggggtcgtgtaccaggcacggctggcagagaccagggaactagt
cgccatcaagaaggttctccaggacaagaggttcaagaaccgagagctgcagatcatgcgta
agctggaccactgcaatattgtgaggctgagatactttttctactccagtggcgagaagaaa
gacgagctttacctaaatctggtgctggaatatgtgcccgagacagtgtaccgggtggcccg
ccacttcaccaaggccaagttgaccatccctatcctctatgtcaaggtgtacatgtaccagc
tcttccgcagcttggcctacatccactcccagggcgtgtgtcaccgcgacatcaagccccag
aacctgctggtggaccctgacactgctgtcctcaagctctgcgattttggcagtgcaaagca
gttggtccgaggggagcccaatgtctcctacatctgttctcgctactaccgggccccagagc
tcatctttggagccactgattacacctcatccatcgatgtttggtcagctggctgtgtactg
gcagagctcctcttgggccagcccatcttccctggggacagtggggtggaccagctggtgga
gatcatcaaggtgctgggaacaccaacccgggaacaaatccgagagatgaaccccaactaca
cggagttcaagttccctcagattaaagctcacccctggacaaaggtgttcaaatctcgaacg
ccgccagaggccatcgcgctctgctctagcctgctggagtacaccccatcctcaaggctctc
cccactagaggcctgtgcgcacagcttctttgatgaactgcgatgtctgggaacccagctgc
ctaacaaccgcccacttccccctctcttcaacttcagtgctggtgaactctccatccaaccg
tctctcaacgccattctcatccctcctcacttgaggtccccagcgggcactaccaccctcac
cccgtcctcacaagctttaactgagactccgaccagctcagactggcagtcgaccgatgcca
cacctaccctcactaactcctccttg
SEQ ID NO: 119
JAZF1 protein sequence
MTGIAAASFFSNTCRFGGCGLHFPTLADLIEHIEDNHIDTDPRVLEKQELQQPTYVALSY
INRFMTDAARREQESLKKKIQPKLSLTLSSSVSRGNVSTPPRHSSGSLTPPVTPPITPSS
SFRSSTPTGSEYGEEEVDYEESDSDESWTTESAISSEAILSSMCMNGGEEKPFACPVPGC
KKRYKNVNGIKYHAKNGHRTQIRVRKPFKCRCGKSYKTAQGLRHHTINFHPPVSAEIIRK
MQQL
SEQ ID NO: 120
JAZF1 DNA sequence
Atgacaggcatcgccgccgcctccttcttctccaatacctgccgattcgggggctgcggact
ccacttccccaccctggccgacctcatcgagcacatcgaggacaaccacatcgatacagatc
cacgggttttagaaaaacaagaattacagcagccaacctatgttgccctgagttacataaat
agattcatgacagatgctgcccgccgagagcaggagtccctaaagaagaagattcagccgaa
gctctcgctgactctgtccagctcagtgtctcgagggaatgtgtccactcccccacgccaca
gcagtggaagccttactccccccgtgaccccacccatcaccccctcctcttcattccgcagc
agcactccgacaggcagcgagtatggcgaggaggaggtggactatgaggagtcggacagcga
tgagtcctggaccacagagagtgccatcagctccgaagccatcctcagctccatgtgcatga
atggaggggaagagaagccttttgcctgcccagttcctggatgtaaaaagagatacaagaat
gtgaatggcataaagtatcacgctaagaatggtcacagaacacagattcgtgtccgcaaacc
attcaagtgtcgctgtgggaagagttacaagacagctcagggcctgcggcaccacacaatca
atttccatcccccggtgtcggctgagattatcaggaagatgcagcaattg
SEQ ID NO: 121
KAT7 protein sequence
MPRRKRNAGSSSDGTEDSDESTDLEHTDSSESDGTSRRSARVTRSSARLSQSSQDSSPVR
NLQSFGTEEPAYSTRRVTRSQQQPTPVTPKKYPLRQTRSSGSETEQVVDESDRETKNTAD
HDESPPRTPTGNAPSSESDIDISSPNVSHDESIAKDMSLKDSGSDLSHRPKRRRFHESYN
FNMKCPTPGCNSLGHLTGKHERHESISGCPLYHNLSADECKVRAQSRDKQIEERMLSHRQ
DDNNRHATRHQAPTERQLRYKEKVAELRKKRNSGLSKEQKEKYMEHRQTYGNTREPLLEN
LTSEYDLDLERRAQARASEDLEKLRLQGQITEGSNMIKTIAFGRYELDTWYHSPYPEEYA
RLGRLYMCEFCLKYMKSQTILRRHMAKCVWKHPPGDEIYRKGSISVFEVDGKKNKIYCQN
LCLLAKLFLDHKTLYYDVEPFLFYVMTEADNTGCHLIGYFSKEKNSELNYNVSCILTMPQ
YMRQGYGKMLIDFSYLLSKVEEKVGSPERPLSDLGLISYRSYWKEVLLRYLHNFQGKEIS
IKEISQETAVNPVDIVSTLQALQMLKYWKGKHLVLKRQDLIDEWIAKEAKRSNSNKTMDP
SCLKWTPPKGT
SEQ ID NO: 122
KAT7 DNA sequence
ATGCCGCGAAGGAAGAGGAATGCAGGCAGTAGTTCAGATGGAACCGAAGATTCCGATTTTTC
TACAGATCTCGAGCACACAGACAGTTCAGAAAGTGATGGCACATCCCGACGATCTGCTCGAG
TCACCCGCTCCTCAGCCAGGCTAAGCCAGAGTTCTCAAGATTCCAGTCCTGTTCGAAATCTG
CAGTCTTTTGGCACTGAGGAGCCTGCTTACTCTACCAGAAGAGTGACCCGTAGTCAGCAGCA
GCCTACCCCAGTGACACCGAAAAAATACCCTCTTCGGCAGACTCGTTCATCTGGTTCAGAAA
CTGAGCAAGTGGTTGATTTTTCAGATAGAGAAACTAAAAATACAGCTGATCATGATGAGTCA
CCGCCTCGAACTCCAACTGGAAATGCGCCTTCTTCTGAGTCTGACATAGACATCTCCAGCCC
CAATGTATCTCACGATGAGAGCATTGCCAAGGACATGTCCCTGAAGGACTCAGGCAGIGATC
TCTCTCATCGCCCCAAGCGCCGTCGCTTCCATGAAAGCTACAACTTCAATATGAAGTGTCCT
ACACCAGGCTGTAACTCTCTAGGACACCTTACAGGAAAACATGAGAGACATTTCTCCATCTC
AGGATGCCCACTGTATCATAACCTCTCAGCTGACGAATGCAAGGTGAGAGCACAGAGCCGGG
ATAAGCAGATAGAAGAAAGGATGCTGTCTCACAGGCAAGATGACAACAACAGGCATGCAACC
AGGCACCAGGCACCAACGGAGAGGCAGCTTCGATATAAGGAAAAAGTGGCTGAACTCAGGAA
GAAAAGAAATTCTGGACTGAGCAAAGAACAGAAAGAGAAATATATGGAACACAGACAGACCT
ATGGGAACACACGGGAACCTCTTTTAGAAAACCTGACAAGCGAGTATGACTTGGATCTTTTC
CGAAGAGCACAAGCCCGGGCTTCAGAGGATTTGGAGAAGTTAAGGCTGCAAGGCCAAATCAC
AGAGGGAAGCAACATGATTAAAACAATTGCTTTTGGCCGCTATGAGCTTGATACCTGGTACC
ATTCTCCATATCCTGAAGAATATGCACGGCTGGGACGTCTCTATATGTGTGAATTCTGTTTA
AAATATATGAAGAGCCAAACGATACTCCGCCGGCACATGGCCAAATGTGTGTGGAAACACCC
ACCTGGTGATGAGATATATCGCAAAGGTTCAATCTCTGTGTTTGAAGTGGATGGCAAGAAAA
ACAAGATCTACTGCCAAAACCTGTGCCTGTTGGCCAAACTTTTTCTGGACCACAAGACATTA
TATTATGATGTGGAGCCCTTCCTGTTCTATGTTATGACAGAGGCGGACAACACTGGCTGTCA
CCTGATTGGATATTTTTCTAAGGAAAAGAATTCATTCCTCAACTACAACGTCTCCTGTATCC
TTACTATGCCTCAGTACATGAGACAGGGCTATGGCAAGATGCTTATTGATTTCAGTTATTTG
CTTTCCAAAGTCGAAGAAAAAGTTGGCTCCCCAGAACGTCCACTCTCAGATCTGGGGCTTAT
AAGCTATCGCAGTTACTGGAAAGAAGTACTTCTCCGCTACCTGCATAATTTTCAAGGCAAAG
AGATTTCTATCAAAGAAATCAGTCAGGAGACGGCTGTGAATCCTGTGGACATTGTCAGCACT
CTGCAAGCCCTTCAGATGCTCAAATACTGGAAGGGAAAACACCTAGTTTTAAAGAGACAGGA
CCTGATTGATGAGTGGATAGCCAAAGAGGCCAAAAGGTCCAACTCCAATAAAACCATGGATC
CCAGCTGCTTAAAATGGACCCCTCCCAAGGGCACTTAA
SEQ ID NO: 123
KEAP1 protein sequence
MQPDPRPSGAGACCRELPLQSQCPEGAGDAVMYASTECKAEVTPSQHGNRTFSYTLEDHT
KQAFGIMNELRLSQQLCDVTLQVKYQDAPAAQFMAHKVVLASSSPVFKAMFTNGLREQGM
EVVSIEGIHPKVMERLIEFAYTASISMGEKCVLHVMNGAVMYQIDSVVRACSDELVQQLD
PSNAIGIANFAEQIGCVELHQRAREYIYMHFGEVAKQEEFFNLSHCQLVTLISRDDLNVR
CESEVFHACINWVKYDCEQRRFYVQALLRAVRCHSLTPNFLQMQLQKCEILQSDSRCKDY
LVKIFEELTLHKPTQVMPCRAPKVGRLIYTAGGYFRQSLSYLEAYNPSDGTWLRLADLQV
PRSGLAGCVVGGLLYAVGGRNNSPDGNTDSSALDCYNPMTNQWSPCAPMSVPRNRIGVGV
IDGHIYAVGGSHGCIHHNSVERYEPERDEWHLVAPMLTRRIGVGVAVLNRLLYAVGGFDG
TNRLNSAECYYPERNEWRMITAMNTIRSGAGVCVLHNCIYAAGGYDGQDQLNSVERYDVE
TETWTFVAPMKHRRSALGITVHQGRIYVLGGYDGHTFLDSVECYDPDTDTWSEVTRMTSG
RSGVGVAVTMEPCRKQIDQQNCTCL
SEQ ID NO: 124
KEAP1 DNA sequence
atgcagccagatcccaggcctagcggggctggggcctgctgccgattcctgcccctgcagtc
acagtgccctgagggggcaggggacgcggtgatgtacgcctccactgagtgcaaggcggagg
tgacgccctcccagcatggcaaccgcaccttcagctacaccctggaggatcataccaagcag
gcctttggcatcatgaacgagctgcggctcagccagcagctgtgtgacgtcacactgcaggt
caagtaccaggatgcaccggccgcccagttcatggcccacaaggtggtgctggcctcatcca
gccctgtcttcaaggccatgttcaccaacgggctgcgggagcagggcatggaggtggtgtcc
attgagggtatccaccccaaggtcatggagcgcctcattgaattcgcctacacggcctccat
ctccatgggcgagaagtgtgtcctccacgtcatgaacggtgctgtcatgtaccagatcgaca
gcgttgtccgtgcctgcagtgacttcctggtgcagcagctggaccccagcaatgccatcggc
atcgccaacttcgctgagcagattggctgtgtggagttgcaccagcgtgcccgggagtacat
ctacatgcattttggggaggtggccaagcaagaggagttcttcaacctgtcccactgccaac
tggtgaccctcatcagccgggacgacctgaacgtgcgctgcgagtccgaggtcttccacgcc
tgcatcaactgggtcaagtacgactgcgaacagcgacggttctacgtccaggcgctgctgcg
ggccgtgcgctgccactcgttgacgccgaacttcctgcagatgcagctgcagaagtgcgaga
tcctgcagtccgactcccgctgcaaggactacctggtcaagatcttcgaggagctcaccctg
cacaagcccacgcaggtgatgccctgccgggcgcccaaggtgggccgcctgatctacaccgc
gggcggctacttccgacagtcgctcagctacctggaggcttacaaccccagtgacggcacct
ggctccggttggcggacctgcaggtgccgcggagcggcctggccggctgcgtggtgggcggg
ctgttgtacgccgtgggcggcaggaacaactcgcccgacggcaacaccgactccagcgccct
ggactgttacaaccccatgaccaatcagtggtcgccctgcgcccccatgagcgtgccccgta
accgcatcggggtgggggtcatcgatggccacatctatgccgtcggcggctcccacggctgc
atccaccacaacagtgtggagaggtatgagccagagcgggatgagtggcacttggtggcccc
aatgctgacacgaaggatcggggtgggcgtggctgtcctcaatcgtctcctttatgccgtgg
ggggctttgacgggacaaaccgccttaattcagctgagtgttactacccagagaggaacgag
tggcgaatgatcacagcaatgaacaccatccgaagcggggcaggcgtctgcgtcctgcacaa
ctgtatctatgctgctgggggctatgatggtcaggaccagctgaacagcgtggagcgctacg
atgtggaaacagagacgtggactttcgtagcccccatgaagcaccggcgaagtgccctgggg
atcactgtccaccaggggagaatctacgtccttggaggctatgatggtcacacgttcctgga
cagtgtggagtgttacgacccagatacagacacctggagcgaggtgacccgaatgacatcgg
gccggagtggggtgggcgtggctgtcaccatggagccctgccggaagcagattgaccagcag
aactgtacctgtttg
SEQ ID NO: 125
MEAF6 protein sequence
MAMHNKAAPPQIPDTRRELAELVKRKQELAETLANLERQIYAFEGSYLEDTQMYGNIIRG
WDRYLTNQKNSNSKNDRRNRKFKEAERLFSKSSVTSAAAVSALAGVQDQLIEKREPGSGT
ESDTSPDFHNQENEPSQEDPEDLDGSVQGVKPQKAASSTSSGSHHSSHKKRKNKNRHRID
LKLNKKPRADY
SEQ ID NO: 126
MEAF6 DNA sequence
ATGGCGATGCACAACAAGGCGGCGCCGCCGCAGATCCCGGACACCCGGCGGGAGCTGGCGGA
GCTCGTGAAGCGGAAGCAGGAGCTGGCGGAAACATTGGCAAATTTGGAGCGACAGATCTATG
CTTTTGAGGGAAGCTACCTGGAAGACACTCAGATGTATGGCAATATTATTCGTGGCTGGGAT
CGGTATCTGACCAACCAAAAAAACTCCAATAGCAAAAATGATCGAAGGAACCGGAAGTTTAA
GGAAGCTGAGCGGCTCTTCAGTAAATCCTCGGTTACCTCAGCAGCTGCAGTAAGTGCATTGG
CAGGAGTTCAGGACCAGCTCATTGAAAAGAGGGAGCCAGGAAGTGGGACGGAAAGTGACACT
TCTCCAGACTTCCACAATCAGGAAAATGAGCCCAGCCAGGAGGACCCTGAGGATCTGGATGG
ATCTGTGCAGGGAGTGAAACCTCAGAAGGCTGCTTCTTCTACTTCCTCAGGGAGTCACCACA
GCAGCCATAAAAAGCGAAAGAATAAAAACCGGCACAGGATTGATCTGAAGTTAAACAAAAAA
CCACGAGCTGACTATTAG
SEQ ID NO: 127
MLLT6 protein sequence
MGAVNPLLSQAESSHTEPDLEDCSFRCRGTSPQESLSSMSPISSLPALFDQTASAPCGGG
QLDPAAPGTTNMEQLLEKQGDGEAGVNIVEMLKALHALQKENQRLQEQILSLTAKKERLQ
ILNVQLSVPFPALPAALPAANGPVPGPYGLPPQAGSSDSLSTSKSPPGKSSLGLDNSLST
SSEDPHSGCPSRSSSSLSFHSTPPPLPLLQQSPATLPLALPGAPAPLPPQPQNGLGRAPG
AAGLGAMPMAEGLLGGLAGSGGLPLNGLLGGLNGAAAPNPASLSQAGGAPTLQLPGCINS
LTEQQRHLLQQQEQQLQQLQQLLASPQLTPEHQTVVYQMIQQIQQKRELQRLQMAGGSQL
PMASLLAGSSTPLLSAGTPGLLPTASAPPLLPAGALVAPSLGNNTSLMAAAAAAAAVAAA
GGPPVLTAQTNPFLSLSGAEGSGGGPKGGTADKGASANQEKG
SEQ ID NO: 128
MLLT6 DNA sequence
ATGGGTGCCGTTAATCCCCTCCTCTCCCAAGCTGAGAGCAGCCACACAGAGCCAGACCTGGA
GGACTGCAGCTTCCGGTGTCGGGGGACCTCCCCTCAGGAGAGTCTGTCTTCCATGTCCCCCA
TCAGCAGCCTCCCCGCACTCTTCGACCAGACAGCCTCTGCACCCTGTGGGGGCGGCCAGTTA
GACCCGGCGGCCCCAGGGACGACTAACATGGAGCAGCTTCTGGAGAAGCAGGGCGACGGGGA
GGCCGGCGTCAACATCGTGGAGATGCTGAAGGCGCTGCACGCGCTGCAGAAGGAGAACCAGC
GGCTGCAAGAGCAGATCCTGAGCCTGACGGCCAAAAAGGAGCGGCTGCAGATTCTCAACGTG
CAGCTCTCTGTGCCCTTCCCTGCCCTGCCTGCTGCCCTGCCTGCCGCCAACGGCCCTGTCCC
TGGGCCCTATGGCCTGCCTCCCCAAGCCGGCAGCAGCGACTCCTTGAGCACCAGCAAGAGCC
CTCCGGGAAAGAGCAGCCTCGGCCTGGACAACTCGCTGTCCACTTCTTCTGAGGACCCACAC
TCAGGCTGCCCGAGCCGCAGCAGCTCGTCGCTGTCCTTCCACAGCACGCCCCCACCGCTGCC
CCTCCTCCAGCAGAGCCCTGCCACTCTGCCCCTGGCCCTGCCTGGGGCCCCTGCCCCACTCC
CGCCCCAGCCGCAGAACGGGTTGGGCCGGGCACCCGGGGCAGCGGGGCTGGGGGCCATGCCC
ATGGCTGAGGGGCTGTTGGGGGGGCTGGCAGGCAGTGGGGGCCTGCCCCTCAATGGGCTCCT
TGGGGGGTTGAATGGGGCCGCTGCCCCCAACCCCGCAAGCTTGAGCCAGGCTGGCGGGGCCC
CCACGCTGCAGCTGCCAGGCTGTCTCAACAGCCTTACAGAGCAGCAGAGACATCTCCTTCAG
CAGCAAGAGCAGCAGCTCCAGCAACTCCAGCAGCTCCTGGCCTCCCCGCAGCTGACCCCGGA
ACACCAGACTGTTGTCTACCAGATGATCCAGCAGATCCAGCAGAAACGGGAGCTGCAGCGCC
TGCAGATGGCTGGGGGCTCCCAGCTGCCCATGGCCAGCCTGCTGGCAGGAAGCTCCACCCCG
CTGCTGTCTGCGGGTACCCCTGGCCTGCTGCCCACAGCGTCTGCTCCACCCCTGCTGCCCGC
TGGAGCCCTAGTGGCTCCCTCGCTTGGCAACAACACAAGTCTCATGGCCGCAGCAGCTGCAG
CTGCAGCAGTAGCAGCAGCAGGCGGACCTCCAGTCCTCACTGCCCAGACCAACCCCTTCCTC
AGCCTGTCGGGAGCAGAGGGCAGTGGCGGTGGCCCCAAAGGAGGGACCGCTGACAAAGGAGC
CTCAGCCAACCAGGAAAAAGGCTAA
SEQ ID NO: 129
MORF4L2 protein sequence
MSSRKQGSQPRGQQSAEEENFKKPTRSNMQRSKMRGASSGKKTAGPQQKNLEPALPGRWG
GRSAENPPSGSVRKTRKNKQKTPGNGDGGSTSEAPQPPRKKRARADPTVESEEAFKNRME
VKVKIPEELKPWLVEDWDLVTRQKQLFQLPAKKNVDAILEEYANCKKSQGNVDNKEYAVN
EVVAGIKEYFNVMLGTQLLYKFERPQYAEILLAHPDAPMSQVYGAPHLLRLFVRIGAMLA
YTPLDEKSLALLLGYLHDFLKYLAKNSASLFTASDYKVASAEYHRKAL
SEQ ID NO: 130
MORF4L2 DNA sequence
ATGAGTTCCAGAAAGCAGGGTTCTCAACCTCGTGGACAGCAATCTGCAGAAGAAGAGAACTT
CAAAAAACCAACTAGAAGCAACATGCAGAGAAGTAAAATGAGAGGGGCCTCCTCAGGAAAGA
AGACAGCTGGTCCACAGCAGAAAAATCTTGAACCAGCTCTCCCAGGAAGATGGGGTGGTCGC
TCTGCAGAGAACCCCCCTTCAGGATCCGTGAGGAAGACCAGAAAGAACAAGCAGAAGACTCC
TGGAAACGGAGATGGTGGCAGTACCAGCGAAGCACCTCAGCCCCCTCGGAAGAAAAGGGCCC
GGGCAGACCCCACTGTTGAAAGTGAGGAGGCGTTTAAGAATAGAATGGAGGTTAAAGTGAAG
ATTCCTGAAGAATTAAAACCATGGCTTGTTGAGGACTGGGACTTAGTTACCAGGCAGAAGCA
GCTGTTTCAACTCCCTGCCAAGAAAAATGTAGATGCAATTCTGGAGGAGTATGCAAATTGCA
AGAAATCGCAGGGAAATGTTGATAATAAGGAATATGCGGTTAATGAAGTTGTGGCAGGAATA
AAAGAATATTTCAATGTGATGTTGGGCACTCAGCTGCTCTACAAATTTGAGAGGCCCCAGTA
TGCTGAAATCCTCTTGGCTCACCCTGATGCTCCAATGTCCCAGGTTTATGGAGCACCACACC
TACTGAGATTATTTGTAAGAATTGGAGCAATGTTGGCCTATACGCCCCTTGATGAGAAAAGC
CTTGCATTATTGTTGGGCTATTTGCATGATTTCCTAAAATATCTGGCAAAGAATTCTGCATC
TCTCTTTACTGCCAGTGATTACAAAGTGGCTTCTGCTGAGTACCACCGCAAAGCCCTGTGA
SEQ ID NO: 131
NFYC protein sequence
MSTEGGFGGTSSSDAQQSLQSFWPRVMEEIRNLTVKDERVQELPLARIKKIMKLDEDVKM
ISAEAPVLFAKAAQIFITELTLRAWIHTEDNKRRTLQRNDIAMAITKFDQFDFLIDIVPR
DELKPPKRQEEVRQSVTPAEPVQYYFTLAQQPTAVQVQGQQQGQQTTSSTTTIQPGQIII
AQPQQGQTTPVTMQVGEGQQVQIVQAQPQGQAQQAQSGTGQTMQVMQQIITNTGEIQQIP
VQLNAGQLQYIRLAQPVSGTQVVQGQIQTLATNAQQITQTEVQQGQQQFSQFTDGQQLYQ
IQQVTMPAGQDLAQPMFIQSANQPSDGQAPQVTGD
SEQ ID NO: 132
NFYC DNA sequence
ATGTCCACAGAAGGAGGATTTGGTGGTACTAGCAGCAGTGATGCCCAGCAAAGCCTACAGTC
GTTCTGGCCTCGGGTCATGGAAGAAATCCGGAATTTAACAGTGAAAGACTTCCGAGTGCAGG
AACTCCCACTGGCTCGTATTAAGAAGATTATGAAACTGGATGAAGATGTGAAGATGATCAGT
GCAGAAGCGCCTGTACTCTTTGCCAAGGCAGCCCAGATTTTTATCACAGAGTTGACTCTTCG
AGCCTGGATTCACACAGAAGATAACAAGCGCCGGACTCTACAGAGAAATGATATCGCCATGG
CAATTACAAAATTTGATCAGTTTGATTTTCTCATCGATATTGTTCCAAGAGATGAACTGAAA
CCTCCAAAGCGTCAGGAGGAGGTGCGCCAGTCTGTAACTCCTGCCGAGCCAGTCCAGTACTA
TTTCACGCTGGCTCAGCAACCCACCGCTGTCCAAGTCCAGGGCCAGCAGCAAGGCCAGCAGA
CCACCAGCTCCACGACCACCATCCAGCCTGGGCAGATCATCATCGCACAGCCTCAGCAGGGC
CAGACCACACCTGTGACAATGCAGGTTGGAGAAGGTCAGCAGGTGCAGATTGTCCAGGCTCA
GCCACAGGGTCAAGCCCAACAGGCCCAGAGTGGCACTGGACAGACCATGCAGGTGATGCAGC
AGATCATCACTAACACAGGAGAGATCCAGCAGATCCCGGTGCAGCTGAATGCCGGCCAGCTG
CAGTATATCCGCTTAGCCCAGCCTGTATCAGGCACTCAAGTTGTGCAGGGACAGATCCAGAC
ACTTGCCACCAATGCTCAACAGATTACACAGACAGAGGTCCAGCAAGGACAGCAGCAGTTCA
GCCAGTTCACAGATGGACAGCAGCTCTACCAGATCCAGCAAGTCACCATGCCTGCGGGCCAG
GACCTCGCCCAGCCCATGTTCATCCAGTCAGCCAACCAGCCCTCCGACGGGCAGGCCCCCCA
GGTGACCGGCGACTGA
SEQ ID NO: 133
PHF 15 protein sequence
MEEKRRKYSISSDNSDTTDSHATSTSASRCSKLPSSTKSGWPRQNEKKPSEVFRTDLITA
MKIPDSYQLSPDDYYILADPWRQEWEKGVQVPAGAEAIPEPVVRILPPLEGPPAQASPSS
TMLGEGSQPDWPGGSRYDLDEIDAYWLELINSELKEMERPELDELTLERVLEELETLCHQ
NMARAIETQEGLGIEYDEDVVCDVCRSPEGEDGNEMVFCDKCNVCVHQACYGILKVPTGS
WLCRTCALGVQPKCLLCPKRGGALKPTRSGTKWVHVSCALWIPEVSIGCPEKMEPITKIS
HIPASRWALSCSLCKECTGTCIQCSMPSCVTAFHVTCAFDHGLEMRTILADNDEVKFKSF
CQEHSDGGPRNEPTSEPTEPSQAGEDLEKVTLRKQRLQQLEEDFYELVEPAEVAERLDLA
EALVDFIYQYWKLKRKANANQPLLTPKTDEVDNLAQQEQDVLYRRLKLFTHLRQDLERVR
NLCYMVTRRERTKHAICKLQEQIFHLQMKLIEQDLCRAGLSTSFPIDGTFFNSWLAQSVQ
ITAENMAMSEWPLNNGHREDPAPGLLSEELLQDEETLLSFMRDPSLRPGDPARKARGRTR
LPAKKKPPPPPPQDGPGSRTTPDKAPKKTWGQDAGSGKGGQGPPTRKPPRRTSSHLPSSP
AAGDCPILATPESPPPLAPETPDEAASVAADSDVQVPGPAASPKPLGRLRPPRESKVTRR
LPGARPDAGMGPPSAVAERPKVSLHFDTETDGYFSDGEMSDSDVEAEDGGVQRGPREAGA
EEVVRMGVLAS
SEQ ID NO: 134
PHF 15 DNA sequence
ATGGAAGAGAAGAGGCGAAAATACTCCATCAGCAGTGACAACTCTGACACCACTGACAGTCA
TGCGACATCTACATCCGCATCAAGATGCTCCAAACTGCCCAGCAGCACCAAGTCGGGCTGGC
CCCGACAGAACGAAAAGAAGCCCTCCGAGGTTTTCCGGACAGACTTGATCACAGCCATGAAG
ATCCCGGACTCATACCAGCTCAGCCCGGATGACTACTACATCCTGGCAGACCCATGGCGACA
GGAATGGGAGAAAGGTGTGCAGGTGCCTGCCGGGGCAGAGGCCATCCCAGAGCCCGTGGTGA
GGATCCTCCCACCACTGGAAGGCCCCCCTGCCCAGGCATCCCCGAGCAGCACCATGCTTGGT
GAGGGCTCCCAGCCTGATTGGCCAGGGGGCAGCCGCTATGACTTGGACGAGATTGATGCCTA
CTGGCTGGAGCTCATCAACTCGGAGCTTAAGGAGATGGAGAGGCCGGAGCTGGACGAGCTGA
CATTAGAGCGTGTGCTGGAGGAGCTGGAGACCCTGTGCCACCAGAATATGGCCAGGGCCATT
GAGACGCAGGAGGGGCTGGGCATCGAGTACGACGAGGATGTTGTCTGCGACGTGTGTCGCTC
TCCTGAGGGCGAGGATGGCAACGAGATGGTCTTCTGTGACAAGTGCAACGTCTGTGTGCATC
AGGCATGCTACGGGATCCTCAAGGTGCCCACGGGCAGCTGGCTGTGCCGGACGTGTGCCCTG
GGTGTCCAGCCAAAGTGCCTGCTCTGCCCCAAGCGAGGAGGAGCCTTGAAGCCCACTAGAAG
TGGGACCAAGTGGGTGCATGTCAGCTGTGCCCTATGGATTCCTGAGGTCAGCATCGGCTGCC
CAGAGAAGATGGAGCCCATCACCAAGATCTCGCATATCCCAGCCAGCCGCTGGGCTCTGTCC
TGCAGCCTCTGCAAGGAATGCACAGGCACCTGCATCCAGTGTTCCATGCCTTCCTGCGTCAC
AGCGTTCCATGTCACATGCGCCTTTGACCACGGCCTGGAAATGCGGACTATATTAGCAGACA
ACGATGAGGTCAAGTTCAAGTCATTCTGCCAGGAGCACAGTGACGGGGGCCCACGTAATGAG
CCCACATCTGAGCCCACGGAACCCAGCCAGGCTGGCGAGGACCTGGAAAAGGTGACCCTGCG
CAAGCAGCGGCTGCAGCAGCTAGAGGAGGACTTCTACGAGCTGGTGGAGCCGGCTGAGGTGG
CTGAGCGGCTGGACCTGGCTGAGGCACTGGTCGACTTCATCTACCAGTACTGGAAGCTGAAG
AGGAAAGCCAATGCCAACCAGCCGCTGCTGACCCCCAAGACCGACGAGGTGGACAACCTGGC
CCAGCAGGAGCAGGACGTCCTCTACCGCCGCCTGAAGCTCTTCACCCATCTGCGGCAGGACC
TAGAGAGGGTTAGAAATCTGTGCTACATGGTGACAAGGCGCGAGAGAACGAAACACGCCATC
TGCAAACTCCAGGAGCAGATATTCCACCTGCAGATGAAACTTATTGAACAGGATCTGTGTCG
AGCAGGCCTGTCCACCTCATTCCCCATCGATGGCACCTTCTTCAACAGCTGGCTGGCACAGT
CGGTGCAGATCACAGCAGAGAACATGGCCATGAGCGAGTGGCCACTGAACAATGGGCACCGC
GAGGACCCTGCTCCAGGGCTGCTGTCAGAGGAACTGCTGCAGGACGAGGAGACACTGCTCAG
CTTCATGCGGGACCCCTCGCTGCGACCTGGTGACCCTGCTAGGAAGGCCCGAGGCCGCACCC
GCCTGCCTGCCAAGAAGAAACCACCACCACCACCACCGCAGGACGGGCCTGGTTCACGGACG
ACTCCAGACAAAGCCCCCAAGAAGACCTGGGGCCAGGATGCAGGCAGTGGCAAGGGGGGTCA
AGGGCCACCTACCAGGAAGCCACCACGTCGGACATCTTCTCACTTGCCGTCCAGCCCTGCAG
CCGGGGACTGTCCCATCCTAGCCACCCCTGAAAGCCCCCCGCCACTGGCCCCTGAGACCCCG
GACGAGGCAGCCTCAGTAGCTGCTGACTCAGATGTCCAAGTGCCTGGCCCTGCAGCAAGCCC
TAAGCCTTTGGGCCGGCTCCGGCCACCCCGCGAGAGCAAGGTAACCCGGAGATTGCCGGGTG
CCAGGCCTGATGCTGGGATGGGACCACCTTCAGCTGTGGCTGAGAGGCCCAAGGTCAGCCTG
CATTTTGACACTGAGACTGATGGCTACTTCTCTGATGGGGAGATGAGCGACTCAGATGTAGA
GGCCGAGGACGGTGGGGTGCAGCGGGGTCCCCGGGAGGCAGGGGCAGAGGAGGTGGTCCGCA
TGGGCGTACTGGCCTCCTAA
SEQ ID NO: 135
PKIB protein sequence
MRTDSSKMTDVESGVANFASSARAGRRNALPDIQSSAATDGTSDLPLKLEALSVKEDAKE
KDEKTTQDQLEKPONEEKCPTFLY
SEQ ID NO: 136
PKIB DNA sequence
Atgaggacagattcatcaaaaatgactgacgtggagtctggggtcgccaattttgcatcttc
agcaagggcaggccgccggaatgccttaccagacatccagagttcagctgccacagacggaa
cctcagatttgcccctcaaactggaggctctctccgtgaaggaagatgcaaaagagaaagat
gaaaaaacaacacaagaccaattggaaaagcctcaaaatgaagaaaaatgcccaactttctt
gtac
SEQ ID NO: 137
POLE4 protein sequence
MAAAAAAGSGTPREEEVPAGEAAASQPQAPTSVPGARLSRLPLARVKALVKADPDVTLAG
QEAIFILARAAELFVETIAKDAYCCAQQGKRKTLQRRDLDNAIEAVDEFAFLEGTLD
SEQ ID NO: 138
POLE4 DNA sequence
ATGGCGGCGGCGGCGGCGGCAGGAAGCGGGACGCCCCGAGAGGAGGAGGTACCTGCTGGGGA
GGCAGCGGCCTCGCAGCCCCAGGCCCCAACGAGTGTGCCTGGGGCTCGTCTCTCGAGGTTGC
CTCTGGCGCGAGTGAAGGCCTTGGTGAAGGCAGATCCCGACGTGACGCTAGCGGGACAGGAA
GCCATCTTCATTCTGGCACGAGCCGCGGAACTGTTTGTGGAGACCATTGCAAAAGATGCCTA
CTGTTGCGCTCAGCAGGGAAAAAGGAAAACCCTTCAGAGGAGAGACTTGGATAATGCAATAG
AAGCTGTGGATGAATTTGCTTTTCTGGAAGGTACTTTAGATTGA
SEQ ID NO: 139
PRKRIR protein sequence
MPNFCAAPNCTRKSTQSDLAFFRFPRDPARCQKWVENCRRADLEDKTPDQLNKHYRLCAK
HFETSMICRTSPYRTVLRDNAIPTIFDLTSHLNNPHSRHRKRIKELSEDEIRTLKQKKID
ETSEQEQKHKETNNSNAQNPSEEEGEGQDEDILPLTLEEKENKEYLKSLFEILILMGKQN
IPLDGHEADEIPEGLFTPDNFQALLECRINSGEEVLRKRFETTAVNTLFCSKTQQRQMLE
ICESCIREETLREVRDSHFFSIITDDVVDIAGEEHLPVLVRFVDESHNLREEFIGELPYE
ADAEILAVKFHTMITEKWGLNMEYCRGQAYIVSSGESSKMKVVASRLLEKYPQAIYTLCS
SCALNMWLAKSVPVMGVSVALGTIEEVCSFFHRSPQLLLELDNVISVLFQNSKERGKELK
EICHSQWTGRHDAFEILVELLQALVLCLDGINSDTNIRWNNYIAGRAFVLCSAVSDFDFI
VTIVVLKNVLSFTRAFGKNLQGQTSDVFFAAGSLTAVLHSLNEVMENIEVYHEFWFEEAT
NLATKLDIQMKLPGKERRAHQGNLESQLTSESYYKETLSVPTVEHIIQELKDIFSEQHLK
ALKCLSLVPSVMGQLKENTSEEHHADMYRSDLPNPDTLSAELHCWRIKWKHRGKDIELPS
TIYEALHLPDIKFFPNVYALLKVLCILPVMKVENERYENGRKRLKAYLRNTLTDQRSSNL
ALLNINFDIKHDLDLMVDTYIKLYTSKSELPTDNSETVENT
SEQ ID NO: 140
PRKRIR DNA sequence
ATGCCGAACTTCTGCGCTGCCCCCAACTGCACGCGGAAGAGCACGCAGTCCGACTTGGCCTT
CTTCAGGTTCCCGCGGGACCCTGCCAGATGCCAGAAGTGGGTGGAGAACTGTAGGAGAGCAG
ACTTAGAAGATAAAACACCTGATCAGCTAAATAAACATTATCGATTATGTGCCAAACATTTT
GAGACCTCTATGATCTGTAGAACTAGTCCTTATAGGACAGTTCTTCGAGATAATGCAATACC
AACAATATTTGATCTTACCAGTCATTTGAACAACCCACATAGTAGACACAGAAAACGAATAA
AAGAACTGAGTGAAGATGAAATCAGGACACTGAAACAGAAAAAAATTGATGAAACTTCTGAG
CAGGAACAAAAACATAAAGAAACCAACAATAGCAATGCTCAGAACCCCAGCGAAGAAGAGGG
TGAAGGGCAAGATGAGGACATTTTACCTCTAACCCTTGAAGAGAAGGAAAACAAAGAATACC
TAAAATCTCTATTTGAAATCTTGATTCTGATGGGAAAGCAAAACATACCTCTGGATGGACAT
GAGGCTGATGAAATCCCAGAAGGTCTCTTTACTCCAGATAACTTTCAGGCACTGCTGGAGTG
TCGGATAAATTCTGGTGAAGAGGTTCTGAGAAAGCGGTTTGAGACAACAGCAGTTAACACGT
TGTTTTGTTCAAAAACACAGCAGAGGCAGATGCTAGAGATCTGTGAGAGCTGTATTCGAGAA
GAAACTCTCAGGGAAGTGAGAGACTCACACTTCTTTTCCATTATCACTGACGATGTAGTGGA
CATAGCAGGGGAAGAGCACCTACCTGTGTTGGTGAGGTTTGTTGATGAATCTCATAACCTAA
GAGAGGAATTTATAGGCTTCCTGCCTTATGAAGCCGATGCAGAAATTTTGGCTGTGAAATTT
CACACTATGATAACTGAGAAGTGGGGATTAAATATGGAGTATTGTCGTGGCCAGGCTTACAT
TGTCTCTAGTGGATTTTCTTCCAAAATGAAAGTTGTTGCTTCTAGACTTTTAGAGAAATATC
CCCAAGCTATCTACACACTCTGCTCTTCCTGTGCCTTAAATATGTGGTTGGCAAAATCAGTA
CCTGTTATGGGAGTATCTGTTGCATTAGGAACAATTGAGGAAGTTTGTTCTTTTTTCCATCG
ATCACCACAACTGCTTTTAGAACTTGACAACGTAATTTCTGTTCTTTTTCAGAACAGTAAAG
AAAGGGGTAAAGAACTGAAGGAAATCTGCCATTCTCAGTGGACAGGCAGGCATGATGCTTTT
GAAATTTTAGTGGAACTCCTGCAAGCACTTGTTTTATGTTTAGATGGTATAAATAGTGACAC
AAATATTAGATGGAATAACTATATAGCTGGCCGAGCATTTGTACTCTGCAGTGCAGTGTCAG
ATTTTGATTTCATTGTTACTATTGTTGTTCTTAAAAATGTCCTATCTTTTACAAGAGCCTTT
GGGAAAAACCTCCAGGGGCAAACCTCTGATGTCTTCTTTGCGGCCGGTAGCTTGACTGCAGT
ACTGCATTCACTCAACGAAGTGATGGAAAATATTGAAGTTTATCATGAATTTTGGTTTGAGG
AAGCCACAAATTTGGCAACCAAACTTGATATTCAAATGAAACTCCCTGGGAAATTCCGCAGA
GCTCACCAGGGTAACTTGGAATCTCAGCTAACCTCTGAGAGTTACTATAAAGAAACCCTAAG
TGTCCCAACAGTGGAGCACATTATTCAGGAACTTAAAGATATATTCTCAGAACAGCACCTCA
AAGCTCTTAAATGCTTATCTCTGGTACCCTCAGTCATGGGACAACTCAAATTCAATACGTCG
GAGGAACACCATGCTGACATGTATAGAAGTGACTTACCCAATCCTGACACGCTGTCAGCTGA
GCTTCATTGTTGGAGAATCAAATGGAAACACAGGGGGAAAGATATAGAGCTTCCGTCCACCA
TCTATGAAGCCCTCCACCTGCCTGACATCAAGTTTTTTCCTAATGTGTATGCATTGCTGAAG
GTCCTGTGTATTCTTCCTGTGATGAAGGTTGAGAATGAGCGGTATGAAAATGGACGAAAGCG
TCTTAAAGCATATTTGAGGAACACTTTGACAGACCAAAGGTCAAGTAACTTGGCTTTGCTTA
ACATAAATTTTGATATAAAACACGACCTGGATTTAATGGTGGACACATATATTAAACTCTAT
ACAAGTAAGTCAGAGCTTCCTACAGATAATTCCGAAACTGTGGAAAATACCTAA
SEQ ID NO: 141
PYGO2 protein sequence
MAASAPPPPDKLEGGGGPAPPPAPPSTGRKQGKAGLQMKSPEKKRRKSNTQGPAYSHLTE
FAPPPTPMVDHLVASNPFEDDFGAPKVGVAAPPFLGSPVPFGGFRVQGGMAGQVPPGYST
GGGGGPQPLRRQPPPFPPNPMGPAFNMPPQGPGYPPPGNMNFPSQPENQPLGQNESPPSG
QMMPGPVGGFGPMISPTMGQPPRAELGPPSLSQRFAQPGAPFGPSPLQRPGQGLPSLPPN
TSPFPGPDPGFPGPGGEDGGKPLNPPASTAFPQEPHSGSPAAAVNGNQPSFPPNSSGRGG
GTPDANSLAPPGKAGGGSGPQPPPGLVYPCGACRSEVNDDQDAILCEASCQKWFHRECTG
MTESAYGLLTTEASAVWACDLCLKTKEIQSVYIREGMGQLVAANDGL
SEQ ID NO: 142
PYGO2 DNA sequence
atggccgcctcggcgccgcccccaccggacaagctggagggaggtggcggccccgcaccgcc
ccctgcgccgcccagcaccgggaggaagcagggcaaggccggtctgcaaatgaagagtccag
aaaagaagcgaaggaagtcaaatactcagggccctgcatactcacatctgacggagtttgca
ccacccccaactcccatggtggatcacctggttgcatccaacccttttgaagatgacttcgg
agcccccaaagtgggggttgcagcccctccattccttggcagtcctgtgcccttcggaggct
tccgtgtgcaggggggcatggcgggccaggtacccccaggctacagcactggaggtggaggg
ggcccccagccactccgtcgacagccaccccccttccctcccaatcctatgggccctgcttt
caacatgcccccccagggtcctggctacccacccccaggcaacatgaactttcccagccaac
ccttcaaccagcctctgggtcaaaactttagtcctcccagtgggcagatgatgccgggccca
gtggggggatttggtcccatgatctcacccaccatgggacagcctcccagagcagagctggg
cccaccttctctgtcccaacgatttgctcagccaggggctccttttggcccttctcctctcc
agagacctggtcaggggctccccagcctgccgcctaacacaagtccctttcctggtccggac
cctggctttcctggccctggtggtgaggatggggggaagcccttgaatccacctgcttctac
tgcttttccccaggagccccactcaggctccccggctgctgctgttaatgggaaccagccca
gtttccccccgaacagcagtgggcggggtgggggcactccagatgccaacagcttggcaccc
cctggcaaggcaggtgggggctccgggccccagcctcccccaggcttggtgtacccatgtgg
tgcctgtcggagtgaggtgaacgatgaccaggatgccattctgtgtgaggcctcctgccaga
aatggttccaccgtgagtgcacaggcatgactgagagcgcctatgggctgctgaccactgaa
gcttctgccgtctgggcctgcgatctctgcctcaagaccaaggagatccagtctgtctacat
ccgtgagggcatggggcagctggtggctgctaacgatgggttg
SEQ ID NO: 143
RANBP1 protein sequence
MAAAKDTHEDHDTSTENTDESNHDPQFEPIVSLPEQEIKTLEEDEEELFKMRAKLERFAS
ENDLPEWKERGTGDVKLLKHKEKGAIRLLMRRDKTLKICANHYITPMMELKPNAGSDRAW
VWNTHADFADECPKPELLAIRFLNAENAQKFKTKFEECRKEIEEREKKAGSGKNDHAEKV
AEKLEALSVKEETKEDAEEKQPTFLY
SEQ ID NO: 144
RANBP1 DNA sequence
atggcggccgccaaggacactcatgaggaccatgatacttccactgagaatacagacgagtc
caaccatgaccctcagtttgagccaatagtttctcttcctgagcaagaaattaaaacactgg
aagaagatgaagaggaactttttaaaatgcgggcaaaactgttccgatttgcctctgagaac
gatctcccagaatggaaggagcgaggcactggtgacgtcaagctcctgaagcacaaggagaa
aggggccatccgcctcctcatgcggagggacaagaccctgaagatctgtgccaaccactaca
tcacgccgatgatggagctgaagcccaacgcaggtagcgaccgtgcctgggtctggaacacc
cacgctgacttcgccgacgagtgccccaagccagagctgctggccatccgcttcctgaatgc
tgagaatgcacagaaattcaaaacaaagtttgaagaatgcaggaaagagatcgaagagagag
aaaagaaagcaggatcaggcaaaaatgatcatgccgaaaaagtggcggaaaagctagaagct
ctctcggtgaaggaggagaccaaggaggatgctgaggagaagcaaccaactttcttgtac
SEQ ID NO: 145
RPRD1B protein sequence
MSSFSESALEKKLSELSNSQHSVQTLSLWLIHHRKHAGPIVSVWHRELRKAKSNRKLTFL
YLANDVIQNSKRKGPEFTREFESVLVDAFSHVAREADEGCKKPLERLLNIWQERSVYGGE
FIQQLKLSMEDSKSPPPKATEEKKSLKRTFQQIQEEEDDDYPGSYSPQDPSAGPLLTEEL
IKALQDLENAASGDATVRQKIASLPQEVQDVSLLEKITDKEAAERLSKTVDEACLLLAEY
NGRLAAELEDRRQLARMLVEYTQNQKDVLSEKEKKLEEYKQKLARVTQVRKELKSHIQSL
PDLSLLPNVTGGLAPLPSAGDLFSTD
SEQ ID NO: 146
RPRD1B DNA sequence
ATGTCCTCCTTCTCTGAGTCGGCGCTGGAGAAGAAGCTCTCGGAGCTGAGCAACTCTCAGCA
CAGCGTGCAGACCCTGTCCCTTTGGCTCATCCACCACCGCAAGCACGCGGGACCCATCGTCT
CCGTGTGGCACCGCGAGCTCCGCAAAGCCAAATCAAATAGAAAGCTTACTTTTCTGTATTTA
GCGAATGATGTCATCCAAAACAGTAAAAGGAAAGGACCTGAATTCACTAGAGAATTTGAATC
TGTCCTTGTGGATGCTTTTTCTCATGTTGCCAGAGAGGCAGATGAAGGCTGTAAAAAACCTT
TAGAAAGATTGCTGAACATCTGGCAAGAACGAAGTGTGTATGGCGGCGAGTTCATACAGCAG
CTGAAGCTGTCTATGGAGGACTCCAAGAGCCCTCCCCCCAAAGCAACAGAAGAGAAGAAATC
TCTGAAACGAACTTTTCAGCAAATTCAGGAGGAGGAGGATGACGACTACCCTGGCAGCTACT
CTCCTCAGGATCCTTCTGCAGGACCCCTCTTGACTGAGGAACTAATCAAAGCTTTGCAGGAT
CTGGAAAATGCCGCATCAGGGGATGCTACTGTCCGACAGAAAATTGCTTCTCTGCCCCAGGA
AGTGCAAGATGTTTCTCTATTGGAAAAAATAACAGACAAAGAGGCAGCTGAACGTCTTTCAA
AAACAGTAGATGAAGCATGTCTGTTACTAGCAGAATATAACGGGCGCCTGGCAGCAGAACTG
GAGGACCGTCGCCAGCTGGCTCGGATGTTGGTGGAGTATACCCAGAATCAGAAAGATGTTTT
GTCGGAGAAGGAGAAAAAACTAGAGGAATACAAACAGAAGCTTGCACGAGTAACCCAGGTCC
GCAAGGAACTGAAATCCCATATTCAGAGCTTGCCAGACCTCTCACTGCTGCCCAACGTCACA
GGGGGCTTAGCCCCCCTGCCCTCTGCTGGGGACCTGTTTTCAACTGACTAG
SEQ ID NO: 147
SPIN1 protein sequence
MKTPFGKTPGQRSRADAGHAGVSANMMKKRTSHKKHRSSVGPSKPVSQPRRNIVGCRIQH
GWKEGNGPVTQWKGTVLDQVPVNPSLYLIKYDGFDCVYGLELNKDERVSALEVLPDRVAT
SRISDAHLADTMIGKAVEHMFETEDGSKDEWRGMVLARAPVMNTWFYITYEKDPVLYMYQ
LLDDYKEGDLRIMPDSNDSPPAEREPGEVVDSLVGKQVEYAKEDGSKRTGMVIHQVEAKP
SVYFIKFDDDFHIYVYDLVKTS
SEQ ID NO: 148
SPIN1 DNA sequence
ATGAAGACCCCATTCGGAAAGACACCTGGCCAGCGGTCCAGAGCTGATGCAGGCCATGCTGG
AGTATCTGCCAACATGATGAAGAAGAGGACATCCCACAAAAAACATCGGAGCAGTGTGGGTC
CGAGCAAACCTGTTTCCCAGCCCCGGCGGAACATCGTAGGCTGCAGGATTCAGCATGGGTGG
AAAGAGGGGAATGGCCCTGTTACCCAGTGGAAAGGAACCGTTCTGGACCAGGTGCCTGTAAA
TCCTTCTTTGTATCTTATAAAATACGATGGATTTGACTGTGTTTATGGACTAGAACTTAATA
AAGATGAAAGAGTTTCTGCGCTTGAAGTCCTCCCTGATAGAGTTGCGACATCTCGAATCAGC
GATGCACACTTGGCAGACACAATGATTGGCAAAGCAGTGGAACATATGTTTGAGACAGAGGA
TGGTTCTAAAGATGAGTGGAGGGGAATGGTCTTAGCACGTGCACCTGTCATGAACACATGGT
TTTACATTACCTATGAGAAAGACCCTGTCTTGTACATGTACCAACTCTTAGATGATTACAAA
GAAGGCGACCTTCGCATTATGCCTGATTCCAATGATTCACCTCCAGCAGAAAGGGAACCAGG
AGAAGTIGTGGACAGCCTGGTAGGCAAACAAGTGGAATATGCCAAAGAAGATGGCTCGAAAA
GGACTGGCATGGTCATTCATCAAGTAGAAGCCAAGCCCTCCGTCTATTTCATCAAGTTTGAT
GATGATTTCCATATTTATGTCTACGATTTGGTGAAAACATCCTAG
SEQ ID NO: 149
SS18L1 protein sequence
MSVAFASARPRGKGEVTQQTIQKMLDENHHLIQCILEYQSKGKTAECTQYQQILHRNLVY
LATIADSNQNMQSLLPAPPTQNMNLGPGALTQSGSSQGLHSQGSLSDAISTGLPPSSLLQ
GQIGNGPSHVSMQQTAPNTLPTTSMSISGPGYSHAGPASQGVPMQGQGTIGNYVSRTNIN
MQSNPVSMIQQQAATSHYSSAQGGSQHYQGQSSIAMMGQGSQGSSMMGQRPMAPYRPSQQ
GSSQQYLGQEEYYGEQYSHSQGAAEPMGQQYYPDGHGDYAYQQSSYTEQSYDRSFEESTQ
HYYEGGNSQYSQQQAGYQQGAAQQQTYSQQQYPSQQSYPGQQQGYGSAQGAPSQYPGYQQ
GQGQQYGSYRAPQTAPSAQQQRPYGYEQGQYGNYQQL
SEQ ID NO: 150
SS18L1 DNA sequence
Atgtccgtggccttcgcgtctgcccggccaagaggcaaaggggaggttacgcagcaaaccat
ccagaagatgctggacgagaaccaccacctgatccagtgcatcctggagtaccagagcaagg
gcaagacggccgagtgcacgcagtaccagcagatcctgcaccggaacctggtatacctggcc
acgatcgcagactccaaccagaacatgcagtccctgcttcctgccccgcccacgcagaacat
gaacctgggccctggagccctgactcagagcggctccagccagggcctgcactctcagggca
gcctgagtgacgccatcagcacgggcctgccaccctcctccctcctgcagggccagattggc
aacgggccgagccacgtgtccatgcagcagacggcgcctaacacgctgcccaccacctccat
gagcatctctgggcccggctacagccacgcgggacccgcctcgcagggcgtccccatgcagg
ggcaaggcaccatcggcaactacgtgtctcggaccaacatcaacatgcagtccaacccagtc
tccatgatacagcagcaggcggccacgtcgcactacagctcggcgcagggcggcagccagca
ctaccagggccagtcgtccatcgccatgatggggcagggcagccaggggagcagcatgatgg
ggcagcggcccatggcgccctaccggccctcccagcaaggctcttcccagcagtacctgggc
caggaggagtactatggcgagcagtacagccacagccagggcgccgcggagcccatgggcca
gcagtactaccccgacggccatggcgattacgcctaccagcagtcatcctacacggagcaga
gctacgaccggtccttcgaggagtccacgcagcactactatgaggggggaaactcccagtac
agccagcagcaggccgggtaccagcagggtgccgcgcagcagcagacgtactcccagcagca
gtaccccagccagcagagctaccccgggcagcagcagggctacgggtctgcccagggagccc
cgtcacagtaccccggctaccagcaaggccaaggccagcagtacggaagctaccgagcaccg
cagacagcgccgtctgcccagcagcagcggccctacggctatgaacagggccagtatggaaa
ttaccagcagttg
SEQ ID NO: 151
TADA3 protein sequence
MSELKDCPLQFHDFKSVDHLKVCPRYTAVLARSEDDGIGIEELDTLQLELETLLSSASRR
LRVLEAETQILTDWQDKKGDRRFLKLGRDHELGAPPKHGKPKKQKLEGKAGHGPGPGPGR
PKSKNLQPKIQEYEFTDDPIDVPRIPKNDAPNRFWASVEPYCADITSEEVRTLEELLKPP
EDEAEHYKIPPLGKHYSQRWAQEDLLEEQKDGARAAAVADKKKGLMGPLTELDTKDVDAL
LKKSEAQHEQPEDGCPFGALTQRLLQALVEENIISPMEDSPIPDMSGKESGADGASTSPR
NQNKPFSVPHTKSLESRIKEELIAQGLLESEDRPAEDSEDEVLAELRKRQAELKALSAHN
RTKKHDLLRLAKEEVSRQELRQRVRMADNEVMDAFRKIMAARQKKRTPTKKEKDQAWKTL
KERESILKLLDG
SEQ ID NO: 152
TADA3 DNA sequence
ATGAGTGAGTTGAAAGACTGCCCCTTGCAGTTCCACGACTTCAAGTCTGTGGATCACCTGAA
GGTCTGTCCCCGCTACACGGCAGTGCTGGCACGCTCTGAGGATGATGGCATCGGCATCGAGG
AGCTGGACACCCTGCAGCTGGAGCTGGAGACCCTGCTGTCTTCTGCCAGCCGGCGCCTGCGT
GTGCTTGAGGCCGAAACCCAGATCCTCACCGACTGGCAGGATAAGAAAGGTGACAGACGATT
CCTGAAGCTGGGTCGAGACCATGAACTTGGAGCTCCCCCCAAACATGGGAAGCCCAAGAAGC
AGAAACTGGAAGGGAAGGCAGGACATGGGCCGGGCCCTGGCCCAGGACGGCCCAAATCCAAA
AACCTTCAGCCCAAGATCCAGGAATATGAATTCACTGATGACCCTATCGACGTGCCACGGAT
CCCCAAAAATGATGCCCCCAACAGGTTCTGGGCTTCAGTGGAGCCCTACTGTGCTGACATCA
CCAGCGAGGAGGTCCGCACACTTGAGGAGTTACTGAAGCCCCCAGAAGATGAGGCTGAGCAT
TACAAGATCCCACCCCTGGGGAAGCACTACTCCCAGCGCTGGGCCCAGGAGGACCTGCTGGA
GGAGCAGAAGGATGGGGCCCGGGCAGCGGCTGTGGCTGACAAGAAGAAAGGCCTCATGGGGC
CACTGACCGAACTGGACACTAAAGATGTGGATGCCCTGCTGAAGAAGTCTGAGGCCCAGCAT
GAACAGCCGGAAGATGGATGCCCCTTTGGTGCCCTGACGCAGCGCCTCCTGCAGGCCCTGGT
GGAGGAAAATATTATTTCCCCTATGGAGGATTCTCCTATTCCTGACATGTCTGGGAAAGAAT
CAGGGGCTGACGGGGCAAGCACCTCCCCTCGCAATCAGAACAAGCCCTTCAGTGTGCCGCAT
ACTAAGTCCCTGGAGAGCCGCATCAAGGAGGAGCTAATTGCCCAGGGCCTTTTGGAGTCTGA
GGACCGCCCCGCAGAGGACTCCGAGGATGAGGTCCTTGCTGAGCTTCGCAAACGGCAGGCTG
AGCTGAAGGCACTTAGTGCCCACAACCGCACCAAGAAGCACGACCTGCTGAGGCTGGCAAAG
GAGGAGGTGAGCCGGCAGGAGCTGAGGCAGCGGGTGCGCATGGCTGACAACGAGGTCATGGA
CGCCTTTCGCAAGATCATGGCTGCCCGGCAGAAGAAGCGGACTCCCACCAAGAAAGAAAAGG
ACCAGGCCTGGAAGACTCTGAAGGAGCGTGAGAGCATCCTGAAGCTGCTGGATGGGTAG
SEQ ID NO: 153
TAF6 protein sequence
MAEEKKLKLSNTVLPSESMKVVAESMGIAQIQEETCQLLTDEVSYRIKEIAQDALKFMHM
GKRQKLTTSDIDYALKLKNVEPLYGFHAQEFIPFRFASGGGRELYFYEEKEVDLSDIINT
PLPRVPLDVCLKAHWLSIEGCQPAIPENPPPAPKEQQKAEATEPLKSAKPGQEEDGPLKG
KGQGATTADGKGKEKKAPPLLEGAPLRLKPRSIHELSVEQQLYYKEITEACVGSCEAKRA
EALQSIATDPGLYQMLPRESTFISEGVRVNVVQNNLALLIYLMRMVKALMDNPTLYLEKY
VHELIPAVMTCIVSRQLCLRPDVDNHWALRDFAARLVAQICKHFSTTINNIQSRITKTFT
KSWVDEKTPWTTRYGSIAGLAELGHDVIKTLILPRLQQEGERIRSVLDGPVLSNIDRIGA
DHVQSLLLKHCAPVLAKLRPPPDNQDAYRAEFGSLGPLLCSQVVKARAQAALQAQQVNRT
TLTITQPRPTLTLSQAPQPGPRTPGLLKVPGSIALPVQTLVSARAAAPPQPSPPPTKFIV
MSSSSSAPSTQQVLSLSTSAPGSGSTTTSPVTTTVPSVQPIVKLVSTATTAPPSTAPSGP
GSVQKYIVVSLPPTGEGKGGPTSHPSPVPPPASSPSPLSGSALCGGKQEAGDSPPPAPGT
PKANGSQPNSGSPQPAPL
SEQ ID NO: 154
TAF6 DNA sequence
atggctgaggagaagaagctgaagcttagcaacactgtgctgccctcggagtccatgaaggt
ggtggctgaatccatgggcatcgcccagattcaggaggagacctgccagctgctaacggatg
aggtcagctaccgcatcaaagagatcgcacaggatgccttgaagttcatgcacatggggaag
cggcagaagctcaccaccagtgacattgactacgccttgaagctaaagaatgtcgagccact
ctatggcttccacgcccaggagttcattcctttccgcttcgcctctggtgggggccgggagc
tttacttctatgaggagaaggaggttgatctgagcgacatcatcaatacccctctgccccgg
gtgcccctggacgtctgcctcaaagctcattggctgagcatcgagggctgccagccagctat
ccccgagaacccgcccccagctcccaaagagcaacagaaggctgaagccacagaacccctga
agtcagccaagccaggccaggaggaagacggacccctgaagggcaaaggtcaaggggccacc
acagccgacggcaaagggaaagagaagaaggcgccgcccttgctggagggggcccccttgcg
actgaagccccggagcatccacgagttgtctgtggagcagcagctctactacaaggagatca
ccgaggcctgcgtgggctcctgcgaggccaagagggcggaagccctgcaaagcattgccacg
gaccctggactgtatcagatgctgccacggttcagtacctttatctcggagggggtccgtgt
gaacgtggttcagaacaacctggccctactcatctacctgatgcgtatggtgaaagcgctga
tggacaaccccacgctctatctagaaaaatacgtccatgagctgattccagctgtgatgacc
tgcatcgtgagcagacagttgtgcctgcgaccagatgtggacaatcactgggcactccgaga
ctttgctgcccgcctggtggcccagatctgcaagcattttagcacaaccactaacaacatcc
agtcccggatcaccaagaccttcaccaagagctgggtggacgagaagacgccctggacgact
cgttatggctccatcgcaggcttggctgagctgggacacgatgttatcaagactctgattct
gccccggctgcagcaggaaggggagcggatccgcagtgtgctggacggccctgtgctgagca
acattgaccggattggagcagaccatgtgcagagcctcctgctgaaacactgtgctcctgtt
ctggcaaagctgcgcccaccgcctgacaatcaggacgcctatcgggcagaattcgggtccct
tgggcccctcctctgctcccaggtggtcaaggctcgggcccaggctgctctgcaggctcagc
aggtcaacaggaccactctgaccatcacgcagccccggcccacgctgaccctctcgcaggcc
ccacagcctggccctcgcacccctggcttgctgaaggttcctggctccatcgcacttcctgt
ccagacactggtgtctgcacgagcggctgccccaccacagccttcccctcctccaaccaagt
ttattgtaatgtcatcgtcctccagcgccccatccacccagcaggtcctgtccctcagcacc
tcggcccccggctcaggttccaccaccacttcgcccgtcaccaccaccgtccccagcgtgca
gcccatcgtcaagttggtctccaccgccaccaccgcaccccccagcactgctccctctggtc
ctgggagtgtccagaagtacatcgtggtctcacttcccccaacaggggagggcaaaggaggc
cccacctcccatccttctccagttcctcccccggcatcgtccccgtccccactcagcggcag
tgccctttgtggggggaagcaggaggctggggacagtccccctccagctccagggactccaa
aagccaatggctcccagcccaactccggctcccctcagcctgctccgttg
SEQ ID NO: 155
TBPL1 protein sequence
MDADSDVALDILITNVVCVERTRCHLNLRKIALEGANVIYKRDAGKVLMKLRKPRITATI
WSSGKIICTGATSEEEAKFGARRLARSLQKLGFQVIFTDFKVVNVLAVCNMPFEIRLPEF
TKNNRPHASYEPELHPAVCYRIKSLRATLQIFSTGSITVTGPNVKAVATAVEQIYPFVFE
SRKEILL
SEQ ID NO: 156
TBPL1 DNA sequence
atggatgcagacagtgatgttgcattggacattctaattacaaatgtagtctgtgtttttag
aacaagatgtcatttaaacttaaggaagattgctttggaaggagcaaatgtaatttataaac
gtgatgctggaaaagtattaatgaagcttagaaaacctagaattacagctacaatttggtcc
tcaggaaaaattatttgcactggagcaacaagtgaagaagaagctaaatttggtgccagacg
cttagcccgtagtctgcagaaactaggttttcaggtaatatttacagattttaaggttgtta
acgttctggcagtgtgtaacatgccatttgaaatccgtttgccagaattcacaaagaacaat
agacctcatgccagttacgaacctgaacttcatcctgctgtgtgctatcggataaaatctct
aagagctacattacagattttttcaacaggaagtatcacagtaacagggcccaatgtaaagg
ctgttgctactgctgtggaacagatttacccatttgtgtttgaaagcaggaaagaaatttta
ttg
SEQ ID NO: 157
VPS72 protein sequence
MSLAGGRAPRKTAGNRLSGLLEAEEEDEFYQTTYGGFTEESGDDEYQGDQSDTEDEVDSD
FDIDEGDEPSSDGEAEEPRRKRRVVTKAYKEPLKSLRPRKVNTPAGSSQKAREEKALLPL
ELQDDGSDSRKSMRQSTAEHTRQTFLRVQERQGQSRRRKGPHCERPLTQEELLREAKITE
ELNLRSLETYERLEADKKKQVHKKRKCPGPIITYHSVTVPLVGEPGPKEENVDIEGLDPA
PSVSALTPHAGTGPVNPPARCSRTFITFSDDATFEEWFPQGRPPKVPVREVCPVTHRPAL
YRDPVTDIPYATARAFKIIREAYKKYITAHGLPPTASALGPGPPPPEPLPGSGPRALRQK
IVIKL
SEQ ID NO: 158
VPS72 DNA sequence
Atgagtttggctgggggccgggcaccccggaagaccgctgggaaccggctttctgggctttt
ggaggcagaggaggaagatgagttctaccagacgacttatgggggtttcacagaggaatccg
gagatgatgagtatcaaggggaccagtcagacacagaggacgaagtggactctgactttgac
attgatgaaggggatgaaccatccagtgatggagaagcagaagagccaagaaggaagcgccg
agtagtcaccaaggcctataaggaacctctcaagagcttaaggcctcgaaaggtcaacaccc
cggctggtagctctcagaaggcgcgagaagagaaggcactactgccattagaactacaagat
gacggctctgacagtcggaagtctatgcgtcagtctacagctgagcatacacgacaaacgtt
ccttcgggtacaggagaggcagggccagtcaagacggcgaaaggggccccactgtgagcggc
cactaacccaggaggaactgctccgggaggccaagatcacagaagagcttaatttacggtca
ctggagacatatgagcggctcgaggctgataaaaagaagcaggttcataagaagcggaagtg
ccccgggcccataatcacctatcattcagtgacagtgccacttgttggggagccaggcccca
aggaagagaacgttgacatagaaggacttgatcctgctccctcggtgtctgcattgactcct
catgctgggactggacccgtcaacccccctgctcgctgctcacgtaccttcatcacttttag
tgatgatgcaactttcgaggaatggttcccccaagggcggcccccaaaagtccctgttcgtg
aggtctgtccagtgacccatcgtccagccctataccgggaccctgttacagacataccctat
gccactgctcgagccttcaagatcattcgtgaggcttacaagaagtacattactgcccatgg
actgccgcccactgcctcagccctgggccccggcccgccacctcctgagcccctccctggct
ctgggccccgagccttgcgccagaaaattgtcattaaattg
SEQ ID NO: 159
ZNF133 protein sequence
MAFRDVAVDETQDEWRLLSPAQRTLYREVMLENYSNLVSLGISFSKPELITQLEQGKETW
REEKKCSPATCPDPEPELYLDPFCPPGFSSQKFPMQHVLCNHPPWIFTCLCAEGNIQPGD
PGPGDQEKQQQASEGRPWSDQAEGPEGEGAMPLFGRTKKRTLGAFSRPPQRQPVSSRNGL
RGVELEASPAQTGNPEETDKLLKRIEVLGFGTVNCGECGLSFSKMTNLLSHQRIHSGEKP
YVCGVCEKGFSLKKSLARHQKAHSGEKPIVCRECGRGENRKSTLIIHERTHSGEKPYMCS
ECGRGFSQKSNLIIHQRTHSGEKPYVCRECGKGFSQKSAVVRHQRTHLEEKTIVCSDCGL
GFSDRSNLISHQRTHSGEKPYACKECGRCFRQRTTLVNHQRTHSKEKPYVCGVCGHSFSQ
NSTLISHRRTHTGEKPYVCGVCGRGFSLKSHLNRHQNIHSGEKPIVCKDCGRGFSQQSNL
IRHQRTHSGEKPMVCGECGRGFSQKSNLVAHQRTHSGERPYVCRECGRGFSHQAGLIRHK
RKHSREKPYMCRQCGLGFGNKSALITHKRAHSEEKPCVCRECGQGFLQKSHLTLHQMTHT
GEKPYVCKTCGRGFSLKSHLSRHRKTTSVHHRLPVQPDPEPCAGQPSDSLYSL
SEQ ID NO: 160
ZNF133 DNA sequence
ATGGCATTCAGGGATGTGGCTGTGGATTTCACCCAGGATGAGTGGAGGCTGCTGAGCCCTGC
TCAAAGGACTCTGTACAGAGAGGTGATGCTGGAGAACTACAGCAACCTGGTCTCACTGGGAA
TTTCATTTTCTAAACCAGAACTCATCACCCAGCTGGAGCAAGGGAAAGAGACCTGGAGAGAG
GAAAAAAAATGTTCACCGGCAACCTGTCCAGATCCAGAGCCAGAGCTCTACCTCGATCCTTT
CTGCCCTCCGGGTTTCTCCAGTCAGAAATTCCCCATGCAGCATGTGCTGTGTAATCATCCCC
CCTGGATCTTCACATGCTTGTGTGCAGAAGGTAACATCCAGCCTGGGGATCCAGGCCCAGGG
GACCAGGAGAAGCAGCAACAAGCCTCTGAGGGGAGACCCTGGAGTGATCAAGCAGAAGGTCC
TGAGGGAGAAGGTGCCATGCCTTTGTTTGGAAGAACCAAGAAAAGGACTCTGGGAGCGTTCT
CCAGGCCACCCCAGAGGCAGCCAGTCAGCTCTCGGAACGGCCTCAGAGGGGTGGAGTTAGAA
GCCAGCCCAGCTCAGACAGGGAACCCTGAGGAAACAGACAAATTGTTGAAGAGGATAGAAGT
CTTAGGATTTGGAACAGTCAACTGTGGAGAGTGTGGACTGAGCTTCAGCAAGATGACAAACC
TGCTCAGTCACCAGCGGATACACTCAGGGGAGAAGCCCTACGTGTGTGGGGTATGTGAGAAG
GGCTTCAGCCTAAAGAAGAGCCTCGCCAGACACCAGAAGGCACACTCGGGGGAGAAGCCAAT
TGTGTGCAGGGAGTGTGGACGAGGCTTTAACCGGAAGTCAACGCTAATCATACACGAACGGA
CACACTCCGGTGAGAAACCTTACATGTGCAGTGAGTGTGGGCGAGGCTTCAGCCAGAAGTCA
AACCTCATCATACACCAGAGGACACACTCAGGGGAAAAGCCTTATGTGTGCCGGGAATGTGG
CAAAGGCTTCAGCCAGAAGTCAGCTGTCGTGAGACACCAGAGGACACACTTGGAGGAGAAGA
CCATCGTGTGCAGTGACTGTGGCCTGGGCTTCAGCGACAGGTCAAACCTCATCTCCCACCAG
AGGACGCACTCTGGGGAGAAGCCCTACGCCTGCAAGGAGTGTGGGCGATGCTTCAGGCAGAG
GACCACCCTTGTCAACCACCAGAGGACACACTCAAAGGAGAAGCCCTATGTGTGCGGGGTGT
GTGGGCACAGCTTCAGCCAGAATTCAACCCTCATCTCTCACAGGCGGACACACACTGGGGAG
AAGCCGTATGTTTGTGGGGTGTGTGGGCGAGGCTTTAGTCTCAAGTCACACCTCAACAGACA
CCAGAACATACACTCAGGAGAGAAGCCCATTGTGTGCAAGGACTGTGGCCGGGGCTTCAGCC
AGCAATCCAACCTCATCAGACACCAGAGGACGCACTCAGGCGAGAAGCCCATGGTGTGTGGG
GAGTGCGGGCGAGGCTTCAGCCAGAAGTCAAACCTTGTTGCACACCAGAGGACGCACTCAGG
GGAGAGGCCGTATGTGTGCCGAGAGTGCGGGCGAGGCTTTAGCCACCAGGCCGGTCTCATCA
GGCACAAGCGGAAGCACTCGAGGGAGAAGCCCTACATGTGCAGGCAGTGTGGACTGGGCTTT
GGCAATAAGTCAGCTCTAATCACACACAAGCGGGCTCACTCGGAAGAGAAGCCTTGTGTGTG
CAGAGAGTGTGGCCAAGGCTTTCTCCAAAAGTCACACCTCACCTTACATCAAATGACACATA
CGGGGGAGAAGCCATATGTGTGCAAGACGTGTGGGCGGGGCTTCAGCCTCAAGTCTCACCTC
AGCAGACACAGGAAGACCACGTCTGTCCACCACAGACTGCCAGTGCAGCCCGACCCTGAGCC
GTGTGCAGGGCAACCTTCGGATTCCTTATACTCTCTCTGA
SEQ ID NO: 161
ZNF 140 protein sequence
MSQGSVTFRDVAIDFSQEEWKWLQPAQRDLYRCVMLENYGHLVSLGLSISKPDVVSLLEQ
GKEPWLGKREVKRDLFSVSESSGEIKDFSPKNVIYDDSSQYLIMERILSQGPVYSSFKGG
WKCKDHTEMLQENQGCIRKVTVSHQEALAQHMNISTVERPYGCHECGKTFGRRFSLVLHQ
RTHTGEKPYACKECGKTFSQISNLVKHQMIHTGKKPHECKDCNKTESYLSFLIEHQRTHT
GEKPYECTECGKAFSRASNLTRHQRIHIGKKQYICRKCGKAFSSGSELIRHQITHTGEKP
YECIECGKAFRRESHLTRHQSIHTTKTPYECNECRKAFRCHSFLIKHQRIHAGEKLYECD
ECGKVFTWHASLIQHTKSHTGEKPYACAECDKAFSRSFSLILHQRTHTGEKPYVCKVCNK
SFSWSSNLAKHQRTHTLDNPYEYENSFNYHSFLTEHQ
SEQ ID NO: 162
ZNF140 DNA sequence
ATGTCTCAGGGGTCAGTGACATTCAGAGATGTGGCCATAGACTTCTCCCAGGAGGAGTGGAA
ATGGCTTCAGCCTGCTCAAAGAGATTTGTACAGATGTGTAATGTTGGAGAACTATGGCCATC
TGGTCTCACTGGGTCTTTCCATTTCTAAGCCAGATGTGGTTTCCTTATTGGAGCAAGGGAAA
GAACCCTGGCTGGGGAAAAGGGAAGTGAAAAGAGATCTGTTTTCAGTTTCAGAGTCAAGTGG
TGAGATCAAAGACTTTTCACCAAAAAATGTCATTTATGATGACTCATCCCAGTATTTGATCA
TGGAAAGAATTCTAAGTCAAGGCCCTGTGTATTCCAGTTTTAAAGGAGGCTGGAAATGCAAG
GATCATACTGAGATGCTGCAAGAAAATCAGGGATGTATTAGGAAAGTAACAGTCTCTCATCA
AGAAGCCCTGGCTCAACATATGAATATCAGTACTGTGGAGAGGCCCTATGGATGCCATGAAT
GTGGAAAAACTTTTGGTCGACGCTTTTCCCTGGTGTTACACCAGAGGACTCATACTGGAGAG
AAACCATATGCATGTAAGGAATGTGGCAAAACCTTTAGCCAGATTTCAAACCTTGTGAAACA
CCAAATGATACATACTGGAAAGAAACCCCATGAGTGTAAGGACTGTAATAAAACATTCAGTT
ACCTTTCATTTCTTATTGAACACCAGAGAACGCACACTGGGGAGAAACCTTATGAATGTACT
GAGTGTGGAAAGGCCTTTAGCCGTGCCTCCAACCTCACTCGACATCAAAGAATTCACATAGG
AAAGAAACAATATATATGTAGGAAATGTGGTAAAGCATTTAGCAGTGGCTCAGAACTCATTC
GCCACCAGATTACACATACTGGAGAGAAACCTTATGAATGCATTGAATGTGGGAAGGCATTT
CGCCGTTTCTCACACCTTACTCGACATCAGAGCATCCATACAACCAAAACCCCGTATGAATG
TAATGAATGTAGGAAAGCTTTCCGTTGTCACTCATTCCTTATTAAACATCAGAGAATTCATG
CTGGAGAAAAGCTCTATGAATGTGATGAATGTGGTAAAGTTTTCACTTGGCATGCATCCCTT
ATTCAACATACGAAGAGTCACACTGGAGAGAAACCCTATGCGTGTGCTGAATGTGATAAAGC
CTTCAGCCGGAGCTTTTCCCTCATTCTACATCAGAGAACTCATACTGGAGAGAAACCCTATG
TATGTAAGGTATGCAACAAATCCTTCAGCTGGAGCTCAAACCTTGCTAAACATCAGAGGACA
CACACTCTTGACAACCCCTATGAATATGAAAATTCATTTAATTACCACTCATTCCTTACTGA
ACACCAGTGA
SEQ ID NO: 163
ZNF 169 protein sequence
MSPGLLTTRKEALMAFRDVAVAFTQKEWKLLSSAQRTLYREVMLENYSHLVSLGIAFSKP
KLIEQLEQGDEPWREENEHLLDLCPGRRITRSGVRD
SEQ ID NO: 164
ZNF 169 DNA sequence
ATGTCACCAGGACTCCTGACAACCAGGAAGGAGGCATTGATGGCCTTCCGGGATGTGGCTGT
GGCCTTCACCCAGAAGGAGTGGAAGCTATTGAGTTCTGCTCAGAGGACCCTGTACAGGGAGG
TGATGCTGGAGAACTACAGCCATCTGGTCTCCCTGGGAATTGCATTTTCCAAACCAAAACTC
ATCGAACAGCTGGAGCAAGGCGACGAACCTTGGAGAGAGGAGAACGAACATCTTCTGGACCT
TTGTCCAGGCAGGCGGATCACAAGGTCGGGAGTTCGAGACTAG
SEQ ID NO: 165
ZNF254 protein sequence
MPGPPRSLEMGLLTFRDVAIEFSLEEWQHLDIAQQNLYRNVMLENYRNLAFLGIAVSKPD
LITCLEQGKEPWNMKRHEMVDEPPGLDESLL
SEQ ID NO: 166
ZNF254 DNA sequence
ATGCCAGGACCCCCTAGAAGCCTAGAAATGGGACTGTTGACATTTAGGGATGTGGCCATAGA
ATTCTCTCTGGAGGAGTGGCAACACCTGGACATTGCACAGCAGAATTTATATAGAAATGTGA
TGTTAGAGAACTACAGAAACCTGGCCTTCCTGGGTATTGCTGTCTCTAAGCCAGACCTGATC
ACCTGTCTGGAACAAGGGAAAGAGCCCTGGAATATGAAGCGACATGAGATGGTGGATGAACC
CCCAGGATTGGATTTTTCATTACTGTGA
SEQ ID NO: 167
ZNF566 protein sequence
MAQESVMFSDVSVDFSQEEWECLNDDQRDLYRDVMLENYSNLVSMGHSISKPNVISYLEQ
GKEPWLADRELTRGQWPVLESRCETKKLFLKKEIYEIESTQWEIMEKLTRRDFQCSSERD
DWECNRQFKKELGSQGGHFNQLVFTHEDLPTLSHHPSFTLQQIINSKKKFCASKEYRKTF
RHGSQFATHEIIHTIEKPYECKECGKSFRHPSRLTHHQKIHTGKKPFECKECGKTFICGS
DLTRHHRIHTGEKPYECKECGKAFSSGSNFTRHQRIHTGEKPYECKECGKAFSSGSNFTQ
HQRIHTGEKPYECKECGNAFSQSSQLIKHQRIHTGEKPYECKECEKAFRSGSDLTRHQRI
HTGEKPYECKICGKAYSQSSQLISHHRIHTSEKPYEYRECGKNFNYDPQLIQHQNLYWL
SEQ ID NO: 168
ZNF566 DNA sequence
atggctcaggagtcagtgatgttcagtgatgtgtccgtagacttctctcaggaggagtggga
atgcctgaatgatgatcagagagatttatacagagatgtgatgttggagaattacagcaacc
tggtttcaatggggcattctatttctaaaccaaatgtgatctcctacttggagcaagggaag
gagccctggttggctgacagagagctaacaagaggccagtggccagtcctggaatcaagatg
tgagaccaagaaattatttctgaagaaagaaatttatgaaatagaatcaacccagtgggaaa
taatggaaaaactcacaagacgtgattttcagtgctccagtttcagagatgattgggaatgt
aatcggcagtttaagaaagaactcggctctcaggggggacatttcaatcaattggtattcac
tcatgaagatctgcccactttgagtcaccatccatccttcacattacagcaaatcattaaca
gtaaaaagaaattctgtgcatctaaagaatataggaaaacctttagacatggctcacagttt
gctacacatgagataattcataccattgagaagccttatgaatgtaaggaatgtggaaagtc
ctttagacatccctcaagactcactcatcatcagaaaattcatactggcaagaaaccctttg
aatgtaaggaatgtggaaaaacctttatttgtggctcagaccttactcgacatcacagaatt
cacactggtgagaaaccctatgaatgtaaggaatgtgggaaagcctttagtagtggttcaaa
cttcactcgacatcagagaattcacacaggtgagaagccttatgaatgcaaagaatgcggga
aggcctttagtagtggctcaaactttactcaacatcagagaattcatactggggaaaaaccc
tatgaatgtaaggaatgtggcaatgcctttagtcagagctcacaacttattaaacatcaaag
aatccatacaggtgagaaaccttacgaatgtaaggaatgtgaaaaggcttttcgttctggct
cagaccttactagacatcagagaattcatactggggagaaaccctatgaatgtaagatatgt
gggaaggcttattctcagagttcacagcttattagtcatcatagaattcatactagtgagaa
accctatgaatatagggaatgtggaaagaactttaattatgacccacaacttattcagcatc
aaaatttgtactggttg
SEQ ID NO: 169
ZNF585A protein sequence
MPANWTSPQKSSALAPEDHGSSYEGSVSFRDVAIDFSREEWRHLDPSQRNLYRDVMLETY
SHLLSIGYQVPEAEVVMLEQGKEPWALQGERPRQSCPAPCLVNSHHLQESFRG
SEQ ID NO: 170
ZNF585A DNA sequence
ATGCCAGCTAATTGGACCTCACCTCAGAAATCCTCAGCCCTGGCTCCAGAGGATCATGGCAG
CTCCTATGAGGGATCAGTGTCCTTCAGGGATGTGGCTATCGATTTCAGCAGAGAGGAATGGC
GGCACCTGGACCCTTCTCAGAGAAACCTGTACCGGGATGTGATGCTGGAGACCTACAGCCAC
CTGCTCTCAATAGGATATCAAGTTCCTGAAGCAGAGGTGGTCATGTTGGAGCAAGGAAAGGA
ACCATGGGCACTGCAGGGTGAGAGGCCACGTCAGAGCTGCCCAGCACCGTGTCTTGTGAACT
CCCATCACCTTCAAGAAAGCTTCCGAGGGTGA
SEQ ID NO: 171
ZNF689 protein sequence
MAPPSAPLPAQGPGKARPSRKRGRRPRALKFVDVAVYFSPEEWGCLRPAQRALYRDVMRE
TYGHLGALGCAGPKPALISWLERNTDDWEPAALDPQEYPRGLTVQRKSRTRKKNGEKEVF
PPKEAPRKGKRGRRPSKPRLIPRQTSGGPICPDCGCTFPDHQALESHKCAQNLKKPYPCP
DCGRRFSYPSLLVSHRRAHSGECPYVCDQCGKRFSQRKNLSQHQVIHTGEKPYHCPDCGR
CFRRSRSLANHRTTHTGEKPHQCPSCGRRFAYPSLLAIHQRTHTGEKPYTCLECNRRFRQ
RTALVIHQRIHTGEKPYPCPDCERRFSSSSRLVSHRRVHSGERPYACEHCEARFSQRSTL
LQHQLLHTGEKPYPCPDCGRAFRRSGSLAIHRSTHTEEKLHACDDCGRRFAYPSLLASHR
RVHSGERPYACDLCSKRFAQWSHLAQHQLLHTGEKPFPCLECGRCFRQRWSLAVHKCSPK
APNCSPRSAIGGSSQRGNAH
SEQ ID NO: 172
ZNF689 DNA sequence
ATGGCGCCACCTTCGGCTCCGCTCCCTGCGCAGGGACCAGGAAAGGCCAGACCCAGTCGGAA
AAGGGGCAGGAGGCCGAGGGCTCTGAAGTTCGTGGACGTGGCCGTGTACTTCTCCCCGGAGG
AGTGGGGCTGCCTGCGGCCCGCGCAGAGGGCCCTGTACCGGGACGTGATGCGGGAGACCTAC
GGTCACCTGGGCGCGCTCGGGTGCGCAGGTCCCAAACCAGCCCTCATCTCCTGGTTGGAACG
AAACACCGATGACTGGGAACCGGCTGCTCTAGATCCGCAGGAGTACCCGAGAGGGCTAACAG
TCCAGAGAAAAAGCAGAACCAGAAAGAAGAATGGGGAGAAGGAAGTATTCCCGCCTAAGGAG
GCACCCCGAAAGGGGAAGCGAGGCCGGAGGCCCAGCAAACCCCGACTGATTCCTAGGCAGAC
GTCCGGGGGCCCCATCTGCCCTGACTGCGGCTGTACCTTCCCTGATCATCAGGCCCTGGAGA
GCCACAAGTGCGCCCAGAATCTAAAAAAGCCTTACCCTTGCCCAGACTGTGGGCGCCGCTTT
TCCTATCCATCCCTGCTGGTCAGTCACCGGCGGGCACACTCCGGCGAGTGCCCCTATGTTTG
TGACCAGTGTGGCAAACGTTTCTCCCAGCGCAAGAACCTCTCCCAGCACCAGGTCATCCATA
CAGGGGAGAAGCCCTATCACTGCCCTGACTGTGGTCGCTGCTTCCGGAGGAGCCGGTCCTTG
GCCAATCACCGGACCACACACACAGGTGAAAAACCCCACCAGTGCCCTAGCTGTGGACGTCG
CTTCGCCTACCCCTCCCTGCTAGCCATCCACCAGCGTACACACACGGGAGAGAAGCCCTACA
CTTGCCTCGAGTGCAACCGCCGCTTCCGCCAGCGCACGGCCCTCGTCATCCACCAGCGCATC
CACACGGGCGAGAAGCCCTACCCGTGCCCGGACTGCGAGCGGCGCTTCTCCTCCTCCTCTCG
CCTGGTCAGTCACCGGCGTGTGCACTCTGGGGAGCGTCCCTATGCCTGCGAGCACTGTGAGG
CCCGCTTCTCCCAGCGCAGCACGCTGCTCCAGCACCAGCTCTTGCACACCGGAGAGAAGCCC
TACCCCTGCCCAGACTGTGGGCGTGCCTTCCGGCGGAGCGGCTCCCTGGCCATCCATCGCAG
CACGCACACAGAGGAGAAGCTGCACGCCTGCGACGACTGTGGTCGCCGCTTTGCCTACCCCT
CACTGCTGGCCAGCCACCGGCGCGTGCACTCGGGCGAGCGGCCCTATGCCTGCGACCTTTGC
TCCAAGCGTTTTGCTCAGTGGAGCCACCTGGCCCAGCACCAGCTGCTGCACACGGGGGAGAA
GCCTTTCCCCTGCCTCGAGTGTGGCCGGTGCTTCCGCCAGAGGTGGTCTCTGGCTGTCCACA
AGTGTAGCCCCAAGGCCCCAAACTGTAGCCCTAGATCTGCTATCGGGGGCTCCAGTCAGAGG
GGCAACGCCCATTAG
SEQ ID NO: 173
ZNF765 protein sequence
MALPQGLLTFRDVAIEFSQEEWKCLDPAQRTLYRDVMLENYRNLVSLELSGECPLAAPAS
LDPAFLC
SEQ ID NO: 174
ZNF765 DNA sequence
atggctcttcctcagggtctattgacattcagggatgtggccatagaattctctcaggagga
gtggaaatgcctggaccctgctcagaggactctatacagggacgtgatgctggagaattata
ggaacctggtctccctggagttgtcaggggagtgtccattggcagcacctgcctccttggac
ccagctttcttgtgc
SEQ ID NO: 175
ZNF81 protein sequence
MPANEDAPQPGEHGSACEVSVSFEDVTVDFSREEWQQLDSTQRRLYQDVMLENYSHLLSV
GFEVPKPEVIFKLEQGEGPWTLEGEAPHQSCSDGKFGIKPSQRRISGKSTFHSEMEGEDT
LCSGLM
SEQ ID NO: 176
ZNF81 DNA sequence
atgccagctaacgaggacgctccccagccaggggaacatggcagtgcctgtgaggtatcagt
gtcatttgaggatgtgactgtggacttcagtagagaggagtggcagcaactggactctactc
aaagacgcctgtaccaggatgtaatgttggagaactacagccacctgctctcagtggggttc
gaagttcctaaaccagaggtcatcttcaagttggagcaaggagaggggccatggacattgga
aggggaagccccacatcagagctgttcagatgggaaatttggaattaagccttcccagagga
gaatttctgggaaatctacatttcatagtgaaatggagggtgaagacacactgtgttcaggc
ctcatggg

1. A Cas effector comprising:
a first polypeptide comprising a Cas protein and at least one peptide epitope; and
a second polypeptide comprising an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, and an antibody to the peptide epitope.

2. The Cas effector of claim 1, wherein the effector is selected from MCRS1, OTUD7B, RelB, LDB1, NFKBIB, CITED2, PHF15, SS18L1, MLLT6, ASH2L, and GSK3A, or a combination thereof.

3. The Cas effector of claim 1 or 2, wherein the effector is capable of increasing or decreasing expression of a gene.

4. The Cas effector of claim 3, wherein the effector reduces expression of a target gene and is selected from MCRS1, OTUD7B, ZNF133, ZNF140, ZNF169, ZNF254, ZNF566, ZNF585A, ZNF689, ZNF765, and ZNF81, or a combination thereof.

5. The Cas effector of claim 3, wherein the effector increases expression of a target gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof.

6. The Cas effector of any one of claims 1-5, wherein the first polypeptide comprises about 2 to about 50 peptide epitopes.

7. The Cas effector of any one of claims 1-6, wherein the first polypeptide comprises more than one copy of the peptide epitope and further comprises at least one linker in between adjacent copies of the peptide epitope.

8. The Cas effector of any one of claims 1-7, wherein the peptide epitope is GCN4 and comprises the amino acid sequence of SEQ ID NO: 85.

9. The Cas effector of any one of claims 1-8, wherein the first polypeptide comprises at least one peptide epitope at the N-terminus and/or at the C-terminus of the Cas protein.

10. The Cas effector of any one of claims 1-9, wherein the first polypeptide comprises an amino acid sequence having at least 80%, 85%, 90%, 95%, or 98% or greater identity to SEQ ID NO: 87 or 89, or any fragment thereof,
or wherein the first polypeptide comprises an amino acid sequence having one, two, three, four, five or more changes selected from amino acid substitutions, insertions, or deletions, relative to SEQ ID NO: 87 or 89, or any fragment thereof,
or wherein the first polypeptide comprises the amino acid sequence of SEQ ID NO: 87 or 89.

11. The Cas effector of any one of claims 1-10, wherein the antibody comprises the amino acid sequence of SEQ ID NO: 81.

12. The Cas effector of any one of claims 1-11, wherein the second polypeptide comprises an amino acid sequence having at least 80%, 85%, 90%, 95%, or 98% or greater identity to a sequence selected from SEQ ID NOs: 69, 71, 73, 75, 77, and 79, or any fragment thereof,
or wherein the second polypeptide comprises an amino acid sequence having one, two, three, four, five or more changes selected from amino acid substitutions, insertions, or deletions, relative to a sequence selected from SEQ ID NOs: 69, 71, 73, 75, 77, and 79, or any fragment thereof,
or wherein the second polypeptide comprises an amino acid sequence selected from SEQ ID NOs: 69, 71, 73, 75, 77, and 79.

13. A Cas fusion protein comprising two heterologous polypeptide domains, wherein the first polypeptide domain comprises a Cas protein, and wherein the second polypeptide domain comprises an effector selected from MCRS1, OTUD7B, RelB, LDB1, NFKBIB, and CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof.

14. The Cas fusion protein of claim 13, wherein the effector is selected from MCRS1, OTUD7B, RelB, LDB1, NFKBIB, CITED2, PHF15, SS18L1, MLLT6, ASH2L, and GSK3A, or a combination thereof.

15. The Cas fusion protein of claim 13 or 14, wherein the effector is capable of increasing or decreasing expression of a gene.

16. The Cas fusion protein of claim 15, wherein the effector reduces expression of a target gene and is selected from MCRS1, OTUD7B, ZNF133, ZNF140, ZNF169, ZNF254, ZNF566, ZNF585A, ZNF689, ZNF765, and ZNF81, or a combination thereof.

17. The Cas fusion protein of claim 15, wherein the effector increases expression of a target gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof.

18. The Cas fusion protein of any one of claims 13-17, wherein the second polypeptide domain has transcription repression activity, transcription activation activity, de-ubiquitinase activity, p300 recruitment activity, enhancer looping mediation activity, or a combination thereof.

19. The Cas effector of any one of claims 1-12 or the Cas fusion protein of any one of claims 13-18, wherein the MCRS1 comprises an amino acid sequence having at least 80%, 85%, 90%, 95%, or 98% or greater identity to SEQ ID NO: 57 or any fragment thereof,
and/or wherein the MCRS1 comprises an amino acid sequence having one, two, three, four, five or more changes selected from amino acid substitutions, insertions, or deletions, relative to SEQ ID NO: 57, or any fragment thereof,
and/or wherein the MCRS1 comprises the amino acid sequence of SEQ ID NO: 57,
and/or wherein the MCRS1 is encoded by a polynucleotide comprising a sequence having at least 80%, 85%, 90%, 95%, or 98% or greater identity to SEQ ID NO: 58, or any fragment thereof,
and/or wherein the MCRS1 is encoded by a polynucleotide comprising a sequence having one, two, three, four, five or more changes selected from nucleotide substitutions, insertions, or deletions, relative to SEQ ID NO: 58, or any fragment thereof,
and/or wherein the MCRS1 is encoded by a polynucleotide comprising the sequence of SEQ ID NO: 58.

20. The Cas effector of any one of claims 1-12 or the Cas fusion protein of any one of claims 13-18, wherein the OTUD7B comprises an amino acid sequence having at least 80%, 85%, 90%, 95%, or 98% or greater identity to a sequence selected from SEQ ID NO: 59, amino acids 167-440 of SEQ ID NO: 59, or amino acids 792-831 of SEQ ID NO: 59, or any fragment thereof,
and/or wherein the OTUD7B comprises an amino acid sequence having one, two, three, four, five or more changes selected from amino acid substitutions, insertions, or deletions, relative to a sequence selected from SEQ ID NO: 59, amino acids 167-440 of SEQ ID NO: 59, or amino acids 792-831 of SEQ ID NO: 59, or any fragment thereof,
and/or wherein the OTUD7B comprises the amino acid sequence selected from SEQ ID NO: 59, amino acids 167-440 of SEQ ID NO: 59, or amino acids 792-831 of SEQ ID NO: 59,
and/or wherein the OTUD7B is encoded by a polynucleotide comprising a sequence having at least 80%, 85%, 90%, 95%, or 98% or greater identity to SEQ ID NO: 60, or any fragment thereof,
and/or wherein the OTUD7B is encoded by a polynucleotide comprising a sequence having one, two, three, four, five or more changes selected from nucleotide substitutions, insertions, or deletions, relative to SEQ ID NO: 60, or any fragment thereof,
and/or wherein the OTUD7B is encoded by a polynucleotide comprising the sequence of SEQ ID NO: 60.

21. The Cas effector of any one of claims 1-12 or the Cas fusion protein of any one of claims 13-18, wherein the RelB comprises an amino acid sequence having at least 80%, 85%, 90%, 95%, or 98% or greater identity to SEQ ID NO: 65, or any fragment thereof,
and/or wherein the RelB comprises an amino acid sequence having one, two, three, four, five or more changes selected from amino acid substitutions, insertions, or deletions, relative to SEQ ID NO: 65, or any fragment thereof,
and/or wherein the RelB comprises the amino acid sequence of SEQ ID NO: 65,
and/or wherein the RelB is encoded by a polynucleotide comprising a sequence having at least 80%, 85%, 90%, 95%, or 98% or greater identity to SEQ ID NO: 66 or any fragment thereof,
and/or wherein the RelB is encoded by a polynucleotide comprising a sequence having one, two, three, four, five or more changes selected from nucleotide substitutions, insertions, or deletions, relative to SEQ ID NO: 66, or any fragment thereof,
and/or wherein the RelB is encoded by a polynucleotide comprising the sequence of SEQ ID NO: 66.

22. The Cas effector of any one of claims 1-12 or the Cas fusion protein of any one of claims 13-18, wherein the LDB1 comprises an amino acid sequence having at least 80%, 85%, 90%, 95%, or 98% or greater identity to SEQ ID NO: 61, or any fragment thereof,
and/or wherein the LDB1 comprises an amino acid sequence having one, two, three, four, five or more changes selected from amino acid substitutions, insertions, or deletions, relative to SEQ ID NO: 61, or any fragment thereof,
and/or wherein the LDB1 comprises the amino acid sequence of SEQ ID NO: 61,
and/or wherein the LDB1 is encoded by a polynucleotide comprising a sequence having at least 80%, 85%, 90%, 95%, or 98% or greater identity to SEQ ID NO: 62, or any fragment thereof,
and/or wherein the LDB1 is encoded by a polynucleotide comprising a sequence having one, two, three, four, five or more changes selected from nucleotide substitutions, insertions, or deletions, relative to SEQ ID NO: 62, or any fragment thereof,
and/or wherein the LDB1 is encoded by a polynucleotide comprising the sequence of SEQ ID NO: 62.

23. The Cas effector of any one of claims 1-12 or the Cas fusion protein of any one of claims 13-18, wherein the NFKBIB comprises an amino acid sequence having at least 80%, 85%, 90%, 95%, or 98% or greater identity to SEQ ID NO: 63, or any fragment thereof,
and/or wherein the NFKBIB comprises an amino acid sequence having one, two, three, four, five or more changes selected from amino acid substitutions, insertions, or deletions, relative to SEQ ID NO: 63, or any fragment thereof,
and/or wherein the NFKBIB comprises the amino acid sequence of SEQ ID NO: 63,
and/or wherein the NFKBIB is encoded by a polynucleotide comprising a sequence having at least 80%, 85%, 90%, 95%, or 98% or greater identity to SEQ ID NO: 64, or any fragment thereof,
and/or wherein the NFKBIB is encoded by a polynucleotide comprising a sequence having one, two, three, four, five or more changes selected from nucleotide substitutions, insertions, or deletions, relative to SEQ ID NO: 64, or any fragment thereof,
and/or wherein the NFKBIB is encoded by a polynucleotide comprising the sequence of SEQ ID NO: 64.

24. The Cas effector of any one of claims 1-12 or the Cas fusion protein of any one of claims 13-18, wherein the CITED2 comprises an amino acid sequence having at least 80%, 85%, 90%, 95%, or 98% or greater identity to SEQ ID NO: 67, or any fragment thereof,
and/or wherein the CITED2 comprises an amino acid sequence having one, two, three, four, five or more changes selected from amino acid substitutions, insertions, or deletions, relative to SEQ ID NO: 67, or any fragment thereof,
and/or wherein the CITED2 comprises the amino acid sequence of SEQ ID NO: 67,
and/or wherein the CITED2 is encoded by a polynucleotide comprising a sequence having at least 80%, 85%, 90%, 95%, or 98% or greater identity to SEQ ID NO: 68, or any fragment thereof,
and/or wherein the CITED2 is encoded by a polynucleotide comprising a sequence having one, two, three, four, five or more changes selected from nucleotide substitutions, insertions, or deletions, relative to SEQ ID NO: 68, or any fragment thereof,
and/or wherein the CITED2 is encoded by a polynucleotide comprising the sequence of SEQ ID NO: 68.

25. The Cas effector of any one of claims 1-12 or the Cas fusion protein of any one of claims 13-18, wherein the PHF15 comprises an amino acid sequence having at least 80%, 85%, 90%, 95%, or 98% or greater identity to SEQ ID NO: 133, or any fragment thereof,
and/or wherein the PHF15 comprises an amino acid sequence having one, two, three, four, five or more changes selected from amino acid substitutions, insertions, or deletions, relative to SEQ ID NO: 133, or any fragment thereof,
and/or wherein the PHF15 comprises the amino acid sequence of SEQ ID NO: 133,
and/or wherein the PHF15 is encoded by a polynucleotide comprising a sequence having at least 80%, 85%, 90%, 95%, or 98% or greater identity to SEQ ID NO: 134, or any fragment thereof,
and/or wherein the PHF15 is encoded by a polynucleotide comprising a sequence having one, two, three, four, five or more changes selected from nucleotide substitutions, insertions, or deletions, relative to SEQ ID NO: 134, or any fragment thereof,
and/or wherein the PHF15 is encoded by a polynucleotide comprising the sequence of SEQ ID NO: 134.

26. The Cas effector of any one of claims 1-12 or the Cas fusion protein of any one of claims 13-18, wherein the SS18L1 comprises an amino acid sequence having at least 80%, 85%, 90%, 95%, or 98% or greater identity to SEQ ID NO: 149, or any fragment thereof,
and/or wherein the SS18L1 comprises an amino acid sequence having one, two, three, four, five or more changes selected from amino acid substitutions, insertions, or deletions, relative to SEQ ID NO: 149, or any fragment thereof,
and/or wherein the SS18L1 comprises the amino acid sequence of SEQ ID NO: 149,
and/or wherein the SS18L1 is encoded by a polynucleotide comprising a sequence having at least 80%, 85%, 90%, 95%, or 98% or greater identity to SEQ ID NO: 150, or any fragment thereof,
and/or wherein the SS18L1 is encoded by a polynucleotide comprising a sequence having one, two, three, four, five or more changes selected from nucleotide substitutions, insertions, or deletions, relative to SEQ ID NO: 150, or any fragment thereof,
and/or wherein the SS18L1 is encoded by a polynucleotide comprising the sequence of SEQ ID NO: 150.

27. The Cas effector of any one of claims 1-12 or the Cas fusion protein of any one of claims 13-18, wherein the MLLT6 comprises an amino acid sequence having at least 80%, 85%, 90%, 95%, or 98% or greater identity to SEQ ID NO: 127, or any fragment thereof,
and/or wherein the MLLT6 comprises an amino acid sequence having one, two, three, four, five or more changes selected from amino acid substitutions, insertions, or deletions, relative to SEQ ID NO: 127, or any fragment thereof,
and/or wherein the MLLT6 comprises the amino acid sequence of SEQ ID NO: 127,
and/or wherein the MLLT6 is encoded by a polynucleotide comprising a sequence having at least 80%, 85%, 90%, 95%, or 98% or greater identity to SEQ ID NO: 128, or any fragment thereof,
and/or wherein the MLLT6 is encoded by a polynucleotide comprising a sequence having one, two, three, four, five or more changes selected from nucleotide substitutions, insertions, or deletions, relative to SEQ ID NO: 128, or any fragment thereof,
and/or wherein the MLLT6 is encoded by a polynucleotide comprising the sequence of SEQ ID NO: 128.

28. The Cas effector of any one of claims 1-12 or the Cas fusion protein of any one of claims 13-18, wherein the ASH2L comprises an amino acid sequence having at least 80%, 85%, 90%, 95%, or 98% or greater identity to SEQ ID NO: 103, or any fragment thereof,
and/or wherein the ASH2L comprises an amino acid sequence having one, two, three, four, five or more changes selected from amino acid substitutions, insertions, or deletions, relative to SEQ ID NO: 103, or any fragment thereof,
and/or wherein the ASH2L comprises the amino acid sequence of SEQ ID NO: 103,
and/or wherein the ASH2L is encoded by a polynucleotide comprising a sequence having at least 80%, 85%, 90%, 95%, or 98% or greater identity to SEQ ID NO: 104, or any fragment thereof,
and/or wherein the ASH2L is encoded by a polynucleotide comprising a sequence having one, two, three, four, five or more changes selected from nucleotide substitutions, insertions, or deletions, relative to SEQ ID NO: 104, or any fragment thereof,
and/or wherein the ASH2L is encoded by a polynucleotide comprising the sequence of SEQ ID NO: 104.

29. The Cas effector of any one of claims 1-12 or the Cas fusion protein of any one of claims 13-18, wherein the GSK3A comprises an amino acid sequence having at least 80%, 85%, 90%, 95%, or 98% or greater identity to SEQ ID NO: 117, or any fragment thereof,
and/or wherein the GSK3A comprises an amino acid sequence having one, two, three, four, five or more changes selected from amino acid substitutions, insertions, or deletions, relative to SEQ ID NO: 117, or any fragment thereof,
and/or wherein the GSK3A comprises the amino acid sequence of SEQ ID NO: 117,
and/or wherein the GSK3A is encoded by a polynucleotide comprising a sequence having at least 80%, 85%, 90%, 95%, or 98% or greater identity to SEQ ID NO: 118, or any fragment thereof,
and/or wherein the GSK3A is encoded by a polynucleotide comprising a sequence having one, two, three, four, five or more changes selected from nucleotide substitutions, insertions, or deletions, relative to SEQ ID NO: 118, or any fragment thereof,
and/or wherein the GSK3A is encoded by a polynucleotide comprising the sequence of SEQ ID NO: 118.

30. The Cas effector of any one of claims 1-12 or the Cas fusion protein of any one of claims 13-18, wherein the effector is selected from BCL7B, C20orf20, DMAP1, DYRK1B, EAF1, FOXR2, JAZF1, KAT7, KEAP1, MEAF6, MORF4L2, NFYC, PKIB, POLE4, PRKRIR, PYGO2, RANBP1, RPRD1B, SPIN1, TADA3, TAF6, TBPL1, VPS72, ZNF133, ZNF140, ZNF169, ZNF254, ZNF566, ZNF585A, ZNF689, ZNF765, and ZNF81, and wherein the effector comprises an amino acid sequence having at least 80%, 85%, 90%, 95%, or 98% or greater identity to a sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175, or any fragment thereof,
and/or wherein the effector comprises an amino acid sequence having one, two, three, four, five or more changes selected from amino acid substitutions, insertions, or deletions, relative to a sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175, or any fragment thereof,
and/or wherein the effector comprises an amino acid sequence selected from SEQ ID NOs: 105, 107, 109, 111, 113, 115, 119, 121, 123, 125, 129, 131, 135, 137, 139, 141, 143, 145, 147, 151, 153, 155, 157, 159, 161, 163, 165, 167, 169, 171, 173, or 175,
and/or wherein the effector is encoded by a polynucleotide comprising a sequence having at least 80%, 85%, 90%, 95%, or 98% or greater identity to a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176, or any fragment thereof,
and/or wherein the effector is encoded by a polynucleotide comprising a sequence having one, two, three, four, five or more changes selected from nucleotide substitutions, insertions, or deletions, relative to a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176, or any fragment thereof,
and/or wherein the effector is encoded by a polynucleotide comprising a sequence selected from SEQ ID NOs: 106, 108, 110, 112, 114, 116, 120, 122, 124, 126, 130, 132, 136, 138, 140, 142, 144, 146, 148, 152, 154, 156, 158, 160, 162, 164, 166, 168, 170, 172, 174, or 176.

31. The Cas effector of any one of claims 1-12 and 19-31 or the Cas fusion protein of claim any one of claims 13-31, wherein the Cas protein comprises at least one amino acid mutation that knocks out nuclease activity of the Cas protein.

32. The Cas effector or the Cas fusion protein of claim 31, wherein the at least one amino acid mutation is at least one of D10A and H840A.

33. The Cas effector of any one of claims 1-12 and 19-32 or the Cas fusion protein of any one of claims 13-32, wherein the Cas protein comprises an amino acid sequence having at least 80%, 85%, 90%, 95%, or 98% or greater identity to one of SEQ ID NOs: 26-29, or any fragment thereof,
or wherein the Cas protein comprises an amino acid sequence having one, two, three, four, five or more changes selected from amino acid substitutions, insertions, or deletions, relative to one of SEQ ID NOs: 26-29, or any fragment thereof,
or wherein the Cas protein comprises the amino acid sequence of one of SEQ ID NOs: 26-29.

34. The Cas effector of any one of claims 1-12 and 19-33 or the Cas fusion protein of any one of claims 13-33, wherein the Cas protein is encoded by a polynucleotide comprising a sequence having at least 80%, 85%, 90%, 95%, or 98% or greater identity to one of SEQ ID NOs: 30-31, or any fragment thereof,
or wherein the Cas protein is encoded by a polynucleotide comprising a sequence having one, two, three, four, five or more changes selected from nucleotide substitutions, insertions, or deletions, relative to one of SEQ ID NOs: 30-31, or any fragment thereof,
or wherein the Cas protein is encoded by a polynucleotide comprising the sequence of one of SEQ ID NOs: 30-31.

35. A DNA targeting composition comprising:
the Cas effector of any one of claims 1-12 and 19-34 or the Cas fusion protein of any one of claims 13-34; and
at least one guide RNA (gRNA) that targets the Cas protein to a target region of a target gene.

36. The DNA targeting composition of claim 35, wherein the gRNA targets the Cas protein to target region selected from a non-open chromatin region, an open chromatin region, a transcribed region of the target gene, a region upstream of a transcription start site of the target gene, a regulatory element of the target gene, an intron of the target gene, or an exon of the target gene.

37. The DNA targeting composition of claim 35 or 36, wherein the gRNA targets the Cas protein to a promoter of the target gene.

38. The DNA targeting composition of any one of claims 35-37, wherein the target region is located between about 1 to about 1000 base pairs upstream of a transcription start site of the target gene.

39. The DNA targeting composition of any one of claims 35-38, wherein the at least one gRNA comprises a sequence selected from SEQ ID NOs: 96-98 and 101-102, or wherein the at least one gRNA is encoded by a polynucleotide comprising a sequence selected from SEQ ID NOs: 93-95 and 99-100, or wherein the at least one gRNA targets and binds a polynucleotide comprising a sequence selected from SEQ ID NOs: 93-95 and 99-100 or a complement thereof, or a combination thereof.

40. The DNA targeting composition of any one of claims 35-39, wherein the DNA targeting composition comprises two or more gRNAs, each gRNA binding to a different target region.

41. An isolated polynucleotide sequence encoding the Cas effector of any one of claims 1-12 and 19-34 or the Cas fusion protein of any one of claims 13-34, or the DNA targeting composition of any one of claims 35-40.

42. A vector comprising: the isolated polynucleotide sequence of claim 41.

43. The vector of claim 42, wherein the vector is an adeno-associated virus (AAV) vector.

44. A cell comprising: the Cas effector of any one of claims 1-12 and 19-34 or the Cas fusion protein of any one of claims 13-34, or the DNA targeting composition of any one of claims 35-40, or the isolated polynucleotide sequence of claim 41, or the vector of claim 42 or 43, or a combination thereof.

45. A pharmaceutical composition comprising: the Cas effector of any one of claims 1-12 and 19-34 or the Cas fusion protein of any one of claims 13-34, or the DNA targeting composition of any one of claims 35-40, or the isolated polynucleotide sequence of claim 41, or the vector of claim 42 or 43, or a combination thereof.

46. A method of modulating expression of a gene in a cell or in a subject, the method comprising administering to the cell or the subject the DNA targeting composition of any one of claims 35-40, or the isolated polynucleotide sequence of claim 41, or the vector of claim 42 or 43, or the pharmaceutical composition of claim 45, or a combination thereof.

47. A method of modulating expression of a gene in a cell or in a subject, the method comprising administering to the cell or the subject an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, or a polynucleotide encoding the effector.

48. The method of claim 47, wherein the effector is targeted to the gene.

49. The method of claim 47 or 48, wherein the effector is selected from MCRS1, OTUD7B, RelB, LDB1, NFKBIB, CITED2, PHF15, SS18L1, MLLT6, ASH2L, and GSK3A, or a combination thereof.

50. The method of any one of claims 47-49, wherein the effector is capable of increasing or decreasing expression of the gene.

51. The method of claim 50, wherein the effector reduces expression of the gene and is selected from MCRS1, OTUD7B, ZNF133, ZNF140, ZNF169, ZNF254, ZNF566, ZNF585A, ZNF689, ZNF765, and ZNF81, or a combination thereof.

52. The method of claim 50, wherein the effector increases expression of the gene and is selected from RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, and VPS72, or a combination thereof.

53. The method of any one of claims 46-50 and 52, wherein the expression of the gene is increased relative to a control.

54. The method of any one of claims 46-51, wherein the expression of the gene is decreased relative to a control.

55. The method of any one of claims 46-54, wherein the gene comprises the dystrophin gene, the CD25 gene, the B2M gene, or the TRAC gene.

56. The method of any one of claims 46-55, wherein the cell is a muscle cell or a T cell.

57. A method of treating a disease in a subject, the method comprising administering to the subject the DNA targeting composition of any one of claims 35-40, or the isolated polynucleotide sequence of claim 41, or the vector of claim 42 or 43, or the cell of claim 44, or the pharmaceutical composition of claim 45, or a combination thereof.

58. A method of treating a disease in a subject, the method comprising administering to the subject an effector selected from MCRS1, OTUD7B, RelB, LDB1, NFKBIB, CITED2, ASH2L, BCL7B, C20orf20, DMAP1, DYRK1B, EAF1, FOXR2, GSK3A, JAZF1, KAT7, KEAP1, MEAF6, MLLT6, MORF4L2, NFYC, PHF15, PKIB, POLE4, PRKRIR, PYGO2, RANBP1, RPRD1B, SPIN1, SS18L1, TADA3, TAF6, TBPL1, VPS72, ZNF133, ZNF140, ZNF169, ZNF254, ZNF566, ZNF585A, ZNF689, ZNF765, and ZNF81, or a combination thereof, or a polynucleotide encoding the effector.

59. The method of claim 58, wherein the effector is targeted to a gene.

60. The method of any one of claims 46-59, wherein the method treats a disease selected from Duchenne muscular dystrophy (DMD), Becker muscular dystrophy (BMD), and cancer.


SEQUENCE LISTING (176 sequences)

SEQ ID NO: 1-17 - skipped (17 entries)

SEQ ID NO: 18 - DNA, 86 bp, synthetic construct
  source 1..86: mol_type = other DNA
gtttaagagctatgctggaaacagcatagcaagtttaaataaggctagtccgttatcaacttgaaaaagtggcaccgagtcggtgc

SEQ ID NO: 19 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 20 - protein, 7 aa, synthetic construct
PKKKRKV

SEQ ID NO: 21 - protein, 5 aa, synthetic construct
  REPEAT 1..5: note = repeat unit: repeats 0 to 10 times
GGGGS

SEQ ID NO: 22 - protein, 5 aa, synthetic construct
GGGGG

SEQ ID NO: 23 - protein, 5 aa, synthetic construct
GGAGG

SEQ ID NO: 24 - protein, 7 aa, synthetic construct
GGGGSSS

SEQ ID NO: 25 - protein, 7 aa, synthetic construct
GGGGAAA

SEQ ID NO: 26 - protein, 1368 aa, Streptococcus pyogenes [sequence omitted]

SEQ ID NO: 27 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 28 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - DNA, 3159 bp, synthetic construct
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 31 - DNA, 3159 bp, synthetic construct
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 32 - DNA, 4107 bp, synthetic construct
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 33 - DNA, 3158 bp, synthetic construct
  source 1..3158: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 34 - DNA, 3159 bp, synthetic construct
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 35 - DNA, 3159 bp, synthetic construct
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 36 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 37 - DNA, 3242 bp, synthetic construct
  source 1..3242: mol_type = other DNA
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 38 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 39 - DNA, 3156 bp, synthetic construct
  source 1..3156: mol_type = other DNA
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 40 - DNA, 7009 bp, synthetic construct
  source 1..7009: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 41 - protein, 2414 aa, synthetic construct
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 42 - protein, 617 aa, synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 43 - protein, 1497 aa, synthetic construct
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 44 - DNA, 4491 bp, synthetic construct
  source 1..4491: mol_type = other DNA
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 45 - protein, 65 aa, synthetic construct
RTLVTFKDVFVDFTREEWKLLDTAQQILYRNVMLENYKNLVSLGYQLTKPDVILRLEKGEEPWLV

SEQ ID NO: 46 - DNA, 195 bp, synthetic construct
  source 1..195: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 47 - protein, 1519 aa, synthetic construct
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 48 - DNA, 4560 bp, synthetic construct
  source 1..4560: mol_type = other DNA
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 49 - protein, 1190 aa, synthetic construct
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 50 - DNA, 3570 bp, synthetic construct
  source 1..3570: mol_type = other DNA
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 51 - protein, 718 aa, synthetic construct
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 52 - DNA, 2154 bp, synthetic construct
  source 1..2154: mol_type = other DNA
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 53 - protein, 378 aa, synthetic construct
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 54 - DNA, 1134 bp, synthetic construct
  source 1..1134: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 55 - protein, 522 aa, synthetic construct
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 56 - DNA, 1566 bp, synthetic construct
  source 1..1566: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 57 - protein, 475 aa, synthetic construct
YKKAGSTMDKDSQGLLDSSLMASGTASRSEDEESLAGQKRASSQALGTIPKRRSSSRFIKRKKFDDELVESSLAKSSTRAKGASGVEPGRCSGSEPSSSEKKKVSKAPSTPVPPSPAPAPGLTKRVKKSKQPLQVTKDLGRWKPADDLLLINAVLQTNDLTSVHLGVKFSCRFTLREVQERWYALLYDPVISKLACQAMRQLHPEAIAAIQSKALFSKAEEQLLSKVGSTSQPTLETFQDLLHRHPDAFYLARTAKALQAHWQLMKQYYLLEDQTVQPLPKGDQVLNFSDAEDLIDDSKLKDMRDEVLEHELMVADRRQKREIRQLEQELHKWQVLVDSITGMSSPDFDNQTLAVLRGRMVRYLMRSREITLGRATKDNQIDVDLSLEGPAWKISRKQGVIKLKNNGDFFIANEGRRPIYIDGRPVLCGSKWRLSNNSVVEIASLRFVFLINQDLIALIRAEAAKITPQLDPAFL

SEQ ID NO: 58 - DNA, 1429 bp, synthetic construct
  source 1..1429: mol_type = other DNA
gtacaaaaaagcaggctccaccatggacaaagattctcaggggctgctagattcatccctgatggcatcaggcactgccagccgctcagaggatgaggagtcactggcagggcagaagcgagcctcctcccaggccttgggcaccatccctaaacggagaagctcctccaggttcatcaagaggaagaagttcgatgatgagctggtggagagcagcctggcaaaatcttctacccgggcaaagggggccagtggggtggaaccagggcgctgttcggggagtgaaccctcctccagtgagaagaagaaggtatccaaagcccccagcactcctgtgccacccagcccagccccagcccctggactcaccaagcgtgtgaagaagagtaaacagccacttcaggtgaccaaggatctgggccgctggaagcctgcagatgacctcctgctcataaatgctgtgttgcagaccaacgacctgacctccgtccacctgggcgtgaaattcagctgccgcttcacccttcgggaggtccaggagcgttggtacgccctgctctacgatcctgtcatctccaagttggcctgtcaggccatgaggcagctgcacccagaggctattgcagccatccagagcaaggccctgtttagcaaggctgaggagcagctgctgagcaaagtgggatcgaccagccagcccaccttggagaccttccaggacctgctgcacagacaccctgatgccttctacctggcccgtaccgcgaaggccctgcaggcccactggcagctcatgaagcagtattacctgctggaggaccagacagtgcagccgctgcccaaaggggaccaagtgctgaacttctctgatgcagaggacctgattgatgacagtaagctcaaggacatgcgagatgaggtcctggaacatgagctgatggtggctgaccggcgccagaagcgagagattcggcagctggaacaggaactgcataagtggcaggtgctagtggacagcatcacaggcatgagctctccggacttcgacaaccagacactggcagtgctgcggggccgcatggtgcggtacctgatgcgctcgcgtgagatcaccctgggcagagcaaccaaggataaccagattgatgtggacctgtctctggagggtccggcctggaagatatcccggaaacaaggtgtcatcaagctgaagaacaacggtgatttcttcattgccaatgagggtcgacggcccatctacatcgatggacggccggtgctctgtggctccaaatggcgcctcagcaacaactctgtggtggagatcgccagcctgcgattcgtcttccttatcaaccaggacctcattgccctcatcagggctgaggctgccaagatcacaccacagttggacccagctttcttgtac

SEQ ID NO: 59 - protein, 850 aa, synthetic construct
MTLDMDAVLSDFVRSTGAEPGLARDLLEGKNWDVNAALSDFEQLRQVHAGNLPPSFSEGSGGSRTPEKGFSDREPTRPPRPILQRQDDIVQEKRLSRGISHASSSIVSLARSHVSSNGGGGGSNEHPLEMPICAFQLPDLTVYNEDFRSFIERDLIEQSMLVALEQAGRLNWWVSVDPTSQRLLPLATTGDGNCLLHAASLGMWGFHDRDLMLRKALYALMEKGVEKEALKRRWRWQQTQQNKESGLVYTEDEWQKEWNELIKLASSEPRMHLGTNGANCGGVESSEEPVYESLEEFHVFVLAHVLRRPIVVVADTMLRDSGGEAFAPIPFGGIYLPLEVPASQCHRSPLVLAYDQAHFSALVSMEQKENTKEQAVIPLTDSEYKLLPLHFAVDPGKGWEWGKDDSDNVRLASVILSLEVKLHLLHSYMNVKWIPLSSDAQAPLAQPESPTASAGDEPRSTPESGDSDKESVGSSSTSNEGGRRKEKSKRDREKDKKRADSVANKLGSFGKTLGSKLKKNMGGLMHSKGSKPGGVGTGLGGSSGTETLEKKKKNSLKSWKGGKEEAAGDGPVSEKPPAESVGNGGSKYSQEVMQSLSILRTAMQGEGKFIFVGTLKMGHRHQYQEEMIQRYLSDAEERFLAEQKQKEAERKIMNGGIGGGPPPAKKPEPDAREEQPTGPPAESRAMAFSTGYPGDFTIPRPSGGGVHCQEPRRQLAGGPCVGGLPPYATFPRQCPPGRPYPHQDSIPSLEPGSHSKDGLHRGALLPPPYRVADSYSNGYREPPEPDGWAGGLRGLPPTQTKCKQPNCSFYGHPETNNFCSCCYREELRRREREPDGELLVHRFLDPAFLY

SEQ ID NO: 60 - DNA, 2572 bp, synthetic construct
  source 1..2572: mol_type = other DNA
gtacaaaaaagcaggctccaccatgaccctggacatggatgctgttctgtcagattttgtccgttccacaggagcagagccagggctagcgcgagatctcctagaaggaaagaattgggatgtgaatgccgccctcagtgattttgaacagctacgtcaagtccatgctggaaacctacccccatcctttagtgaggggagtggtggctccaggacccctgaaaaagggttttctgacagagagcctactcgccctccccgacccatcctccagcggcaggatgacatcgttcaagaaaaacgcctgtctaggggcatctcccacgccagctccagcattgtttccctggcccggtcccatgtctcctccaatggtgggggtggggggagcaatgagcaccccctggaaatgcccatctgtgccttccagcttccagatctcactgtatacaatgaagacttccgcagcttcatagagagagacctcattgagcagtccatgctggttgccttggaacaggcagggcgtttgaactggtgggtgagtgtggatcccacctctcagaggctgcttcctttggcaactactggagatgggaactgcctcctgcatgcagcctcccttggaatgtggggtttccatgatcgggacttgatgctgcggaaagctttgtatgcactgatggagaagggagttgagaaggaagcgttgaaaaggcgctggaggtggcagcagacacagcagaataaagagtcagggctggtatacacagaagatgaatggcagaaggagtggaatgaactgatcaagcttgcctcaagtgaaccccgaatgcatctaggtaccaatggagccaactgtggtggggtggagagttctgaggagcctgtatatgagagccttgaagagtttcacgtctttgtccttgctcatgtgcttaggaggcccatagtcgtcgtggcagacaccatgctgagggactccggaggggaagcatttgcccctattccctttggaggaatctatctgcctttggaggtcccagccagccagtgtcaccgctcccctctggtgctcgcctatgatcaggcccacttttctgcactcgtgtccatggagcagaaggagaataccaaggaacaagctgtgatcccacttacagattcagagtataagctgctgcccttgcactttgctgtggaccctggaaagggctgggagtggggcaaagatgatagtgacaatgtccgattggccagtgtaattctgtccctagaggtcaaattgcatctgctgcatagctacatgaatgtgaagtggatcccactgtcctctgatgcacaggctcctctggcccagcctgagtcccccaccgcctcagctggagatgagccccggtccactcctgagtctggagactcagacaaggagtcagttggcagcagttccaccagcaacgagggcggccggcggaaggagaagtcaaagcgagatcgggagaaggacaagaagagagcagattctgtggctaacaaactgggcagctttggcaaaaccttgggcagcaagctcaagaagaacatggggggcctgatgcacagcaagggttcaaagcctggaggggtggggacagggttgggaggaagcagcggcactgagacactggagaagaagaagaaaaactcactgaagagctggaagggtggcaaggaggaggcagctggggatgggcctgtgtctgagaagcccccagctgagtctgttggtaacggagggagcaagtatagccaggaggtgatgcaaagcctgagcattctgaggactgccatgcaaggggaggggaagtttatttttgttggaaccctgaagatgggtcaccgtcaccagtatcaggaggaaatgatccagcgctacctttctgatgctgaggagagattcctggcagaacagaagcagaaggaggcagagaggaagatcatgaatggaggaatagggggtggccctcctccagccaaaaagccagagccagatgctagggaagagcagccgaccggtcccccagcagagtccagggcaatggcattttccactggctaccctggggactttactatccctcggccgtctgggggcggagtccactgccaggaaccccggaggcagttggcagggggtccatgtgtcgggggcctaccaccatatgccaccttccccagacagtgccctcctgggcgaccctacccccaccaggacagcatcccttctctggagccaggcagccactctaaggatggacttcacaggggtgccttgttaccacccccctaccgagtggctgattcctatagcaatggctacagagagccccctgagccagatggatgggctggaggtctccggggccttcccccaactcagaccaaatgcaaacaaccgaactgcagcttctatggacaccctgagacaaacaacttctgttcctgttgttacagggaagaactgaggaggagggagcgggaaccggatggggagctcctggtgcacaggttcttggacccagctttcttgtac

SEQ ID NO: 61 - protein, 382 aa, synthetic construct
MLDRDVGPTPMYPPTYLEPGIGRHTPYGNQTDYRIFELNKRLQNWTEECDNLWWDAFTTEFFEDDAMLTITFCLEDGPKRYTIGRTLIPRYFRSIFEGGATELYYVLKHPKEAFHSNFVSLDCDQGSMVTQHGKPMFTQVCVEGRLYLEFMFDDMMRIKTWHFSIRQHRELIPRSILAMHAQDPQMLDQLSKNITRCGLSNSTLNYLRLCVILEPMQELMSRHKTYSLSPRDCLKTCLFQKWQRMVAPPAEPTRQQPSKRRKRKMSGGSTMSSGGGNTNNSNSKKKSPASTFALSSQVPDVMVVGEPTLMGGEFGDEDERLITRLENTQFDAANGIDDEDSFNNSPALGANSPWNSKPPSSQESKSENPTSQASQLDPAFLY

SEQ ID NO: 62 - DNA, 1146 bp, synthetic construct
  source 1..1146: mol_type = other DNA
atgctggatagggatgtgggcccaactcccatgtatccgcctacatacctggagccagggattgggaggcacacaccatatggcaaccaaactgactacagaatatttgagcttaacaaacggcttcagaactggacagaggagtgtgacaatctctggtgggatgcattcacgactgagttctttgaggatgatgccatgttgaccatcactttctgcctggaggatggaccaaagagatataccattggccggaccctgatcccacgctacttccgcagcatctttgaggggggtgctacggagctgtactatgttcttaagcaccccaaggaggcattccacagcaactttgtgtccctcgactgtgaccagggcagcatggtgacccagcatggcaagcccatgttcacccaggtgtgtgtggagggccggttgtacctggagttcatgtttgacgacatgatgcggataaagacgtggcacttcagcatccggcagcaccgagagctcatcccccgcagcatccttgccatgcatgcccaagacccccagatgttggatcagctctccaaaaacatcactcggtgtgggctgtccaattccactctcaactacctccgactctgtgtgatactcgagcccatgcaagagctcatgtcacgccacaagacctacagcctcagcccccgcgactgcctcaagacctgccttttccagaagtggcagcgcatggtagcaccccctgcggagcccacacgtcagcagcccagcaaacggcggaaacggaagatgtcagggggcagcaccatgagctctggtggtggcaacaccaacaacagcaacagcaagaagaagagcccagctagcaccttcgccctctccagccaggtacctgatgtgatggtggtgggggagcccaccctgatgggcggggagttcggggacgaggacgagaggctcatcacccggctggagaacacccagtttgacgcagccaacggcattgacgacgaggacagctttaacaactcccctgcactgggcgccaacagcccctggaacagcaagcctccgtccagccaagaaagcaaatcggagaaccccacgtcacaggcctcccagttggacccagctttcttgtac

SEQ ID NO: 63 - protein, 356 aa, synthetic construct
MAGVACLGKAADADEWCDSGLGSLGPDAAAPGGPGLGAELGPGLSWAPLVFGYVTEDGDTALHLAVIHQHEPFLDFLLGFSAGTEYMDLQNDLGQTALHLAAILGETSTVEKLYAAGAGLCVAERRGHTALHLACRVGAHACARALLQPRPRRPREAPDTYLAQGPDRTPDTNHTPVALYPDSDLEKEEEESEEDWKLQLEAENYEGHTPLHVAVIHKDVEMVRLLRDAGADLDKPEPTCGRSPLHLAVEAQAADVLELLLRAGANPAARMYGGRTPLGSAMLRPNPILARLLRAHGAPEPEGEDEKSGPCSSSSDSDSGDEGDEYDDIVVHSSRSQTRLPPTPASKPLPDDPRPV

SEQ ID NO: 64 - DNA, 1071 bp, synthetic construct
  source 1..1071: mol_type = other DNA
atggctggggtcgcgtgcttgggaaaagctgccgacgcagatgaatggtgcgacagcggcctgggctccctgggtccggacgcagcggcccccggaggacctgggttgggcgcggagttgggcccggggctgtcgtgggctcccctcgtcttcggctacgtcactgaggatggggacacggcactgcacttggctgtgattcatcagcatgaacccttcctggattttcttctaggcttctcggccggcactgagtacatggacctgcagaatgacctaggccagacagccctgcacctggcagccatcctgggggagacatccacggtggagaagctgtacgcagcaggcgccgggctgtgtgtggcggagcgtaggggccacacggcgctgcacctggcctgccgtgtgggggcacacgcctgtgcccgtgccctgcttcagccccgcccccggcgccccagggaagcccccgacacctacctcgctcagggccctgaccgtactcccgacaccaaccatacccctgtcgccttgtaccccgattccgacttggagaaggaagaagaggagagtgaggaggactggaagctgcagctggaggctgaaaactacgagggccacaccccactccacgtggccgttatccacaaagatgtggagatggtccggctgctccgagatgctggagctgaccttgacaaaccggagcccacgtgcggccggagcccccttcatttggcagtggaggcccaggcagccgatgtgctggagcttctcctgagggcaggcgcgaaccctgctgcccgcatgtacggtggccgcaccccactcggcagtgccatgctccggcccaaccccatcctcgcccgcctcctccgtgcacacggagcccctgagcccgagggcgaggacgagaaatccggcccctgcagcagcagtagcgacagcgacagcggagacgagggcgatgaatacgacgacattgtggttcacagcagccgcagccaaacccggctgcctcccaccccagcctcaaaacctcttcctgacgacccccgccccgtgtga

SEQ ID NO: 65 - protein, 579 aa, synthetic construct
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 66 - DNA, 1740 bp, synthetic construct
  source 1..1740: mol_type = other DNA
atgcttcggtctgggccagcctctgggccgtccgtccccactggccgggccatgccgagtcgccgcgtcgccagaccgccggctgcgccggagctgggggccttagggtcccccgacctctcctcactctcgctcgccgtttccaggagcacagatgaattggagatcatcgacgagtacatcaaggagaacggcttcggcctggacgggggacagccgggcccgggcgaggggctgccacgcctggtgtctcgcggggctgcgtccctgagcacggtcaccctgggccctgtggcgcccccagccacgccgccgccttggggctgccccctgggccgactagtgtccccagcgccgggcccgggcccgcagccgcacctggtcatcacggagcagcccaagcagcgcggcatgcgcttccgctacgagtgcgagggccgctcggccggcagcatccttggggagagcagcaccgaggccagcaagacgctgcccgccatcgagctccgggattgtggagggctgcgggaggtggaggtgactgcctgcctggtgtggaaggactggcctcaccgagtccacccccacagcctcgtggggaaagactgcaccgacggcatctgcagggtgcggctccggcctcacgtcagcccccggcacagttttaacaacctgggcatccagtgtgtgaggaagaaggagattgaggctgccattgagcggaagattcaactgggcattgacccctacaacgctgggtccctgaagaaccatcaggaagtagacatgaatgtggtgaggatctgcttccaggcctcatatcgggaccagcagggacagatgcgccggatggatcctgtgctttccgagcccgtctatgacaagaaatccacaaacacatcagagctgcggatttgccgaattaacaaggaaagcgggccgtgcaccggtggcgaggagctctacttgctctgcgacaaggtgcagaaagaggacatatcagtggtgttcagcagggcctcctgggaaggtcgggctgacttctcccaggccgacgtgcaccgccagattgccattgtgttcaagacgccgccctacgaggacctggagattgtcgagcccgtgacagtcaacgtcttcctgcagcggctcaccgatggggtctgcagcgagccattgcctttcacgtacctgcctcgcgaccatgacagctacggcgtggacaagaagcggaaacgggggatgcccgacgtccttggggagctgaacagctctgacccccatggcatcgagagcaaacggcggaagaaaaagccggccatcctggaccacttcctgcccaaccacggctcaggcccgttcctcccgccgtcagccctgctgccagaccctgacttcttctctggcaccgtgtccctgcccggcctggagccccctggcgggcctgacctcctggacgatggctttgcctacgaccctacggcccccacactcttcaccatgctggacctgctgcccccggcaccgccacacgctagcgctgttgtgtgcagcggaggtgccggggccgtggttggggagacccccggccctgaaccactgacactggactcgtaccaggccccgggccccggggatggaggcaccgccagccttgtgggcagcaacatgttccccaatcattaccgcgaggcggcctttgggggcggcctcctatccccggggcctgaagccacgtag

SEQ ID NO: 67 - protein, 277 aa, synthetic construct
MADHMMAMNHGRFPDGTNGLHHHPAHRMGMGQFPSPHHHQQQQPQHAFNALMGEHIHYGAGNMNATSGIRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSCLDPAFLY

SEQ ID NO: 68 - DNA, 831 bp, synthetic construct
  source 1..831: mol_type = other DNA
atggcagaccatatgatggcaatgaaccacgggcgcttccccgacggcaccaatgggctgcaccatcaccctgcccaccgcatgggcatggggcagttcccgagcccccatcaccaccagcagcagcagccccagcacgccttcaacgccctaatgggcgagcacatacactacggcgcgggcaacatgaatgccacgagcggcatcaggcatgcgatggggccggggactgtgaacggagggcaccccccgagcgcgctggcccccgcggccaggtttaacaactcccagttcatgggtcccccggtggccagccagggaggctccctgccggccagcatgcagctgcagaagctcaacaaccagtatttcaaccatcacccctacccccacaaccactacatgccggatttgcaccctgctgcaggccaccagatgaacgggacaaaccagcacttccgagattgcaaccccaagcacagcggcggcagcagcacccccggcggctcgggcggcagcagcacccccggcggctctggcagcagctcgggcggcggcgcgggcagcagcaacagcggcggcggcagcggcagcggcaacatgcccgcctccgtggcccacgtccccgctgcaatgctgccgcccaatgtcatagacactgatttcatcgacgaggaagttcttatgtccttggtgatagaaatgggtttggaccgcatcaaggagctgcccgaactctggctggggcaaaacgagtttgattttatgacggacttcgtgtgcaaacagcagcccagcagagtgagctgtttggacccagctttcttgtac

SEQ ID NO: 69 - protein, 1066 aa, synthetic construct
NLSSCFVLINKERSFBFPLINKERMCRSMGPKKKRKVGGMGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPILVELDGDVNGHKFSVRGEGEGDATNGKLTLKFICTTGKLPVPWPTLVTTLTHGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGRTGGGGSGGGGADPKKKRKVARITSLYKKAGSTMDKDSQGLLDSSLMASGTASRSEDEESLAGQKRASSQALGTIPKRRSSSRFIKRKKFDDELVESSLAKSSTRAKGASGVEPGRCSGSEPSSSEKKKVSKAPSTPVPPSPAPAPGLTKRVKKSKQPLQVTKDLGRWKPADDLLLINAVLQTNDLTSVHLGVKFSCRFTLREVQERWYALLYDPVISKLACQAMRQLHPEAIAAIQSKALFSKAEEQLLSKVGSTSQPTLETFQDLLHRHPDAFYLARTAKALQAHWQLMKQYYLLEDQTVQPLPKGDQVLNFSDAEDLIDDSKLKDMRDEVLEHELMVADRRQKREIRQLEQELHKWQVLVDSITGMSSPDFDNQTLAVLRGRMVRYLMRSREITLGRATKDNQIDVDLSLEGPAWKISRKQGVIKLKNNGDFFIANEGRRPIYIDGRPVLCGSKWRLSNNSVVEIASLRFVFLINQDLIALIRAEAAKITPQLDPAFL

SEQ ID NO: 70 - DNA, 3139 bp, synthetic construct
  source 1..3139: mol_type = other DNA
atgggtcccaagaaaaagagaaaggtcggtggcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggtggaagcggaggcggtgggtctggtggaggcggcagctctggcggaggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacccatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtggaggtcggaccggtggcggtggcagcggtggaggcggtgctgaccccaagaagaagaggaaggtggctaggatcacaagtttgtacaaaaaagcaggctccaccgtacaaaaaagcaggctccaccatggacaaagattctcaggggctgctagattcatccctgatggcatcaggcactgccagccgctcagaggatgaggagtcactggcagggcagaagcgagcctcctcccaggccttgggcaccatccctaaacggagaagctcctccaggttcatcaagaggaagaagttcgatgatgagctggtggagagcagcctggcaaaatcttctacccgggcaaagggggccagtggggtggaaccagggcgctgttcggggagtgaaccctcctccagtgagaagaagaaggtatccaaagcccccagcactcctgtgccacccagcccagccccagcccctggactcaccaagcgtgtgaagaagagtaaacagccacttcaggtgaccaaggatctgggccgctggaagcctgcagatgacctcctgctcataaatgctgtgttgcagaccaacgacctgacctccgtccacctgggcgtgaaattcagctgccgcttcacccttcgggaggtccaggagcgttggtacgccctgctctacgatcctgtcatctccaagttggcctgtcaggccatgaggcagctgcacccagaggctattgcagccatccagagcaaggccctgtttagcaaggctgaggagcagctgctgagcaaagtgggatcgaccagccagcccaccttggagaccttccaggacctgctgcacagacaccctgatgccttctacctggcccgtaccgcgaaggccctgcaggcccactggcagctcatgaagcagtattacctgctggaggaccagacagtgcagccgctgcccaaaggggaccaagtgctgaacttctctgatgcagaggacctgattgatgacagtaagctcaaggacatgcgagatgaggtcctggaacatgagctgatggtggctgaccggcgccagaagcgagagattcggcagctggaacaggaactgcataagtggcaggtgctagtggacagcatcacaggcatgagctctccggacttcgacaaccagacactggcagtgctgcggggccgcatggtgcggtacctgatgcgctcgcgtgagatcaccctgggcagagcaaccaaggataaccagattgatgtggacctgtctctggagggtccggcctggaagatatcccggaaacaaggtgtcatcaagctgaagaacaacggtgatttcttcattgccaatgagggtcgacggcccatctacatcgatggacggccggtgctctgtggctccaaatggcgcctcagcaacaactctgtggtggagatcgccagcctgcgattcgtcttccttatcaaccaggacctcattgccctcatcagggctgaggctgccaagatcacaccacagttggacccagctttcttgtac

SEQ ID NO: 71 - protein, 1423 aa, synthetic construct
MGPKKKRKVGGMGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPILVELDGDVNGHKFSVRGEGEGDATNGKLTLKFICTTGKLPVPWPTLVTTLTHGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGRTGGGGSGGGGADPKKKRKVARITSLYKKAGSTMTLDMDAVLSDFVRSTGAEPGLARDLLEGKNWDVNAALSDFEQLRQVHAGNLPPSFSEGSGGSRTPEKGFSDREPTRPPRPILQRQDDIVQEKRLSRGISHASSSIVSLARSHVSSNGGGGGSNEHPLEMPICAFQLPDLTVYNEDFRSFIERDLIEQSMLVALEQAGRLNWWVSVDPTSQRLLPLATTGDGNCLLHAASLGMWGFHDRDLMLRKALYALMEKGVEKEALKRRWRWQQTQQNKESGLVYTEDEWQKEWNELIKLASSEPRMHLGTNGANCGGVESSEEPVYESLEEFHVFVLAHVLRRPIVVVADTMLRDSGGEAFAPIPFGGIYLPLEVPASQCHRSPLVLAYDQAHFSALVSMEQKENTKEQAVIPLTDSEYKLLPLHFAVDPGKGWEWGKDDSDNVRLASVILSLEVKLHLLHSYMNVKWIPLSSDAQAPLAQPESPTASAGDEPRSTPESGDSDKESVGSSSTSNEGGRRKEKSKRDREKDKKRADSVANKLGSFGKTLGSKLKKNMGGLMHSKGSKPGGVGTGLGGSSGTETLEKKKKNSLKSWKGGKEEAAGDGPVSEKPPAESVGNGGSKYSQEVMQSLSILRTAMQGEGKFIFVGTLKMGHRHQYQEEMIQRYLSDAEERFLAEQKQKEAERKIMNGGIGGGPPPAKKPEPDAREEQPTGPPAESRAMAFSTGYPGDFTIPRPSGGGVHCQEPRRQLAGGPCVGGLPPYATFPRQCPPGRPYPHQDSIPSLEPGSHSKDGLHRGALLPPPYRVADSYSNGYREPPEPDGWAGGLRGLPPTQTKCKQPNCSFYGHPETNNFCSCCYREELRRREREPDGELLVHRFLDPAFLYKVV

SEQ ID NO: 72 - DNA, 4282 bp, synthetic construct
  source 1..4282: mol_type = other DNA
atgggtcccaagaaaaagagaaaggtcggtggcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggtggaagcggaggcggtgggtctggtggaggcggcagctctggcggaggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacccatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtggaggtcggaccggtggcggtggcagcggtggaggcggtgctgaccccaagaagaagaggaaggtggctaggatcacaagtttgtacaaaaaagcaggctccaccgtacaaaaaagcaggctccaccatgaccctggacatggatgctgttctgtcagattttgtccgttccacaggagcagagccagggctagcgcgagatctcctagaaggaaagaattgggatgtgaatgccgccctcagtgattttgaacagctacgtcaagtccatgctggaaacctacccccatcctttagtgaggggagtggtggctccaggacccctgaaaaagggttttctgacagagagcctactcgccctccccgacccatcctccagcggcaggatgacatcgttcaagaaaaacgcctgtctaggggcatctcccacgccagctccagcattgtttccctggcccggtcccatgtctcctccaatggtgggggtggggggagcaatgagcaccccctggaaatgcccatctgtgccttccagcttccagatctcactgtatacaatgaagacttccgcagcttcatagagagagacctcattgagcagtccatgctggttgccttggaacaggcagggcgtttgaactggtgggtgagtgtggatcccacctctcagaggctgcttcctttggcaactactggagatgggaactgcctcctgcatgcagcctcccttggaatgtggggtttccatgatcgggacttgatgctgcggaaagctttgtatgcactgatggagaagggagttgagaaggaagcgttgaaaaggcgctggaggtggcagcagacacagcagaataaagagtcagggctggtatacacagaagatgaatggcagaaggagtggaatgaactgatcaagcttgcctcaagtgaaccccgaatgcatctaggtaccaatggagccaactgtggtggggtggagagttctgaggagcctgtatatgagagccttgaagagtttcacgtctttgtccttgctcatgtgcttaggaggcccatagtcgtcgtggcagacaccatgctgagggactccggaggggaagcatttgcccctattccctttggaggaatctatctgcctttggaggtcccagccagccagtgtcaccgctcccctctggtgctcgcctatgatcaggcccacttttctgcactcgtgtccatggagcagaaggagaataccaaggaacaagctgtgatcccacttacagattcagagtataagctgctgcccttgcactttgctgtggaccctggaaagggctgggagtggggcaaagatgatagtgacaatgtccgattggccagtgtaattctgtccctagaggtcaaattgcatctgctgcatagctacatgaatgtgaagtggatcccactgtcctctgatgcacaggctcctctggcccagcctgagtcccccaccgcctcagctggagatgagccccggtccactcctgagtctggagactcagacaaggagtcagttggcagcagttccaccagcaacgagggcggccggcggaaggagaagtcaaagcgagatcgggagaaggacaagaagagagcagattctgtggctaacaaactgggcagctttggcaaaaccttgggcagcaagctcaagaagaacatggggggcctgatgcacagcaagggttcaaagcctggaggggtggggacagggttgggaggaagcagcggcactgagacactggagaagaagaagaaaaactcactgaagagctggaagggtggcaaggaggaggcagctggggatgggcctgtgtctgagaagcccccagctgagtctgttggtaacggagggagcaagtatagccaggaggtgatgcaaagcctgagcattctgaggactgccatgcaaggggaggggaagtttatttttgttggaaccctgaagatgggtcaccgtcaccagtatcaggaggaaatgatccagcgctacctttctgatgctgaggagagattcctggcagaacagaagcagaaggaggcagagaggaagatcatgaatggaggaatagggggtggccctcctccagccaaaaagccagagccagatgctagggaagagcagccgaccggtcccccagcagagtccagggcaatggcattttccactggctaccctggggactttactatccctcggccgtctgggggcggagtccactgccaggaaccccggaggcagttggcagggggtccatgtgtcgggggcctaccaccatatgccaccttccccagacagtgccctcctgggcgaccctacccccaccaggacagcatcccttctctggagccaggcagccactctaaggatggacttcacaggggtgccttgttaccacccccctaccgagtggctgattcctatagcaatggctacagagagccccctgagccagatggatgggctggaggtctccggggccttcccccaactcagaccaaatgcaaacaaccgaactgcagcttctatggacaccctgagacaaacaacttctgttcctgttgttacagggaagaactgaggaggagggagcgggaaccggatggggagctcctggtgcacaggttcttggacccagctttcttgtac

SEQ ID NO: 73 - protein, 952 aa, synthetic construct
MGPKKKRKVGGMGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPILVELDGDVNGHKFSVRGEGEGDATNGKLTLKFICTTGKLPVPWPTLVTTLTHGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGRTGGGGSGGGGADPKKKRKVARITSLYKKAGSTMLDRDVGPTPMYPPTYLEPGIGRHTPYGNQTDYRIFELNKRLQNWTEECDNLWWDAFTTEFFEDDAMLTITFCLEDGPKRYTIGRTLIPRYFRSIFEGGATELYYVLKHPKEAFHSNFVSLDCDQGSMVTQHGKPMFTQVCVEGRLYLEFMFDDMMRIKTWHFSIRQHRELIPRSILAMHAQDPQMLDQLSKNITRCGLSNSTLNYLRLCVILEPMQELMSRHKTYSLSPRDCLKTCLFQKWQRMVAPPAEPTRQQPSKRRKRKMSGGSTMSSGGGNTNNSNSKKKSPASTFALSSQVPDVMVVGEPTLMGGEFGDEDERLITRLENTQFDAANGIDDEDSFNNSPALGANSPWNSKPPSSQESKSENPTSQASQLDPAFLY

SEQ ID NO: 74 - DNA, 2856 bp, synthetic construct
  source 1..2856: mol_type = other DNA
atgggtcccaagaaaaagagaaaggtcggtggcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggtggaagcggaggcggtgggtctggtggaggcggcagctctggcggaggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacccatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtggaggtcggaccggtggcggtggcagcggtggaggcggtgctgaccccaagaagaagaggaaggtggctaggatcacaagtttgtacaaaaaagcaggctccaccatgctggatagggatgtgggcccaactcccatgtatccgcctacatacctggagccagggattgggaggcacacaccatatggcaaccaaactgactacagaatatttgagcttaacaaacggcttcagaactggacagaggagtgtgacaatctctggtgggatgcattcacgactgagttctttgaggatgatgccatgttgaccatcactttctgcctggaggatggaccaaagagatataccattggccggaccctgatcccacgctacttccgcagcatctttgaggggggtgctacggagctgtactatgttcttaagcaccccaaggaggcattccacagcaactttgtgtccctcgactgtgaccagggcagcatggtgacccagcatggcaagcccatgttcacccaggtgtgtgtggagggccggttgtacctggagttcatgtttgacgacatgatgcggataaagacgtggcacttcagcatccggcagcaccgagagctcatcccccgcagcatccttgccatgcatgcccaagacccccagatgttggatcagctctccaaaaacatcactcggtgtgggctgtccaattccactctcaactacctccgactctgtgtgatactcgagcccatgcaagagctcatgtcacgccacaagacctacagcctcagcccccgcgactgcctcaagacctgccttttccagaagtggcagcgcatggtagcaccccctgcggagcccacacgtcagcagcccagcaaacggcggaaacggaagatgtcagggggcagcaccatgagctctggtggtggcaacaccaacaacagcaacagcaagaagaagagcccagctagcaccttcgccctctccagccaggtacctgatgtgatggtggtgggggagcccaccctgatgggcggggagttcggggacgaggacgagaggctcatcacccggctggagaacacccagtttgacgcagccaacggcattgacgacgaggacagctttaacaactcccctgcactgggcgccaacagcccctggaacagcaagcctccgtccagccaagaaagcaaatcggagaaccccacgtcacaggcctcccagttggacccagctttcttgtac

SEQ ID NO: 75 - protein, 926 aa, synthetic construct
MGPKKKRKVGGMGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPILVELDGDVNGHKFSVRGEGEGDATNGKLTLKFICTTGKLPVPWPTLVTTLTHGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGRTGGGGSGGGGADPKKKRKVARITSLYKKAGSTMAGVACLGKAADADEWCDSGLGSLGPDAAAPGGPGLGAELGPGLSWAPLVFGYVTEDGDTALHLAVIHQHEPFLDFLLGFSAGTEYMDLQNDLGQTALHLAAILGETSTVEKLYAAGAGLCVAERRGHTALHLACRVGAHACARALLQPRPRRPREAPDTYLAQGPDRTPDTNHTPVALYPDSDLEKEEEESEEDWKLQLEAENYEGHTPLHVAVIHKDVEMVRLLRDAGADLDKPEPTCGRSPLHLAVEAQAADVLELLLRAGANPAARMYGGRTPLGSAMLRPNPILARLLRAHGAPEPEGEDEKSGPCSSSSDSDSGDEGDEYDDIVVHSSRSQTRLPPTPASKPLPDDPRPV

SEQ ID NO: 76 - DNA, 2781 bp, synthetic construct
  source 1..2781: mol_type = other DNA
atgggtcccaagaaaaagagaaaggtcggtggcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggtggaagcggaggcggtgggtctggtggaggcggcagctctggcggaggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacccatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtggaggtcggaccggtggcggtggcagcggtggaggcggtgctgaccccaagaagaagaggaaggtggctaggatcacaagtttgtacaaaaaagcaggctccaccatggctggggtcgcgtgcttgggaaaagctgccgacgcagatgaatggtgcgacagcggcctgggctccctgggtccggacgcagcggcccccggaggacctgggttgggcgcggagttgggcccggggctgtcgtgggctcccctcgtcttcggctacgtcactgaggatggggacacggcactgcacttggctgtgattcatcagcatgaacccttcctggattttcttctaggcttctcggccggcactgagtacatggacctgcagaatgacctaggccagacagccctgcacctggcagccatcctgggggagacatccacggtggagaagctgtacgcagcaggcgccgggctgtgtgtggcggagcgtaggggccacacggcgctgcacctggcctgccgtgtgggggcacacgcctgtgcccgtgccctgcttcagccccgcccccggcgccccagggaagcccccgacacctacctcgctcagggccctgaccgtactcccgacaccaaccatacccctgtcgccttgtaccccgattccgacttggagaaggaagaagaggagagtgaggaggactggaagctgcagctggaggctgaaaactacgagggccacaccccactccacgtggccgttatccacaaagatgtggagatggtccggctgctccgagatgctggagctgaccttgacaaaccggagcccacgtgcggccggagcccccttcatttggcagtggaggcccaggcagccgatgtgctggagcttctcctgagggcaggcgcgaaccctgctgcccgcatgtacggtggccgcaccccactcggcagtgccatgctccggcccaaccccatcctcgcccgcctcctccgtgcacacggagcccctgagcccgagggcgaggacgagaaatccggcccctgcagcagcagtagcgacagcgacagcggagacgagggcgatgaatacgacgacattgtggttcacagcagccgcagccaaacccggctgcctcccaccccagcctcaaaacctcttcctgacgacccccgccccgtgtga

SEQ ID NO: 77 - protein, 1149 aa, synthetic construct
MGPKKKRKVGGMGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPILVELDGDVNGHKFSVRGEGEGDATNGKLTLKFICTTGKLPVPWPTLVTTLTHGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGRTGGGGSGGGGADPKKKRKVARITSLYKKAGSTMLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 78 - DNA, 3450 bp, synthetic construct
  source 1..3450: mol_type = other DNA
atgggtcccaagaaaaagagaaaggtcggtggcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggtggaagcggaggcggtgggtctggtggaggcggcagctctggcggaggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacccatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtggaggtcggaccggtggcggtggcagcggtggaggcggtgctgaccccaagaagaagaggaaggtggctaggatcacaagtttgtacaaaaaagcaggctccaccatgcttcggtctgggccagcctctgggccgtccgtccccactggccgggccatgccgagtcgccgcgtcgccagaccgccggctgcgccggagctgggggccttagggtcccccgacctctcctcactctcgctcgccgtttccaggagcacagatgaattggagatcatcgacgagtacatcaaggagaacggcttcggcctggacgggggacagccgggcccgggcgaggggctgccacgcctggtgtctcgcggggctgcgtccctgagcacggtcaccctgggccctgtggcgcccccagccacgccgccgccttggggctgccccctgggccgactagtgtccccagcgccgggcccgggcccgcagccgcacctggtcatcacggagcagcccaagcagcgcggcatgcgcttccgctacgagtgcgagggccgctcggccggcagcatccttggggagagcagcaccgaggccagcaagacgctgcccgccatcgagctccgggattgtggagggctgcgggaggtggaggtgactgcctgcctggtgtggaaggactggcctcaccgagtccacccccacagcctcgtggggaaagactgcaccgacggcatctgcagggtgcggctccggcctcacgtcagcccccggcacagttttaacaacctgggcatccagtgtgtgaggaagaaggagattgaggctgccattgagcggaagattcaactgggcattgacccctacaacgctgggtccctgaagaaccatcaggaagtagacatgaatgtggtgaggatctgcttccaggcctcatatcgggaccagcagggacagatgcgccggatggatcctgtgctttccgagcccgtctatgacaagaaatccacaaacacatcagagctgcggatttgccgaattaacaaggaaagcgggccgtgcaccggtggcgaggagctctacttgctctgcgacaaggtgcagaaagaggacatatcagtggtgttcagcagggcctcctgggaaggtcgggctgacttctcccaggccgacgtgcaccgccagattgccattgtgttcaagacgccgccctacgaggacctggagattgtcgagcccgtgacagtcaacgtcttcctgcagcggctcaccgatggggtctgcagcgagccattgcctttcacgtacctgcctcgcgaccatgacagctacggcgtggacaagaagcggaaacgggggatgcccgacgtccttggggagctgaacagctctgacccccatggcatcgagagcaaacggcggaagaaaaagccggccatcctggaccacttcctgcccaaccacggctcaggcccgttcctcccgccgtcagccctgctgccagaccctgacttcttctctggcaccgtgtccctgcccggcctggagccccctggcgggcctgacctcctggacgatggctttgcctacgaccctacggcccccacactcttcaccatgctggacctgctgcccccggcaccgccacacgctagcgctgttgtgtgcagcggaggtgccggggccgtggttggggagacccccggccctgaaccactgacactggactcgtaccaggccccgggccccggggatggaggcaccgccagccttgtgggcagcaacatgttccccaatcattaccgcgaggcggcctttgggggcggcctcctatccccggggcctgaagccacgtag

SEQ ID NO: 79 - protein, 847 aa, synthetic construct
MGPKKKRKVGGMGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPILVELDGDVNGHKFSVRGEGEGDATNGKLTLKFICTTGKLPVPWPTLVTTLTHGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGGRTGGGGSGGGGADPKKKRKVARITSLYKKAGSTMADHMMAMNHGRFPDGTNGLHHHPAHRMGMGQFPSPHHHQQQQPQHAFNALMGEHIHYGAGNMNATSGIRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSCLDPAFLY

SEQ ID NO: 80 - DNA, 2541 bp, synthetic construct
  source 1..2541: mol_type = other DNA
atgggtcccaagaaaaagagaaaggtcggtggcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggtggaagcggaggcggtgggtctggtggaggcggcagctctggcggaggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacccatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtggaggtcggaccggtggcggtggcagcggtggaggcggtgctgaccccaagaagaagaggaaggtggctaggatcacaagtttgtacaaaaaagcaggctccaccatggcagaccatatgatggcaatgaaccacgggcgcttccccgacggcaccaatgggctgcaccatcaccctgcccaccgcatgggcatggggcagttcccgagcccccatcaccaccagcagcagcagccccagcacgccttcaacgccctaatgggcgagcacatacactacggcgcgggcaacatgaatgccacgagcggcatcaggcatgcgatggggccggggactgtgaacggagggcaccccccgagcgcgctggcccccgcggccaggtttaacaactcccagttcatgggtcccccggtggccagccagggaggctccctgccggccagcatgcagctgcagaagctcaacaaccagtatttcaaccatcacccctacccccacaaccactacatgccggatttgcaccctgctgcaggccaccagatgaacgggacaaaccagcacttccgagattgcaaccccaagcacagcggcggcagcagcacccccggcggctcgggcggcagcagcacccccggcggctctggcagcagctcgggcggcggcgcgggcagcagcaacagcggcggcggcagcggcagcggcaacatgcccgcctccgtggcccacgtccccgctgcaatgctgccgcccaatgtcatagacactgatttcatcgacgaggaagttcttatgtccttggtgatagaaatgggtttggaccgcatcaaggagctgcccgaactctggctggggcaaaacgagtttgattttatgacggacttcgtgtgcaaacagcagcccagcagagtgagctgtttggacccagctttcttgtac

SEQ ID NO: 81 - protein, 247 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSS

SEQ ID NO: 82 - DNA, 858 bp, synthetic construct
  source 1..858: mol_type = other DNA
atgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggtggaagcggaggcggtgggtctggtggaggcggcagctctggcggaggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaagcggt

SEQ ID NO: 83 - protein, 237 aa, synthetic construct
SKGEELFTGVVPILVELDGDVNGHKFSVRGEGEGDATNGKLTLKFICTTGKLPVPWPTLVTTLTHGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 84 - DNA, 711 bp, synthetic construct
  source 1..711: mol_type = other DNA
agcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacccatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaa

SEQ ID NO: 85 - protein, 19 aa, synthetic construct
EELLSKNYHLENEVARLKK

SEQ ID NO: 86 - DNA, 57 bp, synthetic construct
  source 1..57: mol_type = other DNA
gaggagcttctgagcaaaaactatcacctcgaaaacgaggttgcgcgactgaagaaa

SEQ ID NO: 87 - protein, 1607 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDGIGSGSNGSSGSNGPTDAAEEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSGEELLSKNYHLENEVARLKKGSGSGGSGSGSGGSGSGGSGSG

SEQ ID NO: 88 - DNA, 4755 bp, synthetic construct
  source 1..4755: mol_type = other DNA
atggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgacggcattggtagtgggagcaacggcagcagcggatccaacggtccgactgacgccgcggaagaggagcttctgagcaaaaactatcacctcgaaaacgaggttgcgcgactgaagaaaggaagcgggtccggtggaagtggctccggatctggaggttctggcagcggaggtagcggcagtggcgaagagctccttagtaagaactatcatctggaaaatgaggtagcgcgcttaaagaaagggtcgggaagtggcggcagcggaagtgggagtggagggagcggttctggcggttccggcagtggagaggagttgctgtctaagaactaccacttagaaaacgaagtcgcacggctaaaaaaaggttccggctccggcggctccggttctggaagcgggggctcgggatcaggtggatctggatcaggagaggaattgctttccaaaaactaccaccttgagaatgaggtggccaggttaaagaaggggagcggctcggggggtagtggatcggggtcgggcgggtcaggaagcggtggtagcggatctggggaggagctgctctcgaagaattaccatttggagaacgaagtggcgagactaaagaag

SEQ ID NO: 89 - protein, 1997 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSNGPGGSGGGGSGGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKDYHLENEVARLKKGSGSGEELLSKNYHLENEVARLKK

SEQ ID NO: 90 - DNA, 5991 bp, synthetic construct
  source 1..5991: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgggtggatctggaggtggaggttctggaggagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaagagttgctatcgaaaaattatcatcttgagaacgaagttgctaggctcaaaaagggctcaggctcaggcgaggagttgctctcgaaaaactaccacttggaaaatgaggtcgcgaggttgaaaaaggggagcgggtcgggcgaggagttattgagcaaaaactatcatttagagaacgaagtcgcgcgcttaaagaaaggctcgggctcgggcgaagaactcttatcgaagaactaccacctcgaaaatgaggtcgccaggttgaaaaagggcagtggcagcggggaggaactcttgagcaagaactaccacttggagaatgaggtcgcgagattgaagaaagggtcggggagcggcgaggaattgctcagcaagaattatcatttggagaacgaagtcgccaggctcaagaaaggctcggggtcgggggaggaattgttgagtaaaaactaccacttggaaaatgaagtcgccaggctcaaaaaagggagtgggagcggcgaagagttattgagcaaaaattaccacttggagaacgaagtggcaaggctcaagaaagggagcggcagcggggaggagctcttatcgaagaactaccacttagagaatgaagtcgcccgcttgaagaaaggctcggggagcggggaagagctcttgagcaagaactaccacttggaaaatgaggtggcgcgcttgaagaaagggagcgggagcggggaagagttactatctaagaattatcatctcgagaacgaggtggctcgactaaagaagggctccggcagtggggaggaactcctgtcgaagaactatcatcttgaaaatgaggttgcaagacttaaaaaggggtccggatcaggtgaggaactactcagtaagaattaccacctggaaaacgaagttgcacgtttgaagaaaggatcaggatcaggcgaagaactgctctcaaaagattatcatttggaaaatgaggttgcacgtttaaaaaagggaagtggcagtggtgaggaacttctgtcgaaaaattatcatctcgagaatgaagtagcccgacttaaaaag

SEQ ID NO: 91 - protein, 5 aa, synthetic construct
GSGSG

SEQ ID NO: 92 - protein, 22 aa, synthetic construct
GSGSGGSGSGSGGSGSGGSGSG

SEQ ID NO: 93 - DNA, 20 bp, synthetic construct
  source 1..20: mol_type = other DNA
gggccagtctgcaaagcgag

SEQ ID NO: 94 - DNA, 20 bp, synthetic construct
  source 1..20: mol_type = other DNA
ttatgggcgtagctgaagaa

SEQ ID NO: 95 - DNA, 20 bp, synthetic construct
  source 1..20: mol_type = other DNA
gtatggagggctggatctgc

SEQ ID NO: 96 - RNA, 20 nt, synthetic construct
  source 1..20: mol_type = other RNA
gggccagtctgcaaagcgag

SEQ ID NO: 97 - RNA, 20 nt, synthetic construct
  source 1..20: mol_type = other RNA
ttatgggcgtagctgaagaa

SEQ ID NO: 98 - RNA, 20 nt, synthetic construct
  source 1..20: mol_type = other RNA
gtatggagggctggatctgc

SEQ ID NO: 99 - DNA, 20 bp, synthetic construct
  source 1..20: mol_type = other DNA
tacgttctctatcactgata

SEQ ID NO: 100 - DNA, 20 bp, synthetic construct
  source 1..20: mol_type = other DNA
tatggagggctggatctgcg

SEQ ID NO: 101 - RNA, 20 nt, synthetic construct
  source 1..20: mol_type = other RNA
tacgttctctatcactgata

SEQ ID NO: 102 - RNA, 20 nt, synthetic construct
  source 1..20: mol_type = other RNA
tatggagggctggatctgcg

SEQ ID NO: 103 - protein, 534 aa, synthetic construct
MDTQAGSVDEENGRQLGEVELQCGICTKWFTADTFGIDTSSCLPFMTNYSFHCNVCHHSGNTYFLRKQANLKEMCLSALANLTWQSRTQDEHPKTMFSKDKDIIPFIDKYWECMTTRQRPGKMTWPNNIVKTMSKERDVFLVKEHPDPGSKDPEEDYPKFGLLDQDLSNIGPAYDNQKQSSAVSTSGNLNGGIAAGSSGKGRGAKRKQQDGGTTGTTKKARSDPLFSAQRLPPHGYPLEHPFNKDGYRYILAEPDPHAPDPEKLELDCWAGKPIPGDLYRACLYERVLLALHDRAPQLKISDDRLTVVGEKGYSMVRASHGVRKGAWYFEITVDEMPPDTAARLGWSQPLGNLQAPLGYDKFSYSWRSKKGTKFHQSIGKHYSSGYGQGDVLGFYINLPEDTETAKSLPDTYKDKALIKFKSYLYFEEKDFVDKAEKSLKQTPHSEIIFYKNGVNQGVAYKDIFEGVYFPAISLYKSCTVSINFGPCFKYPPKDLTYRPMSDMGWGAVVEHTLADVLYHVETEVDGRRSPPWEP

SEQ ID NO: 104 - DNA, 1605 bp, synthetic construct
  source 1..1605: mol_type = other DNA
atggatactcaggcgggctccgtggatgaagagaatggccgacagttgggtgaggtagagctgcaatgtgggatttgtacaaaatggttcacggctgacacatttggcatagatacctcatcctgtctacctttcatgaccaactacagttttcattgcaacgtctgccatcacagtgggaatacctatttcctccggaagcaagcaaacttgaaggaaatgtgccttagtgctttggccaacctgacatggcagtcccgaacacaggatgaacatccgaagacaatgttctccaaagataaggatattataccatttattgataaatactgggagtgcatgacaaccagacagagacctgggaaaatgacttggccaaataacattgttaaaacaatgagtaaagaaagagatgtattcttggtaaaggaacacccagatccaggcagtaaagatccagaagaagattaccccaaatttggacttttggatcaggaccttagtaacattggtcctgcttatgacaaccaaaaacagagcagtgctgtgtctactagtgggaatttaaatgggggaattgcagcaggaagcagcggaaaaggacgaggagccaagcgcaaacagcaggatggagggaccacagggaccaccaagaaggcccggagtgaccctttgttttctgctcagcgccttccccctcatggctacccattggaacacccgtttaacaaagatggctatcggtatattctagctgagcctgatccgcacgcccctgaccccgagaagctggaacttgactgctgggcaggaaaacctattcctggagacctctacagagcctgcttgtatgaacgggttttgttagccctacatgatcgagctccccagttaaagatctcagatgaccggctgactgtggttggagagaagggctactctatggtgagggcctctcatggagtacggaagggtgcctggtattttgaaatcactgtggatgagatgccaccagataccgctgccagactgggttggtcccagcccctaggaaaccttcaagctcctttaggttatgataaatttagctattcttggcggagcaaaaagggaaccaagttccaccagtccattggcaaacactactcttctggctatggacagggagacgtcctgggattttatattaatcttcctgaagacacagagacagccaagtcattgccagacacatacaaagataaggctttgataaaattcaagagttatttgtattttgaggaaaaagactttgtggataaagcagagaagagcctgaagcagactccccatagtgagataatattttataaaaatggtgtcaatcaaggtgtggcttacaaagatatttttgagggggtttacttcccagccatctcactgtacaagagctgcacggtttccattaactttggaccatgcttcaagtatcctccgaaggatctcacttaccgccctatgagtgacatgggctggggcgccgtggtagagcacaccctggctgacgtcttgtatcacgtggagacagaagtggatgggaggcgcagtcccccatgggaaccctga

SEQ ID NO: 105 - protein, 203 aa, synthetic construct
MSGRSVRAETRSRAKDDIKKVMAAIEKVRKWEKKWVTVGDTSLRIFKWVPVTDSKEKEKSKSNSSAAREPNGFPSDASANSSLLLEFQDENSNQSSVSDVYQLKVDSSTNSSPSPQQSESLSPAHTSDFRTDDSQPPTLGQEILEEPSLPSSEVADEPPTLTKEEPVPLETQVVEEEEDSGAPPLKRFCVDQPTVPQTASESL

SEQ ID NO: 106 - DNA, 609 bp, synthetic construct
  source 1..609: mol_type = other DNA
atgtcgggccggtcggtccgggcggagacccgcagccgggccaaggacgacatcaagaaggtgatggcggccatcgagaaagtgcggaaatgggagaagaagtgggtgactgtgggtgacacgtccctgaggatatttaagtgggttcctgtgacagacagcaaggagaaagaaaagtcaaaatcgaacagttcagcagcccgagaacctaatggctttccttctgatgcctcagccaattcctctctccttcttgaattccaggacgaaaacagcaaccagagttccgtgtctgacgtctatcagcttaaggtggacagcagcaccaactcaagccccagcccccagcagagtgagtccctgagcccagcacacacctccgacttccgcacggatgactcccagcccccaacgctgggccaggagatcctggaggagccctccctgccctcctcggaagttgctgatgaacctcctaccctcaccaaggaagaaccagttccactagagacacaggtcgttgaggaagaggaagactcaggtgccccgcccctgaagcgcttctgtgtggaccaacccacagtgccgcagacggcgtcagaaagcttg

SEQ ID NO: 107 - protein, 204 aa, synthetic construct
MGEAEVGGGGAAGDKGPGEAATSPAEETVVWSPEVEVCLFHAMLGHKPVGVNRHFHMICIRDKFSQNIGRQVPSKVIWDHLSTMYDMQALHESEILPFPNPERNFVLPEEIIQEVREGKVMIEEEMKEEMKEDVDPHNGADDVFSSSGSLGKASEKSSKDKEKNSSDLGCKEGADKRKRSRVTDKVLTANSNPSSPSAAKRRRT

SEQ ID NO: 108 - DNA, 615 bp, synthetic construct
  source 1..615: mol_type = other DNA
atgggagaggccgaggtgggcggcgggggcgccgcaggcgacaagggcccgggggaggcggccaccagcccggcggaggagacagtggtgtggagccccgaggtggaggtgtgcctcttccacgccatgctgggccacaagcccgtcggtgtgaaccgacacttccacatgatttgtattcgggacaagttcagccagaacatcgggcggcaggtcccatccaaggtcatctgggaccatctgagcaccatgtacgacatgcaggcgctgcatgagtctgagattcttccattcccgaatccagagaggaacttcgtccttccagaagagatcattcaggaggtccgagaaggaaaagtgatgatagaagaggagatgaaagaggagatgaaggaagacgtggacccccacaatggggctgacgatgttttttcatcttcagggagtttggggaaagcatcagaaaaatccagcaaagacaaagagaagaactcctcagacttggggtgcaaagaaggcgcagacaagcggaagcgcagccgggtcaccgacaaagtcctgaccgcaaacagcaacccttccagtcccagtgctgccaagcggcgccgcacgtag

SEQ ID NO: 109 - protein, 468 aa, synthetic construct
MATGADVRDILELGGPEGDAASGTISKKDIINPDKKKSKKSSETLTFKRPEGMHREVYALLYSDKKDAPPLLPSDTGQGYRTVKAKLGSKKVRPWKWMPFTNPARKDGAMFFHWRRAAEEGKDYPFARFNKTVQVPVYSEQEYQLYLHDDAWTKAETDHLFDLSRRFDLRFVVIHDRYDHQQFKKRSVEDLKERYYHICAKLANVRAVPGTDLKIPVFDAGHERRRKEQLERLYNRTPEQVAEEEYLLQELRKIEARKKEREKRSQDLQKLITAADTTAEQRRTERKAPKKKLPQKKEAEKPAVPETAGIKFPDFKSAGVTLRSQRMKLPSSVGQKKIKALEQMLLELGVELSPTPTEELVHMFNELRSDLVLLYELKQACANCEYELQMLRHRHEALARAGVLGGPATPASGPGPASAEPAVTEPGLGPDPKDTIIDVVGAPLTPNSRKRRESASSSSSVKKAKKPL

SEQ ID NO: 110 - DNA, 1404 bp, synthetic construct
  source 1..1404: mol_type = other DNA
atggctacgggcgcggatgtacgggacattctagaactcgggggtccagaaggggatgcagcctctgggaccatcagcaagaaggacattatcaacccggacaagaaaaaatccaagaagtcctctgagacactgactttcaagaggcccgagggcatgcaccgggaagtctatgccttgctctactctgacaagaaggatgcacccccactgctacccagtgacactggccagggataccgtacagtgaaggccaagttgggctccaagaaggtgcggccttggaagtggatgccattcaccaacccggcccgcaaggacggagcaatgttcttccactggcgacgtgcagcggaggagggcaaggactacccctttgccaggttcaataagactgtgcaggtgcctgtgtactcggagcaggagtaccagctttatctccacgatgatgcttggactaaggcagaaactgaccacctctttgacctcagccgccgctttgacctgcgttttgttgttatccatgaccggtatgaccaccagcagttcaagaagcgttctgtggaagacctgaaggagcggtactaccacatctgtgctaagcttgccaacgtgcgggctgtgccaggcacagaccttaagataccagtatttgatgctgggcacgaacgacggcggaaggaacagcttgagcgtctctacaaccggaccccagagcaggtggcagaggaggagtacctgctacaggagctgcgcaagattgaggcccggaagaaggagcgggagaaacgcagccaggacctgcagaagctgatcacagcggcagacaccactgcagagcagcggcgcacggaacgcaaggcccccaaaaagaagctaccccagaaaaaggaggctgagaagccggctgttcctgagactgcaggcatcaagtttccagacttcaagtctgcaggtgtcacgctgcggagccaacggatgaagctgccaagctctgtgggacagaagaagatcaaggccctggaacagatgctgctggagcttggtgtggagctgagcccgacacctacggaggagctggtgcacatgttcaatgagctgcgaagcgacctggtgctgctctacgagctcaagcaggcctgtgccaactgcgagtatgagctgcagatgctgcggcaccgtcatgaggcactggcccgggctggtgtgctagggggccctgccacaccagcatcaggcccaggcccggcctctgctgagccggcagtgactgaacccggacttggtcctgaccccaaggacaccatcattgatgtggtgggcgcacccctcacgcccaattcgagaaagcgacgggagtcggcctccagctcatcttccgtgaagaaagccaagaagccgttg

SEQ ID NO: 111 - protein, 630 aa, synthetic construct
MAVPPGHGPFSGFPGPQEHTQVLPDVRLLPRRLPLAFRDATSAPLRKLSVDLIKTYKHINEVYYAKKKRRAQQAPPQDSSNKKEKKVLNHGYDDDNHDYIVRSGERWLERYEIDSLIGKGSFGQVVKAYDHQTQELVAIKIIKNKKAFLNQAQIELRLLELMNQHDTEMKYYIVHLKRHFMFRNHLCLVFELLSYNLYDLLRNTHFRGVSLNLTRKLAQQLCTALLFLATPELSIIHCDLKPENILLCNPKRSAIKIVDFGSSCQLGQRIYQYIQSRFYRSPEVLLGTPYDLAIDMWSLGCILVEMHTGEPLFSGSNEVDQMNRIVEVLGIPPAAMLDQAPKARKYFERLPGGGWTLRRTKELRKDYQGPGTRRLQEVLGVQTGGPGGRRAGEPGHSPADYLRFQDLVLRMLEYEPAARISPLGALQHGFFRRTADEATNTGPAGSSASTSPAPLDTCPSSSTASSISSSGGSSGSSSDNRTYRYSNRYCGGPGPPITDCEMNSPQVPPSQPLRPWAGGDVPHKTHQAPASASSLPGTGAQLPPQPRYLGRPPSPTSPPPPELMDVSLVGGPADCSPPHPAPAPQHPAASALRTRMTGGRPPLPPPDDPATLGPHLGLRGVPQSTAASSL

SEQ ID NO: 112 - DNA, 1890 bp, synthetic construct
  source 1..1890: mol_type = other DNA
atggccgtcccaccgggccatggtcccttctctggcttcccagggccccaggagcacacgcaggtattgcctgatgtgcggctactgcctcggaggctgcccctggccttccgggatgcaacctcagccccgctgcgtaagctctctgtggacctcatcaagacctacaagcacatcaatgaggtatactatgcgaagaagaagcggcgggcccagcaggcgccaccccaggattcgagcaacaagaaggagaagaaggtcctgaaccatggttatgatgacgacaaccatgactacatcgtgcgcagtggcgagcgctggctggagcgctacgaaattgactcgctcattggcaaaggctcctttggccaggtggtgaaagcctatgatcatcagacccaggagcttgtggccatcaagatcatcaagaacaaaaaggctttcctgaaccaggcccagattgagctgcggctgctggagctgatgaaccagcatgacacggagatgaagtactatatagtacacctgaagcggcacttcatgttccggaaccacctgtgcctggtatttgagctgctgtcctacaacctgtacgacctcctgcgcaacacccacttccgcggcgtctcgctgaacctgacccggaagctggcgcagcagctctgcacggcactgctctttctggccacgcctgagctcagcatcattcactgcgacctcaagcccgaaaacatcttgctgtgcaaccccaagcgcagcgccatcaagattgtggacttcggcagctcctgccagcttggccagaggatctaccagtatatccagagccgcttctaccgctcacctgaggtgctcctgggcacaccctacgacctggccattgacatgtggtccctgggctgcatccttgtggagatgcacaccggagagcccctcttcagtggctccaatgaggtcgaccagatgaaccgcattgtggaggtgctgggcatcccaccggccgccatgctggaccaggcgcccaaggctcgcaagtactttgaacggctgcctgggggtggctggaccctacgaaggacgaaagaactcaggaaggattaccagggccccgggacacggcggctgcaggaggtgctgggcgtgcagacgggcgggcccgggggccggcgggcgggggagccgggccacagccccgccgactacctccgcttccaggacctggtgctgcgcatgctggagtatgagcccgccgcccgcatcagccccctgggggctctgcagcacggcttcttccgccgcacggccgacgaggccaccaacacgggcccggcaggcagcagtgcctccacctcgcccgcgcccctcgacacctgcccctcttccagcaccgccagctccatctccagttctggaggctccagtggctcctccagtgacaaccggacctaccgctacagcaaccgatattgtgggggccctgggccccctatcacagactgtgagatgaacagcccccaggtcccaccctcccagccgctgcggccctgggcagggggtgatgtgccccacaagacacatcaagcccctgcctctgcctcgtcactgcctgggaccggggcccagttacccccccagccccgataccttggtcgtcccccatcaccaacctcaccaccacccccggagctgatggatgtgagcctggtgggcggccctgctgactgctccccacctcacccagcgcctgccccccagcacccggctgcctcagccctccggactcggatgactggaggtcgtccacccctcccgcctcctgatgaccctgccactctggggcctcacctgggcctccgtggtgtaccccagagcacagcagccagctcgttg

SEQ ID NO: 113 - protein, 268 aa, synthetic construct
MNGTANPLLDREEHCLRLGESFEKRPRASFHTIRYDFKPASIDTSCEGELQVGKGDEVTITLPHIPGSTPPMTVFKGNKRPYQKDCVLIINHDTGEFVLEKLSSSIQVKKTRAEGSSKIQARMEQQPTRPPQTSQPPPPPPPMPFRAPTKPPVGPKTSPLKDNPSPEPQLDDIKRELRAEVDIIEQMSSSSGSSSSDSESSSGSDDDSSSSGGEDNGPASPPQPSHQQPYNSRPAVANGTSRPQGSNQLMNALRNDLQLSESGSDSDD

SEQ ID NO: 114 - DNA, 807 bp, synthetic construct
  source 1..807: mol_type = other DNA
atgaatgggaccgcaaacccgctgctggaccgcgaggaacattgcctgaggctcggggagagcttcgagaagcggccgcgggcctccttccacactattcgttatgattttaaaccagcatctatagacacttcctgtgaaggagagcttcaagttggcaaaggagatgaagtcacaattacacttccacatatccctggatccacaccacccatgactgtgttcaaggggaacaaacggccttaccagaaagactgtgtgcttattattaatcatgacactggtgaatttgtgctggaaaaactcagtagcagcattcaggtgaagaaaacaagagctgagggcagcagtaaaatccaggcccgaatggaacagcagcccactcgtcctccacagacgtcacagccaccaccacctccaccacctatgccattcagagctccaacgaagcctccagttggacccaaaacttctcccttgaaagataacccctcacctgaacctcagttggatgacatcaaaagagagctgagggctgaagttgacattattgaacaaatgagcagcagcagtgggagcagctcttcagactctgagagctcttcgggaagtgatgacgatagctccagcagtggaggcgaggacaatggcccagcctctcctccgcagccttcacaccagcagccctacaacagtaggcctgccgttgccaatggaaccagccggccacaaggaagcaaccagctcatgaacgccctcagaaatgacttgcagttgagtgagtctggcagtgacagtgatgactag

SEQ ID NO: 115 - protein, 311 aa, synthetic construct
MDLKLKDCEFWYSLHGQVPGLLDWDMRNELFLPCTTDQCSLAEQILAKYRVGVMKPPEMPQKRRPSPDGDGPPCEPNLWMWVDPNILCPLGSQEAPKPSGKEDLTNISPFPQPPQKDEGSNCSEDKVVESLPSSSSEQSPLQKQGIHSPSDFELTEEEAEEPDDNSLQSPEMKCYQSQKLWQINNQEKSWQRPPLNCSHLIALALRNNPHCGLSVQEIYNFTRQHFPFFWTAPDGWKSTIHYNLCFLDSFEKVPDSLKDEDNARPRSCLWKLTKEGHRRFWEETRVLAFAQRERIQECMSQPELLTSLFDL

SEQ ID NO: 116 - DNA, 936 bp, synthetic construct
  source 1..936: mol_type = other DNA
atggacttaaaactaaaagactgtgaattttggtatagtctccatggccaggtcccagggctgctggactgggacatgaggaatgagttatttctgccttgtaccacagaccagtgctctttagctgagcaaatccttgccaaatacagagtcggagtaatgaagcccccagaaatgcctcagaagaggagacccagtcctgatggagatggtcctccctgtgaacccaatctgtggatgtgggtggaccccaatatcctgtgcccccttggcagccaggaggccccaaagcccagtggaaaagaggatctgacaaacatttctcctttccctcagcccccacaaaaagacgaagggtctaactgctcagaggacaaagtggtagagtctctgccatcttcctccagtgagcagtctcctttacagaagcagggtatccattcccccagtgactttgagctcacagaagaggaggctgaggaaccagacgacaactccctccagtcccctgaaatgaaatgttaccagagccagaaactatggcaaatcaacaaccaagagaagtcctggcaaaggccccctctcaattgtagccaccttattgccctagcattaagaaacaacccccactgtggcctcagtgtgcaggagatctacaatttcacccgacagcatttcccctttttctggacagctccggatggctggaagagcaccattcattacaacctctgcttcctggacagctttgagaaggtgccagacagccttaaggatgaagataatgcaagacctcgctcttgcctttggaagctcactaaggaggggcaccgccgcttttgggaggagactcgtgtcttagcctttgctcaaagggagagaatccaagagtgcatgagtcagccagagttgttgacctctctctttgatctttga

SEQ ID NO: 117 - protein, 484 aa, synthetic construct
MSGGGPSGGGPGGSGRARTSSFAEPGGGGGGGGGGPGGSASGPGGTGGGKASVGAMGGGVGASSSGGGPGGSGGGGSGGPGAGTSFPPPGVKLGRDSGKVTTVVATLGQGPERSQEVAYTDIKVIGNGSFGVVYQARLAETRELVAIKKVLQDKRFKNRELQIMRKLDHCNIVRLRYFFYSSGEKKDELYLNLVLEYVPETVYRVARHFTKAKLTIPILYVKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFKSRTPPEAIALCSSLLEYTPSSRLSPLEACAHSFFDELRCLGTQLPNNRPLPPLFNFSAGELSIQPSLNAILIPPHLRSPAGTTTLTPSSQALTETPTSSDWQSTDATPTLTNSSL

SEQ ID NO: 118 - DNA, 1452 bp, synthetic construct
  source 1..1452: mol_type = other DNA
atgagcggcggcgggccttcgggaggcggccctgggggctcgggcagggcgcggactagctcgttcgcggagcccggcggcggaggcggaggaggcggcggcggccccggaggctcggcctccggcccaggcggcaccggcggcggaaaggcatctgtcggggccatgggtgggggcgtcggggcctcgagctccgggggtggacccggcggcagcggcggaggaggcagcggaggccccggcgcaggcactagcttcccgccgcccggggtgaagctgggccgtgacagcgggaaggtgaccacagtcgtagccactctaggccaaggcccagagcgctcccaagaagtggcttacacggacatcaaagtgattggcaatggctcatttggggtcgtgtaccaggcacggctggcagagaccagggaactagtcgccatcaagaaggttctccaggacaagaggttcaagaaccgagagctgcagatcatgcgtaagctggaccactgcaatattgtgaggctgagatactttttctactccagtggcgagaagaaagacgagctttacctaaatctggtgctggaatatgtgcccgagacagtgtaccgggtggcccgccacttcaccaaggccaagttgaccatccctatcctctatgtcaaggtgtacatgtaccagctcttccgcagcttggcctacatccactcccagggcgtgtgtcaccgcgacatcaagccccagaacctgctggtggaccctgacactgctgtcctcaagctctgcgattttggcagtgcaaagcagttggtccgaggggagcccaatgtctcctacatctgttctcgctactaccgggccccagagctcatctttggagccactgattacacctcatccatcgatgtttggtcagctggctgtgtactggcagagctcctcttgggccagcccatcttccctggggacagtggggtggaccagctggtggagatcatcaaggtgctgggaacaccaacccgggaacaaatccgagagatgaaccccaactacacggagttcaagttccctcagattaaagctcacccctggacaaaggtgttcaaatctcgaacgccgccagaggccatcgcgctctgctctagcctgctggagtacaccccatcctcaaggctctccccactagaggcctgtgcgcacagcttctttgatgaactgcgatgtctgggaacccagctgcctaacaaccgcccacttccccctctcttcaacttcagtgctggtgaactctccatccaaccgtctctcaacgccattctcatccctcctcacttgaggtccccagcgggcactaccaccctcaccccgtcctcacaagctttaactgagactccgaccagctcagactggcagtcgaccgatgccacacctaccctcactaactcctccttg

SEQ ID NO: 119 - protein, 244 aa, synthetic construct
MTGIAAASFFSNTCRFGGCGLHFPTLADLIEHIEDNHIDTDPRVLEKQELQQPTYVALSYINRFMTDAARREQESLKKKIQPKLSLTLSSSVSRGNVSTPPRHSSGSLTPPVTPPITPSSSFRSSTPTGSEYGEEEVDYEESDSDESWTTESAISSEAILSSMCMNGGEEKPFACPVPGCKKRYKNVNGIKYHAKNGHRTQIRVRKPFKCRCGKSYKTAQGLRHHTINFHPPVSAEIIRKMQQL

SEQ ID NO: 120 - DNA, 732 bp, synthetic construct
  source 1..732: mol_type = other DNA
atgacaggcatcgccgccgcctccttcttctccaatacctgccgattcgggggctgcggactccacttccccaccctggccgacctcatcgagcacatcgaggacaaccacatcgatacagatccacgggttttagaaaaacaagaattacagcagccaacctatgttgccctgagttacataaatagattcatgacagatgctgcccgccgagagcaggagtccctaaagaagaagattcagccgaagctctcgctgactctgtccagctcagtgtctcgagggaatgtgtccactcccccacgccacagcagtggaagccttactccccccgtgaccccacccatcaccccctcctcttcattccgcagcagcactccgacaggcagcgagtatggcgaggaggaggtggactatgaggagtcggacagcgatgagtcctggaccacagagagtgccatcagctccgaagccatcctcagctccatgtgcatgaatggaggggaagagaagccttttgcctgcccagttcctggatgtaaaaagagatacaagaatgtgaatggcataaagtatcacgctaagaatggtcacagaacacagattcgtgtccgcaaaccattcaagtgtcgctgtgggaagagttacaagacagctcagggcctgcggcaccacacaatcaatttccatcccccggtgtcggctgagattatcaggaagatgcagcaattg

SEQ ID NO: 121 - protein, 611 aa, synthetic construct
MPRRKRNAGSSSDGTEDSDFSTDLEHTDSSESDGTSRRSARVTRSSARLSQSSQDSSPVRNLQSFGTEEPAYSTRRVTRSQQQPTPVTPKKYPLRQTRSSGSETEQVVDFSDRETKNTADHDESPPRTPTGNAPSSESDIDISSPNVSHDESIAKDMSLKDSGSDLSHRPKRRRFHESYNFNMKCPTPGCNSLGHLTGKHERHFSISGCPLYHNLSADECKVRAQSRDKQIEERMLSHRQDDNNRHATRHQAPTERQLRYKEKVAELRKKRNSGLSKEQKEKYMEHRQTYGNTREPLLENLTSEYDLDLFRRAQARASEDLEKLRLQGQITEGSNMIKTIAFGRYELDTWYHSPYPEEYARLGRLYMCEFCLKYMKSQTILRRHMAKCVWKHPPGDEIYRKGSISVFEVDGKKNKIYCQNLCLLAKLFLDHKTLYYDVEPFLFYVMTEADNTGCHLIGYFSKEKNSFLNYNVSCILTMPQYMRQGYGKMLIDFSYLLSKVEEKVGSPERPLSDLGLISYRSYWKEVLLRYLHNFQGKEISIKEISQETAVNPVDIVSTLQALQMLKYWKGKHLVLKRQDLIDEWIAKEAKRSNSNKTMDPSCLKWTPPKGT

SEQ ID NO: 122 - DNA, 1836 bp, synthetic construct
  source 1..1836: mol_type = other DNA
atgccgcgaaggaagaggaatgcaggcagtagttcagatggaaccgaagattccgatttttctacagatctcgagcacacagacagttcagaaagtgatggcacatcccgacgatctgctcgagtcacccgctcctcagccaggctaagccagagttctcaagattccagtcctgttcgaaatctgcagtcttttggcactgaggagcctgcttactctaccagaagagtgacccgtagtcagcagcagcctaccccagtgacaccgaaaaaataccctcttcggcagactcgttcatctggttcagaaactgagcaagtggttgatttttcagatagagaaactaaaaatacagctgatcatgatgagtcaccgcctcgaactccaactggaaatgcgccttcttctgagtctgacatagacatctccagccccaatgtatctcacgatgagagcattgccaaggacatgtccctgaaggactcaggcagtgatctctctcatcgccccaagcgccgtcgcttccatgaaagctacaacttcaatatgaagtgtcctacaccaggctgtaactctctaggacaccttacaggaaaacatgagagacatttctccatctcaggatgcccactgtatcataacctctcagctgacgaatgcaaggtgagagcacagagccgggataagcagatagaagaaaggatgctgtctcacaggcaagatgacaacaacaggcatgcaaccaggcaccaggcaccaacggagaggcagcttcgatataaggaaaaagtggctgaactcaggaagaaaagaaattctggactgagcaaagaacagaaagagaaatatatggaacacagacagacctatgggaacacacgggaacctcttttagaaaacctgacaagcgagtatgacttggatcttttccgaagagcacaagcccgggcttcagaggatttggagaagttaaggctgcaaggccaaatcacagagggaagcaacatgattaaaacaattgcttttggccgctatgagcttgatacctggtaccattctccatatcctgaagaatatgcacggctgggacgtctctatatgtgtgaattctgtttaaaatatatgaagagccaaacgatactccgccggcacatggccaaatgtgtgtggaaacacccacctggtgatgagatatatcgcaaaggttcaatctctgtgtttgaagtggatggcaagaaaaacaagatctactgccaaaacctgtgcctgttggccaaactttttctggaccacaagacattatattatgatgtggagcccttcctgttctatgttatgacagaggcggacaacactggctgtcacctgattggatatttttctaaggaaaagaattcattcctcaactacaacgtctcctgtatccttactatgcctcagtacatgagacagggctatggcaagatgcttattgatttcagttatttgctttccaaagtcgaagaaaaagttggctccccagaacgtccactctcagatctggggcttataagctatcgcagttactggaaagaagtacttctccgctacctgcataattttcaaggcaaagagatttctatcaaagaaatcagtcaggagacggctgtgaatcctgtggacattgtcagcactctgcaagcccttcagatgctcaaatactggaagggaaaacacctagttttaaagagacaggacctgattgatgagtggatagccaaagaggccaaaaggtccaactccaataaaaccatggatcccagctgcttaaaatggacccctcccaagggcacttaa

SEQ ID NO: 123 - protein, 625 aa, synthetic construct
MQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTCL

SEQ ID NO: 124 - DNA, 1875 bp, synthetic construct
  source 1..1875: mol_type = other DNA
atgcagccagatcccaggcctagcggggctggggcctgctgccgattcctgcccctgcagtcacagtgccctgagggggcaggggacgcggtgatgtacgcctccactgagtgcaaggcggaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttcctggtgcagcagctggaccccagcaatgccatcggcatcgccaacttcgctgagcagattggctgtgtggagttgcaccagcgtgcccgggagtacatctacatgcattttggggaggtggccaagcaagaggagttcttcaacctgtcccactgccaactggtgaccctcatcagccgggacgacctgaacgtgcgctgcgagtccgaggtcttccacgcctgcatcaactgggtcaagtacgactgcgaacagcgacggttctacgtccaggcgctgctgcgggccgtgcgctgccactcgttgacgccgaacttcctgcagatgcagctgcagaagtgcgagatcctgcagtccgactcccgctgcaaggactacctggtcaagatcttcgaggagctcaccctgcacaagcccacgcaggtgatgccctgccgggcgcccaaggtgggccgcctgatctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgtttg

SEQ ID NO: 125 - protein, 191 aa, synthetic construct
MAMHNKAAPPQIPDTRRELAELVKRKQELAETLANLERQIYAFEGSYLEDTQMYGNIIRGWDRYLTNQKNSNSKNDRRNRKFKEAERLFSKSSVTSAAAVSALAGVQDQLIEKREPGSGTESDTSPDFHNQENEPSQEDPEDLDGSVQGVKPQKAASSTSSGSHHSSHKKRKNKNRHRIDLKLNKKPRADY

SEQ ID NO: 126 - DNA, 576 bp, synthetic construct
  source 1..576: mol_type = other DNA
atggcgatgcacaacaaggcggcgccgccgcagatcccggacacccggcgggagctggcggagctcgtgaagcggaagcaggagctggcggaaacattggcaaatttggagcgacagatctatgcttttgagggaagctacctggaagacactcagatgtatggcaatattattcgtggctgggatcggtatctgaccaaccaaaaaaactccaatagcaaaaatgatcgaaggaaccggaagtttaaggaagctgagcggctcttcagtaaatcctcggttacctcagcagctgcagtaagtgcattggcaggagttcaggaccagctcattgaaaagagggagccaggaagtgggacggaaagtgacacttctccagacttccacaatcaggaaaatgagcccagccaggaggaccctgaggatctggatggatctgtgcagggagtgaaacctcagaaggctgcttcttctacttcctcagggagtcaccacagcagccataaaaagcgaaagaataaaaaccggcacaggattgatctgaagttaaacaaaaaaccacgagctgactattag

SEQ ID NO: 127 - protein, 462 aa, synthetic construct
MGAVNPLLSQAESSHTEPDLEDCSFRCRGTSPQESLSSMSPISSLPALFDQTASAPCGGGQLDPAAPGTTNMEQLLEKQGDGEAGVNIVEMLKALHALQKENQRLQEQILSLTAKKERLQILNVQLSVPFPALPAALPAANGPVPGPYGLPPQAGSSDSLSTSKSPPGKSSLGLDNSLSTSSEDPHSGCPSRSSSSLSFHSTPPPLPLLQQSPATLPLALPGAPAPLPPQPQNGLGRAPGAAGLGAMPMAEGLLGGLAGSGGLPLNGLLGGLNGAAAPNPASLSQAGGAPTLQLPGCLNSLTEQQRHLLQQQEQQLQQLQQLLASPQLTPEHQTVVYQMIQQIQQKRELQRLQMAGGSQLPMASLLAGSSTPLLSAGTPGLLPTASAPPLLPAGALVAPSLGNNTSLMAAAAAAAAVAAAGGPPVLTAQTNPFLSLSGAEGSGGGPKGGTADKGASANQEKG

SEQ ID NO: 128 - DNA, 1389 bp, synthetic construct
  source 1..1389: mol_type = other DNA
atgggtgccgttaatcccctcctctcccaagctgagagcagccacacagagccagacctggaggactgcagcttccggtgtcgggggacctcccctcaggagagtctgtcttccatgtcccccatcagcagcctccccgcactcttcgaccagacagcctctgcaccctgtgggggcggccagttagacccggcggccccagggacgactaacatggagcagcttctggagaagcagggcgacggggaggccggcgtcaacatcgtggagatgctgaaggcgctgcacgcgctgcagaaggagaaccagcggctgcaagagcagatcctgagcctgacggccaaaaaggagcggctgcagattctcaacgtgcagctctctgtgcccttccctgccctgcctgctgccctgcctgccgccaacggccctgtccctgggccctatggcctgcctccccaagccggcagcagcgactccttgagcaccagcaagagccctccgggaaagagcagcctcggcctggacaactcgctgtccacttcttctgaggacccacactcaggctgcccgagccgcagcagctcgtcgctgtccttccacagcacgcccccaccgctgcccctcctccagcagagccctgccactctgcccctggccctgcctggggcccctgccccactcccgccccagccgcagaacgggttgggccgggcacccggggcagcggggctgggggccatgcccatggctgaggggctgttgggggggctggcaggcagtgggggcctgcccctcaatgggctccttggggggttgaatggggccgctgcccccaaccccgcaagcttgagccaggctggcggggcccccacgctgcagctgccaggctgtctcaacagccttacagagcagcagagacatctccttcagcagcaagagcagcagctccagcaactccagcagctcctggcctccccgcagctgaccccggaacaccagactgttgtctaccagatgatccagcagatccagcagaaacgggagctgcagcgcctgcagatggctgggggctcccagctgcccatggccagcctgctggcaggaagctccaccccgctgctgtctgcgggtacccctggcctgctgcccacagcgtctgctccacccctgctgcccgctggagccctagtggctccctcgcttggcaacaacacaagtctcatggccgcagcagctgcagctgcagcagtagcagcagcaggcggacctccagtcctcactgcccagaccaaccccttcctcagcctgtcgggagcagagggcagtggcggtggccccaaaggagggaccgctgacaaaggagcctcagccaaccaggaaaaaggctaa

SEQ ID NO: 129 - protein, 288 aa, synthetic construct
MSSRKQGSQPRGQQSAEEENFKKPTRSNMQRSKMRGASSGKKTAGPQQKNLEPALPGRWGGRSAENPPSGSVRKTRKNKQKTPGNGDGGSTSEAPQPPRKKRARADPTVESEEAFKNRMEVKVKIPEELKPWLVEDWDLVTRQKQLFQLPAKKNVDAILEEYANCKKSQGNVDNKEYAVNEVVAGIKEYFNVMLGTQLLYKFERPQYAEILLAHPDAPMSQVYGAPHLLRLFVRIGAMLAYTPLDEKSLALLLGYLHDFLKYLAKNSASLFTASDYKVASAEYHRKAL

SEQ ID NO: 130 - DNA, 867 bp, synthetic construct
  source 1..867: mol_type = other DNA
atgagttccagaaagcagggttctcaacctcgtggacagcaatctgcagaagaagagaacttcaaaaaaccaactagaagcaacatgcagagaagtaaaatgagaggggcctcctcaggaaagaagacagctggtccacagcagaaaaatcttgaaccagctctcccaggaagatggggtggtcgctctgcagagaaccccccttcaggatccgtgaggaagaccagaaagaacaagcagaagactcctggaaacggagatggtggcagtaccagcgaagcacctcagccccctcggaagaaaagggcccgggcagaccccactgttgaaagtgaggaggcgtttaagaatagaatggaggttaaagtgaagattcctgaagaattaaaaccatggcttgttgaggactgggacttagttaccaggcagaagcagctgtttcaactccctgccaagaaaaatgtagatgcaattctggaggagtatgcaaattgcaagaaatcgcagggaaatgttgataataaggaatatgcggttaatgaagttgtggcaggaataaaagaatatttcaatgtgatgttgggcactcagctgctctacaaatttgagaggccccagtatgctgaaatcctcttggctcaccctgatgctccaatgtcccaggtttatggagcaccacacctactgagattatttgtaagaattggagcaatgttggcctatacgccccttgatgagaaaagccttgcattattgttgggctatttgcatgatttcctaaaatatctggcaaagaattctgcatctctctttactgccagtgattacaaagtggcttctgctgagtaccaccgcaaagccctgtga

SEQ ID NO: 131 - protein, 335 aa, synthetic construct
MSTEGGFGGTSSSDAQQSLQSFWPRVMEEIRNLTVKDFRVQELPLARIKKIMKLDEDVKMISAEAPVLFAKAAQIFITELTLRAWIHTEDNKRRTLQRNDIAMAITKFDQFDFLIDIVPRDELKPPKRQEEVRQSVTPAEPVQYYFTLAQQPTAVQVQGQQQGQQTTSSTTTIQPGQIIIAQPQQGQTTPVTMQVGEGQQVQIVQAQPQGQAQQAQSGTGQTMQVMQQIITNTGEIQQIPVQLNAGQLQYIRLAQPVSGTQVVQGQIQTLATNAQQITQTEVQQGQQQFSQFTDGQQLYQIQQVTMPAGQDLAQPMFIQSANQPSDGQAPQVTGD

SEQ ID NO: 132 - DNA, 1008 bp, synthetic construct
  source 1..1008: mol_type = other DNA
atgtccacagaaggaggatttggtggtactagcagcagtgatgcccagcaaagcctacagtcgttctggcctcgggtcatggaagaaatccggaatttaacagtgaaagacttccgagtgcaggaactcccactggctcgtattaagaagattatgaaactggatgaagatgtgaagatgatcagtgcagaagcgcctgtactctttgccaaggcagcccagatttttatcacagagttgactcttcgagcctggattcacacagaagataacaagcgccggactctacagagaaatgatatcgccatggcaattacaaaatttgatcagtttgattttctcatcgatattgttccaagagatgaactgaaacctccaaagcgtcaggaggaggtgcgccagtctgtaactcctgccgagccagtccagtactatttcacgctggctcagcaacccaccgctgtccaagtccagggccagcagcaaggccagcagaccaccagctccacgaccaccatccagcctgggcagatcatcatcgcacagcctcagcagggccagaccacacctgtgacaatgcaggttggagaaggtcagcaggtgcagattgtccaggctcagccacagggtcaagcccaacaggcccagagtggcactggacagaccatgcaggtgatgcagcagatcatcactaacacaggagagatccagcagatcccggtgcagctgaatgccggccagctgcagtatatccgcttagcccagcctgtatcaggcactcaagttgtgcagggacagatccagacacttgccaccaatgctcaacagattacacagacagaggtccagcaaggacagcagcagttcagccagttcacagatggacagcagctctaccagatccagcaagtcaccatgcctgcgggccaggacctcgcccagcccatgttcatccagtcagccaaccagccctccgacgggcaggccccccaggtgaccggcgactga

SEQ ID NO: 133 - protein, 791 aa, synthetic construct
MEEKRRKYSISSDNSDTTDSHATSTSASRCSKLPSSTKSGWPRQNEKKPSEVFRTDLITAMKIPDSYQLSPDDYYILADPWRQEWEKGVQVPAGAEAIPEPVVRILPPLEGPPAQASPSSTMLGEGSQPDWPGGSRYDLDEIDAYWLELINSELKEMERPELDELTLERVLEELETLCHQNMARAIETQEGLGIEYDEDVVCDVCRSPEGEDGNEMVFCDKCNVCVHQACYGILKVPTGSWLCRTCALGVQPKCLLCPKRGGALKPTRSGTKWVHVSCALWIPEVSIGCPEKMEPITKISHIPASRWALSCSLCKECTGTCIQCSMPSCVTAFHVTCAFDHGLEMRTILADNDEVKFKSFCQEHSDGGPRNEPTSEPTEPSQAGEDLEKVTLRKQRLQQLEEDFYELVEPAEVAERLDLAEALVDFIYQYWKLKRKANANQPLLTPKTDEVDNLAQQEQDVLYRRLKLFTHLRQDLERVRNLCYMVTRRERTKHAICKLQEQIFHLQMKLIEQDLCRAGLSTSFPIDGTFFNSWLAQSVQITAENMAMSEWPLNNGHREDPAPGLLSEELLQDEETLLSFMRDPSLRPGDPARKARGRTRLPAKKKPPPPPPQDGPGSRTTPDKAPKKTWGQDAGSGKGGQGPPTRKPPRRTSSHLPSSPAAGDCPILATPESPPPLAPETPDEAASVAADSDVQVPGPAASPKPLGRLRPPRESKVTRRLPGARPDAGMGPPSAVAERPKVSLHFDTETDGYFSDGEMSDSDVEAEDGGVQRGPREAGAEEVVRMGVLAS

SEQ ID NO: 134 - DNA, 2376 bp, synthetic construct
  source 1..2376: mol_type = other DNA
atggaagagaagaggcgaaaatactccatcagcagtgacaactctgacaccactgacagtcatgcgacatctacatccgcatcaagatgctccaaactgcccagcagcaccaagtcgggctggccccgacagaacgaaaagaagccctccgaggttttccggacagacttgatcacagccatgaagatcccggactcataccagctcagcccggatgactactacatcctggcagacccatggcgacaggaatgggagaaaggtgtgcaggtgcctgccggggcagaggccatcccagagcccgtggtgaggatcctcccaccactggaaggcccccctgcccaggcatccccgagcagcaccatgcttggtgagggctcccagcctgattggccagggggcagccgctatgacttggacgagattgatgcctactggctggagctcatcaactcggagcttaaggagatggagaggccggagctggacgagctgacattagagcgtgtgctggaggagctggagaccctgtgccaccagaatatggccagggccattgagacgcaggaggggctgggcatcgagtacgacgaggatgttgtctgcgacgtgtgtcgctctcctgagggcgaggatggcaacgagatggtcttctgtgacaagtgcaacgtctgtgtgcatcaggcatgctacgggatcctcaaggtgcccacgggcagctggctgtgccggacgtgtgccctgggtgtccagccaaagtgcctgctctgccccaagcgaggaggagccttgaagcccactagaagtgggaccaagtgggtgcatgtcagctgtgccctatggattcctgaggtcagcatcggctgcccagagaagatggagcccatcaccaagatctcgcatatcccagccagccgctgggctctgtcctgcagcctctgcaaggaatgcacaggcacctgcatccagtgttccatgccttcctgcgtcacagcgttccatgtcacatgcgcctttgaccacggcctggaaatgcggactatattagcagacaacgatgaggtcaagttcaagtcattctgccaggagcacagtgacgggggcccacgtaatgagcccacatctgagcccacggaacccagccaggctggcgaggacctggaaaaggtgaccctgcgcaagcagcggctgcagcagctagaggaggacttctacgagctggtggagccggctgaggtggctgagcggctggacctggctgaggcactggtcgacttcatctaccagtactggaagctgaagaggaaagccaatgccaaccagccgctgctgacccccaagaccgacgaggtggacaacctggcccagcaggagcaggacgtcctctaccgccgcctgaagctcttcacccatctgcggcaggacctagagagggttagaaatctgtgctacatggtgacaaggcgcgagagaacgaaacacgccatctgcaaactccaggagcagatattccacctgcagatgaaacttattgaacaggatctgtgtcgagcaggcctgtccacctcattccccatcgatggcaccttcttcaacagctggctggcacagtcggtgcagatcacagcagagaacatggccatgagcgagtggccactgaacaatgggcaccgcgaggaccctgctccagggctgctgtcagaggaactgctgcaggacgaggagacactgctcagcttcatgcgggacccctcgctgcgacctggtgaccctgctaggaaggcccgaggccgcacccgcctgcctgccaagaagaaaccaccaccaccaccaccgcaggacgggcctggttcacggacgactccagacaaagcccccaagaagacctggggccaggatgcaggcagtggcaaggggggtcaagggccacctaccaggaagccaccacgtcggacatcttctcacttgccgtccagccctgcagccggggactgtcccatcctagccacccctgaaagccccccgccactggcccctgagaccccggacgaggcagcctcagtagctgctgactcagatgtccaagtgcctggccctgcagcaagccctaagcctttgggccggctccggccaccccgcgagagcaaggtaacccggagattgccgggtgccaggcctgatgctgggatgggaccaccttcagctgtggctgagaggcccaaggtcagcctgcattttgacactgagactgatggctacttctctgatggggagatgagcgactcagatgtagaggccgaggacggtggggtgcagcggggtccccgggaggcaggggcagaggaggtggtccgcatgggcgtactggcctcctaa

SEQ ID NO: 135 - protein, 84 aa, synthetic construct
MRTDSSKMTDVESGVANFASSARAGRRNALPDIQSSAATDGTSDLPLKLEALSVKEDAKEKDEKTTQDQLEKPQNEEKCPTFLY

SEQ ID NO: 136 - DNA, 252 bp, synthetic construct
  source 1..252: mol_type = other DNA
atgaggacagattcatcaaaaatgactgacgtggagtctggggtcgccaattttgcatcttcagcaagggcaggccgccggaatgccttaccagacatccagagttcagctgccacagacggaacctcagatttgcccctcaaactggaggctctctccgtgaaggaagatgcaaaagagaaagatgaaaaaacaacacaagaccaattggaaaagcctcaaaatgaagaaaaatgcccaactttcttgtac

SEQ ID NO: 137 - protein, 117 aa, synthetic construct
MAAAAAAGSGTPREEEVPAGEAAASQPQAPTSVPGARLSRLPLARVKALVKADPDVTLAGQEAIFILARAAELFVETIAKDAYCCAQQGKRKTLQRRDLDNAIEAVDEFAFLEGTLD

SEQ ID NO: 138 - DNA, 354 bp, synthetic construct
  source 1..354: mol_type = other DNA
atggcggcggcggcggcggcaggaagcgggacgccccgagaggaggaggtacctgctggggaggcagcggcctcgcagccccaggccccaacgagtgtgcctggggctcgtctctcgaggttgcctctggcgcgagtgaaggccttggtgaaggcagatcccgacgtgacgctagcgggacaggaagccatcttcattctggcacgagccgcggaactgtttgtggagaccattgcaaaagatgcctactgttgcgctcagcagggaaaaaggaaaacccttcagaggagagacttggataatgcaatagaagctgtggatgaatttgcttttctggaaggtactttagattga

SEQ ID NO: 139 - protein, 761 aa, synthetic construct
MPNFCAAPNCTRKSTQSDLAFFRFPRDPARCQKWVENCRRADLEDKTPDQLNKHYRLCAKHFETSMICRTSPYRTVLRDNAIPTIFDLTSHLNNPHSRHRKRIKELSEDEIRTLKQKKIDETSEQEQKHKETNNSNAQNPSEEEGEGQDEDILPLTLEEKENKEYLKSLFEILILMGKQNIPLDGHEADEIPEGLFTPDNFQALLECRINSGEEVLRKRFETTAVNTLFCSKTQQRQMLEICESCIREETLREVRDSHFFSIITDDVVDIAGEEHLPVLVRFVDESHNLREEFIGFLPYEADAEILAVKFHTMITEKWGLNMEYCRGQAYIVSSGFSSKMKVVASRLLEKYPQAIYTLCSSCALNMWLAKSVPVMGVSVALGTIEEVCSFFHRSPQLLLELDNVISVLFQNSKERGKELKEICHSQWTGRHDAFEILVELLQALVLCLDGINSDTNIRWNNYIAGRAFVLCSAVSDFDFIVTIVVLKNVLSFTRAFGKNLQGQTSDVFFAAGSLTAVLHSLNEVMENIEVYHEFWFEEATNLATKLDIQMKLPGKFRRAHQGNLESQLTSESYYKETLSVPTVEHIIQELKDIFSEQHLKALKCLSLVPSVMGQLKFNTSEEHHADMYRSDLPNPDTLSAELHCWRIKWKHRGKDIELPSTIYEALHLPDIKFFPNVYALLKVLCILPVMKVENERYENGRKRLKAYLRNTLTDQRSSNLALLNINFDIKHDLDLMVDTYIKLYTSKSELPTDNSETVENT

SEQ ID NO: 140 - DNA, 2286 bp, synthetic construct
  source 1..2286: mol_type = other DNA
atgccgaacttctgcgctgcccccaactgcacgcggaagagcacgcagtccgacttggccttcttcaggttcccgcgggaccctgccagatgccagaagtgggtggagaactgtaggagagcagacttagaagataaaacacctgatcagctaaataaacattatcgattatgtgccaaacattttgagacctctatgatctgtagaactagtccttataggacagttcttcgagataatgcaataccaacaatatttgatcttaccagtcatttgaacaacccacatagtagacacagaaaacgaataaaagaactgagtgaagatgaaatcaggacactgaaacagaaaaaaattgatgaaacttctgagcaggaacaaaaacataaagaaaccaacaatagcaatgctcagaaccccagcgaagaagagggtgaagggcaagatgaggacattttacctctaacccttgaagagaaggaaaacaaagaatacctaaaatctctatttgaaatcttgattctgatgggaaagcaaaacatacctctggatggacatgaggctgatgaaatcccagaaggtctctttactccagataactttcaggcactgctggagtgtcggataaattctggtgaagaggttctgagaaagcggtttgagacaacagcagttaacacgttgttttgttcaaaaacacagcagaggcagatgctagagatctgtgagagctgtattcgagaagaaactctcagggaagtgagagactcacacttcttttccattatcactgacgatgtagtggacatagcaggggaagagcacctacctgtgttggtgaggtttgttgatgaatctcataacctaagagaggaatttataggcttcctgccttatgaagccgatgcagaaattttggctgtgaaatttcacactatgataactgagaagtggggattaaatatggagtattgtcgtggccaggcttacattgtctctagtggattttcttccaaaatgaaagttgttgcttctagacttttagagaaatatccccaagctatctacacactctgctcttcctgtgccttaaatatgtggttggcaaaatcagtacctgttatgggagtatctgttgcattaggaacaattgaggaagtttgttcttttttccatcgatcaccacaactgcttttagaacttgacaacgtaatttctgttctttttcagaacagtaaagaaaggggtaaagaactgaaggaaatctgccattctcagtggacaggcaggcatgatgcttttgaaattttagtggaactcctgcaagcacttgttttatgtttagatggtataaatagtgacacaaatattagatggaataactatatagctggccgagcatttgtactctgcagtgcagtgtcagattttgatttcattgttactattgttgttcttaaaaatgtcctatcttttacaagagcctttgggaaaaacctccaggggcaaacctctgatgtcttctttgcggccggtagcttgactgcagtactgcattcactcaacgaagtgatggaaaatattgaagtttatcatgaattttggtttgaggaagccacaaatttggcaaccaaacttgatattcaaatgaaactccctgggaaattccgcagagctcaccagggtaacttggaatctcagctaacctctgagagttactataaagaaaccctaagtgtcccaacagtggagcacattattcaggaacttaaagatatattctcagaacagcacctcaaagctcttaaatgcttatctctggtaccctcagtcatgggacaactcaaattcaatacgtcggaggaacaccatgctgacatgtatagaagtgacttacccaatcctgacacgctgtcagctgagcttcattgttggagaatcaaatggaaacacagggggaaagatatagagcttccgtccaccatctatgaagccctccacctgcctgacatcaagttttttcctaatgtgtatgcattgctgaaggtcctgtgtattcttcctgtgatgaaggttgagaatgagcggtatgaaaatggacgaaagcgtcttaaagcatatttgaggaacactttgacagaccaaaggtcaagtaacttggctttgcttaacataaattttgatataaaacacgacctggatttaatggtggacacatatattaaactctatacaagtaagtcagagcttcctacagataattccgaaactgtggaaaatacctaa

SEQ ID NO: 141 - protein, 407 aa, synthetic construct
MAASAPPPPDKLEGGGGPAPPPAPPSTGRKQGKAGLQMKSPEKKRRKSNTQGPAYSHLTEFAPPPTPMVDHLVASNPFEDDFGAPKVGVAAPPFLGSPVPFGGFRVQGGMAGQVPPGYSTGGGGGPQPLRRQPPPFPPNPMGPAFNMPPQGPGYPPPGNMNFPSQPFNQPLGQNFSPPSGQMMPGPVGGFGPMISPTMGQPPRAELGPPSLSQRFAQPGAPFGPSPLQRPGQGLPSLPPNTSPFPGPDPGFPGPGGEDGGKPLNPPASTAFPQEPHSGSPAAAVNGNQPSFPPNSSGRGGGTPDANSLAPPGKAGGGSGPQPPPGLVYPCGACRSEVNDDQDAILCEASCQKWFHRECTGMTESAYGLLTTEASAVWACDLCLKTKEIQSVYIREGMGQLVAANDGL

SEQ ID NO: 142 - DNA, 1221 bp, synthetic construct
  source 1..1221: mol_type = other DNA
atggccgcctcggcgccgcccccaccggacaagctggagggaggtggcggccccgcaccgccccctgcgccgcccagcaccgggaggaagcagggcaaggccggtctgcaaatgaagagtccagaaaagaagcgaaggaagtcaaatactcagggccctgcatactcacatctgacggagtttgcaccacccccaactcccatggtggatcacctggttgcatccaacccttttgaagatgacttcggagcccccaaagtgggggttgcagcccctccattccttggcagtcctgtgcccttcggaggcttccgtgtgcaggggggcatggcgggccaggtacccccaggctacagcactggaggtggagggggcccccagccactccgtcgacagccaccccccttccctcccaatcctatgggccctgctttcaacatgcccccccagggtcctggctacccacccccaggcaacatgaactttcccagccaacccttcaaccagcctctgggtcaaaactttagtcctcccagtgggcagatgatgccgggcccagtggggggatttggtcccatgatctcacccaccatgggacagcctcccagagcagagctgggcccaccttctctgtcccaacgatttgctcagccaggggctccttttggcccttctcctctccagagacctggtcaggggctccccagcctgccgcctaacacaagtccctttcctggtccggaccctggctttcctggccctggtggtgaggatggggggaagcccttgaatccacctgcttctactgcttttccccaggagccccactcaggctccccggctgctgctgttaatgggaaccagcccagtttccccccgaacagcagtgggcggggtgggggcactccagatgccaacagcttggcaccccctggcaaggcaggtgggggctccgggccccagcctcccccaggcttggtgtacccatgtggtgcctgtcggagtgaggtgaacgatgaccaggatgccattctgtgtgaggcctcctgccagaaatggttccaccgtgagtgcacaggcatgactgagagcgcctatgggctgctgaccactgaagcttctgccgtctgggcctgcgatctctgcctcaagaccaaggagatccagtctgtctacatccgtgagggcatggggcagctggtggctgctaacgatgggttg

SEQ ID NO: 143 - protein, 206 aa, synthetic construct
MAAAKDTHEDHDTSTENTDESNHDPQFEPIVSLPEQEIKTLEEDEEELFKMRAKLFRFASENDLPEWKERGTGDVKLLKHKEKGAIRLLMRRDKTLKICANHYITPMMELKPNAGSDRAWVWNTHADFADECPKPELLAIRFLNAENAQKFKTKFEECRKEIEEREKKAGSGKNDHAEKVAEKLEALSVKEETKEDAEEKQPTFLY

SEQ ID NO: 144 - DNA, 618 bp, synthetic construct
  source 1..618: mol_type = other DNA
atggcggccgccaaggacactcatgaggaccatgatacttccactgagaatacagacgagtccaaccatgaccctcagtttgagccaatagtttctcttcctgagcaagaaattaaaacactggaagaagatgaagaggaactttttaaaatgcgggcaaaactgttccgatttgcctctgagaacgatctcccagaatggaaggagcgaggcactggtgacgtcaagctcctgaagcacaaggagaaaggggccatccgcctcctcatgcggagggacaagaccctgaagatctgtgccaaccactacatcacgccgatgatggagctgaagcccaacgcaggtagcgaccgtgcctgggtctggaacacccacgctgacttcgccgacgagtgccccaagccagagctgctggccatccgcttcctgaatgctgagaatgcacagaaattcaaaacaaagtttgaagaatgcaggaaagagatcgaagagagagaaaagaaagcaggatcaggcaaaaatgatcatgccgaaaaagtggcggaaaagctagaagctctctcggtgaaggaggagaccaaggaggatgctgaggagaagcaaccaactttcttgtac

SEQ ID NO: 145 - protein, 326 aa, synthetic construct
MSSFSESALEKKLSELSNSQHSVQTLSLWLIHHRKHAGPIVSVWHRELRKAKSNRKLTFLYLANDVIQNSKRKGPEFTREFESVLVDAFSHVAREADEGCKKPLERLLNIWQERSVYGGEFIQQLKLSMEDSKSPPPKATEEKKSLKRTFQQIQEEEDDDYPGSYSPQDPSAGPLLTEELIKALQDLENAASGDATVRQKIASLPQEVQDVSLLEKITDKEAAERLSKTVDEACLLLAEYNGRLAAELEDRRQLARMLVEYTQNQKDVLSEKEKKLEEYKQKLARVTQVRKELKSHIQSLPDLSLLPNVTGGLAPLPSAGDLFSTD

SEQ ID NO: 146 - DNA, 981 bp, synthetic construct
  source 1..981: mol_type = other DNA
atgtcctccttctctgagtcggcgctggagaagaagctctcggagctgagcaactctcagcacagcgtgcagaccctgtccctttggctcatccaccaccgcaagcacgcgggacccatcgtctccgtgtggcaccgcgagctccgcaaagccaaatcaaatagaaagcttacttttctgtatttagcgaatgatgtcatccaaaacagtaaaaggaaaggacctgaattcactagagaatttgaatctgtccttgtggatgctttttctcatgttgccagagaggcagatgaaggctgtaaaaaacctttagaaagattgctgaacatctggcaagaacgaagtgtgtatggcggcgagttcatacagcagctgaagctgtctatggaggactccaagagccctccccccaaagcaacagaagagaagaaatctctgaaacgaacttttcagcaaattcaggaggaggaggatgacgactaccctggcagctactctcctcaggatccttctgcaggacccctcttgactgaggaactaatcaaagctttgcaggatctggaaaatgccgcatcaggggatgctactgtccgacagaaaattgcttctctgccccaggaagtgcaagatgtttctctattggaaaaaataacagacaaagaggcagctgaacgtctttcaaaaacagtagatgaagcatgtctgttactagcagaatataacgggcgcctggcagcagaactggaggaccgtcgccagctggctcggatgttggtggagtatacccagaatcagaaagatgttttgtcggagaaggagaaaaaactagaggaatacaaacagaagcttgcacgagtaacccaggtccgcaaggaactgaaatcccatattcagagcttgccagacctctcactgctgcccaacgtcacagggggcttagcccccctgccctctgctggggacctgttttcaactgactag

SEQ ID NO: 147 - protein, 262 aa, synthetic construct
MKTPFGKTPGQRSRADAGHAGVSANMMKKRTSHKKHRSSVGPSKPVSQPRRNIVGCRIQHGWKEGNGPVTQWKGTVLDQVPVNPSLYLIKYDGFDCVYGLELNKDERVSALEVLPDRVATSRISDAHLADTMIGKAVEHMFETEDGSKDEWRGMVLARAPVMNTWFYITYEKDPVLYMYQLLDDYKEGDLRIMPDSNDSPPAEREPGEVVDSLVGKQVEYAKEDGSKRTGMVIHQVEAKPSVYFIKFDDDFHIYVYDLVKTS

SEQ ID NO: 148 - DNA, 789 bp, synthetic construct
  source 1..789: mol_type = other DNA
atgaagaccccattcggaaagacacctggccagcggtccagagctgatgcaggccatgctggagtatctgccaacatgatgaagaagaggacatcccacaaaaaacatcggagcagtgtgggtccgagcaaacctgtttcccagccccggcggaacatcgtaggctgcaggattcagcatgggtggaaagaggggaatggccctgttacccagtggaaaggaaccgttctggaccaggtgcctgtaaatccttctttgtatcttataaaatacgatggatttgactgtgtttatggactagaacttaataaagatgaaagagtttctgcgcttgaagtcctccctgatagagttgcgacatctcgaatcagcgatgcacacttggcagacacaatgattggcaaagcagtggaacatatgtttgagacagaggatggttctaaagatgagtggaggggaatggtcttagcacgtgcacctgtcatgaacacatggttttacattacctatgagaaagaccctgtcttgtacatgtaccaactcttagatgattacaaagaaggcgaccttcgcattatgcctgattccaatgattcacctccagcagaaagggaaccaggagaagttgtggacagcctggtaggcaaacaagtggaatatgccaaagaagatggctcgaaaaggactggcatggtcattcatcaagtagaagccaagccctccgtctatttcatcaagtttgatgatgatttccatatttatgtctacgatttggtgaaaacatcctag

SEQ ID NO: 149 - protein, 397 aa, synthetic construct
MSVAFASARPRGKGEVTQQTIQKMLDENHHLIQCILEYQSKGKTAECTQYQQILHRNLVYLATIADSNQNMQSLLPAPPTQNMNLGPGALTQSGSSQGLHSQGSLSDAISTGLPPSSLLQGQIGNGPSHVSMQQTAPNTLPTTSMSISGPGYSHAGPASQGVPMQGQGTIGNYVSRTNINMQSNPVSMIQQQAATSHYSSAQGGSQHYQGQSSIAMMGQGSQGSSMMGQRPMAPYRPSQQGSSQQYLGQEEYYGEQYSHSQGAAEPMGQQYYPDGHGDYAYQQSSYTEQSYDRSFEESTQHYYEGGNSQYSQQQAGYQQGAAQQQTYSQQQYPSQQSYPGQQQGYGSAQGAPSQYPGYQQGQGQQYGSYRAPQTAPSAQQQRPYGYEQGQYGNYQQL

SEQ ID NO: 150 - DNA, 1191 bp, synthetic construct
  source 1..1191: mol_type = other DNA
atgtccgtggccttcgcgtctgcccggccaagaggcaaaggggaggttacgcagcaaaccatccagaagatgctggacgagaaccaccacctgatccagtgcatcctggagtaccagagcaagggcaagacggccgagtgcacgcagtaccagcagatcctgcaccggaacctggtatacctggccacgatcgcagactccaaccagaacatgcagtccctgcttcctgccccgcccacgcagaacatgaacctgggccctggagccctgactcagagcggctccagccagggcctgcactctcagggcagcctgagtgacgccatcagcacgggcctgccaccctcctccctcctgcagggccagattggcaacgggccgagccacgtgtccatgcagcagacggcgcctaacacgctgcccaccacctccatgagcatctctgggcccggctacagccacgcgggacccgcctcgcagggcgtccccatgcaggggcaaggcaccatcggcaactacgtgtctcggaccaacatcaacatgcagtccaacccagtctccatgatacagcagcaggcggccacgtcgcactacagctcggcgcagggcggcagccagcactaccagggccagtcgtccatcgccatgatggggcagggcagccaggggagcagcatgatggggcagcggcccatggcgccctaccggccctcccagcaaggctcttcccagcagtacctgggccaggaggagtactatggcgagcagtacagccacagccagggcgccgcggagcccatgggccagcagtactaccccgacggccatggcgattacgcctaccagcagtcatcctacacggagcagagctacgaccggtccttcgaggagtccacgcagcactactatgaggggggaaactcccagtacagccagcagcaggccgggtaccagcagggtgccgcgcagcagcagacgtactcccagcagcagtaccccagccagcagagctaccccgggcagcagcagggctacgggtctgcccagggagccccgtcacagtaccccggctaccagcaaggccaaggccagcagtacggaagctaccgagcaccgcagacagcgccgtctgcccagcagcagcggccctacggctatgaacagggccagtatggaaattaccagcagttg

SEQ ID NO: 151 - protein, 432 aa, synthetic construct
MSELKDCPLQFHDFKSVDHLKVCPRYTAVLARSEDDGIGIEELDTLQLELETLLSSASRRLRVLEAETQILTDWQDKKGDRRFLKLGRDHELGAPPKHGKPKKQKLEGKAGHGPGPGPGRPKSKNLQPKIQEYEFTDDPIDVPRIPKNDAPNRFWASVEPYCADITSEEVRTLEELLKPPEDEAEHYKIPPLGKHYSQRWAQEDLLEEQKDGARAAAVADKKKGLMGPLTELDTKDVDALLKKSEAQHEQPEDGCPFGALTQRLLQALVEENIISPMEDSPIPDMSGKESGADGASTSPRNQNKPFSVPHTKSLESRIKEELIAQGLLESEDRPAEDSEDEVLAELRKRQAELKALSAHNRTKKHDLLRLAKEEVSRQELRQRVRMADNEVMDAFRKIMAARQKKRTPTKKEKDQAWKTLKERESILKLLDG

SEQ ID NO: 152 - DNA, 1299 bp, synthetic construct
  source 1..1299: mol_type = other DNA
atgagtgagttgaaagactgccccttgcagttccacgacttcaagtctgtggatcacctgaaggtctgtccccgctacacggcagtgctggcacgctctgaggatgatggcatcggcatcgaggagctggacaccctgcagctggagctggagaccctgctgtcttctgccagccggcgcctgcgtgtgcttgaggccgaaacccagatcctcaccgactggcaggataagaaaggtgacagacgattcctgaagctgggtcgagaccatgaacttggagctccccccaaacatgggaagcccaagaagcagaaactggaagggaaggcaggacatgggccgggccctggcccaggacggcccaaatccaaaaaccttcagcccaagatccaggaatatgaattcactgatgaccctatcgacgtgccacggatccccaaaaatgatgcccccaacaggttctgggcttcagtggagccctactgtgctgacatcaccagcgaggaggtccgcacacttgaggagttactgaagcccccagaagatgaggctgagcattacaagatcccacccctggggaagcactactcccagcgctgggcccaggaggacctgctggaggagcagaaggatggggcccgggcagcggctgtggctgacaagaagaaaggcctcatggggccactgaccgaactggacactaaagatgtggatgccctgctgaagaagtctgaggcccagcatgaacagccggaagatggatgcccctttggtgccctgacgcagcgcctcctgcaggccctggtggaggaaaatattatttcccctatggaggattctcctattcctgacatgtctgggaaagaatcaggggctgacggggcaagcacctcccctcgcaatcagaacaagcccttcagtgtgccgcatactaagtccctggagagccgcatcaaggaggagctaattgcccagggccttttggagtctgaggaccgccccgcagaggactccgaggatgaggtccttgctgagcttcgcaaacggcaggctgagctgaaggcacttagtgcccacaaccgcaccaagaagcacgacctgctgaggctggcaaaggaggaggtgagccggcaggagctgaggcagcgggtgcgcatggctgacaacgaggtcatggacgcctttcgcaagatcatggctgcccggcagaagaagcggactcccaccaagaaagaaaaggaccaggcctggaagactctgaaggagcgtgagagcatcctgaagctgctggatgggtag

SEQ ID NO: 153 - protein, 678 aa, synthetic construct
MAEEKKLKLSNTVLPSESMKVVAESMGIAQIQEETCQLLTDEVSYRIKEIAQDALKFMHMGKRQKLTTSDIDYALKLKNVEPLYGFHAQEFIPFRFASGGGRELYFYEEKEVDLSDIINTPLPRVPLDVCLKAHWLSIEGCQPAIPENPPPAPKEQQKAEATEPLKSAKPGQEEDGPLKGKGQGATTADGKGKEKKAPPLLEGAPLRLKPRSIHELSVEQQLYYKEITEACVGSCEAKRAEALQSIATDPGLYQMLPRFSTFISEGVRVNVVQNNLALLIYLMRMVKALMDNPTLYLEKYVHELIPAVMTCIVSRQLCLRPDVDNHWALRDFAARLVAQICKHFSTTTNNIQSRITKTFTKSWVDEKTPWTTRYGSIAGLAELGHDVIKTLILPRLQQEGERIRSVLDGPVLSNIDRIGADHVQSLLLKHCAPVLAKLRPPPDNQDAYRAEFGSLGPLLCSQVVKARAQAALQAQQVNRTTLTITQPRPTLTLSQAPQPGPRTPGLLKVPGSIALPVQTLVSARAAAPPQPSPPPTKFIVMSSSSSAPSTQQVLSLSTSAPGSGSTTTSPVTTTVPSVQPIVKLVSTATTAPPSTAPSGPGSVQKYIVVSLPPTGEGKGGPTSHPSPVPPPASSPSPLSGSALCGGKQEAGDSPPPAPGTPKANGSQPNSGSPQPAPL

SEQ ID NO: 154 - DNA, 2034 bp, synthetic construct
  source 1..2034: mol_type = other DNA
atggctgaggagaagaagctgaagcttagcaacactgtgctgccctcggagtccatgaaggtggtggctgaatccatgggcatcgcccagattcaggaggagacctgccagctgctaacggatgaggtcagctaccgcatcaaagagatcgcacaggatgccttgaagttcatgcacatggggaagcggcagaagctcaccaccagtgacattgactacgccttgaagctaaagaatgtcgagccactctatggcttccacgcccaggagttcattcctttccgcttcgcctctggtgggggccgggagctttacttctatgaggagaaggaggttgatctgagcgacatcatcaatacccctctgccccgggtgcccctggacgtctgcctcaaagctcattggctgagcatcgagggctgccagccagctatccccgagaacccgcccccagctcccaaagagcaacagaaggctgaagccacagaacccctgaagtcagccaagccaggccaggaggaagacggacccctgaagggcaaaggtcaaggggccaccacagccgacggcaaagggaaagagaagaaggcgccgcccttgctggagggggcccccttgcgactgaagccccggagcatccacgagttgtctgtggagcagcagctctactacaaggagatcaccgaggcctgcgtgggctcctgcgaggccaagagggcggaagccctgcaaagcattgccacggaccctggactgtatcagatgctgccacggttcagtacctttatctcggagggggtccgtgtgaacgtggttcagaacaacctggccctactcatctacctgatgcgtatggtgaaagcgctgatggacaaccccacgctctatctagaaaaatacgtccatgagctgattccagctgtgatgacctgcatcgtgagcagacagttgtgcctgcgaccagatgtggacaatcactgggcactccgagactttgctgcccgcctggtggcccagatctgcaagcattttagcacaaccactaacaacatccagtcccggatcaccaagaccttcaccaagagctgggtggacgagaagacgccctggacgactcgttatggctccatcgcaggcttggctgagctgggacacgatgttatcaagactctgattctgccccggctgcagcaggaaggggagcggatccgcagtgtgctggacggccctgtgctgagcaacattgaccggattggagcagaccatgtgcagagcctcctgctgaaacactgtgctcctgttctggcaaagctgcgcccaccgcctgacaatcaggacgcctatcgggcagaattcgggtcccttgggcccctcctctgctcccaggtggtcaaggctcgggcccaggctgctctgcaggctcagcaggtcaacaggaccactctgaccatcacgcagccccggcccacgctgaccctctcgcaggccccacagcctggccctcgcacccctggcttgctgaaggttcctggctccatcgcacttcctgtccagacactggtgtctgcacgagcggctgccccaccacagccttcccctcctccaaccaagtttattgtaatgtcatcgtcctccagcgccccatccacccagcaggtcctgtccctcagcacctcggcccccggctcaggttccaccaccacttcgcccgtcaccaccaccgtccccagcgtgcagcccatcgtcaagttggtctccaccgccaccaccgcaccccccagcactgctccctctggtcctgggagtgtccagaagtacatcgtggtctcacttcccccaacaggggagggcaaaggaggccccacctcccatccttctccagttcctcccccggcatcgtccccgtccccactcagcggcagtgccctttgtggggggaagcaggaggctggggacagtccccctccagctccagggactccaaaagccaatggctcccagcccaactccggctcccctcagcctgctccgttg

SEQ ID NO: 155 - protein, 187 aa, synthetic construct
MDADSDVALDILITNVVCVFRTRCHLNLRKIALEGANVIYKRDAGKVLMKLRKPRITATIWSSGKIICTGATSEEEAKFGARRLARSLQKLGFQVIFTDFKVVNVLAVCNMPFEIRLPEFTKNNRPHASYEPELHPAVCYRIKSLRATLQIFSTGSITVTGPNVKAVATAVEQIYPFVFESRKEILL

SEQ ID NO: 156 - DNA, 561 bp, synthetic construct
  source 1..561: mol_type = other DNA
atggatgcagacagtgatgttgcattggacattctaattacaaatgtagtctgtgtttttagaacaagatgtcatttaaacttaaggaagattgctttggaaggagcaaatgtaatttataaacgtgatgctggaaaagtattaatgaagcttagaaaacctagaattacagctacaatttggtcctcaggaaaaattatttgcactggagcaacaagtgaagaagaagctaaatttggtgccagacgcttagcccgtagtctgcagaaactaggttttcaggtaatatttacagattttaaggttgttaacgttctggcagtgtgtaacatgccatttgaaatccgtttgccagaattcacaaagaacaatagacctcatgccagttacgaacctgaacttcatcctgctgtgtgctatcggataaaatctctaagagctacattacagattttttcaacaggaagtatcacagtaacagggcccaatgtaaaggctgttgctactgctgtggaacagatttacccatttgtgtttgaaagcaggaaagaaattttattg

SEQ ID NO: 157 - protein, 365 aa, synthetic construct
MSLAGGRAPRKTAGNRLSGLLEAEEEDEFYQTTYGGFTEESGDDEYQGDQSDTEDEVDSDFDIDEGDEPSSDGEAEEPRRKRRVVTKAYKEPLKSLRPRKVNTPAGSSQKAREEKALLPLELQDDGSDSRKSMRQSTAEHTRQTFLRVQERQGQSRRRKGPHCERPLTQEELLREAKITEELNLRSLETYERLEADKKKQVHKKRKCPGPIITYHSVTVPLVGEPGPKEENVDIEGLDPAPSVSALTPHAGTGPVNPPARCSRTFITFSDDATFEEWFPQGRPPKVPVREVCPVTHRPALYRDPVTDIPYATARAFKIIREAYKKYITAHGLPPTASALGPGPPPPEPLPGSGPRALRQKIVIKL

SEQ ID NO: 158 - DNA, 1095 bp, synthetic construct
  source 1..1095: mol_type = other DNA
atgagtttggctgggggccgggcaccccggaagaccgctgggaaccggctttctgggcttttggaggcagaggaggaagatgagttctaccagacgacttatgggggtttcacagaggaatccggagatgatgagtatcaaggggaccagtcagacacagaggacgaagtggactctgactttgacattgatgaaggggatgaaccatccagtgatggagaagcagaagagccaagaaggaagcgccgagtagtcaccaaggcctataaggaacctctcaagagcttaaggcctcgaaaggtcaacaccccggctggtagctctcagaaggcgcgagaagagaaggcactactgccattagaactacaagatgacggctctgacagtcggaagtctatgcgtcagtctacagctgagcatacacgacaaacgttccttcgggtacaggagaggcagggccagtcaagacggcgaaaggggccccactgtgagcggccactaacccaggaggaactgctccgggaggccaagatcacagaagagcttaatttacggtcactggagacatatgagcggctcgaggctgataaaaagaagcaggttcataagaagcggaagtgccccgggcccataatcacctatcattcagtgacagtgccacttgttggggagccaggccccaaggaagagaacgttgacatagaaggacttgatcctgctccctcggtgtctgcattgactcctcatgctgggactggacccgtcaacccccctgctcgctgctcacgtaccttcatcacttttagtgatgatgcaactttcgaggaatggttcccccaagggcggcccccaaaagtccctgttcgtgaggtctgtccagtgacccatcgtccagccctataccgggaccctgttacagacataccctatgccactgctcgagccttcaagatcattcgtgaggcttacaagaagtacattactgcccatggactgccgcccactgcctcagccctgggccccggcccgccacctcctgagcccctccctggctctgggccccgagccttgcgccagaaaattgtcattaaattg

SEQ ID NO: 159 - protein, 653 aa, synthetic construct
MAFRDVAVDFTQDEWRLLSPAQRTLYREVMLENYSNLVSLGISFSKPELITQLEQGKETWREEKKCSPATCPDPEPELYLDPFCPPGFSSQKFPMQHVLCNHPPWIFTCLCAEGNIQPGDPGPGDQEKQQQASEGRPWSDQAEGPEGEGAMPLFGRTKKRTLGAFSRPPQRQPVSSRNGLRGVELEASPAQTGNPEETDKLLKRIEVLGFGTVNCGECGLSFSKMTNLLSHQRIHSGEKPYVCGVCEKGFSLKKSLARHQKAHSGEKPIVCRECGRGFNRKSTLIIHERTHSGEKPYMCSECGRGFSQKSNLIIHQRTHSGEKPYVCRECGKGFSQKSAVVRHQRTHLEEKTIVCSDCGLGFSDRSNLISHQRTHSGEKPYACKECGRCFRQRTTLVNHQRTHSKEKPYVCGVCGHSFSQNSTLISHRRTHTGEKPYVCGVCGRGFSLKSHLNRHQNIHSGEKPIVCKDCGRGFSQQSNLIRHQRTHSGEKPMVCGECGRGFSQKSNLVAHQRTHSGERPYVCRECGRGFSHQAGLIRHKRKHSREKPYMCRQCGLGFGNKSALITHKRAHSEEKPCVCRECGQGFLQKSHLTLHQMTHTGEKPYVCKTCGRGFSLKSHLSRHRKTTSVHHRLPVQPDPEPCAGQPSDSLYSL

SEQ ID NO: 160 - DNA, 1962 bp, synthetic construct
  source 1..1962: mol_type = other DNA
atggcattcagggatgtggctgtggatttcacccaggatgagtggaggctgctgagccctgctcaaaggactctgtacagagaggtgatgctggagaactacagcaacctggtctcactgggaatttcattttctaaaccagaactcatcacccagctggagcaagggaaagagacctggagagaggaaaaaaaatgttcaccggcaacctgtccagatccagagccagagctctacctcgatcctttctgccctccgggtttctccagtcagaaattccccatgcagcatgtgctgtgtaatcatcccccctggatcttcacatgcttgtgtgcagaaggtaacatccagcctggggatccaggcccaggggaccaggagaagcagcaacaagcctctgaggggagaccctggagtgatcaagcagaaggtcctgagggagaaggtgccatgcctttgtttggaagaaccaagaaaaggactctgggagcgttctccaggccaccccagaggcagccagtcagctctcggaacggcctcagaggggtggagttagaagccagcccagctcagacagggaaccctgaggaaacagacaaattgttgaagaggatagaagtcttaggatttggaacagtcaactgtggagagtgtggactgagcttcagcaagatgacaaacctgctcagtcaccagcggatacactcaggggagaagccctacgtgtgtggggtatgtgagaagggcttcagcctaaagaagagcctcgccagacaccagaaggcacactcgggggagaagccaattgtgtgcagggagtgtggacgaggctttaaccggaagtcaacgctaatcatacacgaacggacacactccggtgagaaaccttacatgtgcagtgagtgtgggcgaggcttcagccagaagtcaaacctcatcatacaccagaggacacactcaggggaaaagccttatgtgtgccgggaatgtggcaaaggcttcagccagaagtcagctgtcgtgagacaccagaggacacacttggaggagaagaccatcgtgtgcagtgactgtggcctgggcttcagcgacaggtcaaacctcatctcccaccagaggacgcactctggggagaagccctacgcctgcaaggagtgtgggcgatgcttcaggcagaggaccacccttgtcaaccaccagaggacacactcaaaggagaagccctatgtgtgcggggtgtgtgggcacagcttcagccagaattcaaccctcatctctcacaggcggacacacactggggagaagccgtatgtttgtggggtgtgtgggcgaggctttagtctcaagtcacacctcaacagacaccagaacatacactcaggagagaagcccattgtgtgcaaggactgtggccggggcttcagccagcaatccaacctcatcagacaccagaggacgcactcaggcgagaagcccatggtgtgtggggagtgcgggcgaggcttcagccagaagtcaaaccttgttgcacaccagaggacgcactcaggggagaggccgtatgtgtgccgagagtgcgggcgaggctttagccaccaggccggtctcatcaggcacaagcggaagcactcgagggagaagccctacatgtgcaggcagtgtggactgggctttggcaataagtcagctctaatcacacacaagcgggctcactcggaagagaagccttgtgtgtgcagagagtgtggccaaggctttctccaaaagtcacacctcaccttacatcaaatgacacatacgggggagaagccatatgtgtgcaagacgtgtgggcggggcttcagcctcaagtctcacctcagcagacacaggaagaccacgtctgtccaccacagactgccagtgcagcccgaccctgagccgtgtgcagggcaaccttcggattccttatactctctctga

SEQ ID NO: 161 - protein, 457 aa, synthetic construct
MSQGSVTFRDVAIDFSQEEWKWLQPAQRDLYRCVMLENYGHLVSLGLSISKPDVVSLLEQGKEPWLGKREVKRDLFSVSESSGEIKDFSPKNVIYDDSSQYLIMERILSQGPVYSSFKGGWKCKDHTEMLQENQGCIRKVTVSHQEALAQHMNISTVERPYGCHECGKTFGRRFSLVLHQRTHTGEKPYACKECGKTFSQISNLVKHQMIHTGKKPHECKDCNKTFSYLSFLIEHQRTHTGEKPYECTECGKAFSRASNLTRHQRIHIGKKQYICRKCGKAFSSGSELIRHQITHTGEKPYECIECGKAFRRFSHLTRHQSIHTTKTPYECNECRKAFRCHSFLIKHQRIHAGEKLYECDECGKVFTWHASLIQHTKSHTGEKPYACAECDKAFSRSFSLILHQRTHTGEKPYVCKVCNKSFSWSSNLAKHQRTHTLDNPYEYENSFNYHSFLTEHQ

SEQ ID NO: 162 - DNA, 1374 bp, synthetic construct
  source 1..1374: mol_type = other DNA
atgtctcaggggtcagtgacattcagagatgtggccatagacttctcccaggaggagtggaaatggcttcagcctgctcaaagagatttgtacagatgtgtaatgttggagaactatggccatctggtctcactgggtctttccatttctaagccagatgtggtttccttattggagcaagggaaagaaccctggctggggaaaagggaagtgaaaagagatctgttttcagtttcagagtcaagtggtgagatcaaagacttttcaccaaaaaatgtcatttatgatgactcatcccagtatttgatcatggaaagaattctaagtcaaggccctgtgtattccagttttaaaggaggctggaaatgcaaggatcatactgagatgctgcaagaaaatcagggatgtattaggaaagtaacagtctctcatcaagaagccctggctcaacatatgaatatcagtactgtggagaggccctatggatgccatgaatgtggaaaaacttttggtcgacgcttttccctggtgttacaccagaggactcatactggagagaaaccatatgcatgtaaggaatgtggcaaaacctttagccagatttcaaaccttgtgaaacaccaaatgatacatactggaaagaaaccccatgagtgtaaggactgtaataaaacattcagttacctttcatttcttattgaacaccagagaacgcacactggggagaaaccttatgaatgtactgagtgtggaaaggcctttagccgtgcctccaacctcactcgacatcaaagaattcacataggaaagaaacaatatatatgtaggaaatgtggtaaagcatttagcagtggctcagaactcattcgccaccagattacacatactggagagaaaccttatgaatgcattgaatgtgggaaggcatttcgccgtttctcacaccttactcgacatcagagcatccatacaaccaaaaccccgtatgaatgtaatgaatgtaggaaagctttccgttgtcactcattccttattaaacatcagagaattcatgctggagaaaagctctatgaatgtgatgaatgtggtaaagttttcacttggcatgcatcccttattcaacatacgaagagtcacactggagagaaaccctatgcgtgtgctgaatgtgataaagccttcagccggagcttttccctcattctacatcagagaactcatactggagagaaaccctatgtatgtaaggtatgcaacaaatccttcagctggagctcaaaccttgctaaacatcagaggacacacactcttgacaacccctatgaatatgaaaattcatttaattaccactcattccttactgaacaccagtga

SEQ ID NO: 163 - protein, 96 aa, synthetic construct
MSPGLLTTRKEALMAFRDVAVAFTQKEWKLLSSAQRTLYREVMLENYSHLVSLGIAFSKPKLIEQLEQGDEPWREENEHLLDLCPGRRITRSGVRD

SEQ ID NO: 164 - DNA, 291 bp, synthetic construct
  source 1..291: mol_type = other DNA
atgtcaccaggactcctgacaaccaggaaggaggcattgatggccttccgggatgtggctgtggccttcacccagaaggagtggaagctattgagttctgctcagaggaccctgtacagggaggtgatgctggagaactacagccatctggtctccctgggaattgcattttccaaaccaaaactcatcgaacagctggagcaaggcgacgaaccttggagagaggagaacgaacatcttctggacctttgtccaggcaggcggatcacaaggtcgggagttcgagactag

SEQ ID NO: 165 - protein, 91 aa, synthetic construct
MPGPPRSLEMGLLTFRDVAIEFSLEEWQHLDIAQQNLYRNVMLENYRNLAFLGIAVSKPDLITCLEQGKEPWNMKRHEMVDEPPGLDFSLL

SEQ ID NO: 166 - DNA, 276 bp, synthetic construct
  source 1..276: mol_type = other DNA
atgccaggaccccctagaagcctagaaatgggactgttgacatttagggatgtggccatagaattctctctggaggagtggcaacacctggacattgcacagcagaatttatatagaaatgtgatgttagagaactacagaaacctggccttcctgggtattgctgtctctaagccagacctgatcacctgtctggaacaagggaaagagccctggaatatgaagcgacatgagatggtggatgaacccccaggattggatttttcattactgtga

SEQ ID NO: 167 - protein, 419 aa, synthetic construct
MAQESVMFSDVSVDFSQEEWECLNDDQRDLYRDVMLENYSNLVSMGHSISKPNVISYLEQGKEPWLADRELTRGQWPVLESRCETKKLFLKKEIYEIESTQWEIMEKLTRRDFQCSSFRDDWECNRQFKKELGSQGGHFNQLVFTHEDLPTLSHHPSFTLQQIINSKKKFCASKEYRKTFRHGSQFATHEIIHTIEKPYECKECGKSFRHPSRLTHHQKIHTGKKPFECKECGKTFICGSDLTRHHRIHTGEKPYECKECGKAFSSGSNFTRHQRIHTGEKPYECKECGKAFSSGSNFTQHQRIHTGEKPYECKECGNAFSQSSQLIKHQRIHTGEKPYECKECEKAFRSGSDLTRHQRIHTGEKPYECKICGKAYSQSSQLISHHRIHTSEKPYEYRECGKNFNYDPQLIQHQNLYWL

SEQ ID NO: 168 - DNA, 1257 bp, synthetic construct
  source 1..1257: mol_type = other DNA
atggctcaggagtcagtgatgttcagtgatgtgtccgtagacttctctcaggaggagtgggaatgcctgaatgatgatcagagagatttatacagagatgtgatgttggagaattacagcaacctggtttcaatggggcattctatttctaaaccaaatgtgatctcctacttggagcaagggaaggagccctggttggctgacagagagctaacaagaggccagtggccagtcctggaatcaagatgtgagaccaagaaattatttctgaagaaagaaatttatgaaatagaatcaacccagtgggaaataatggaaaaactcacaagacgtgattttcagtgctccagtttcagagatgattgggaatgtaatcggcagtttaagaaagaactcggctctcaggggggacatttcaatcaattggtattcactcatgaagatctgcccactttgagtcaccatccatccttcacattacagcaaatcattaacagtaaaaagaaattctgtgcatctaaagaatataggaaaacctttagacatggctcacagtttgctacacatgagataattcataccattgagaagccttatgaatgtaaggaatgtggaaagtcctttagacatccctcaagactcactcatcatcagaaaattcatactggcaagaaaccctttgaatgtaaggaatgtggaaaaacctttatttgtggctcagaccttactcgacatcacagaattcacactggtgagaaaccctatgaatgtaaggaatgtgggaaagcctttagtagtggttcaaacttcactcgacatcagagaattcacacaggtgagaagccttatgaatgcaaagaatgcgggaaggcctttagtagtggctcaaactttactcaacatcagagaattcatactggggaaaaaccctatgaatgtaaggaatgtggcaatgcctttagtcagagctcacaacttattaaacatcaaagaatccatacaggtgagaaaccttacgaatgtaaggaatgtgaaaaggcttttcgttctggctcagaccttactagacatcagagaattcatactggggagaaaccctatgaatgtaagatatgtgggaaggcttattctcagagttcacagcttattagtcatcatagaattcatactagtgagaaaccctatgaatatagggaatgtggaaagaactttaattatgacccacaacttattcagcatcaaaatttgtactggttg

SEQ ID NO: 169 - protein, 113 aa, synthetic construct
MPANWTSPQKSSALAPEDHGSSYEGSVSFRDVAIDFSREEWRHLDPSQRNLYRDVMLETYSHLLSIGYQVPEAEVVMLEQGKEPWALQGERPRQSCPAPCLVNSHHLQESFRG

SEQ ID NO: 170 - DNA, 342 bp, synthetic construct
  source 1..342: mol_type = other DNA
atgccagctaattggacctcacctcagaaatcctcagccctggctccagaggatcatggcagctcctatgagggatcagtgtccttcagggatgtggctatcgatttcagcagagaggaatggcggcacctggacccttctcagagaaacctgtaccgggatgtgatgctggagacctacagccacctgctctcaataggatatcaagttcctgaagcagaggtggtcatgttggagcaaggaaaggaaccatgggcactgcagggtgagaggccacgtcagagctgcccagcaccgtgtcttgtgaactcccatcaccttcaagaaagcttccgagggtga

SEQ ID NO: 171 - protein, 500 aa, synthetic construct
MAPPSAPLPAQGPGKARPSRKRGRRPRALKFVDVAVYFSPEEWGCLRPAQRALYRDVMRETYGHLGALGCAGPKPALISWLERNTDDWEPAALDPQEYPRGLTVQRKSRTRKKNGEKEVFPPKEAPRKGKRGRRPSKPRLIPRQTSGGPICPDCGCTFPDHQALESHKCAQNLKKPYPCPDCGRRFSYPSLLVSHRRAHSGECPYVCDQCGKRFSQRKNLSQHQVIHTGEKPYHCPDCGRCFRRSRSLANHRTTHTGEKPHQCPSCGRRFAYPSLLAIHQRTHTGEKPYTCLECNRRFRQRTALVIHQRIHTGEKPYPCPDCERRFSSSSRLVSHRRVHSGERPYACEHCEARFSQRSTLLQHQLLHTGEKPYPCPDCGRAFRRSGSLAIHRSTHTEEKLHACDDCGRRFAYPSLLASHRRVHSGERPYACDLCSKRFAQWSHLAQHQLLHTGEKPFPCLECGRCFRQRWSLAVHKCSPKAPNCSPRSAIGGSSQRGNAH

SEQ ID NO: 172 - DNA, 1503 bp, synthetic construct
  source 1..1503: mol_type = other DNA
atggcgccaccttcggctccgctccctgcgcagggaccaggaaaggccagacccagtcggaaaaggggcaggaggccgagggctctgaagttcgtggacgtggccgtgtacttctccccggaggagtggggctgcctgcggcccgcgcagagggccctgtaccgggacgtgatgcgggagacctacggtcacctgggcgcgctcgggtgcgcaggtcccaaaccagccctcatctcctggttggaacgaaacaccgatgactgggaaccggctgctctagatccgcaggagtacccgagagggctaacagtccagagaaaaagcagaaccagaaagaagaatggggagaaggaagtattcccgcctaaggaggcaccccgaaaggggaagcgaggccggaggcccagcaaaccccgactgattcctaggcagacgtccgggggccccatctgccctgactgcggctgtaccttccctgatcatcaggccctggagagccacaagtgcgcccagaatctaaaaaagccttacccttgcccagactgtgggcgccgcttttcctatccatccctgctggtcagtcaccggcgggcacactccggcgagtgcccctatgtttgtgaccagtgtggcaaacgtttctcccagcgcaagaacctctcccagcaccaggtcatccatacaggggagaagccctatcactgccctgactgtggtcgctgcttccggaggagccggtccttggccaatcaccggaccacacacacaggtgaaaaaccccaccagtgccctagctgtggacgtcgcttcgcctacccctccctgctagccatccaccagcgtacacacacgggagagaagccctacacttgcctcgagtgcaaccgccgcttccgccagcgcacggccctcgtcatccaccagcgcatccacacgggcgagaagccctacccgtgcccggactgcgagcggcgcttctcctcctcctctcgcctggtcagtcaccggcgtgtgcactctggggagcgtccctatgcctgcgagcactgtgaggcccgcttctcccagcgcagcacgctgctccagcaccagctcttgcacaccggagagaagccctacccctgcccagactgtgggcgtgccttccggcggagcggctccctggccatccatcgcagcacgcacacagaggagaagctgcacgcctgcgacgactgtggtcgccgctttgcctacccctcactgctggccagccaccggcgcgtgcactcgggcgagcggccctatgcctgcgacctttgctccaagcgttttgctcagtggagccacctggcccagcaccagctgctgcacacgggggagaagcctttcccctgcctcgagtgtggccggtgcttccgccagaggtggtctctggctgtccacaagtgtagccccaaggccccaaactgtagccctagatctgctatcgggggctccagtcagaggggcaacgcccattag

SEQ ID NO: 173 - protein, 67 aa, synthetic construct
MALPQGLLTFRDVAIEFSQEEWKCLDPAQRTLYRDVMLENYRNLVSLELSGECPLAAPASLDPAFLC

SEQ ID NO: 174 - DNA, 201 bp, synthetic construct
  source 1..201: mol_type = other DNA
atggctcttcctcagggtctattgacattcagggatgtggccatagaattctctcaggaggagtggaaatgcctggaccctgctcagaggactctatacagggacgtgatgctggagaattataggaacctggtctccctggagttgtcaggggagtgtccattggcagcacctgcctccttggacccagctttcttgtgc

SEQ ID NO: 175 - protein, 126 aa, synthetic construct
MPANEDAPQPGEHGSACEVSVSFEDVTVDFSREEWQQLDSTQRRLYQDVMLENYSHLLSVGFEVPKPEVIFKLEQGEGPWTLEGEAPHQSCSDGKFGIKPSQRRISGKSTFHSEMEGEDTLCSGLM

SEQ ID NO: 176 - DNA, 380 bp, synthetic construct
  source 1..380: mol_type = other DNA
atgccagctaacgaggacgctccccagccaggggaacatggcagtgcctgtgaggtatcagtgtcatttgaggatgtgactgtggacttcagtagagaggagtggcagcaactggactctactcaaagacgcctgtaccaggatgtaatgttggagaactacagccacctgctctcagtggggttcgaagttcctaaaccagaggtcatcttcaagttggagcaaggagaggggccatggacattggaaggggaagccccacatcagagctgttcagatgggaaatttggaattaagccttcccagaggagaatttctgggaaatctacatttcatagtgaaatggagggtgaagacacactgtgttcaggcctcatggg